(19) United States
(12) Patent Application Publication
(10) Pub. No.: US 2022/0127754 A1
(43) Pub. Date: Apr. 28, 2022

(54) METHODS AND COMPOSITIONS OF ACCELERATING REACTIONS FOR POLYPEPTIDE ANALYSIS AND RELATED USES

(71) Applicant: Encodia, Inc., San Diego, CA (US)

(72) Inventors: Stephen Verespy, III, San Diego, CA (US); Mark S. Chee, San Diego, CA (US)

(73) Assignee: Encodia, Inc., San Diego, CA (US)

(21) Appl. No.: 17/423,094

(22) PCT Filed: Jan. 17, 2020
(86) PCT No.: PCT/US2020/014199

§ 371 Date: Jul. 14, 2021

Related U.S. Application Data
(60) Provisional application No. 62/794,807, filed on Jan. 21, 2019.
(60) Provisional application No. 62/896,872, filed on Sep. 6, 2019.

(51) Int. Cl. C40B 20/04; G01N 33/68; C40B 70/00

(52) U.S. Cl. CPC C40B 20/04; C40B 70/00; G01N 33/6818

(57) ABSTRACT

The present disclosure relates to methods of accelerating reactions involving macromolecules, e.g., peptides, polypeptides, and proteins for sequencing and/or analysis. In some embodiments, the methods include the application of radiation, e.g., electromagnetic radiation or microwave energy. In some embodiments, the methods and uses are for modifying a polypeptide or a plurality of polypeptides (e.g., peptides and proteins) for sequencing and/or analysis that employ barcoding and nucleic acid encoding of molecular recognition events, and/or detectable labels.

### RELATED APPLICATIONS

The present application claims priority to U.S. provisional patent application Nos. 62/794,807, filed on Jan. 21, 2019, and 62/896,872, filed on Sep. 6, 2019, the disclosures and contents of which are incorporated by reference in their entireties for all purposes.

### SUBMISSION OF SEQUENCE LISTING ON ASCII TEXT FILE

This patent or application file contains a Sequence Listing submitted in computer readable ASCII text format (file name: 4614-2001140_SeqList_20200115_ST25.txt, date recorded: Jan. 15, 2020, size: 5,530 bytes). The content of the Sequence Listing file is incorporated herein by reference in its entirety.

### TECHNICAL FIELD

The present disclosure relates to methods and compositions for accelerating reactions involving macromolecules, e.g., peptides, polypeptides, and proteins. In some embodiments, the methods include the application of radiation, e.g., electromagnetic radiation or microwave energy. In some embodiments, the provided methods are for use with polypeptide sequencing and/or polypeptide analysis. In some embodiments, the methods and uses are for modifying a polypeptide or a plurality of polypeptides (e.g., peptides and proteins) for sequencing and/or analysis that employ barcoding and nucleic acid encoding of molecular recognition events, and/or detectable labels. Compositions, e.g., kits or systems, for treating, analyzing and/or sequencing a polypeptide are also provided.

### BACKGROUND

Proteins play an integral role in cell biology and physiology, performing and facilitating many different biological functions. The repertoire of different protein molecules is extensive, much more complex than the transcriptome, due to additional diversity introduced by post-translational modifications (PTMs). Additionally, proteins within a cell dynamically change (in expression level and modification state) in response to the environment, physiological state, and disease state. Thus, proteins contain a vast amount of relevant information that is largely unexplored, especially relative to genomic information. In general, innovation has been lagging in proteomics analysis relative to genomics analysis. In the field of genomics, next-generation sequencing (NGS) has transformed the field by enabling analysis of billions of DNA sequences in a single instrument run, whereas in protein analysis and peptide sequencing, throughput is still limited.

Yet this protein information is direly needed for a better understanding of proteome dynamics in health and disease and to help enable precision medicine. As such, there is great interest in developing “next-generation” tools to miniaturize and highly-parallelize collection of this proteomic information.

Highly-parallel macromolecular characterization and recognition of proteins is challenging for several reasons. The use of affinity-based assays is often difficult due to several key challenges. One significant challenge is multiplexing the readout of a collection of affinity agents to a collection of cognate macromolecules; another challenge is minimizing cross-reactivity between the affinity agents and off-target macromolecules; a third challenge is developing an efficient high-throughput read out platform. An example of this problem occurs in proteomics in which one goal is to identify and quantitate most or all the proteins in a sample. Additionally, it is desirable to characterize various post-translational modifications (PTMs) on the proteins at a single molecule level. Currently this is a formidable task to accomplish in a high-throughput way.

Accordingly, there remains a need in the art for improved techniques relating to treating, analyzing and/or sequencing a polypeptide. The present disclosure fulfills these and other related needs. Provided herein are methods and compositions of accelerating a reaction involving polypeptides, including modifying a polypeptide that meets such needs.

These and other aspects of the invention will be apparent upon reference to the following detailed description. To this end, various references are set forth herein which describe in more detail certain background information, procedures, compounds and/or compositions, and are each hereby incorporated by reference in their entirety.

### BRIEF SUMMARY

The summary is not intended to be used to limit the scope of the claimed subject matter. Other features, details, utilities, and advantages of the claimed subject matter will be apparent from the detailed description including those aspects disclosed in the accompanying drawings and in the appended claims.

Provided herein is a method for sequencing a polypeptide including a) contacting a polypeptide with a functionalizing reagent to modify an amino acid of said polypeptide, a binding agent capable of binding to said polypeptide, and/or a removing reagent to remove an amino acid from said polypeptide; b) applying a microwave energy to said polypeptide; and c) determining the sequence of at least a portion of said polypeptide. Also provided herein is a method for treating a polypeptide including a) contacting a polypeptide with a functionalizing reagent to modify an amino acid of said polypeptide, a binding agent capable of binding to said polypeptide, and/or a removing reagent to remove an amino acid from said polypeptide; and b) applying a microwave energy to said polypeptide; wherein the functionalizing reagent modifies an N-terminal amino acid (NTAA), the binding agent binds to an N-terminal amino acid (NTAA), and/or the removing reagent removes an N-terminal amino acid (NTAA). In some embodiments, the step a) is conducted before the step b). In some embodiments, the step a) is conducted after the step b). In some embodiments, the step a) and the step b) are conducted in the same step or simultaneously.

In some of any of the provided embodiments, the polypeptide is contacted with the functionalizing reagent, the binding agent, and/or the removing reagent in the presence of the microwave energy.

In some of any of the provided embodiments, the polypeptide is contacted with the functionalizing reagent. In some aspects, the polypeptide is contacted with the functionalizing reagent to modify a single amino acid of the polypeptide. In some embodiments, the polypeptide is contacted with the functionalizing reagent to modify multiple amino acids of the polypeptide.

In some of any of the provided embodiments, the method includes, preparing a mixture comprising one or more of polypeptides and a functionalizing reagent to modify one or more amino acids of the one or more of polypeptides; subjecting the mixture to a microwave energy; and determining the sequence of at least a portion of the one or more of polypeptides. In some of any of the provided embodiments, the modified amino acid is an amino acid at a terminus of the polypeptide, e.g., an N-terminal amino acid (NTAA), or a C-terminal amino acid (CTAA). In some examples, the method includes contacting the polypeptide with a functionalizing reagent to modify an N-terminal amino acid (NTAA) of the polypeptide and applying a microwave energy. In some embodiments, the method includes preparing a mixture comprising one or more polypeptides and a functionalizing reagent to modify an N-terminal amino acid (NTAA) and subjecting the mixture to a microwave energy.

Any suitable functionalizing reagent can be used. In some embodiments, the functionalizing reagent comprises a chemical agent, an enzyme, and/or a biological agent. In some embodiments, the functionalizing reagent adds a chemical moiety to an amino acid of the polypeptide. In some cases, the functionalizing reagent selectively or specifically modifies the N-terminal amino acid (NTAA) of the polypeptide. In some embodiments, the chemical moiety is added via a chemical reaction or an enzymatic reaction. In some embodiments, the chemical moiety and attached NTAA are eliminated chemically. In other embodiments the chemical moiety and attached NTAA are eliminated enzymatically. In still other embodiments the chemical moiety and attached NTAA are eliminated chemically and enzymatically.

In some embodiments, the chemical moiety is a phenylthiocarbamoyl (PTC or derivatized PTC) moiety, a dinitrophenol (DNP) moiety, a sulfonyloxynitrophenyl (SNP) moiety, a dansyl moiety, a 7-methoxy coumarin moiety, a thioacyl moiety, a thioacetyl moiety, an acetyl moiety, a guanidinyl moiety, or a thiobenzyl moiety. In some examples, the functionalizing reagent comprises an isothiocyanate derivative (e.g., PITC, sulfo-PITC, nitro-PITC, methyl-PITC and methoxy-PITC), 2,4-dinitrobenzenesulfonic (DNBS), 4-sulfonyl-2-nitrofluorobenzene (SNFB) 1-fluoro-2,4-dinitrobenzene, dansyl chloride, 7-methoxycoumarin acetic acid, a thioacylation reagent, a guanidinylation reagent (e.g. PCA or PCA derivative), a thioacetylation reagent, and/or a thiobenzylation reagent.

In some of any of the provided embodiments, the functionalizing reagent comprises a compound selected from the group consisting of:

(i) a compound of Formula (I):

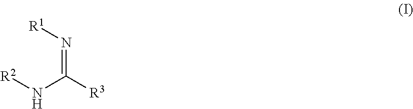

or a salt or conjugate thereof, wherein R¹ and R² are each independently H, C₁₋₆ alkyl, cycloalkyl, —C(O)R^{a}, —C(O)OR^{b}, or —S(O)₂R^{c}; R^{a}, R^{b}, and R^{c} are each independently H, C₁₋₆alkyl, C₁₋₆ haloalkyl, arylalkyl, aryl, or heteroaryl, wherein the C₁₋₆ alkyl, C₁₋₆ haloalkyl, arylalkyl, aryl, and heteroaryl are each unsubstituted or substituted, R³ is heteroaryl, —NR^{d}C(O)OR^{e}, or —SR^{f}, wherein the heteroaryl is unsubstituted or substituted; R^{d}, R^{e}, and R^{f} are each independently H or C₁₋₆alkyl; and optionally wherein when R³ is

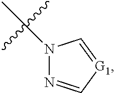

wherein G₁ is N, CH, or CX where X is halo, C₁₋₃ alkyl, C₁₋₃ haloalkyl, or nitro, R¹ and R² are not both H;

(ii) a compound of Formula (II):

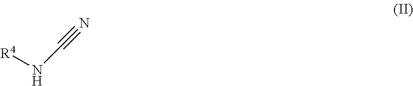

or a salt or conjugate thereof, wherein R⁴ is H, C₁₋₆alkyl, cycloalkyl, —C(O)R^{g}, or —C(O)OR^{g}; and R^{g} is H, C₁₋₆ alkyl, C₂₋₆alkenyl, C₁₋₆ haloalkyl, or arylalkyl, wherein the C₁₋₆ alkyl, C₂₋₆alkenyl, C₁₋₆ haloalkyl, and arylalkyl are each unsubstituted or substituted;

(iii) a compound of Formula (III):

R⁵—N═C═S (III)

or a salt or conjugate thereof, wherein R⁵ is C₁₋₆ alkyl, C₂₋₆alkenyl, cycloalkyl, heterocycloalkyl, aryl or heteroaryl; wherein the C₁₋₆ alkyl, C₂₋₆alkenyl, cycloalkyl, heterocycloalkyl, aryl or heteroaryl are each unsubstituted or substituted with one or more groups selected from the group consisting of halo, —NR^{h}Rⁱ, —S(O)₂R^{j}, or heterocyclyl; R^{h}, Rⁱ, and R^{j} are each independently H, C₁₋₆ alkyl, C₁₋₆ haloalkyl, arylalkyl, aryl, or heteroaryl, wherein the C₁₋₆ alkyl, C₁₋₆ haloalkyl, arylalkyl, aryl, and heteroaryl are each unsubstituted or substituted;

(iv) a compound of Formula (IV):

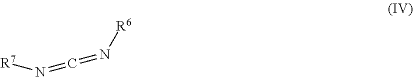

or a salt or conjugate thereof, wherein R⁶ and R⁷ are each independently H, C₁₋₆alkyl, —CO₂C₁₋₄alkyl, —OR^{k}, aryl, or cycloalkyl, wherein the C₁₋₆alkyl, —CO₂C₁₋₄alkyl, —OR^{k}, aryl, and cycloalkyl are each unsubstituted or substituted; and R^{k} is H, C₁₋₆alkyl, or heterocyclyl, wherein the C₁₋₆alkyl and heterocyclyl are each unsubstituted or substituted;

(v) a compound of Formula (V):

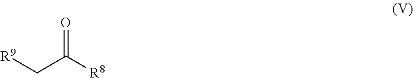

or a salt or conjugate thereof, wherein R⁸ is halo or —OR^{m}; R^{m} is H, C₁₋₆alkyl, or heterocyclyl; and R⁹ is hydrogen, halo, or C₁₋₆haloalkyl;

(vi) a metal complex of Formula (VI):

MLₙ (VI)

or a salt or conjugate thereof, wherein M is a metal selected from the group consisting of Co, Cu, Pd, Pt, Zn, and Ni; L is a ligand selected from the group consisting of —OH, —OH₂, 2,2′-bipyridine (bpy), 1,5dithiacyclooctane (dtco), 1,2-bis(diphenylphosphino)ethane (dppe), ethylenediamine (en), and triethylenetetramine (trien); and n is an integer from 1-8, inclusive; wherein each L can be the same or different; and

(vii) a compound of Formula (VII):

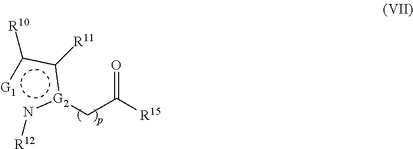

or a salt or conjugate thereof, wherein G¹ is N, NR¹³, or CR¹³R¹⁴—; G² is N or CH; p is 0 or 1; R¹⁰, R¹¹, R¹², R¹³, and R¹⁴ are each independently selected from the group consisting of H, C₁₋₆alkyl, C₁₋₆ haloalkyl, C₁₋₆ alkylamine, and C₁₋₆ alkylhydroxylamine, wherein the C₁₋₆alkyl, C₁₋₆ haloalkyl, C₁₋₆alkylamine, and C₁₋₆ alkylhydroxylamine are each unsubstituted or substituted, and R¹⁰ and R¹¹ can optionally come together to form a ring; and R¹⁵ is H or OH.

In some of any such embodiments, the method includes contacting the polypeptide with a reagent for removing the functionalized amino acid from the polypeptide to expose the immediately adjacent amino acid residue in the polypeptide. In some embodiments, modification of the amino acid of the polypeptide is accelerated due to the application of the microwave energy to the polypeptide. In some embodiments, the modification of the amino acid of the polypeptide due to the application of the microwave energy to the polypeptide is accelerated by at least 5% as compared to modification of the amino acid of the polypeptide without application of the microwave energy to the polypeptide.

In some of any of the provided embodiments, the polypeptide is contacted with a binding agent capable of binding to the polypeptide. In some embodiments, the polypeptide is contacted with a single binding agent capable of binding to the polypeptide. In some cases, the polypeptide is contacted with multiple binding agents capable of binding to the polypeptide.

In some embodiments, the method includes preparing a mixture comprising one or more polypeptides and one or more binding agents capable of binding to at least a portion of the one or more polypeptides; subjecting the mixture to a microwave energy; and determining the sequence of at least a portion of the one or more polypeptides.

Any suitable binding agent can be used. In some embodiments, each binding agent comprises a binding moiety capable of binding to: an internal polypeptide; a terminal amino acid residue; terminal di-amino-acid residues; terminal triple-amino-acid residues; an N-terminal amino acid (NTAA); a C-terminal amino acid (CTAA), a functionalized NTAA; or a functionalized CTAA. In some examples, the method includes contacting the polypeptide with one or more binding agents and applying a microwave energy, wherein each of the binding agents comprises a binding moiety capable of binding to a terminal amino acid residue, terminal di-amino-acid residues, or terminal triple-amino-acid residues of the polypeptide.

In some embodiments, the method includes preparing a mixture comprising one or more polypeptides and one or more binding agents, wherein each of the binding agents comprises a binding moiety capable of binding to a terminal amino acid residue, terminal di-amino-acid residues, or terminal triple-amino-acid residues; and subjecting the mixture to a microwave energy.

In some of any of the provided embodiments, each of the binding agents further comprises a coding tag comprising identifying information regarding the binding moiety. In some aspects, the binding agent and the coding tag are joined by a linker or a binding pair. In some embodiments, the binding agent binds to an N-terminal amino acid (NTAA), a C-terminal amino acid (CTAA) or a functionalized NTAA or CTAA of the polypeptide. In some cases, the binding agent binds to a post-translationally modified amino acid. In some embodiments, the binding agent is a polypeptide or a protein.

In some of any of the provided embodiments, the binding agent comprises an aminopeptidase or a variant, mutant, or modified protein thereof; an aminoacyl tRNA synthetase or a variant, mutant, or modified protein thereof; an anticalin or a variant, mutant, or modified protein thereof; a ClpS (such as ClpS2) or a variant, mutant, or modified protein thereof; a UBR box protein or a variant, mutant, or modified protein thereof; or a small molecule that binds to an amino acid, i.e. vancomycin or a variant, mutant, or modified molecule thereof; or an antibody or a binding fragment thereof; or any combination thereof. In some embodiments, the binding agent binds to a single amino acid residue (e.g., an N-terminal amino acid residue, a C-terminal amino acid residue, or an internal amino acid residue), a dipeptide (e.g., an N-terminal dipeptide, a C-terminal dipeptide, or an internal dipeptide), a tripeptide (e.g., an N-terminal tripeptide, a C-terminal tripeptide, or an internal tripeptide), or a post-translational modification of the analyte or polypeptide.

In some embodiments, binding between or among the binding agent and the polypeptide is accelerated due to the application of the microwave energy to the polypeptide. In some cases, binding between or among the binding agent and the polypeptide due to the application of the microwave energy to the polypeptide is accelerated by at least 5% as compared to binding between or among the binding agent and the polypeptide without application of the microwave energy to the polypeptide.

In some of any of the provided embodiments, the polypeptide is contacted with a removing reagent to remove an amino acid from the polypeptide. In some cases, the polypeptide is contacted with the removing reagent to remove a single amino acid from the polypeptide. In some aspects, the polypeptide is contacted with the removing reagent to remove multiple amino acids from the polypeptide.

In some embodiments, the method includes contacting the polypeptide with a reagent to remove one or more amino acids from the polypeptide and applying a microwave energy; and determining the sequence of at least a portion of the polypeptide. In some embodiments, the method includes preparing a mixture comprising one or more polypeptides and reagents for removing one or more amino acids from the one or more polypeptides; subjecting the mixture to a microwave energy; and determining the sequence of at least a portion of the one or more polypeptides.

In some embodiments, the removed amino acid includes (i) an N-terminal amino acid (NTAA); (ii) an N-terminal dipeptide sequence; (iii) an N-terminal tripeptide sequence; (iv) an internal amino acid; (v) an internal dipeptide sequence; (vi) an internal tripeptide sequence; (vii) a C-terminal amino acid (CTAA); (viii) a C-terminal dipeptide sequence; or (ix) a C-terminal tripeptide sequence, or any combination thereof. In some embodiments, any one or more of the amino acid residues in (i)-(ix) are modified or functionalized.

In some embodiments, the method includes contacting the polypeptide with a reagent to remove one or more N-terminal amino acids (NTAA) from the polypeptide and applying a microwave energy. In some embodiments, the method includes preparing a mixture comprising one or more polypeptides and one or more reagents for removing one or more N-terminal amino acids (NTAA) from the one or more polypeptides; and subjecting the mixture to a microwave energy. In some embodiments, the removing reagent selectively or specifically removes the N-terminal amino acid (NTAA) of the polypeptide. In some cases, the removing reagent removes one amino acid. In some aspects, the removing reagent removes two amino acids. In some embodiments, removing the one or more amino acids exposes a new N-terminal amino acid of the polypeptide.

In some of any of the provided embodiments, the amino acid is removed from the polypeptide by a chemical cleavage or an enzymatic cleavage. In some embodiments, the removing reagent removes a functionalized amino acid residue from the polypeptide.

Any suitable removing reagent can be used. In some cases, the removing reagent comprises trifluoroacetic acid or hydrochloric acid. In some examples, the removing reagent comprises an enzymatic reagent. In some embodiments, the removing reagent includes a carboxypeptidase, an aminopeptidase, a peptidase (e.g., dipeptidylpeptidase (DPP) or dipeptidyl aminopeptidase, for example, DPP1-11 (MEROPS; Rawlings et al., Nucleic Acids Research, (2017) 46(D1): D624-D632)) or a variant, mutant, or modified protein thereof; a hydrolase (e.g. an acylpeptide hydrolase (APH)), or a variant, mutant, or modified protein thereof; a mild Edman degradation reagent; an Edmanase enzyme; anhydrous TFA, a base; or any combination thereof. In some embodiments, the mild Edman degradation uses a dichloro or monochloro acid; the mild Edman degradation uses TFA, TCA, or DCA; or the mild Edman degradation uses triethylamine, triethanolamine, or triethylammonium acetate (Et₃NHOAc).

In some cases, the reagent for removing the amino acid comprises a base. In some embodiments, the base is a hydroxide, an alkylated amine, a cyclic amine, a carbonate buffer, trisodium phosphate buffer, or a metal salt. In some examples, the hydroxide is sodium hydroxide; the alkylated amine is selected from methylamine, ethylamine, propylamine, dimethylamine, diethylamine, dipropylamine, trimethylamine, triethylamine, tripropylamine, cyclohexylamine, benzylamine, aniline, diphenylamine, N,N-Diisopropylethylamine (DIPEA), and lithium diisopropylamide (LDA); the cyclic amine is selected from pyridine, pyrimidine, imidazole, pyrrole, indole, piperidine, prolidine, 1,8-diazabicyclo[5.4.0]undec-7-ene (DBU), and 1,5-diazabicyclo[4.3.0]non-5-ene (DBN); the carbonate buffer comprises sodium carbonate, potassium carbonate, calcium carbonate, sodium bicarbonate, potassium bicarbonate, or calcium bicarbonate; the metal salt comprises silver; or the metal salt is AgClO₄.

In some embodiments, the method further includes contacting the polypeptide with a peptide coupling reagent. In some embodiments, the peptide coupling reagent is a carbodiimide compound. In some examples, the carbodiimide compound is diisopropylcarbodiimide (DIC) or 1-ethyl-3-(3-dimethylaminopropyl)carbodiimide (EDC).

In some embodiments, the removed amino acid is an amino acid modified using any of the methods provided herein. In some embodiments, removal of an amino acid from the polypeptide is accelerated due to the application of the microwave energy to the polypeptide. In some cases, removal of an amino acid from the polypeptide due to the application of the microwave energy to the polypeptide is accelerated by at least 5% as compared to removal of an amino acid from the polypeptide without application of the microwave energy to the polypeptide. In some examples, the sequence of at least a portion of the polypeptide is determined by Edman degradation.

In some embodiments, the method includes (a) modifying the N-terminal amino acid (NTAA) of a polypeptide with a functionalizing reagent; and (b) contacting the polypeptide with a removing reagent to remove the modified NTAA; wherein step (a) and/or step (b) are performed in the presence of a microwave energy. In some embodiments, the method further includes (a1) contacting the polypeptide with a binding agent that binds to the modified NTAA, optionally in the presence of microwave energy. In some embodiments, the method further includes (c) determining the sequence of at least a portion of the polypeptide.

In some of any of the provided embodiments, the method includes (a) contacting a plurality of polypeptides with a functionalizing reagent to modify an amino acid of each of the polypeptides; (b) contacting the polypeptides with a removing reagent to remove the modified amino acids; and (c) determining the sequence of at least a portion of each of the polypeptides; wherein step (a) and/or step (b) are performed in the presence of a microwave energy. In some embodiments, the method further includes (a1) contacting the polypeptides with a binding agent, optionally in the presence of a microwave energy. In some embodiments, at least one of the modified and removed amino acids is an N-terminal amino acid (NTAA) or a C-terminal amino acid (CTAA) of the polypeptide.

In some of any of the provided embodiments, step (a) and step (b) are performed sequentially; step (a), (a1), and step (b) are performed sequentially; step (a), (a1), step (b) and step (c) are performed sequentially; step (a) is performed before step (a1); step (a) is performed before step (b); step (a1) is performed before step (b); step (a) is performed before step (c); step (a1) is performed before step (c); step (a) and step (b) are repeated; step (a), (a1), and step (b) are repeated; or step (b) is performed before step (c).

Provided herein is a method for analyzing a polypeptide including the steps: (a) providing a polypeptide optionally associated directly or indirectly with a recording tag; (b) functionalizing the N-terminal amino acid (NTAA) of said polypeptide with a functionalizing reagent to yield a functionalized NTAA, (c) contacting said polypeptide with a first binding agent comprising a first binding portion capable of binding to said functionalized NTAA and (c1) a first coding tag with identifying information regarding said first binding agent, or (c2) a first detectable label; (d) (d1) transferring the information of said first coding tag to said recording tag to generate a first extended recording tag and analyzing said extended recording tag, or (d2) detecting said first detectable label, and wherein said polypeptide is contacted with a microwave energy before any of said steps (b), (c), (d1) and (d2), or any one or more of steps (b), (c), (d1), and/or (d2) are performed in the presence of a microwave energy.

In some embodiments, the method further includes contacting the polypeptide with a proline aminopeptidase under conditions suitable to cleave an N-terminal proline before step (b). In some cases, the method further includes (e) contacting the polypeptide with a reagent to remove the functionalized NTAA to expose a new NTAA. In some aspects, the method further includes between steps (d) and (e), repeating steps (b) to (d) to determine the sequence of at least a portion of the polypeptide.

In some of any of the provided embodiments, the binding agent binds to the N-terminal amino acid residue of the polypeptide and the N-terminal amino acid residue is removed after each binding cycle. In some embodiments, the N-terminal amino acid residue is removed via Edman degradation. In some of any of the provided embodiments, the functionalizing reagent comprises a chemical agent, an enzyme, and/or a biological agent.

In some embodiments, the functionalizing reagent adds a chemical moiety to the amino acid. In some embodiments, the functionalizing reagent selectively or specifically modifies the N-terminal amino acid (NTAA) of the polypeptide. In some embodiments, the chemical moiety is added via a chemical reaction or an enzymatic reaction. In some examples, the chemical moiety is a phenylthiocarbamoyl (PTC or derivatized PTC), a dinitrophenol (DNP) moiety; a sulfonyloxynitrophenyl (SNP) moiety, a dansyl moiety; a 7-methoxy coumarin moiety; a thioacyl moiety; a thioacetyl moiety; an acetyl moiety; a guanidinyl moiety; or a thiobenzyl moiety. In some embodiments, the functionalizing reagent comprises an isothiocyanate derivative, a phenylisothiocyanate, PITC, 2,4-dinitrobenzenesulfonic (DNBS), benzyloxycarbonyl chloride or carbobenzoxy chloride (Cbz-Cl), N-(Benzyloxycarbonyloxy)succinimide (Cbz-OSu or Cbz-O-NHS), dansyl chloride (DNS-Cl, or 1-dimethylaminonaphthalene-5-sulfonyl chloride), 4-sulfonyl-2-nitrofluorobenzene (SNFB), 1-fluoro-2,4-dinitrobenzene (Sanger's reagent, DNFB), dansyl chloride, 7-methoxycoumarin acetic acid, N-Acetyl-Isatoic Anhydride, Isatoic Anhydride, 2-Pyridinecarboxaldehyde, 2-Formylphenylboronic acid, 2-Acetylphenylboronic acid, 1-Fluoro-2,4-dinitrobenzene, Succinic anhydride, 4-Chloro-7-nitrobenzofurazan, Pentafluorophenylisothiocyanate, 4-(Trifluoromethoxy)-phenylisothiocyanate, 4-(Trifluoromethyl)-phenylisothiocyanate, 3-(Carboxylic acid)-phenylisothiocyanate, 3-(Trifluoromethyl)-phenylisothiocyanate, 1-Naphthylisothiocyanate, N-nitroimidazole-1-carboximidamide, N,N,Ä≤-Bis(pivaloyl)-1H-pyrazole-1-carboxamidine, N,N,Ä≤-Bis(benzyloxycarbonyl)-1H-pyrazole-1-carboxamidine, an acetylating reagent, a guanidinylation reagent, a thioacylation reagent, a thioacetylation reagent, a thiobenzylation reagent, and/or a diheterocyclic methanimine reagent. In some examples, the binding agent binds an amino acid labeled with a reagent or using a method as described in International Patent Publication No. WO 2019/089846. In some cases, the binding agent binds an amino acid labeled by an amine modifying reagent.

In some of any of the provided embodiments, the functionalizing reagent comprises a compound selected from the group consisting of:

(i) a compound of Formula (I):

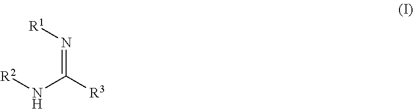

or a salt or conjugate thereof, wherein R¹ and R² are each independently H, C₁₋₆ alkyl, cycloalkyl, —C(O)R^{a}, —C(O)OR^{b}, or —S(O)₂R^{c}; R^{a}, R^{b}, and R^{c} are each independently H, C₁₋₆alkyl, C₁₋₆haloalkyl, arylalkyl, aryl, or heteroaryl, wherein the C₁₋₆ alkyl, C₁₋₆ haloalkyl, arylalkyl, aryl, and heteroaryl are each unsubstituted or substituted, R³ is heteroaryl, —NR^{d}C(O)OR^{e}, or —SR^{f}, wherein the heteroaryl is unsubstituted or substituted; R^{d}, R^{e}, and R^{f} are each independently H or C₁₋₆alkyl; and optionally wherein when R³ is

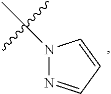 [SMILES: *n1cccn1]

R¹ and R² are not both H;

(ii) a compound of Formula (II):

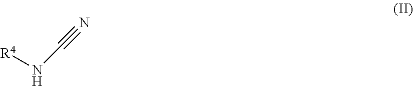

or a salt or conjugate thereof, wherein R⁴ is H, C₁₋₆alkyl, cycloalkyl, —C(O)R^{g}, or —C(O)OR^{g}; and R^{g} is H, C₁₋₆alkyl, C₂₋₆alkenyl, C₁₋₆ haloalkyl, or arylalkyl, wherein the C₁₋₆ alkyl, C₂₋₆alkenyl, C₁₋₆haloalkyl, and arylalkyl are each unsubstituted or substituted;

(iii) a compound of Formula (III):

R⁵—N═C═S (III)

or a salt or conjugate thereof, wherein R⁵ is C₁₋₆alkyl, C₂₋₆alkenyl, cycloalkyl, heterocycloalkyl, aryl or heteroaryl; wherein the C₁₋₆ alkyl, C₂₋₆alkenyl, cycloalkyl, heterocycloalkyl, aryl or heteroaryl are each unsubstituted or substituted with one or more groups selected from the group consisting of halo, —NR^{h}Rⁱ, —S(O)₂R^{j}, or heterocyclyl; R^{h}, Rⁱ, and R^{j} are each independently H, C₁₋₆ alkyl, C₁₋₆haloalkyl, arylalkyl, aryl, or heteroaryl, wherein the C₁₋₆alkyl, C₁₋₆ haloalkyl, arylalkyl, aryl, and heteroaryl are each unsubstituted or substituted;

(iv) a compound of Formula (IV):

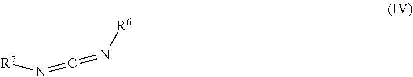

or a salt or conjugate thereof, wherein R⁶ and R⁷ are each independently H, C₁₋₆alkyl, —CO₂C₁₋₄alkyl, —OR^{k}, aryl, or cycloalkyl, wherein the C₁₋₆alkyl, —CO₂C₁₋₄alkyl, —OR^{k}, aryl, and cycloalkyl are each unsubstituted or substituted; and R^{k} is H, C₁₋₆alkyl, or heterocyclyl, wherein the C₁₋₆alkyl and heterocyclyl are each unsubstituted or substituted;

(v) a compound of Formula (V):

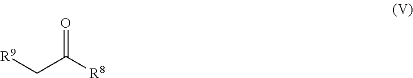

or a salt or conjugate thereof, wherein R⁸ is halo or —OR^{m}; R^{m} is H, C₁₋₆alkyl, or heterocyclyl; and R⁹ is hydrogen, halo, or C₁₋₆ haloalkyl;

(vi) a metal complex of Formula (VI):

MLₙ (VI)

or a salt or conjugate thereof, wherein M is a metal selected from the group consisting of Co, Cu, Pd, Pt, Zn, and Ni; L is a ligand selected from the group consisting of —OH, —OH₂, 2,2′-bipyridine (bpy), 1,5dithiacyclooctane (dtco), 1,2-bis(diphenylphosphino)ethane (dppe), ethylenediamine (en), and triethylenetetramine (trien); and n is an integer from 1-8, inclusive; wherein each L can be the same or different; and

(vii) a compound of Formula (VII):

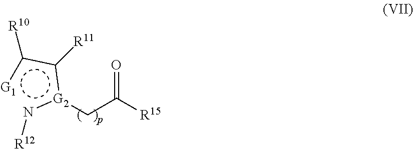

or a salt or conjugate thereof, wherein G¹ is N, NR¹³, or CR¹³R¹⁴; G² is N or CH; p is 0 or 1; R¹⁰, R¹¹, R¹², R¹³, and R¹⁴ are each independently selected from the group consisting of H, C₁₋₆ alkyl, C₁₋₆ haloalkyl, C₁₋₆alkylamine, and C₁₋₆alkylhydroxylamine, wherein the C₁₋₆alkyl, C₁₋₆ haloalkyl, C₁₋₆alkylamine, and C₁₋₆alkylhydroxylamine are each unsubstituted or substituted, and R¹⁰ and R¹¹ can optionally come together to form a ring; and R¹⁵ is H or OH.

In some embodiments, the binding agents each further include a coding polymer containing identifying information regarding the first binding moiety. In some embodiments, the binding agent and the coding tag are joined by a linker or a binding pair. In some aspects, the binding agent binds to an N-terminal amino acid (NTAA), a C-terminal amino acid (CTAA) or a functionalized NTAA or CTAA of the polypeptide. In some cases, the binding agent binds to a post-translationally modified amino acid.

In some embodiments, the binding agent is a polypeptide or a protein. In some examples, the binding agent includes an aminopeptidase or a variant, mutant, or modified protein thereof; an aminoacyl tRNA synthetase or a variant, mutant, or modified protein thereof; an anticalin or a variant, mutant, or modified protein thereof; a ClpS (such as ClpS2) or a variant, mutant, or modified protein thereof; a UBR box protein or a variant, mutant, or modified protein thereof; or a small molecule that binds to an amino acid, i.e. vancomycin or a variant, mutant, or modified molecule thereof; or an antibody or a derivative or binding fragment thereof; or any combination thereof. In some embodiments, the binding agent binds to a single amino acid residue (e.g., an N-terminal amino acid residue, a C-terminal amino acid residue, or an internal amino acid residue), a dipeptide (e.g., an N-terminal dipeptide, a C-terminal dipeptide, or an internal dipeptide), a tripeptide (e.g., an N-terminal tripeptide, a C-terminal tripeptide, or an internal tripeptide), or a post-translational modification of the analyte or polypeptide.

In some of any of the provided embodiments, the method further includes determining the sequence of at least a portion of the polypeptide.

In some embodiments, the removing reagent selectively removes the N-terminal amino acid (NTAA) of the polypeptide. In some embodiments, the removing reagent removes one amino acid. In some cases, the removing reagent removes two amino acids. In some aspects, removing the one or more amino acid(s) exposes a new N-terminal amino acid of the polypeptide. In some embodiments, the amino acid is removed from the polypeptide by a chemical cleavage or an enzymatic cleavage. In some cases, the removing reagent is for removing a functionalized amino acid residue from the polypeptide. In some embodiments, the removing reagent for removing the functionalized amino acid residue comprises trifluoroacetic acid or hydrochloric acid. In some examples, the removing reagent for removing the functionalized NTAA comprises acylpeptide hydrolase (APH), a peptidase (e.g., dipeptidyl peptidase (DPP) or dipeptidyl aminopeptidase, including DPP1-11 (MEROPS; Rarylings et al., Nucleic Acids Research, (2017) 46(D1): D624-D632)) or a variant, mutant, or modified protein thereof. In some cases, the removing reagent to remove an amino acid comprises a carboxypeptidase or an aminopeptidase or a variant, mutant, or modified protein thereof; a hydrolase or a variant, mutant, or modified protein thereof; a mild Edman degradation reagent; an Edmanase enzyme; anhydrous TFA, a base; or any combination thereof. In some embodiments, the mild Edman degradation uses a dichloro or monochloro acid; the mild Edman degradation uses TFA, TCA, or DCA; or the mild Edman degradation uses triethylammonium acetate (Et₃NHOAc).

In some of any of the provided embodiments, the removing reagent for removing the amino acid(s) comprises a base. In some embodiments, the base is a hydroxide, an alkylated amine, a cyclic amine, a carbonate buffer, or a metal salt. In some examples, the hydroxide is sodium hydroxide; the alkylated amine is selected from methylamine, ethylamine, propylamine, dimethylamine, diethylamine, dipropylamine, trimethylamine, triethylamine, tripropylamine, cyclohexylamine, benzylamine, aniline, diphenylamine, N,N-Diisopropylethylamine (DIPEA), and lithium diisopropylamide (LDA); the cyclic amine is selected from pyridine, pyrimidine, imidazole, pyrrole, indole, piperidine, prolidine, 1,8-diazabicyclo[5.4.0]undec-7-ene (DBU), and 1,5-diazabicyclo[4.3.0]non-5-ene (DBN); the carbonate buffer comprises sodium carbonate, potassium carbonate, calcium carbonate, sodium bicarbonate, potassium bicarbonate, or calcium bicarbonate; or the metal salt comprises silver; or the metal salt is AgClO₄.

In some of any of the provided embodiments, the method further includes contacting the polypeptide with a peptide coupling reagent. In some examples, the peptide coupling reagent is a carbodiimide compound. In some embodiments, the carbodiimide compound is diisopropylcarbodiimide (DIC) or 1-ethyl-3-(3-dimethylaminopropyl)carbodiimide (EDC).

Any suitable microwave energy can be used. In some of any of the provided embodiments, the microwave energy has a wavelength from about one meter to about one millimeter, e.g., a wavelength from about 0.3 m to about 3 mm. In some embodiments, the microwave energy has a frequency from about 300 MHz (1 m) to about 300 GHz (1 mm). In some cases, the microwave energy has a frequency from about 1 GHz to about 100 GHz. In some embodiments, the microwave energy has a frequency with an IEEE radar band designation of S, C, X, Kᵤ, K or Kₐ band. In some embodiments, the microwave energy has a photon energy (eV) from about 1.24 μeV to about 1.24 meV, e.g., at about 1.24 μeV to about 12.4 μeV, about 12.4 μeV to about 124 μeV, about 124 μeV to about 1.24 meV. In some cases, the microwave energy is applied at about 5 watts, about 10 watts, about 15 watts, about 20 watts, about 25 watts, about 30 watts, about 35 watts, about 40 watts, about 45 watts, about 50 watts, about 60 watts, about 70 watts, about 80 watts, about 90 watts, about 100 watts, about 110 watts, about 120 watts, about 130 watts, about 140 watts, about 150 watts, about 300 watts or higher watts. In some examples, the microwave energy is applied for a time period of about 1 minute, 2 minutes, 3 minutes, 4 minutes, 5 minutes, 10 minutes, 15 minutes, 20 minutes, 25 minutes, 30 minutes, 35 minutes, 40 minutes, 45 minutes, 50 minutes, 1 hour, or a loner time period for any or each of the step(s).

In some of any of the provided embodiments, the microwave energy is applied for a duration of time effective to achieve modification of, binding to and/or removal of an amino acid in at least 20%, 30%, 40%, 50%, 60%, 70%, 80%, 90% or greater percentage of the polypeptides. In some embodiments, the microwave energy is applied by a non-uniform microwave field. In some embodiments, the microwave energy is applied by a uniform microwave field, e.g., applied by microwave volumetric heating (MVH). In some embodiments, the microwave energy is applied in the presence one or more ionic liquids. In some embodiments, the method further includes monitoring and/or controlling the temperature at which any or all step(s) of the method is or are conducted. In some of any of the provided embodiments, the method further includes applying cooling. In some examples, the method further includes applying active cooling.

In some of any of the provided embodiments, the method is performed in a vessel or a container. In some embodiments, the method is performed in a cavity in communication with a microwave radiation source.

In some embodiments, the method is performed in a microwave chamber. In some cases, the polypeptide is joined to the support via a linker. In some embodiments, the polypeptide is joined to the support at the N-terminal end of the polypeptide. In some embodiments, the polypeptide is joined to the support at the C-terminal end of the polypeptide. In some embodiments, the polypeptide is joined to the support via a side chain of the polypeptide.

In some of any of the provided embodiments, the polypeptide is joined to a recording tag. Any suitable recording tag can be used. In some cases, the recording tag is a sequenceable polymer. In some embodiments, the recording tag comprises a polynucleotide or a non-nucleic acid sequenceable polymer. In some embodiments, the polypeptide and associated recording tag are covalently immobilized to the support (e.g., via a linker), or non-covalently immobilized to the support (e.g., via a binding pair).

In some embodiments, the polypeptide and associated recording tag are directly or indirectly attached to an immobilizing linker. In some of any such embodiments, the immobilizing linker is immobilized directly or indirectly to the support, thereby immobilizing the at least one polypeptide and/or its associated recording tag to the support. Any suitable support can be used. In some examples, the support comprises a bead, a porous bead, a porous matrix, an array, a glass surface, a silicon surface, a plastic surface, a filter, a membrane, a nylon, a silicon wafer chip, a flow through chip, a biochip including signal transducing electronic, a microtitre well, an ELISA plate, a spinning interferometry disc, a nitrocellulose membrane, a nitrocellulose-based polymer surface, a nanoparticle, or a microsphere. In some examples, the support comprises a polystyrene bead, a polymer bead, an agarose bead, an acrylamide bead, a solid core bead, a porous bead, a paramagnetic bead, glass bead, or a controlled pore bead.

In some of any of the provided embodiments, the method further includes analyzing the recording tag. The recording tag can be analyzed using any suitable technique or method. For example, the recording tag can be analyzed using nucleic acid sequence analysis. In some embodiments, the nucleic acid sequence analysis comprises sequencing by synthesis, sequencing by ligation, sequencing by hybridization, polony sequencing, ion semiconductor sequencing, pyro sequencing, single molecule real-time sequencing, nanopore-based sequencing, or direct imaging of DNA using advanced microscopy, or any combination thereof. In some embodiments, the method includes contacting a polypeptide with a functionalizing reagent to modify an amino acid of the polypeptide, a binding agent capable of binding to the polypeptide, and a removing reagent to remove an amino acid from the polypeptide.

In some of any of the provided embodiments, modification of the amino acid of the polypeptide, binding between or among the binding agent and the polypeptide and/or removal of an amino acid from the polypeptide is accelerated due to the application of the microwave energy to the polypeptide. In some cases, the time required for conducting any or all steps of the method is shortened due to the application of the microwave energy to the polypeptide. In some examples, the time required for conducting any or all steps of the method due to the application of the microwave energy to the polypeptide is shortened by at least 5% as compared to a time required for conducting any or all steps of the method without application of the microwave energy to the polypeptide. In some embodiments, the level or percentage of modification of the amino acid of the polypeptide, binding between or among the binding agent and the polypeptide and/or removal of an amino acid from the polypeptide is enhanced or increased due to the application of the microwave energy to the polypeptide. In some embodiments, the level or percentage of modification of the amino acid of the polypeptide, binding between or among the binding agent and the polypeptide and/or removal of an amino acid from the polypeptide due to the application of the microwave energy to the polypeptide is enhanced or increased by at least 5% as compared to the level or percentage of modification of the amino acid of the polypeptide, binding between or among the binding agent and the polypeptide and/or removal of an amino acid from the polypeptide without application of the microwave energy to the polypeptide.

In some embodiments, bias of functionalization and/or removal of different amino acids is reduced or eliminated due to the application of the microwave energy to the polypeptide. In some cases, the bias of functionalization and/or removal between hydrophobic amino acids and non-hydrophobic amino acids is reduced or eliminated due to the application of the microwave energy to the polypeptide. In some embodiments, the bias of functionalization and/or removal of different amino acids due to the application of the microwave energy to the polypeptide is reduced by at least 5% as compared to the bias of functionalization and/or removal of different amino acids without application of the microwave energy to the polypeptide.

Provided herein is a kit or system for sequencing a polypeptide, which contains a functionalizing reagent to modify an amino acid of a polypeptide, a binding agent capable of binding to said polypeptide, and/or a removing reagent to remove an amino acid from said polypeptide; a microwave energy source, e.g., a microwave energy source configured for applying a micro wave energy to said polypeptide; and a reagent or a device for determining the sequence of at least a portion of said polypeptide.

Provided herein is a kit or system for treating a polypeptide, including, a functionalizing reagent to modify an amino acid of a polypeptide, a binding agent capable of binding to said polypeptide, and/or a removing reagent to remove an amino acid from said polypeptide; and a microwave energy source, e.g., a microwave energy source configured for applying a microwave energy to said polypeptide; wherein the functionalizing reagent modifies an N-terminal amino acid (NTAA), the binding agent binds to an N-terminal amino acid (NTAA), and/or the removing reagent removes an N-terminal amino acid (NTAA).

Also provided herein is a kit or system for analyzing a polypeptide, which comprises a recording tag configured to be associated directly or indirectly with a polypeptide; a functionalizing reagent for modifying the N-terminal amino acid (NTAA) of said polypeptide to yield a functionalized NTAA; a first binding agent comprising a first binding portion capable of binding to said functionalized NTAA and a first coding tag with identifying information regarding said first binding agent, or a first detectable label; and a microwave energy source, e.g., a microwave energy source configured for applying a microwave energy to said polypeptide. In some embodiments, the kit or system further includes a reagent or a device for transferring the information of the first coding tag to the recording tag to generate a first extended recording tag and/or for analyzing said extended recording tag, or for detecting the first detectable label.

### BRIEF DESCRIPTION OF THE DRAWINGS

Non-limiting embodiments of the present invention will be described by way of example with reference to the accompanying figures, which are schematic and are not intended to be drawn to scale. For purposes of illustration, not every component is labeled in every figure, nor is every component of each embodiment of the invention shown where illustration is not necessary to allow those of ordinary skill in the art to understand the invention.

FIG. 1 depicts an exemplary process for a degradation-based polypeptide sequencing assay by construction of an extended recording tag (e.g., DNA sequence) representing the polypeptide sequence (ProteoCode assay). This is accomplished through an Edman degradation-like approach using a cyclic process such as terminal amino acid functionalization (e.g., N-terminal amino acid (NTAA) functionalization), coding tag information transfer to a recording tag attached to the polypeptide, terminal amino acid elimination (e.g., NTAA elimination), and repeating the process in a cyclic manner, for example, on a solid support. The polypeptide is immobilized on a solid support via a capture agent and optionally cross-linked. Either the protein or capture agent may co-localize or be labeled with a recording tag, and proteins with associated recording tags are directly immobilized on a solid support. In an exemplary first step, the N-terminal amino acid (NTAA) is labeled with a functionalization reagent to enable removal of the NTAA in a later step; the functionalizing reagent generates an NTAA residue containing a functionalization moiety (e.g., a phenylthiocarbamoyl (PTC or derivatized PTC), dinitrophenyl (DNP), sulfonyl nitrophenyl (SNP), acetyl, or guanidinyl moiety). A second step includes contacting the polypeptide with a binding agent that is attached to a unique DNA tag. Upon binding of the binding agent to the NTAA of the polypeptide, information of the coding tag is transferred to the recording tag (e.g., via primer extension or ligation) to generate an extended recording tag. Lastly, the functionalized NTAA is eliminated via chemical or biological (e.g., enzymatic) means to expose a new NTAA. As illustrated, the cycle is repeated “n” times to generate a final extended recording tag. The final extended recording tag is optionally flanked by universal priming sites to facilitate downstream amplification and/or DNA sequencing. The forward universal priming site (e.g., Illumina's P5-S1 sequence) can be part of the original recording tag design and the reverse universal priming site (e.g., Illumina's P7-S2′ sequence) can be added as a final step in the extension of the recording tag. This final step may be done independently of a binding agent. In some embodiments, the order in the steps in the process for a degradation-based peptide polypeptide sequencing assay can be reversed or moved around. For example, in some embodiments, the terminal amino acid functionalization can be conducted after the polypeptide is bound to the binding agent and/or associated coding tag. In some embodiments, the terminal amino acid functionalization can be conducted after the polypeptide is bound a support.

FIG. 2 shows results of microwave-assisted NTAA functionalization (NTF) with various exemplary guanidinylating reagents and microwave-assisted NTAA removal (elimination, NTE). For comparison, functionalization and elimination reactions were performed in the absence of microwave energy with conventional thermal heating applied.

FIG. 3A-3D depicts results from performing exemplary ProteoCode assay showing encoding efficiency of a two cycle of binding and encoding with a binding agent that recognizes the amino acid residue, Phenylalanine, (F binder). The results show encoding pre-NTF/NTE chemistry reactions and post-NTF/NTE chemistry reactions, in the presence (FIGS. 3B and 3D) or absence of microwave energy (FIGS. 3A and 3C).

FIG. 4 shows the results from exemplary gel electrophoresis analysis of oligonucleotide molecules tested with heat treatment and microwave treatment in the presence of the various reagents as indicated.

### DETAILED DESCRIPTION

Provided herein are methods of treating a macromolecule or a plurality of macromolecules, e.g., peptides, polypeptides, and proteins, in the presence of radiation energy. Also provided herein are methods for accelerating a sequencing reaction including preparing and/or treating a polypeptide. In some embodiments, the methods are for preparing polypeptides for sequencing and/or sequence analysis. In some embodiments, the methods provided include accelerating reactions with polypeptides. In some embodiments, the methods for accelerating reactions includes the application of radiation, e.g., electromagnetic radiation or microwave energy. In some embodiments, the methods are for reacting or contacting a plurality of polypeptides with a functionalizing reagent to modify one or more amino acids of the polypeptide. In some embodiments, the methods are for contacting the polypeptides with one or more binding agents. In some embodiments, the methods are for reacting or contacting a plurality of polypeptides with a reagent to remove one or more amino acids of the polypeptide. In some aspects, the methods include accelerating reactions including polypeptides with functionalizing reagents, binding agents, and/or agents for removing one or more amino acids. In some embodiments, the method further includes determining the sequence of at least a portion of the polypeptide.

Some chemistries and reactions involving polypeptides require a lengthy amount of time. In some cases, it has been shown that elevating temperature by applying heat may improve efficiency of a reaction. However, conventional methods of applying heat may create a temperature gradient in the sample and/or may not introduce heat in a controlled manner (e.g., side reactions).

Accordingly, there is a need for alternative technologies in performing reactions (e.g., chemical reactions and/or enzymatic reactions) that increase the efficiency and/or reduce or avoid the problems associated with currently used protocols. In some aspects, a desired method for accelerating reactions with polypeptides may improve reactions to occur in a controlled manner that is able to maintain integrity of the reagents, components, and desired reaction and products.

In a particular application, protein analysis and/or sequencing relies on the ability to modify a plurality of polypeptides in an efficient manner. For example, direct protein characterization can be achieved via peptide sequencing (Edman degradation or mass spectroscopy). Peptide sequencing based on Edman degradation includes stepwise degradation of the N-terminal amino acid on a peptide through a series of chemical modifications and downstream HPLC analysis (later replaced by mass spectrometry analysis). In a first step, the N-terminal amino acid is modified with phenyl isothiocyanate (PITC) under mildly basic conditions (NMP/methanol/H₂O) to form a phenylthiocarbamoyl (PTC or derivatized PTC) derivative. In a second step, the PTC or derivatized PTC-modified amino group is treated with acid (anhydrous trifluoroacetic acid, TFA) to create a cleaved cyclic ATZ (2-anilino-5(4)-thiozolinone) modified amino acid, leaving a new N-terminus on the peptide. The cleaved cyclic ATZ-amino acid is converted to a phenylthiohydantoin (PTH)-amino acid derivative and analyzed by reverse phase HPLC. This process is continued in an iterative fashion until all or a partial number of the amino acids comprising a peptide sequence has been removed from the N-terminal end and identified. However, in general, Edman degradation peptide sequencing method is slow and has a limited throughput of only a few peptides per day, therefore, this approach is not parallel or high-throughput.

Thus, there remains a need in the art for improved techniques relating to macromolecule (e.g., polypeptide or polynucleotide) processing, including to increase efficiency and/or improve currently used protocols. In some embodiments, this need is related to applications in protein sequencing and/or analysis, as well as to products, methods, articles of manufacture, and kits for accomplishing the same. In some embodiments, such needed improvements may allow a highly-parallelized, accurate, sensitive, and/or high-throughput method applicable for protein analysis and/or sequencing. Also needed are products, methods and kits for accomplishing the same. The present disclosure fulfills these and other related needs.

Provided herein are methods of increasing the rate of reactions that meets such needs, e.g., methods for increasing the rate of chemical reactions and/or biological or enzymatic reactions with polypeptides. In some embodiments, application of microwave energy may improve reactions (Collins et al., Org. Biomol. Chem., (2007) 5:1141-1150; Kappe et al., Angew. Chem. Int. Ed. (2013) 52, 1088-1094; Lill et al., Mass Spectrometry Reviews (2007) 26:657-671; Bose et al., J Am Soc Mass Spectrom. (2002) 13(7):839-850). The provided methods meets such needs by applying sufficient microwave radiation to the mixture of polypeptides and reagents. In some embodiments, microwave radiation may offer a number of advantages over conventional heating methods, such as noncontact heating, instantaneous and rapid heating, and highly specific heating.

In some embodiments, the present disclosure provides, in part, improved methods for treating or preparing reactions with polypeptides. In some embodiments, provided herein are methods for preparing polypeptides by application of radiation energy. For example, radiation energy may be applied in the form of microwave energy or other electromagnetic radiation sources. For microwave energy, the molecules in the sample are exposed to electromagnetic radiation. In some cases, application of microwave energy supplies heat throughout the sample. In some aspects, applying microwave energy enables acute, precise and/or even heating of the reaction, and/or allows for even distribution of heat throughout the vessel containing the reaction. In some cases, compared to conventional heating methods, heating using by applying microwave may result in more uniform heating. Other exemplary advantages of applying microwave energy includes accelerating reaction rates, improving reaction yields, and achieve more reproducible reactions. In some embodiments, microwave instruments available may provide controllable, reproducible and fast heating, such as of a fixed temperature, under certain conditions. In some aspects, rapid cooling down of the reaction can take place. In some embodiments, application of microwave energy allows for reactions to occur with greater uniformity, with reduced side reactions (e.g., reduced degradation of reactants or products). In some embodiments, the provided methods include a reaction that is temperature-monitored. In some embodiments, active cooling is applied to the reaction.

Numerous specific details are set forth in the following description in order to provide a thorough understanding of the present disclosure. These details are provided for the purpose of example and the claimed subject matter may be practiced according to the claims without some or all of these specific details. It is to be understood that other embodiments can be used and structural changes can be made without departing from the scope of the claimed subject matter. It should be understood that the various features and functionality described in one or more of the individual embodiments are not limited in their applicability to the particular embodiment with which they are described. They instead can, be applied, alone or in some combination, to one or more of the other embodiments of the disclosure, whether or not such embodiments are described, and whether or not such features are presented as being a part of a described embodiment. For the purpose of clarity, technical material that is known in the technical fields related to the claimed subject matter has not been described in detail so that the claimed subject matter is not unnecessarily obscured.

All publications, including patent documents, scientific articles and databases, referred to in this application are incorporated by reference in their entireties for all purposes to the same extent as if each individual publication were individually incorporated by reference. Citation of the publications or documents is not intended as an admission that any of them is pertinent prior art, nor does it constitute any admission as to the contents or date of these publications or documents.

All headings are for the convenience of the reader and should not be used to limit the meaning of the text that follows the heading, unless so specified.

### Definitions

Unless defined otherwise, all technical and scientific terms used herein have the same meaning as is commonly understood by one of ordinary skill in the art to which the present disclosure belongs. If a definition set forth in this section is contrary to or otherwise inconsistent with a definition set forth in the patents, applications, published applications and other publications that are herein incorporated by reference, the definition set forth in this section prevails over the definition that is incorporated herein by reference.

As used herein, the singular forms “a,” “an” and “the” include plural referents unless the context clearly dictates otherwise. Thus, for example, reference to “a peptide” includes one or more peptides, or mixtures of peptides. Also, and unless specifically stated or obvious from context, as used herein, the term “or” is understood to be inclusive and covers both “or” and “and”.

The term “about” as used herein refers to the usual error range for the respective value readily known to the skilled person in this technical field. Reference to “about” a value or parameter herein includes (and describes) embodiments that are directed to that value or parameter per se. For example, description referring to “about X” includes description of “X.

The term “antibody” herein is used in the broadest sense and includes polyclonal and monoclonal antibodies, including intact antibodies and functional (antigen-binding) antibody fragments, including fragment antigen binding (Fab) fragments, F(ab′)2 fragments, Fab′ fragments, Fv fragments, recombinant IgG (rIgG) fragments, single chain antibody fragments, including single chain variable fragments (scFv), and single domain antibodies (e.g., sdAb, sdFv, nanobody) fragments. The term encompasses genetically engineered and/or otherwise modified forms of immunoglobulins, such as intrabodies, peptibodies, chimeric antibodies, fully human antibodies, humanized antibodies, and heteroconjugate antibodies, multispecific, e.g., bispecific, antibodies, diabodies, triabodies, and tetrabodies, tandem di-scFv, tandem tri-scFv. Unless otherwise stated, the term “antibody” should be understood to encompass functional antibody fragments thereof. The term also encompasses intact or full-length antibodies, including antibodies of any class or sub-class, including IgG and sub-classes thereof, IgM, IgE, IgA, and IgD.

An “individual” or “subject” includes a mammal. Mammals include, but are not limited to, domesticated animals (e.g., cows, sheep, cats, dogs, and horses), primates (e.g., humans and non-human primates such as monkeys), rabbits, and rodents (e.g., mice and rats). An “individual” or “subject” may include birds such as chickens, vertebrates such as fish and mammals such as mice, rats, rabbits, cats, dogs, pigs, cows, ox, sheep, goats, horses, monkeys and other non-human primates. In certain embodiments, the individual or subject is a human.

As used herein, the term “sample” refers to anything which may contain an analyte for which an analyte assay is desired. As used herein, a “sample” can be a solution, a suspension, liquid, powder, a paste, aqueous, non-aqueous or any combination thereof. The sample may be a biological sample, such as a biological fluid or a biological tissue. Examples of biological fluids include urine, blood, plasma, serum, saliva, semen, stool, sputum, cerebral spinal fluid, tears, mucus, amniotic fluid or the like. Biological tissues are aggregate of cells, usually of a particular kind together with their intercellular substance that form one of the structural materials of a human, animal, plant, bacterial, fungal or viral structure, including connective, epithelium, muscle and nerve tissues. Examples of biological tissues also include organs, tumors, lymph nodes, arteries and individual cell(s).

In some embodiments, the sample is a biological sample. A biological sample of the present disclosure encompasses a sample in the form of a solution, a suspension, a liquid, a powder, a paste, an aqueous sample, or a non-aqueous sample. As used herein, a “biological sample” includes any sample obtained from a living or viral (or prion) source or other source of macromolecules and biomolecules, and includes any cell type or tissue of a subject from which nucleic acid, protein and/or other macromolecule can be obtained. The biological sample can be a sample obtained directly from a biological source or a sample that is processed. For example, isolated nucleic acids that are amplified constitute a biological sample. Biological samples include, but are not limited to, body fluids, such as blood, plasma, serum, cerebrospinal fluid, synovial fluid, urine and sweat, tissue and organ samples from animals and plants and processed samples derived therefrom. In some embodiments, the sample can be derived from a tissue or a body fluid, for example, a connective, epithelium, muscle or nerve tissue; a tissue selected from the group consisting of brain, lung, liver, spleen, bone marrow, thymus, heart, lymph, blood, bone, cartilage, pancreas, kidney, gall bladder, stomach, intestine, testis, ovary, uterus, rectum, nervous system, gland, and internal blood vessels; or a body fluid selected from the group consisting of blood, urine, saliva, bone marrow, sperm, an ascitic fluid, and subfractions thereof, e.g., serum or plasma.

The terms “level” or “levels” are used to refer to the presence and/or amount of a target, e.g., a substance or an organism that is part of the etiology of a disease or disorder, and can be determined qualitatively or quantitatively. A “qualitative” change in the target level refers to the appearance or disappearance of a target that is not detectable or is present in samples obtained from normal controls. A “quantitative” change in the levels of one or more targets refers to a measurable increase or decrease in the target levels when compared to a healthy control.

As used herein, the term “macromolecule” encompasses large molecules composed of smaller subunits. Examples of macromolecules include, but are not limited to peptides, polypeptides, proteins, nucleic acids, carbohydrates, lipids, macrocycles. A macromolecule also includes a chimeric macromolecule composed of a combination of two or more types of macromolecules, covalently linked together (e.g., a peptide linked to a nucleic acid). A macromolecule may also include a “macromolecule assembly”, which is composed of non-covalent complexes of two or more macromolecules. A macromolecule assembly may be composed of the same type of macromolecule (e.g., protein-protein) or of two more different types of macromolecules (e.g., protein-DNA).

As used herein, the term “polypeptide” encompasses peptides and proteins, and refers to a molecule comprising a chain of two or more amino acids joined by peptide bonds. In some embodiments, a polypeptide comprises 2 to 50 amino acids, e.g., having more than 20-30 amino acids. In some embodiments, a peptide does not comprise a secondary, tertiary, or higher structure. In some embodiments, the polypeptide is a protein. In some embodiments, a protein comprises 30 or more amino acids, e.g. having more than 50 amino acids. In some embodiments, in addition to a primary structure, a protein comprises a secondary, tertiary, or higher structure. The amino acids of the polypeptides are most typically L-amino acids, but may also be D-amino acids, modified amino acids, amino acid analogs, amino acid mimetics, or any combination thereof. Polypeptides may be naturally occurring, synthetically produced, or recombinantly expressed. Polypeptides may be synthetically produced, isolated, recombinantly expressed, or be produced by a combination of methodologies as described above. Polypeptides may also comprise additional groups modifying the amino acid chain, for example, functional groups added via post-translational modification. The polymer may be linear or branched, it may comprise modified amino acids, and it may be interrupted by non-amino acids. The term also encompasses an amino acid polymer that has been modified naturally or by intervention; for example, disulfide bond formation, glycosylation, lipidation, acetylation, phosphorylation, or any other manipulation or modification, such as conjugation with a labeling component.

As used herein, the term “amino acid” refers to an organic compound comprising an amine group, a carboxylic acid group, and a side-chain specific to each amino acid, which serve as a monomeric subunit of a peptide. An amino acid includes the 20 standard, naturally occurring or canonical amino acids as well as non-standard amino acids. The standard, naturally-occurring amino acids include Alanine (A or Ala), Cysteine (C or Cys), Aspartic Acid (D or Asp), Glutamic Acid (E or Glu), Phenylalanine (F or Phe), Glycine (G or Gly), Histidine (H or His), Isoleucine (I or Ile), Lysine (K or Lys), Leucine (L or Leu), Methionine (M or Met), Asparagine (N or Asn), Proline (P or Pro), Glutamine (Q or Gln), Arginine (R or Arg), Serine (S or Ser), Threonine (T or Thr), Valine (V or Val), Tryptophan (W or Trp), and Tyrosine (Y or Tyr). An amino acid may be an L-amino acid or a D-amino acid. Non-standard amino acids may be modified amino acids, amino acid analogs, amino acid mimetics, non-standard proteinogenic amino acids, or non-proteinogenic amino acids that occur naturally or are chemically synthesized. Examples of non-standard amino acids include, but are not limited to, selenocysteine, pyrrolysine, and N-formylmethionine, n-amino acids, Homo-amino acids, Proline and Pyruvic acid derivatives, 3-substituted alanine derivatives, glycine derivatives, ring-substituted phenylalanine and tyrosine derivatives, linear core amino acids, N-methyl amino acids.

As used herein, the term “post-translational modification” refers to modifications that occur on a peptide after its translation by ribosomes is complete. A post-translational modification may be a covalent chemical modification or enzymatic modification. Examples of post-translation modifications include, but are not limited to, acylation, acetylation, alkylation (including methylation), biotinylation, butyrylation, carbamylation, carbonylation, deamidation, deiminiation, diphthamide formation, disulfide bridge formation, eliminylation, flavin attachment, formylation, gamma-carboxylation, glutamylation, glycylation, glycosylation, glypiation, heme C attachment, hydroxylation, hypusine formation, iodination, isoprenylation, lipidation, lipoylation, malonylation, methylation, myristolylation, oxidation, palmitoylation, pegylation, phosphopantetheinylation, phosphorylation, prenylation, propionylation, retinylidene Schiff base formation, S-glutathionylation, S-nitrosylation, S-sulfenylation, selenation, succinylation, sulfination, ubiquitination, and C-terminal amidation. A post-translational modification includes modifications of the amino terminus and/or the carboxyl terminus of a peptide. Modifications of the terminal amino group include, but are not limited to, des-amino, N-lower alkyl, N-di-lower alkyl, and N-acyl modifications. Modifications of the terminal carboxy group include, but are not limited to, amide, lower alkyl amide, dialkyl amide, and lower alkyl ester modifications (e.g., wherein lower alkyl is C₁-C₄ alkyl). A post-translational modification also includes modifications, such as but not limited to those described above, of amino acids falling between the amino and carboxy termini. The term post-translational modification can also include peptide modifications that include one or more detectable labels.

As used herein, the term “binding agent” refers to a nucleic acid molecule, a peptide, a polypeptide, a protein, a carbohydrate, or a small molecule that binds to, associates, unites with, recognizes, or combines with a polypeptide or a component or feature of a polypeptide. A binding agent may form a covalent association or non-covalent association with the polypeptide or component or feature of a polypeptide. A binding agent may also be a chimeric binding agent, composed of two or more types of molecules, such as a nucleic acid molecule-peptide chimeric binding agent or a carbohydrate-peptide chimeric binding agent. A binding agent may be a naturally occurring, synthetically produced, or recombinantly expressed molecule. A binding agent may bind to a single monomer or subunit of a polypeptide (e.g., a single amino acid of a polypeptide) or bind to a plurality of linked subunits of a polypeptide (e.g., a di-peptide, tri-peptide, or higher order peptide of a longer peptide, polypeptide, or protein molecule). A binding agent may bind to a linear molecule or a molecule having a three-dimensional structure (also referred to as conformation). For example, an antibody binding agent may bind to linear peptide, polypeptide, or protein, or bind to a conformational peptide, polypeptide, or protein. A binding agent may bind to an N-terminal peptide, a C-terminal peptide, or an intervening peptide of a peptide, polypeptide, or protein molecule. A binding agent may bind to an N-terminal amino acid, C-terminal amino acid, or an intervening amino acid of a peptide molecule. A binding agent may bind to an N-terminal or C-terminal diamino acid moiety. A binding agent may preferably bind to a chemically modified or labeled amino acid (e.g., an amino acid that has been functionalized by a reagent comprising a compound of any one of Formula (I)-(VII) as described herein) over a non-modified or unlabeled amino acid. For example, a binding agent may preferably bind to an amino acid that has been functionalized with an acetyl moiety, guanyl moiety, dansyl moiety, PTC or derivatized PTC moiety, DNP moiety, SNP moiety, guanidinyl moiety, etc., over an amino acid that does not possess said moiety. A binding agent may bind to a post-translational modification of a peptide molecule. A binding agent may exhibit selective binding to a component or feature of a polypeptide (e.g., a binding agent may selectively bind to one of the 20 possible natural amino acid residues and with bind with very low affinity or not at all to the other 19 natural amino acid residues). A binding agent may exhibit less selective binding, where the binding agent is capable of binding a plurality of components or features of a polypeptide (e.g., a binding agent may bind with similar affinity to two or more different amino acid residues). A binding agent comprises a coding tag, which may be joined to the binding agent by a linker.

As used herein, the term “linker” refers to one or more of a nucleotide, a nucleotide analog, an amino acid, a peptide, a polypeptide, or a non-nucleotide chemical moiety that is used to join two molecules. A linker may be used to join a binding agent with a coding tag, a recording tag with a polypeptide, a polypeptide with a solid support, a recording tag with a solid support, etc. In certain embodiments, a linker joins two molecules via enzymatic reaction or chemistry reaction (e.g., click chemistry).

As used herein, the term “proteome” can include the entire set of proteins, polypeptides, or peptides (including conjugates or complexes thereof) expressed by a genome, cell, tissue, or organism at a certain time, of any organism. In one aspect, it is the set of expressed proteins in a given type of cell or organism, at a given time, under defined conditions. Proteomics is the study of the proteome. For example, a “cellular proteome” may include the collection of proteins found in a particular cell type under a particular set of environmental conditions, such as exposure to hormone stimulation. An organism's complete proteome may include the complete set of proteins from all of the various cellular proteomes. A proteome may also include the collection of proteins in certain sub-cellular biological systems. For example, all of the proteins in a virus can be called a viral proteome. As used herein, the term “proteome” include subsets of a proteome, including but not limited to a kinome; a secretome; a receptome (e.g., GPCRome); an immunoproteome; a nutriproteome; a proteome subset defined by a post-translational modification (e.g., phosphorylation, ubiquitination, methylation, acetylation, glycosylation, oxidation, lipidation, and/or nitrosylation), such as a phosphoproteome (e.g., phosphotyrosine-proteome, tyrosine-kinome, and tyrosine-phosphatome), a glycoproteome, etc.; a proteome subset associated with a tissue or organ, a developmental stage, or a physiological or pathological condition; a proteome subset associated a cellular process, such as cell cycle, differentiation (or de-differentiation), cell death, senescence, cell migration, transformation, or metastasis; or any combination thereof. As used herein, the term “proteomics” refers to quantitative analysis of the proteome within cells, tissues, and bodily fluids, and the corresponding spatial distribution of the proteome within the cell and within tissues. Additionally, proteomics studies include the dynamic state of the proteome, continually changing in time as a function of biology and defined biological or chemical stimuli.

As used herein, the term “non-cognate binding agent” refers to a binding agent that is not capable of binding or binds with low affinity to a polypeptide feature, component, or subunit being interrogated in a particular binding cycle reaction as compared to a “cognate binding agent”, which binds with high affinity to the corresponding polypeptide feature, component, or subunit. For example, if a tyrosine residue of a peptide molecule is being interrogated in a binding reaction, non-cognate binding agents are those that bind with low affinity or not at all to the tyrosine residue, such that the non-cognate binding agent does not efficiently transfer coding tag information to the recording tag under conditions that are suitable for transferring coding tag information from cognate binding agents to the recording tag. Alternatively, if a tyrosine residue of a peptide molecule is being interrogated in a binding reaction, non-cognate binding agents are those that bind with low affinity or not at all to the tyrosine residue, such that recording tag information does not efficiently transfer to the coding tag under suitable conditions for those embodiments involving extended coding tags rather than extended recording tags.

The terminal amino acid at one end of the peptide chain that has a free amino group is referred to herein as the “N-terminal amino acid” (NTAA). The terminal amino acid at the other end of the chain that has a free carboxyl group is referred to herein as the “C-terminal amino acid” (CTAA). The amino acids making up a peptide may be numbered in order, with the peptide being “n” amino acids in length. As used herein, NTAA is considered the n^{th} amino acid (also referred to herein as the “n NTAA”). Using this nomenclature, the next amino acid is the n-1 amino acid, then the n-2 amino acid, and so on down the length of the peptide from the N-terminal end to C-terminal end. In certain embodiments, an NTAA, CTAA, or both may be functionalized with a chemical moiety.

As used herein, the term “barcode” refers to a nucleic acid molecule of about 2 to about 30 bases (e.g., 2, 3, 4, 5, 6, 7, 8, 9, 10, 11, 12, 13, 14, 15, 16, 17, 18, 19, 20, 21, 22, 23, 24, 25, 26, 27, 28, 29 or 30 bases) providing a unique identifier tag or origin information for a polypeptide, a binding agent, a set of binding agents from a binding cycle, a sample polypeptides, a set of samples, polypeptides within a compartment (e.g., droplet, bead, or separated location), polypeptides within a set of compartments, a fraction of polypeptides, a set of polypeptide fractions, a spatial region or set of spatial regions, a library of polypeptides, or a library of binding agents. A barcode can be an artificial sequence or a naturally occurring sequence. In certain embodiments, each barcode within a population of barcodes is different. In other embodiments, a portion of barcodes in a population of barcodes is different, e.g, at least about 10%, 15%, 20%, 25%, 30%, 35%, 40%, 45%, 50%, 55%, 60%, 65%, 70%, 75%, 80%, 85%, 90%, 95%, 97%, or 99% of the barcodes in a population of barcodes is different. A population of barcodes may be randomly generated or non-randomly generated. In certain embodiments, a population of barcodes are error correcting barcodes. Barcodes can be used to computationally deconvolute the multiplexed sequencing data and identify sequence reads derived from an individual polypeptide, sample, library, etc. A barcode can also be used for deconvolution of a collection of polypeptides that have been distributed into small compartments for enhanced mapping. For example, rather than mapping a peptide back to the proteome, the peptide is mapped back to its originating protein molecule or protein complex.

A “sample barcode”, also referred to as “sample tag” identifies from which sample a polypeptide derives.

A “spatial barcode” identifies which region of a 2-D or 3-D tissue section from which a polypeptide derives. Spatial barcodes may be used for molecular pathology on tissue sections. A spatial barcode allows for multiplex sequencing of a plurality of samples or libraries from tissue section(s).

As used herein, the term “coding tag” refers to a polynucleotide with any suitable length, e.g., a nucleic acid molecule of about 2 bases to about 100 bases, including any integer including 2 and 100 and in between, that comprises identifying information for its associated binding agent. A “coding tag” may also be made from a “sequenceable polymer” (see, e.g., Niu et al., 2013, Nat. Chem. 5:282-292; Roy et al., 2015, Nat. Commun. 6:7237; Lutz et al., 2015, Macromolecules 48:4759-4767; each of which are incorporated by reference in its entirety). A coding tag may comprise an encoder sequence, which is optionally flanked by one spacer on one side or optionally flanked by a spacer on each side. A coding tag may also be comprised of an optional UMI and/or an optional binding cycle-specific barcode. A coding tag may be single stranded or double stranded. A double stranded coding tag may comprise blunt ends, overhanging ends, or both. A coding tag may refer to the coding tag that is directly attached to a binding agent, to a complementary sequence hybridized to the coding tag directly attached to a binding agent (e.g., for double stranded coding tags), or to coding tag information present in an extended recording tag. In certain embodiments, a coding tag may further comprise a binding cycle specific spacer or barcode, a unique molecular identifier, a universal priming site, or any combination thereof.

As used herein, the term “spacer” (Sp) refers to a nucleic acid molecule of about 1 base to about 20 bases (e.g., 1, 2, 3, 4, 5, 6, 7, 8, 9, 10, 11, 12, 13, 14, 15, 16, 17, 18, 19, or 20 bases) in length that is present on a terminus of a recording tag or coding tag. In certain embodiments, a spacer sequence flanks an encoder sequence of a coding tag on one end or both ends. Following binding of a binding agent to a polypeptide, annealing between complementary spacer sequences on their associated coding tag and recording tag, respectively, allows transfer of binding information through a primer extension reaction or ligation to the recording tag, coding tag, or a di-tag construct. Sp′ refers to spacer sequence complementary to Sp. Preferably, spacer sequences within a library of binding agents possess the same number of bases. A common (shared or identical) spacer may be used in a library of binding agents. A spacer sequence may have a “cycle specific” sequence in order to track binding agents used in a particular binding cycle. The spacer sequence (Sp) can be constant across all binding cycles, be specific for a particular class of polypeptides, or be binding cycle number specific. Polypeptide class-specific spacers permit annealing of a cognate binding agent's coding tag information present in an extended recording tag from a completed binding/extension cycle to the coding tag of another binding agent recognizing the same class of polypeptides in a subsequent binding cycle via the class-specific spacers. Only the sequential binding of correct cognate pairs results in interacting spacer elements and effective primer extension. A spacer sequence may comprise sufficient number of bases to anneal to a complementary spacer sequence in a recording tag to initiate a primer extension (also referred to as polymerase extension) reaction, or provide a “splint” for a ligation reaction, or mediate a “sticky end” ligation reaction. A spacer sequence may comprise a fewer number of bases than the encoder sequence within a coding tag.

As used herein, the term “recording tag” refers to a moiety, e.g., a chemical coupling moiety, a nucleic acid molecule, or a sequenceable polymer molecule (see, e.g., Niu et al., 2013, Nat. Chem. 5:282-292; Roy et al., 2015, Nat. Commun. 6:7237; Lutz, 2015, Macromolecules 48:4759-4767; each of which are incorporated by reference in its entirety) to which identifying information of a coding tag can be transferred, or from which identifying information about the macromolecule (e.g., UMI information) associated with the recording tag can be transferred to the coding tag. Identifying information can comprise any information characterizing a molecule such as information pertaining to sample, fraction, partition, spatial location, interacting neighboring molecule(s), cycle number, etc. Additionally, the presence of UMI information can also be classified as identifying information. In certain embodiments, after a binding agent binds a polypeptide, information from a coding tag linked to a binding agent can be transferred to the recording tag associated with the polypeptide while the binding agent is bound to the polypeptide. In other embodiments, after a binding agent binds a polypeptide, information from a recording tag associated with the polypeptide can be transferred to the coding tag linked to the binding agent while the binding agent is bound to the polypeptide. A recoding tag may be directly linked to a polypeptide, linked to a polypeptide via a multifunctional linker, or associated with a polypeptide by virtue of its proximity (or co-localization) on a solid support. A recording tag may be linked via its 5′ end or 3′ end or at an internal site, as long as the linkage is compatible with the method used to transfer coding tag information to the recording tag or vice versa. A recording tag may further comprise other functional components, e.g., a universal priming site, unique molecular identifier, a barcode (e.g., a sample barcode, a fraction barcode, spatial barcode, a compartment tag, etc.), a spacer sequence that is complementary to a spacer sequence of a coding tag, or any combination thereof. The spacer sequence of a recording tag is preferably at the 3′-end of the recording tag in embodiments where polymerase extension is used to transfer coding tag information to the recording tag.

As used herein, the term “primer extension”, also referred to as “polymerase extension”, refers to a reaction catalyzed by a nucleic acid polymerase (e.g., DNA polymerase) whereby a nucleic acid molecule (e.g., oligonucleotide primer, spacer sequence) that anneals to a complementary strand is extended by the polymerase, using the complementary strand as template.

As used herein, the term “unique molecular identifier” or “UMI” refers to a nucleic acid molecule of about 3 to about 40 bases (3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 bases in length providing a unique identifier tag for each polypeptide or binding agent to which the UMI is linked. A polypeptide UMI can be used to computationally deconvolute sequencing data from a plurality of extended recording tags to identify extended recording tags that originated from an individual polypeptide. A polypeptide UMI can be used to accurately count originating polypeptide molecules by collapsing NGS reads to unique UMIs. A binding agent UMI can be used to identify each individual molecular binding agent that binds to a particular polypeptide. For example, a UMI can be used to identify the number of individual binding events for a binding agent specific for a single amino acid that occurs for a particular peptide molecule. It is understood that when UMI and barcode are both referenced in the context of a binding agent or polypeptide, that the barcode refers to identifying information other that the UMI for the individual binding agent or polypeptide (e.g., sample barcode, compartment barcode, binding cycle barcode).

As used herein, the term “universal priming site” or “universal primer” or “universal priming sequence” refers to a nucleic acid molecule, which may be used for library amplification and/or for sequencing reactions. A universal priming site may include, but is not limited to, a priming site (primer sequence) for PCR amplification, flow cell adaptor sequences that anneal to complementary oligonucleotides on flow cell surfaces enabling bridge amplification in some next generation sequencing platforms, a sequencing priming site, or a combination thereof. Universal priming sites can be used for other types of amplification, including those commonly used in conjunction with next generation digital sequencing. For example, extended recording tag molecules may be circularized and a universal priming site used for rolling circle amplification to form DNA nanoballs that can be used as sequencing templates (Drmanac et al., 2009, Science 327:78-81). Alternatively, recording tag molecules may be circularized and sequenced directly by polymerase extension from universal priming sites (Korlach et al., 2008, Proc. Natl. Acad. Sci. 105:1176-1181). The term “forward” when used in context with a “universal priming site” or “universal primer” may also be referred to as “5” or “sense”. The term “reverse” when used in context with a “universal priming site” or “universal primer” may also be referred to as “3′” or “antisense”.

As used herein, the term “extended recording tag” refers to a recording tag to which information of at least one binding agent's coding tag (or its complementary sequence) has been transferred following binding of the binding agent to a polypeptide. Information of the coding tag may be transferred to the recording tag directly (e.g., ligation) or indirectly (e.g., primer extension). Information of a coding tag may be transferred to the recording tag enzymatically or chemically. An extended recording tag may comprise binding agent information of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 55, 60, 65, 70, 75, 80, 85, 90, 95, 100, 125, 150, 175, 200 or more coding tags. The base sequence of an extended recording tag may reflect the temporal and sequential order of binding of the binding agents identified by their coding tags, may reflect a partial sequential order of binding of the binding agents identified by the coding tags, or may not reflect any order of binding of the binding agents identified by the coding tags. In certain embodiments, the coding tag information present in the extended recording tag represents with at least 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, or 100% identity the polypeptide sequence being analyzed. In certain embodiments where the extended recording tag does not represent the polypeptide sequence being analyzed with 100% identity, errors may be due to off-target binding by a binding agent, or to a “missed” binding cycle (e.g., because a binding agent fails to bind to a polypeptide during a binding cycle, because of a failed primer extension reaction), or both.

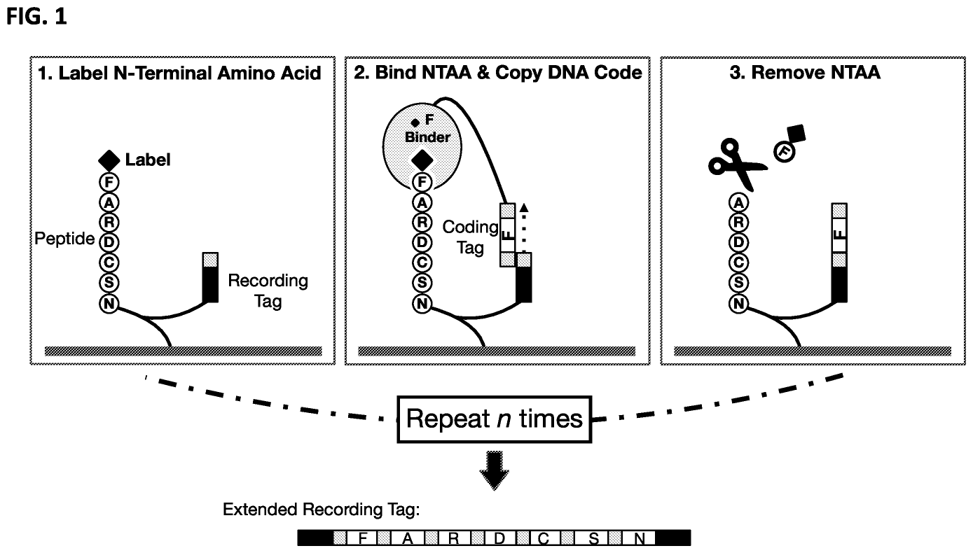

As used herein, the term “extended coding tag” refers to a coding tag to which information of at least one recording tag (or its complementary sequence) has been transferred following binding of a binding agent, to which the coding tag is joined, to a polypeptide, to which the recording tag is associated. Information of a recording tag may be transferred to the coding tag directly (e.g., ligation), or indirectly (e.g., primer extension). Information of a recording tag may be transferred enzymatically or chemically. In certain embodiments, an extended coding tag comprises information of one recording tag, reflecting one binding event. As used herein, the term “di-tag” or “di-tag construct” or “di-tag molecule” refers to a nucleic acid molecule to which information of at least one recording tag (or its complementary sequence) and at least one coding tag (or its complementary sequence) has been transferred following binding of a binding agent, to which the coding tag is joined, to a polypeptide, to which the recording tag is associated (see, e.g., FIG. 1). Information of a recording tag and coding tag may be transferred to the di-tag indirectly (e.g., primer extension). Information of a recording tag may be transferred enzymatically or chemically. In certain embodiments, a di-tag comprises a UMI of a recording tag, a compartment tag of a recording tag, a universal priming site of a recording tag, a UMI of a coding tag, an encoder sequence of a coding tag, a binding cycle specific barcode, a universal priming site of a coding tag, or any combination thereof.

As used herein, the term “solid support”, “solid surface”, or “solid substrate”, or “sequencing substrate”, or “substrate” refers to any solid material, including porous and non-porous materials, to which a polypeptide can be associated directly or indirectly, by any means known in the art, including covalent and non-covalent interactions, or any combination thereof. A solid support may be two-dimensional (e.g., planar surface) or three-dimensional (e.g., gel matrix or bead). A solid support can be any support surface including, but not limited to, a bead, a microbead, an array, a glass surface, a silicon surface, a plastic surface, a filter, a membrane, a PTFE membrane, nylon, a silicon wafer chip, a flow through chip, a flow cell, a biochip including signal transducing electronics, a channel, a microtiter well, an ELISA plate, a spinning interferometry disc, a nitrocellulose membrane, a nitrocellulose-based polymer surface, a polymer matrix, a nanoparticle, or a microsphere. Materials for a solid support include but are not limited to acrylamide, agarose, cellulose, nitrocellulose, glass, gold, quartz, polystyrene, polyethylene vinyl acetate, polypropylene, polyester, polymethacrylate, polyacrylate, polyethylene, polyethylene oxide, polysilicates, polycarbonates, poly vinyl alcohol (PVA), Teflon, fluorocarbons, nylon, silicon rubber, polyanhydrides, polyglycolic acid, polylactic acid, polyorthoesters, functionalized silane, polypropylfumerate, collagen, glycosaminoglycans, polyamino acids, dextran, or any combination thereof. Solid supports further include thin film, membrane, bottles, dishes, fibers, woven fibers, shaped polymers such as tubes, particles, beads, microspheres, microparticles, or any combination thereof. For example, when solid surface is a bead, the bead can include, but is not limited to, a ceramic bead, polystyrene bead, a polymer bead, a methylstyrene bead, a polyacrylate bead, an agarose bead, a cellulose bead, a dextran bead, an acrylamide bead, a solid core bead, a porous bead, a paramagnetic bead, a glass bead, a silica-based bead, a controlled pore bead, or any combinations thereof. A bead may be spherical or an irregularly shaped. A bead or support may be porous. A bead's size may range from nanometers, e.g., 100 nm, to millimeters, e.g., 1 mm. In certain embodiments, beads range in size from about 0.2 micron to about 200 microns, or from about 0.5 micron to about 5 micron. In some embodiments, beads can be about 1, 1.5, 2, 2.5, 2.8, 3, 3.5, 4, 4.5, 5, 5.5, 6, 6.5, 7, 7.5, 8, 8.5, 9, 9.5, 10, 10.5, 15, or 20 μm in diameter. In certain embodiments, “a bead” solid support may refer to an individual bead or a plurality of beads. In some embodiments, the solid surface is a nanoparticle. In certain embodiments, the nanoparticles range in size from about 1 nm to about 500 nm in diameter, for example, between about 1 nm and about 20 nm, between about 1 nm and about 50 nm, between about 1 nm and about 100 nm, between about 10 nm and about 50 nm, between about 10 nm and about 100 nm, between about 10 nm and about 200 nm, between about 50 nm and about 100 nm, between about 50 nm and about 150, between about 50 nm and about 200 nm, between about 100 nm and about 200 nm, or between about 200 nm and about 500 nm in diameter. In some embodiments, the nanoparticles can be about 10 nm, about 50 nm, about 100 nm, about 150 nm, about 200 nm, about 300 nm, or about 500 nm in diameter. In some embodiments, the nanoparticles are less than about 200 nm in diameter.

As used herein, the term “nucleic acid molecule” or “polynucleotide” refers to a single- or double-stranded polynucleotide containing deoxyribonucleotides or ribonucleotides that are linked by 3 ‘-5’ phosphodiester bonds, as well as polynucleotide analogs. A nucleic acid molecule includes, but is not limited to, DNA, RNA, and cDNA. A polynucleotide analog may possess a backbone other than a standard phosphodiester linkage found in natural polynucleotides and, optionally, a modified sugar moiety or moieties other than ribose or deoxyribose. Polynucleotide analogs contain bases capable of hydrogen bonding by Watson-Crick base pairing to standard polynucleotide bases, where the analog backbone presents the bases in a manner to permit such hydrogen bonding in a sequence-specific fashion between the oligonucleotide analog molecule and bases in a standard polynucleotide. Examples of polynucleotide analogs include, but are not limited to xeno nucleic acid (XNA), bridged nucleic acid (BNA), glycol nucleic acid (GNA), peptide nucleic acids (PNAs), yPNAs, morpholino polynucleotides, locked nucleic acids (LNAs), threose nucleic acid (TNA), 2′-O-Methyl polynucleotides, 2′-O-alkyl ribosyl substituted polynucleotides, phosphorothioate polynucleotides, and boronophosphate polynucleotides. A polynucleotide analog may possess purine or pyrimidine analogs, including for example, 7-deaza purine analogs, 8-halopurine analogs, 5-halopyrimidine analogs, or universal base analogs that can pair with any base, including hypoxanthine, nitroazoles, isocarbostyril analogues, azole carboxamides, and aromatic triazole analogues, or base analogs with additional functionality, such as a biotin moiety for affinity binding. In some embodiments, the nucleic acid molecule or oligonucleotide is a modified oligonucleotide. In some embodiments, the nucleic acid molecule or oligonucleotide is a DNA with pseudo-complementary bases, a DNA with protected bases, an RNA molecule, a BNA molecule, an XNA molecule, a LNA molecule, a PNA molecule, a γPNA molecule, or a morpholino DNA, or a combination thereof. In some embodiments, the nucleic acid molecule or oligonucleotide is backbone modified, sugar modified, or nucleobase modified. In some embodiments, the nucleic acid molecule or oligonucleotide has nucleobase protecting groups such as Alloc, electrophilic protecting groups such as thiranes, acetyl protecting groups, nitrobenzyl protecting groups, sulfonate protecting groups, or traditional base-labile protecting groups.

As used herein, “nucleic acid sequencing” means the determination of the order of nucleotides in a nucleic acid molecule or a sample of nucleic acid molecules.

As used herein, “next generation sequencing” refers to high-throughput sequencing methods that allow the sequencing of millions to billions of molecules in parallel. Examples of next generation sequencing methods include sequencing by synthesis, sequencing by ligation, sequencing by hybridization, polony sequencing, ion semiconductor sequencing, and pyrosequencing. By attaching primers to a solid substrate and a complementary sequence to a nucleic acid molecule, a nucleic acid molecule can be hybridized to the solid substrate via the primer and then multiple copies can be generated in a discrete area on the solid substrate by using polymerase to amplify (these groupings are sometimes referred to as polymerase colonies or polonies). Consequently, during the sequencing process, a nucleotide at a particular position can be sequenced multiple times (e.g., hundreds or thousands of times)—this depth of coverage is referred to as “deep sequencing.” Examples of high throughput nucleic acid sequencing technology include platforms provided by Illumina, BGI, Qiagen, Thermo-Fisher, and Roche, including formats such as parallel bead arrays, sequencing by synthesis, sequencing by ligation, capillary electrophoresis, electronic microchips, “biochips,” microarrays, parallel microchips, and single-molecule arrays (Service (2006) Science 311:1544-1546,).

As used herein, “single molecule sequencing” or “third generation sequencing” refers to next-generation sequencing methods wherein reads from single molecule sequencing instruments are generated by sequencing of a single molecule of DNA. Unlike next generation sequencing methods that rely on amplification to clone many DNA molecules in parallel for sequencing in a phased approach, single molecule sequencing interrogates single molecules of DNA and does not require amplification or synchronization. Single molecule sequencing includes methods that need to pause the sequencing reaction after each base incorporation (‘wash-and-scan’ cycle) and methods which do not need to halt between read steps. Examples of single molecule sequencing methods include single molecule real-time sequencing (Pacific Biosciences), nanopore-based sequencing (Oxford Nanopore), duplex interrupted nanopore sequencing, and direct imaging of DNA using advanced microscopy.

As used herein, “analyzing” the polypeptide means to determine the presence or absence, identify, quantify, characterize, distinguish, or a combination thereof, all or a portion of the components of the polypeptide. For example, analyzing a peptide, polypeptide, or protein includes determining all or a portion of the amino acid sequence (contiguous or non-continuous) of the peptide. Analyzing a polypeptide also includes partial identification of a component of the polypeptide. For example, partial identification of amino acids in the polypeptide protein sequence can identify an amino acid in the protein as belonging to a subset of possible amino acids. Analysis typically begins with analysis of the n NTAA, and then proceeds to the next amino acid of the peptide (i.e., n-1, n-2, n-3, and so forth). This is accomplished by elimination of the n NTAA, thereby converting the n-1 amino acid of the peptide to an N-terminal amino acid (referred to herein as the “n-1 NTAA”). Analyzing the peptide may also include determining the presence and frequency of post-translational modifications on the peptide, which may or may not include information regarding the sequential order of the post-translational modifications on the peptide. Analyzing the peptide may also include determining the presence and frequency of epitopes in the peptide, which may or may not include information regarding the sequential order or location of the epitopes within the peptide. Analyzing the peptide may include combining different types of analysis, for example obtaining epitope information, amino acid sequence information, post-translational modification information, or any combination thereof.

As used herein, the term “compartment” refers to a physical area or volume that separates or isolates a subset of polypeptides from a sample of polypeptides. For example, a compartment may separate an individual cell from other cells, or a subset of a sample's proteome from the rest of the sample's proteome. A compartment may be an aqueous compartment (e.g., microfluidic droplet), a solid compartment (e.g., picotiter well or microtiter well on a plate, tube, vial, gel bead), a bead surface, a porous bead interior, or a separated region on a surface. A compartment may comprise one or more beads to which polypeptides may be immobilized.

As used herein, the term “compartment tag” or “compartment barcode” refers to a single or double stranded nucleic acid molecule of about 4 bases to about 100 bases (including 4 bases, 100 bases, and any integer between) that comprises identifying information for the constituents (e.g., a single cell's proteome), within one or more compartments (e.g., microfluidic droplet, bead surface). A compartment barcode identifies a subset of polypeptides in a sample that have been separated into the same physical compartment or group of compartments from a plurality (e.g., millions to billions) of compartments. Thus, a compartment tag can be used to distinguish constituents derived from one or more compartments having the same compartment tag from those in another compartment having a different compartment tag, even after the constituents are pooled together. By labeling the proteins and/or peptides within each compartment or within a group of two or more compartments with a unique compartment tag, peptides derived from the same protein, protein complex, or cell within an individual compartment or group of compartments can be identified. A compartment tag comprises a barcode, which is optionally flanked by a spacer sequence on one or both sides, and an optional universal primer. The spacer sequence can be complementary to the spacer sequence of a recording tag, enabling transfer of compartment tag information to the recording tag. A compartment tag may also comprise a universal priming site, a unique molecular identifier (for providing identifying information for the peptide attached thereto), or both, particularly for embodiments where a compartment tag comprises a recording tag to be used in downstream peptide analysis methods described herein. A compartment tag can comprise a functional moiety (e.g., aldehyde, NHS, mTet, alkyne, etc.) for coupling to a peptide. Alternatively, a compartment tag can comprise a peptide comprising a recognition sequence for a protein ligase to allow ligation of the compartment tag to a peptide of interest. A compartment can comprise a single compartment tag, a plurality of identical compartment tags save for an optional UMI sequence, or two or more different compartment tags. In certain embodiments each compartment comprises a unique compartment tag (one-to-one mapping). In other embodiments, multiple compartments from a larger population of compartments comprise the same compartment tag (many-to-one mapping). A compartment tag may be joined to a solid support within a compartment (e.g., bead) or joined to the surface of the compartment itself (e.g., surface of a picotiter well). Alternatively, a compartment tag may be free in solution within a compartment.

As used herein, the term “partition” refers to an assignment, e.g., random assignment, of a unique barcode to a subpopulation of polypeptides from a population of polypeptides within a sample. In certain embodiments, partitioning may be achieved by distributing polypeptides into compartments. A partition may be comprised of the polypeptides within a single compartment or the polypeptides within multiple compartments from a population of compartments.

As used herein, a “partition tag” or “partition barcode” refers to a single or double stranded nucleic acid molecule of about 4 bases to about 100 bases (including 4 bases, 100 bases, and any integer between) that comprises identifying information for a partition. In certain embodiments, a partition tag for a polypeptide refers to identical compartment tags arising from the partitioning of polypeptides into compartment(s) labeled with the same barcode.

As used herein, the term “fraction” refers to a subset of polypeptides within a sample that have been sorted from the rest of the sample or organelles using physical or chemical separation methods, such as fractionating by size, hydrophobicity, isoelectric point, affinity, and so on. Separation methods include HPLC separation, gel separation, affinity separation, cellular fractionation, cellular organelle fractionation, tissue fractionation, etc. Physical properties such as fluid flow, magnetism, electrical current, mass, density, or the like can also be used for separation.

As used herein, the term “fraction barcode” refers to a single or double stranded nucleic acid molecule of about 4 bases to about 100 bases (including 4 bases, 100 bases, and any integer therebetween) that comprises identifying information for the polypeptides within a fraction.

As used herein, the term “alkyl” refers to and includes saturated linear and branched univalent hydrocarbon structures and combination thereof, having the number of carbon atoms designated (i.e., C₁-C₁₀ means one to ten carbons). Particular alkyl groups are those having 1 to 20 carbon atoms (a “C₁-C₂₀ alkyl”). More particular alkyl groups are those having 1 to 8 carbon atoms (a “C₁-C₈ alkyl”), 3 to 8 carbon atoms (a “C₃-C₈ alkyl”), 1 to 6 carbon atoms (a “C₁-C₆ alkyl”), 1 to 5 carbon atoms (a “C₁-C₅ alkyl”), or 1 to 4 carbon atoms (a “C₁-C₄ alkyl”). Examples of alkyl include, but are not limited to, groups such as methyl, ethyl, n-propyl, isopropyl, n-butyl, t-butyl, isobutyl, sec-butyl, homologs and isomers of, for example, n-pentyl, n-hexyl, n-heptyl, n-octyl, and the like.

As used herein, “alkenyl” as used herein refers to an unsaturated linear or branched univalent hydrocarbon chain or combination thereof, having at least one site of olefinic unsaturation (i.e., having at least one moiety of the formula C═C) and having the number of carbon atoms designated (i.e., C₂-C₁₀ means two to ten carbon atoms). The alkenyl group may be in “cis” or “trans” configurations, or alternatively in “E” or “Z” configurations. Particular alkenyl groups are those having 2 to 20 carbon atoms (a “C₂-C₂₀ alkenyl”), having 2 to 8 carbon atoms (a “C₂-C₈ alkenyl”), having 2 to 6 carbon atoms (a “C₂-C₆ alkenyl”), or having 2 to 4 carbon atoms (a “C₂-C₄ alkenyl”). Examples of alkenyl include, but are not limited to, groups such as ethenyl (or vinyl), prop-1-enyl, prop-2-enyl (or allyl), 2-methylprop-1-enyl, but-1-enyl, but-2-enyl, but-3-enyl, buta-1,3-dienyl, 2-methylbuta-1,3-dienyl, homologs and isomers thereof, and the like.

The term “aminoalkyl” refers to an alkyl group that is substituted with one or more —NH₂ groups. In certain embodiments, an aminoalkyl group is substituted with one, two, three, four, five or more —NH₂ groups. An aminoalkyl group may optionally be substituted with one or more additional substituents as described herein.

As used herein, “aryl” or “Ar” refers to an unsaturated aromatic carbocyclic group having a single ring (e.g., phenyl) or multiple condensed rings (e.g., naphthyl or anthryl) which condensed rings may or may not be aromatic. In one variation, the aryl group contains from 6 to 14 annular carbon atoms. An aryl group having more than one ring where at least one ring is non-aromatic may be connected to the parent structure at either an aromatic ring position or at a non-aromatic ring position. In one variation, an aryl group having more than one ring where at least one ring is non-aromatic is connected to the parent structure at an aromatic ring position.

As used herein, the term “arylalkyl” refers to an aryl group, as defined herein, appended to the parent molecular moiety through an alkyl group, as defined herein. Representative examples of arylalkyl include, but are not limited to, benzyl, 2-phenylethyl, 3-phenylpropyl, 2-naphth-2-ylethyl, and the like.

As used herein, the term “cycloalkyl” refers to and includes cyclic univalent hydrocarbon structures, which may be fully saturated, mono- or polyunsaturated, but which are non-aromatic, having the number of carbon atoms designated (e.g., C₁-C₁₀ means one to ten carbons). Cycloalkyl can consist of one ring, such as cyclohexyl, or multiple rings, such as adamantly, but excludes aryl groups. A cycloalkyl comprising more than one ring may be fused, spiro or bridged, or combinations thereof. In some embodiments, the cycloalkyl is a cyclic hydrocarbon having from 3 to 13 annular carbon atoms. In some embodiments, the cycloalkyl is a cyclic hydrocarbon having from 3 to 8 annular carbon atoms (a “C₃-C₈ cycloalkyl”). Examples of cycloalkyl include, but are not limited to, cyclopropyl, cyclobutyl, cyclopentyl, cyclohexyl, 1-cyclohexenyl, 3-cyclohexenyl, cycloheptyl, norbomyl, and the like.

As used herein, the “halogen” represents chlorine, fluorine, bromine, or iodine. The term “halo” represents chloro, fluoro, bromo, or iodo.

The term “haloalkyl” refers to an alkyl group as described above, wherein one or more hydrogen atoms on the alkyl group have been substituted with a halo group. Examples of such groups include, without limitation, fluoroalkyl groups, such as fluoroethyl, trifluoromethyl, difluoromethyl, trifluoroethyl and the like.

As used herein, the term “heteroaryl” refers to and includes unsaturated aromatic cyclic groups having from 1 to 10 annular carbon atoms and at least one annular heteroatom, including but not limited to heteroatoms such as nitrogen, oxygen and sulfur, wherein the nitrogen and sulfur atoms are optionally oxidized, and the nitrogen atom(s) are optionally quaternized. A heteroaryl group can be attached to the remainder of the molecule at an annular carbon or at an annular heteroatom. Heteroaryl may contain additional fused rings (e.g., from 1 to 3 rings), including additionally fused aryl, heteroaryl, cycloalkyl, and/or heterocyclyl rings. Examples of heteroaryl groups include, but are not limited to, pyridyl, pyrimidyl, thiophenyl, furanyl, thiazolyl, and the like.

As used herein, the term “heterocycle”, “heterocyclic”, or “heterocyclyl” refers to a saturated or an unsaturated non-aromatic group having from 1 to 10 annular carbon atoms and from 1 to 4 annular heteroatoms, such as nitrogen, sulfur or oxygen, and the like, wherein the nitrogen and sulfur atoms are optionally oxidized, and the nitrogen atom(s) are optionally quaternized. A heterocyclyl group may have a single ring or multiple condensed rings, but excludes heteroaryl groups. A heterocycle comprising more than one ring may be fused, spiro or bridged, or any combination thereof. In fused ring systems, one or more of the fused rings can be aryl or heteroaryl. Examples of heterocyclyl groups include, but are not limited to, tetrahydropyranyl, dihydropyranyl, piperidinyl, piperazinyl, pynolidinyl, thiazolinyl, thiazolidinyl, tetrahydrofuranyl, tetrahydrothiophenyl, 2,3-dihydrobenzo[b]thiophen-2-yl, 4-amino-2-oxopyrimidin-1(2H)-yl, and the like.

The term “substituted” means that the specified group or moiety bears one or more substituents including, but not limited to, substituents such as alkoxy, acyl, acyloxy, carbonylalkoxy, acylamino, amino, aminoacyl, aminocarbonylamino, aminocarbonyloxy, cycloalkyl, cycloalkenyl, aryl, heteroaryl, aryloxy, cyano, azido, halo, hydroxyl, nitro, carboxyl, thiol, thioalkyl, cycloalkyl, cycloalkenyl, alkyl, alkenyl, alkynyl, heterocyclyl, aralkyl, aminosulfonyl, sulfonylamino, sulfonyl, oxo, carbonylalkylenealkoxy and the like. The term “unsubstituted” means that the specified group bears no substituents. The term “optionally substituted” means that the specified group is unsubstituted or substituted by one or more substituents. Where the term “substituted” is used to describe a structural system, the substitution is meant to occur at any valency-allowed position on the system.

It is understood that aspects and embodiments of the invention described herein include “consisting” and/or “consisting essentially of” aspects and embodiments.

Throughout this disclosure, various aspects of this invention are presented in a range format. It should be understood that the description in range format is merely for convenience and brevity and should not be construed as an inflexible limitation on the scope of the invention. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Other objects, advantages and features of the present invention will become apparent from the following specification taken in conjunction with the accompanying drawings

### I. Method of Accelerating a Reaction

Provided herein are methods of accelerating a reaction involving a polypeptide by applying radiation, e.g., electromagnetic radiation or microwave energy. In some embodiments, the accelerating is achieved with the application of microwave radiation. Also provided herein are methods for accelerating a sequencing reaction including preparing and/or treating a polypeptide. In some embodiments, the microwave energy is applied in the presence of ionic liquids. For example, the contacting of the polypeptide with a functionalizing reagent, binding agent, and/or removing reagents is performed in the presence of ionic liquids. In some embodiments, microwave energy is applied to the mixture of the polypeptides in ionic liquids. In some embodiments, the methods are for preparing polypeptides for sequencing and/or sequence analysis. In some embodiments, the provided methods are for treating one or more polypeptides in the presence microwave energy. In some examples, applying microwave energy to polypeptides denatures the polypeptides (e.g., melting, alter folding of the polypeptide, or denature the structure of the protein). In some cases, the provided methods are for applying microwave energy to denature polypeptides to prepare the polypeptides for sequencing and/or for sequence analysis.

In some embodiments, the application of microwave energy to the polypeptides is before contacting a polypeptide with a functionalizing reagent to modify an amino acid of said polypeptide, a binding agent capable of binding to said polypeptide, and/or a removing reagent to remove an amino acid from said polypeptide. In some embodiments, the application of microwave energy to the polypeptides is after contacting a polypeptide with a functionalizing reagent to modify an amino acid of said polypeptide, a binding agent capable of binding to said polypeptide, and/or a removing reagent to remove an amino acid from said polypeptide. In some embodiments, the application of microwave energy to the polypeptides is at the same time or simultaneously performed with contacting a polypeptide with a functionalizing reagent to modify an amino acid of said polypeptide, a binding agent capable of binding to said polypeptide, and/or a removing reagent to remove an amino acid from said polypeptide.

Provided herein is a method for sequencing a polypeptide comprising contacting a polypeptide with a functionalizing reagent to modify an amino acid of said polypeptide, a binding agent capable of binding to said polypeptide, and/or a removing reagent to remove an amino acid from said polypeptide; and applying a microwave energy to said polypeptide. The application of the microwave energy may be in sequence with each of the reagents/materials contacted by the polypeptide. For example, a polypeptide is first contacted with the functionalizing reagent to modify an amino acid of said polypeptide and then microwave energy is applied. In another example, a polypeptide is first contacted with the binding agent and then microwave energy is applied. In another example, a polypeptide is first contacted with the removing reagent to remove an amino acid from said polypeptide, and then microwave energy is applied. In some particular examples, the polypeptide is contacted with a functionalizing reagent, binding agent, and removing reagent in sequential order (the order may be switched around), and microwave energy is applied after some of the three contacting steps or each of the three contacting steps.

In some embodiments, the method further comprises determining the sequence of at least a portion of said polypeptide. Also provided herein is a method for treating a polypeptide comprising contacting a polypeptide with a functionalizing reagent to modify an amino acid of said polypeptide, a binding agent capable of binding to said polypeptide, and/or a removing reagent to remove an amino acid from said polypeptide; and applying a microwave energy to said polypeptide, wherein the functionalizing reagent modifies an N-terminal amino acid (NTAA), the binding agent binds to an N-terminal amino acid (NTAA), and/or the removing reagent removes an N-terminal amino acid (NTAA). In some embodiments, the methods provided include accelerating reactions with polypeptides. In some embodiments, the methods for accelerating reactions includes the application of radiation, e.g., electromagnetic radiation or microwave energy. In some embodiments, the methods are for reacting or contacting a plurality of polypeptides with a functionalizing reagent to modify one or more amino acids of the polypeptide. In some embodiments, the methods are for contacting the polypeptides with one or more binding agents. In some embodiments, the methods are for reacting or contacting a plurality of polypeptides with a removing reagent to remove one or more amino acids of the polypeptide. In some aspects, the methods include accelerating reactions including polypeptides with functionalizing reagents, binding agents, and/or removing agents. In some of any such embodiments, one or more of the steps with the polypeptide are performed in the presence of microwave energy.

In some embodiments, the methods for contacting a plurality of polypeptides with a functionalizing reagent to modify one or more amino acids of the polypeptide in the presence of microwave energy is more efficient compared to the reacting performed in the absence of microwave energy. In some embodiments, the methods for contacting the polypeptides with one or more binding agents in the presence of microwave energy is more efficient compared to contacting in the absence of microwave energy. In some embodiments, the methods for reacting or contacting a plurality of polypeptides with a reagent to remove one or more amino acids of the polypeptide in the presence of microwave energy is more efficient than removal performed in the absence of microwave energy. In some aspects, the methods accelerate reactions including polypeptides with functionalizing reagents, binding agents, and/or removing agents when microwave energy is applied compared to in the absence of microwave energy.

In some embodiments, modification of the amino acid of the polypeptide, binding between or among the binding agent and the polypeptide and/or removal of an amino acid from the polypeptide is accelerated due to the application of the microwave energy to the polypeptide. In some examples, time required for conducting any or all steps of the method is shortened due to the application of the microwave energy to the polypeptide. In some embodiments, the time required for conducting any or all steps of the method due to the application of the microwave energy to the polypeptide is shortened by at least 5%, at least 10%, at least 15%, at least 20%, at least 25%, at least 30%, at least 35%, at least 40%, at least 45%, at least 50%, at least 60%, at least 70%, at least 80%, or at least 90% or more, as compared to a time required for conducting any or all steps of the method without application of the microwave energy to the polypeptide. In some embodiments, the time required for conducting any or all steps of the method due to the application of the microwave energy to the polypeptide is shortened by at least 5% as compared to a time required for conducting any or all steps of the method without application of the microwave energy to the polypeptide.

In some embodiments, the level or percentage of modification of the amino acid of the polypeptide, binding between or among the binding agent and the polypeptide and/or removal of an amino acid from the polypeptide is enhanced or increased due to the application of the microwave energy to the polypeptide. In some examples, the level or percentage of modification of the amino acid of the polypeptide, binding between or among the binding agent and the polypeptide and/or removal of an amino acid from the polypeptide due to the application of the microwave energy to the polypeptide is enhanced or increased by at least 5%, at least 10%, at least 15%, at least 20%, at least 25%, at least 30%, at least 35%, at least 40%, at least 45%, at least 50%, at least 60%, at least 70%, at least 80%, or at least 90% or more, as compared to the level or percentage of modification of the amino acid of the polypeptide, binding between or among the binding agent and the polypeptide and/or removal of an amino acid from the polypeptide without application of the microwave energy to the polypeptide. In some examples, the level or percentage of modification of the amino acid of the polypeptide, binding between or among the binding agent and the polypeptide and/or removal of an amino acid from the polypeptide due to the application of the microwave energy to the polypeptide is enhanced or increased by at least 5% as compared to the level or percentage of modification of the amino acid of the polypeptide, binding between or among the binding agent and the polypeptide and/or removal of an amino acid from the polypeptide without application of the microwave energy to the polypeptide.

In some embodiments, the provided methods may reduce or eliminate bias of functionalization and/or removal of different amino acids due to the application of the microwave energy to the polypeptide. In some embodiments, the bias of functionalization and/or removal is between hydrophobic amino acids vs. non-hydrophobic amino acids, charged vs. non-charged amino acids, and/or polar vs. non-polar amino acids. In some embodiments, the bias of functionalization and/or removal between hydrophobic amino acids and non-hydrophobic amino acids is reduced or eliminated due to the application of the microwave energy to the polypeptide. In some examples, the bias of functionalization and/or removal of different amino acids due to the application of the microwave energy to the polypeptide is reduced by at least 5%, at least 10%, at least 15%, at least 20%, at least 25%, at least 30%, at least 35%, at least 40%, at least 45%, at least 50%, at least 60%, at least 70%, at least 80%, or at least 90% or more, as compared to the bias of functionalization and/or removal of different amino acids without application of the microwave energy to the polypeptide. In some cases, the bias of functionalization and/or removal of different amino acids due to the application of the microwave energy to the polypeptide is reduced by at least 5% as compared to the bias of functionalization and/or removal of different amino acids without application of the microwave energy to the polypeptide. In some aspects, the methods of acceleration provided herein are compatible with nucleic acid encoding macromolecules.

Provided herein is a method for analyzing a plurality of polypeptides including: (a) contacting a plurality of polypeptides with a functionalizing reagent to modify an amino acid of the polypeptide; (b) contacting the polypeptide with a reagent to remove the functionalized amino acid; and (c) determining the sequence of at least a portion of the polypeptide. In some embodiments, the method further comprises (a1) contacting the polypeptide with a binding reagent. In some embodiments, step (a), (a1), (b), and/or (c), or any combination thereof, is performed in the presence of applied microwave energy. In some embodiments, step (a) and step (b) are performed sequentially. In some cases, step (a), (a1), and step (b) are performed sequentially. In some cases, step (a), (a1), step (b) and step (c) are performed sequentially. In some embodiments, step (a) is performed before step (a1) and/or before step (b). In some embodiments, step (a1) is performed before step (b) and/or step (c). In some cases, step (b) is performed before step (c). In some embodiments, step (a1) and/or (a1) is performed before step (c). In some embodiments, step (a) and step (b) are repeated. In some cases, step (a), (a1), and step (b) are repeated.

In some embodiments, the method further includes determining the sequence of at least a portion of the polypeptide. In some embodiments, determining the sequence of at least a portion of the polypeptide includes performing any of the methods as described in International Patent Publication No. WO 2017/192633.

In certain embodiments, an agent or reagent for binding, recognizing, removing, or modifying one or more amino acid residues may be a selective agent or reagent. As used herein, selectivity refers to the ability of the reagent or agent to preferentially bind to a specific target (e.g., amino acid or class of amino acids) relative to binding to a different ligand (e.g., amino acid or class of amino acids). Selectivity is commonly referred to as the equilibrium constant for the reaction of displacement of one ligand by another ligand in a complex with a reagent or agent. Typically, such selectivity is associated with the spatial geometry of the ligand and/or the manner and degree by which the ligand binds to a reagent or agent, such as by hydrogen bonding or Van der Waals forces (non-covalent interactions) or by reversible or non-reversible covalent attachment to the reagent or agent. It should also be understood that selectivity may be relative, and as opposed to absolute, and that different factors can affect the same, including ligand concentration. Thus, in one example, a reagent or agent for binding, recognizing, removing, or modifying one or more amino acid residues may selectively bind one of the twenty standard amino acids. In an example of non-selective binding, a reagent or agent may bind or modify to two or more of the twenty standard amino acids. In some embodiments, for example, a reagent or agent (e.g., binding agent, functionalizing reagent, reagent that removes an amino acid) may selectively or specifically bind or modify an NTAA and may not bind or modify a CTAA.

In some embodiments, the contacting of the polypeptide with a functionalizing reagent, a binding agent, and/or a removing reagent is performed with the polypeptide in solution. In some embodiments, the contacting of the polypeptide with a functionalizing reagent, a binding agent, and/or a removing reagent is performed with the polypeptide that is attached to a support.

A. Polypeptide Modification e.g., Functionalization

Provided herein are methods for modifying a polypeptide, such as by contacting one or more polypeptides with a functionalizing reagent. Also provided herein is a method of accelerating a sequencing reaction with a polypeptide comprising contacting the polypeptide with a functionalizing reagent to modify one or more amino acids of the polypeptide and applying microwave energy; and determining the sequence of at least a portion of the polypeptide. In some embodiments, the method for treating a polypeptide for sequence analysis includes (a) preparing a mixture comprising one or more polypeptides and functionalizing reagents to modify one or more amino acids; (b) subjecting the mixture to microwave energy; and (c) determining the sequence of at least a portion of the polypeptide. In some cases, the modified amino acid is an amino acid at the terminus of the polypeptide, an N-terminal amino acid (NTAA), or a C-terminal amino acid (CTAA). In some embodiments, the modification is guanidinylation of an amino acid (e.g., guanidinylation of an NTAA).

In some embodiments, the methods are for accelerating a reaction with a polypeptide comprising contacting the polypeptide with a functionalizing reagent to modify an N-terminal amino acid (NTAA) of the polypeptide and applying microwave energy. In some embodiments, the provided methods for treating a polypeptide for sequence analysis includes the steps of (a) preparing a mixture comprising one or more polypeptides and a functionalizing reagent to modify an N-terminal amino acid (NTAA); and (b) subjecting the mixture to microwave energy. In some embodiments, the functionalizing reagent is a guanidinylating reagent. In some embodiments, step (a) is conducted before step (b). In some embodiments, step (b) is conducted before step (a). In some embodiments, wherein the step (a) and the step (b) are conducted in the same step or simultaneously.

In some embodiments, the functionalizing reagent comprises one or more of any compound of Formula (I), (II), (III), (IV), (V), (VI), or (VII) described herein, or a salt or conjugate thereof. In some embodiments, the methods provided herein comprises using a reagent described in PCT Publication No. WO 2019/089846.

In some embodiments, microwave-assisted modification (e.g., functionalization) of one or more amino acid(s) may be performed at any acceptable reaction times, such as about 60 minutes or below. In some embodiments, the reaction time for functionalization is below about 30 minutes, such as below about 10 minutes. In some embodiments, the reaction time for functionalization is below about 20 minutes, below about 15 minutes, below about 10 minutes, or below about 5 minutes. In some embodiments, the In some aspects, the reaction time may be shortened by optimization of microwave conditions. In some embodiments, the microwave energy is applied for a duration of time effective to achieve modification or functionalization in 20%, 30%, 40%, 50%, 60%, 70%, 80%, 90% or more or greater polypeptides.

In some embodiments, the microwave energy is applied at about 5 watts, about 10 watts, about 15 watts, about 20 watts, about 25 watts, about 30 watts, about 35 watts, about 40 watts, about 45 watts, about 50 watts, about 60 watts, about 70 watts, about 80 watts, about 90 watts, about 100 watts, about 110 watts, about 120 watts, about 130 watts, about 140 watts, or about 150 or higher watts. In some examples, the microwave energy applied to the functionalization reaction is at or about 30 watts.

In some embodiments, the contacting with the functionalizing reagent or treating of the polypeptide with a functionalizing reagent are performed in the presence of microwave energy that maintains the reaction at a fixed temperature. In some examples, the contacting with the functionalizing reagent or treating of the polypeptide with a functionalizing reagent is performed in the presence of microwave energy that maintains the reaction at a temperature of about at least about 10° C., 20° C., 30° C., 40° C., 50° C., 60° C., 70° C., 80° C., 90° C., or 100° C. or higher, or any range thereof. In some cases, the methods provided herein are performed in a vessel that provides a microwave energy to maintain the reaction at a temperature of about 30° C., 60° C., or 80° C., or any range thereof.

In some embodiments, microwave-assisted modification (e.g., functionalization) of one or more amino acid(s) achieves greater uniformity in modifying of the amino acids compared to in the absence of microwave energy. In some embodiments, application of microwave energy reduces bias of functionalization or modification of different amino acids. For example, in some cases, some amino acid residues may exhibit bias or show decreased modification compared to other residues when reactions are performed in the absence of microwave energy (e.g., based on hydrophobicity, charge, polarity, or other characteristics). In some cases, application of microwave energy eliminates the bias of amino acid functionalization (e.g., functionalization of hydrophobic vs non-hydrophobic residues).

In certain embodiments, a terminal amino acid (e.g., NTAA or CTAA) of a polypeptide is modified (e.g., functionalized). In some embodiments, the terminal amino acid is functionalized prior to contacting the polypeptide with a binding agent in the methods described herein. In some embodiments, the terminal amino acid is functionalized after contacting the polypeptide with a binding agent in the methods described herein. In some embodiments, the terminal amino acid is functionalized prior to contacting the polypeptide with a removing reagent such as described in the methods herein.

In some embodiments, the terminal amino acid is modified by contacting the polypeptide with a functionalizing reagent. In some embodiments, the polypeptide is first contacted with a proline aminopeptidase or variant/mutant thereof under conditions suitable to remove an N-terminal proline, before using the method(s) of the invention.

Provided in some aspects are methods for treating a polypeptide including contacting with a reagent for functionalizing one or more amino acids of the polypeptide. In some embodiments, the functionalized amino acid is at the terminus of the polypeptide. In some embodiments, the functionalized amino acid is the N-terminal amino acid (NTAA) of the polypeptide. In some cases, the functionalized amino acid is the C-terminal amino acid (CTAA). In some embodiments, the method selectively or specifically modifies the N-terminal amino acid (NTAA) of the polypeptide.

In some embodiments, the provided methods further comprise contacting the polypeptide with a reagent for removing the functionalized amino acid from the polypeptide to expose the immediately adjacent amino acid residue. In some embodiments, the functionalized amino acid is removed in a subsequent reaction.

Provided herein in some aspects are functionalizing reagents used to modify the terminal amino acid of a polypeptide. In some embodiments, terminal amino acid of a polypeptide (e.g., the NTAA of a polypeptide) is functionalized via guanidinylation. In some embodiments, the functionalizing reagent comprises a derivative of guanidine. (See, e.g., Bhattacharjree et al., 2016, J. Chem. Sci. 128(6):875-881; Chi et al., 2015, Chem. Eur. J. 2015, 21, 10369-10378, incorporated by reference in their entireties). In some embodiments, the functionalizing reagent comprises a guanidinylation reagent (See e.g., U.S. Pat. No. 6,072,075, incorporated by reference in its entirety).

In some embodiments, the functionalizing reagent is or comprises a chemical agent, an enzyme, and/or a biological agent. In some embodiments, the functionalizing reagent adds a chemical moiety to the amino acid. For example, the chemical moiety is added to one or more amino acids of the polypeptide via a chemical reaction or enzymatic reaction. In some examples, the chemical moiety added to the polypeptide is phenylthiocarbamoyl (PTC or derivatized PTC), dinitrophenol (DNP) moiety; a sulfonyloxynitrophenyl (SNP) moiety, a dansyl moiety; a 7-methoxy coumarin moiety; a thioacyl moiety; a thioacetyl moiety; an acetyl moiety; a Cbz moiety; a guanidinyl moiety; or a thiobenzyl moiety. In some embodiments, the functionalizing reagent is or comprises an isothiocyanate derivative, a phenylisothiocyanate, PITC, 2,4-dinitrobenzenesulfonic (DNB S), 4-sulfonyl-2-nitrofluorobenzene (SNFB), 1-fluoro-2,4-dinitrobenzene (Sanger's reagent, DNFB), polypeptidebenzyloxycarbonyl chloride or carbobenzoxy chloride (Cbz-Cl), N-(Benzyloxycarbonyloxy)succinimide (Cbz-OSu or Cbz-O-NHS), dansyl chloride (DNS-Cl, or 1-dimethylaminonaphthalene-5-sulfonyl chloride), 7-methoxycoumarin acetic acid, N-Acetyl-Isatoic Anhydride, Isatoic Anhydride, 2-Pyridinecarboxaldehyde, 2-Formylphenylboronic acid, 2-Acetylphenylboronic acid, 1-Fluoro-2,4-dinitrobenzene, Succinic anhydride, 4-Chloro-7-nitrobenzofurazan, Pentafluorophenylisothiocyanate, 4-(Trifluoromethoxy)-phenylisothiocyanate, 4-(Trifluoromethyl)-phenylisothiocyanate, 3-(Carboxylic acid)-phenylisothiocyanate, 3-(Trifluoromethyl)-phenylisothiocyanate, 1-Naphthylisothiocyanate, N-nitroimidazole-1-carboximidamide, N,N,Ä≤-Bis(pivaloyl)-1H-pyrazole-1-carboxamidine, N,N,Ä≤-Bis(benzyloxycarbonyl)-1H-pyrazole-1-carboxamidine, an acetylating reagent, a guanidinylation reagent, a thioacylation reagent, a thioacetylation reagent, a thiobenzylation reagent, and/or a diheterocyclic methanimine reagent. In some particular examples, the chemical moiety added to the polypeptide is a guanidinyl moiety. In some embodiments, the functionalizing reagent selectively or specifically modifies the N-terminal amino acid (NTAA) of the polypeptide.

In some embodiments, the functionalizing reagent comprises a compound selected from the group consisting of a compound of Formula (I):

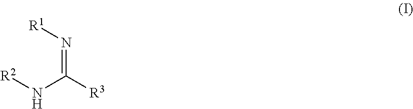

or a salt or conjugate thereof,

wherein
R¹ and R² are each independently H, C₁₋₆ alkyl, cycloalkyl, —C(O)R^{a}, —C(O)OR^{b}, or —S(O)₂R^{c};
   R^{a}, R^{b}, and R^{c} are each independently H, C₁₋₆alkyl, C₁₋₆haloalkyl, arylalkyl, aryl, or heteroaryl, wherein the C₁₋₆alkyl, C₁₋₆haloalkyl, arylalkyl, aryl, and heteroaryl are each unsubstituted or substituted;
R³ is heteroaryl, —NR^{d}C(O)OR^{e}, or —SR^{f}, wherein the heteroaryl is unsubstituted or substituted;
   R^{d}, R^{e}, and R^{f} are each independently H or C₁₋₆alkyl.

In some embodiments, when R³ is

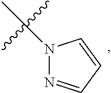 [SMILES: *n1cccn1]

R¹ and R² are not both H. In some embodiments of Formula (I), both R¹ and R² are H. In some embodiments, neither R¹ nor R² are H. In some embodiments, one of R¹ and R² is C₁₋₆alkyl. In some embodiments, one of R¹ and R² is H, and the other is C₁₋₆alkyl, cycloalkyl, —C(O)R^{a}, —C(O)OR^{b}, or —S(O)₂R^{c}. In some embodiments, one or both of R¹ and R² is C₁₋₆alkyl. In some embodiments, one or both of R¹ and R² is cycloalkyl. In some embodiments, one or both of R¹ and R² is —C(O)R^{a}. In some embodiments, one or both of R¹ and R² is —C(O)OR^{b}. In some embodiments, one or both of R¹ and R² is —S(O)₂R^{c}. In some embodiments, one or both of R¹ and R² is —S(O)₂R^{c}, wherein R^{c} is C₁₋₆alkyl, C₁₋₆haloalkyl, arylalkyl, aryl, or heteroaryl. In some embodiments, R¹ is

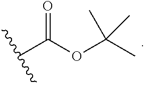 [SMILES: *C(=O)OC(C)(C)C]

In some embodiments, R² is

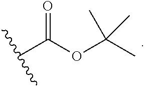 [SMILES: *C(=O)OC(C)(C)C]

In some embodiments, both R¹ and R² are

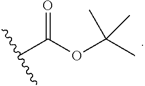 [SMILES: *C(=O)OC(C)(C)C]

In some embodiments, R¹ or R² is

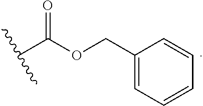 [SMILES: *C(=O)OCc1ccccc1]

In some embodiments of the compound of Formula (I), R³ is a monocyclic heteroaryl group. In some embodiments of Formula (I), R³ is a 5- or 6-membered monocyclic heteroaryl group. In some embodiments of Formula (I), R³ is a 5- or 6-membered monocyclic heteroaryl group containing one or more N. Preferably, R³ is selected from pyrazole, imidazole, triazole and tetrazole, and is linked to the amidine of Formula (I) via a nitrogen atom of the pyrazole, imidazole, triazole or tetrazole ring, and R³ is optionally substituted by a group selected from halo, C₁₋₃ alkyl, C₁₋₃ haloalkyl, and nitro. In some embodiments, R³ is

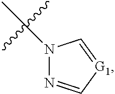

wherein G₁ is N, CH, or CX where X is halo, C₁₋₃ alkyl, C₁₋₃ haloalkyl, or nitro. In some embodiments, R³ is

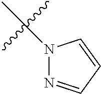 [SMILES: *n1cccn1]

or, where X is Me, F, C₁, CF₃, or NO₂. In some embodiments, R³ is

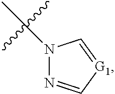

wherein G₁ is N or CH. In some embodiments, R³ is

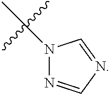 [SMILES: *n1cncn1]

In some embodiments, R³ is a bicyclic heteroaryl group. In some embodiments, R³ is a 9- or 10-membered bicyclic heteroaryl group. In some embodiments, R³ is

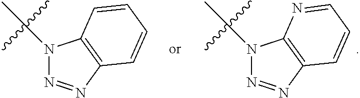 [SMILES: *n1nnc2ccccc21 ; *n1nnc2cccnc21]

In some embodiments, the compound of Formula (I) is

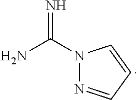

In some embodiments, the compound of Formula (I) is not

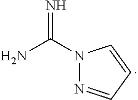

In some embodiments, the compound of Formula (I) for use in the methods and kits disclosed herein is selected from the group consisting of

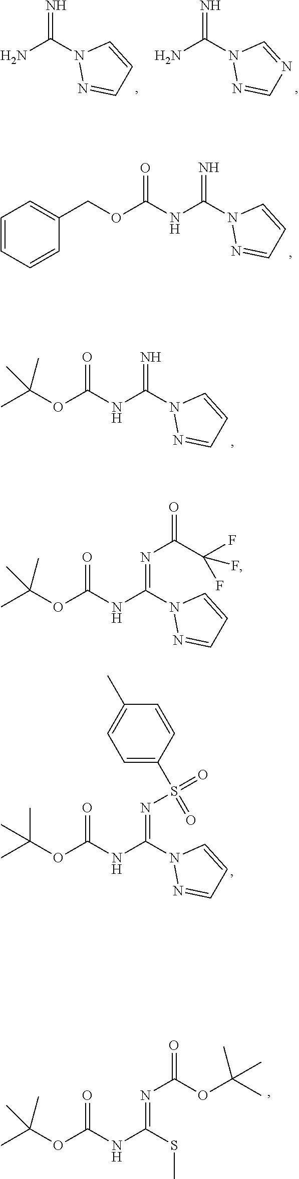

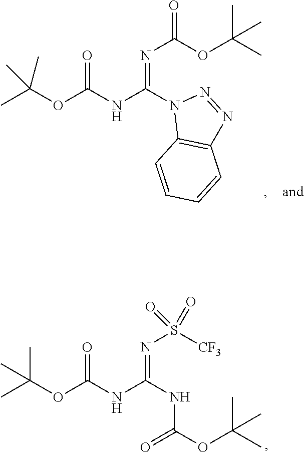

and optionally also including

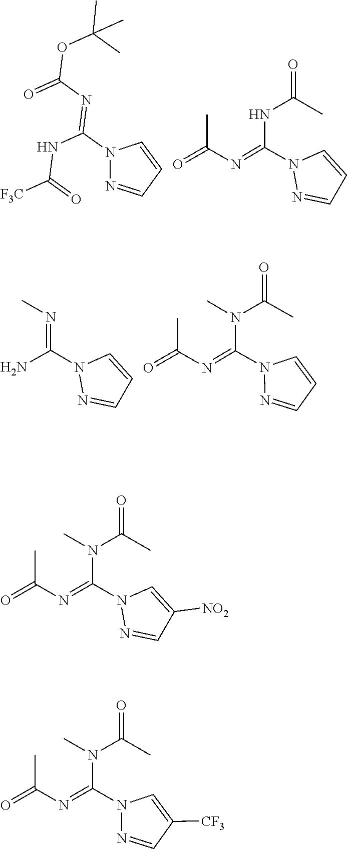

(N-Boc,N′-trifluoroacetyl-pyrazolecarboxamidine, N,N′-bisacetyl-pyrazolecarboxamidine, N-methyl-pyrazolecarboxamidine, N,N′-bisacetyl-N-methyl-pyrazolecarboxamidine, N,N′-bisacetyl-N-methyl-4-nitro-pyrazolecarboxamidine, and N,N′-bisacetyl-N-methyl-4-trifluoromethyl-pyrazolecarboxamidine), or a salt or conjugate of any of these.

In some embodiments, the functionalizing reagent additionally comprises Mukaiyama's reagent (2-chloro-1-methylppidinium iodide). In some embodiments, the functionalizing reagent comprises at least one compound of Formula (I) and Mukaiyama's reagent.

In some embodiments, modification of the terminal amino acid (e.g., NTAA) using a functionalizing reagent comprising a compound of Formula (I) and the subsequent elimination are as depicted in the following scheme:

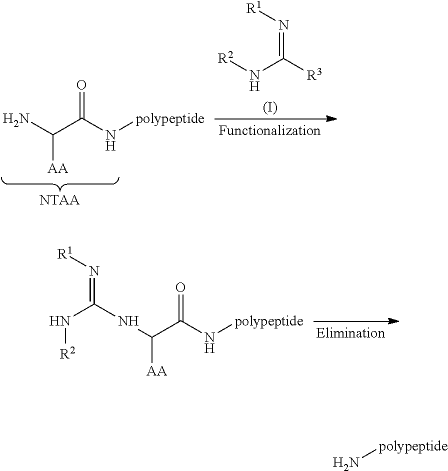

wherein R¹, R², and R³ are as defined above and AA is the side chain of the NTAA.

In some embodiments, the product of the elimination step comprises the functionalized NTAA that has been eliminated from the polypeptide. In some embodiments, the product of the functionalized NTAA that has been eliminated from the polypeptide is in linear form. In some embodiments, the product of the elimination step is comprised of the two terminal amino acids. In some embodiments, the functionalized NTAA that has been eliminated from the polypeptide comprises a ring. In some embodiments, the elimination product of a NTAA functionalized with a compound of Formula (I) comprises

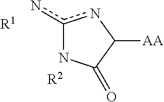

and/or

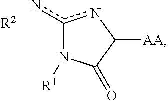

wherein R¹ and R² are as defined above and AA is the side chain of the NTAA.

In some embodiments, the functionalizing reagent comprising a cyanamide derivative is used to functionalize one or more amino acids of the polypeptide. (See, e.g., Kwon et al., Org. Lett. 2014, 16, 6048-6051, incorporated by reference in its entirety).

In some embodiments, the functionalizing reagent comprises a compound selected from the group consisting of a compound of Formula (II):

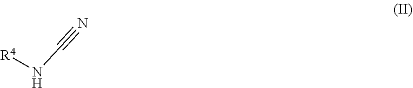

or a salt or conjugate thereof,

wherein

R⁴ is H, C₁₋₆ alkyl, cycloalkyl, —C(O)R^{g}, or —C(O)ORg; and
R^{g} is H, C₁₋₆alkyl, C₂₋₆alkenyl, C₁₋₆haloalkyl, or arylalkyl, wherein the C₁₋₆alkyl, C₂₋₆alkenyl, C₁₋₆haloalkyl, and arylalkyl are each unsubstituted or substituted.

In some embodiments of Formula (II), R⁴ is H. In some embodiments, R⁴ is C₁₋₆alkyl. In some embodiments, R⁴ is cycloalkyl. In some embodiments, R⁴ is —C(O)R^{g} and R^{g} is C₂₋₆alkenyl, optionally substituted with aryl, heteroaryl, or heterocyclyl. In some embodiments, R⁴ is —C(O)OR^{g} and R^{g} is C₂₋₆alkenyl, optionally substituted with C₁₋₆ alkyl, aryl, heteroaryl, or heterocyclyl. In some embodiments, R^{g} is C₂alkenyl, substituted with C₁₋₆alkyl, aryl, heteroaryl, or heterocyclyl, wherein the C₁₋₆ alkyl, aryl, heteroaryl, or heterocyclyl are optionally further substituted. In some embodiments, R⁴ is —C(O)R^{g} or —C(O)OR^{g}, R^{g} is C₂alkenyl, substituted with C₁₋₆ alkyl, aryl, heteroaryl, or heterocyclyl, wherein the C₁₋₆alkyl, aryl, heteroaryl, or heterocyclyl are optionally further substituted with halo, C₁₋₆ alkyl, haloalkyl, hydroxyl, or alkoxy. In some embodiments, R⁴ is carboxybenzyl. In some embodiments, the compound is selected from the group consisting of

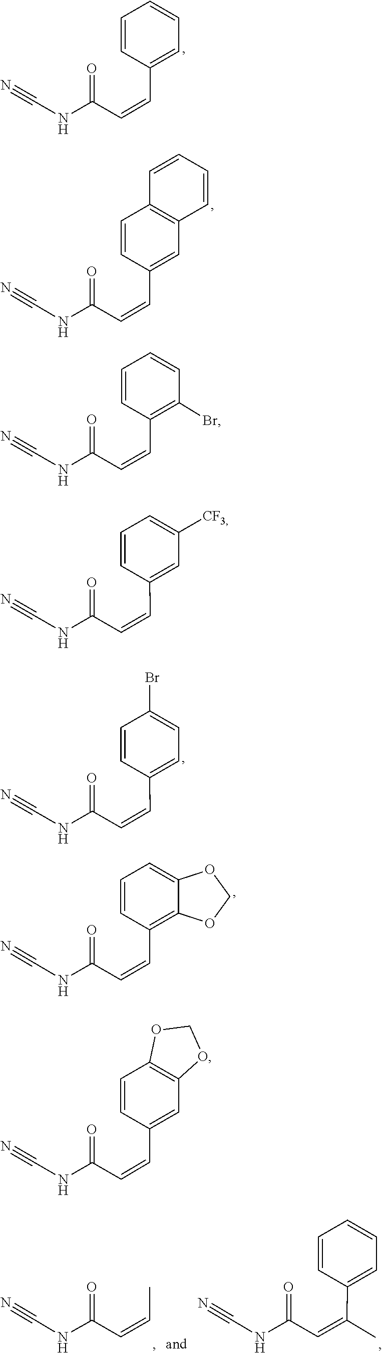

or a salt or conjugate thereof.

In some embodiments, the functionalizing reagent additionally comprises TMS-Cl, Sc(OTf)₂, Zn(OTf)₂, or a lanthanide-containing reagent. In some embodiments, the functionalizing reagent comprises at least one compound of Formula (II) and TMS-Cl, Sc(OTf)₂, Zn(OTf)₂, or a lanthanide-containing reagent.

In some embodiments, functionalization of the terminal amino acid comprises contacting with a compound of Formula (II) and the subsequent elimination are as depicted in the following scheme:

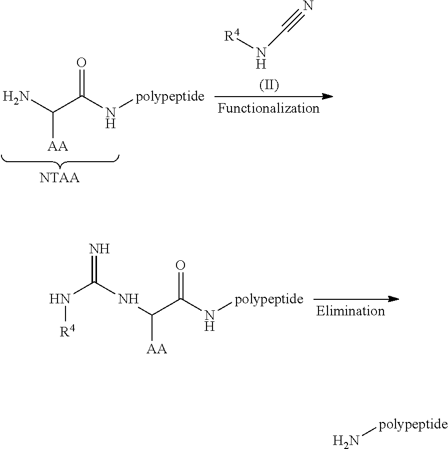

wherein R⁴ is as defined above and AA is the side chain of the NTAA.

In some embodiments, the elimination product of a NTAA functionalized with a compound of Formula (II) comprises

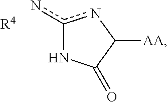

wherein R⁴ is as defined above and AA is the side chain of the NTAA. In some embodiments, the product of the functionalized NTAA that has been eliminated from the polypeptide is in linear form. In some embodiments, the product of the elimination step is comprised of two terminal amino acids.

In some embodiments, a functionalizing reagent comprising an isothiocyanate derivative is used to functionalize the terminal amino acid (e.g., NTAA) of a polypeptide. (See, e.g., Martin et al., Organometallics. 2006, 34, 1787-1801, incorporated by reference in its entirety).

In some embodiments, the functionalizing reagent comprises a compound selected from the group consisting of a compound of Formula (III):

R⁵—N═C═S (III)

or a salt or conjugate thereof,
wherein

R⁵ is C₂₋₆alkyl, C₂₋₆alkenyl, cycloalkyl, heterocyclyl, aryl or heteroaryl;
wherein the C₁₋₆ alkyl, C₂₋₆alkenyl, cycloalkyl, heterocyclyl, aryl or heteroaryl are each unsubstituted or substituted with one or more groups selected from the group consisting of halo, —NR^{h}Rⁱ, —S(O)₂R^{j}, or heterocyclyl;
R^{h}, Rⁱ, and R^{j} are each independently H, C₁₋₆alkyl, C₁₋₆haloalkyl, arylalkyl, aryl, or heteroaryl, wherein the C₁₋₆alkyl, C₁₋₆haloalkyl, arylalkyl, aryl, and heteroaryl are each unsubstituted or substituted.

In some embodiments of Formula (III), R⁵ is substituted phenyl. In some embodiments, R⁵ is substituted phenyl substituted with one or more groups selected from halo, —NR^{h}Rⁱ, —S(O)₂R^{j}, or heterocyclyl. In some embodiments, R⁵ is unsubstituted C₁₋₆alkyl. In some embodiments, R⁵ is substituted C₁₋₆ alkyl. In some embodiments, R⁵ is substituted C₁₋₆alkyl, substituted with one or more groups selected from halo, —NR^{h}Rⁱ, —S(O)₂R^{j}, or heterocyclyl. In some embodiments, R⁵ is unsubstituted C₂₋₆alkenyl. In some embodiments, R⁵ is C₂₋₆alkenyl. In some embodiments, R⁵ is substituted C₂₋₆alkenyl, substituted with one or more groups selected from halo, —NR^{h}Rⁱ, —S(O)₂R^{j}, or heterocyclyl. In some embodiments, R⁵ is unsubstituted aryl. In some embodiments, R⁵ is substituted aryl. In some embodiments, R⁵ is aryl, substituted with one or more groups selected from halo, —NR^{h}Rⁱ, —S(O)₂R^{j}, or heterocyclyl. In some embodiments, R⁵ is unsubstituted cycloalkyl. In some embodiments, R⁵ is substituted cycloalkyl. In some embodiments, R⁵ is cycloalkyl, substituted with one or more groups selected from halo, —NR^{h}Rⁱ, —S(O)₂R^{j}, or heterocyclyl. In some embodiments, R⁵ is unsubstituted heterocyclyl. In some embodiments, R⁵ is substituted heterocyclyl. In some embodiments, R⁵ is heterocyclyl, substituted with one or more groups selected from halo, —NR^{h}Rⁱ, —S(O)₂R^{j}, or heterocyclyl. In some embodiments, R⁵ is unsubstituted heteroaryl. In some embodiments, R⁵ is substituted heteroaryl. In some embodiments, R⁵ is heteroaryl, substituted with one or more groups selected from halo, —NR^{h}Rⁱ, —S(O)₂R^{j}, or heterocyclyl.

In some embodiments, the compound of Formula (III) is trimethylsilyl isothiocyanate (TMSITC) or pentafluorophenyl isothiocyanate (PFPITC).

In some embodiments, the compound is not trifluoromethyl isothiocyanate, allyl isothiocyanate, dimethylaminoazobenzene isothiocyanate, 4-sulfophenyl isothiocyanate, 3-pyridyl isothiocyanate, 2-piperidinoethyl isothiocyanate, 3-(4-morpholino) propyl isothiocyanate, or 3-(diethylamino)propyl isothiocyanate.

In some embodiments, the method includes contacting with a reagent that is or comprises an alkyl amine. In some embodiments, the reagent additionally comprises DIPEA, trimethylamine, pyridine, and/or N-methylpiperidine. In some embodiments, the reagent additionally comprises pyridine and triethylamine in acetonitrile. In some embodiments, the reagent additionally comprises N-methylpiperidine in water and/or methanol.

In some embodiments, the method further includes contacting the polypeptide with a carbodiimide compound.

In some embodiments, functionalization using a reagent comprising a compound of Formula (III) and the subsequent elimination are as depicted in the following exemplary scheme:

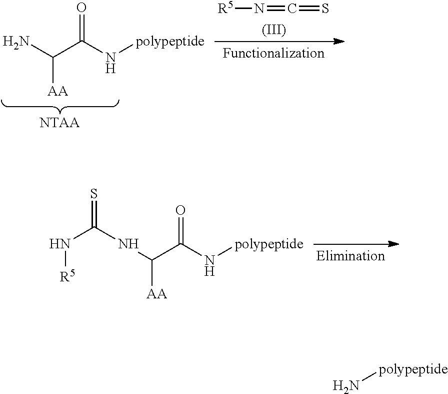

wherein R⁵ is as defined above and AA is the side chain of the NTAA.

In some embodiments, the elimination product of an amino acid functionalized with a compound of Formula (III) comprises

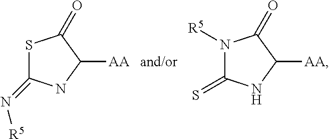

wherein R⁵ is as defined above and AA is the side chain of the amino acid.

In some embodiments, a functionalizing reagent comprising a carbodiimide derivative is used to functionalize the terminal amino acid (e.g., NTAA) of a polypeptide. (See, e.g., Chi et al., 2015, Chem. Eur. J. 2015, 21, 10369-10378, incorporated by reference in their entireties).

In some embodiments, the functionalizing reagent comprises a compound selected from the group consisting of a compound of Formula (IV):

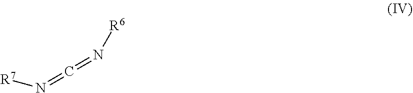

or a salt or conjugate thereof,

wherein

R⁶ and R⁷ are each independently H, C₁₋₆ alkyl, —CO₂C₁₋₄ alkyl, —OR^{k}, aryl, heteroaryl, cycloalkyl or heterocyclyl, wherein the C₁₋₆alkyl, —CO₂C₁₋₄ alkyl, —OR^{k}, aryl, and cycloalkyl are each unsubstituted or substituted; and

R^{k} is H, C₁₋₆alkyl, or heterocyclyl, wherein the C₁₋₆alkyl and heterocyclyl are each unsubstituted or substituted.

In some embodiments of Formula (IV), R⁶ and R⁷ are each independently H, C₁₋₆alkyl, cycloalkyl, —CO₂C₁₋₄alkyl, aryl. In some embodiments, R⁶ and R⁷ are each independently H, C₁₋₆alkyl, cycloalkyl. In some embodiments, R⁶ and R⁷ are the same. In some embodiments, R⁶ and R⁷ are different.

In some embodiments, one of R⁶ and R⁷ is C₁₋₆alkyl and the other is selected from the group consisting of C₁₋₆alkyl, —CO₂C₁₋₄alkyl, and —OR^{k}, wherein the C₁₋₆alkyl, —CO₂C₁₋₄alkyl, and —OR^{k} are each unsubstituted or substituted. In some embodiments, one or both of R⁶ and R⁷ is C₁₋₆alkyl, optionally substituted with aryl, such as phenyl. In some embodiments, one or both of R⁶ and R⁷ is C₁₋₆alkyl, optionally substituted with heterocyclyl. In some embodiments, one of R⁶ and R⁷ is —CO₂C₁₋₄ alkyl and the other is selected from the group consisting of C₁₋₆alkyl, —CO₂C₁₋₄ alkyl, and —OR^{k}, wherein the C₁₋₆alkyl, —CO₂C₁₋₄alkyl, and —OR^{k} are each unsubstituted or substituted. In some embodiments, one of R⁶ and R⁷ is optionally substituted aryl and the other is selected from the group consisting of C₁₋₆ alkyl, —CO₂C₁₋₄alkyl, —OR^{k}, aryl, heteroaryl, cycloalkyl or heterocyclyl, wherein the C₁₋₆ alkyl, —CO₂C₁₋₄alkyl, —OR^{k}, aryl, and cycloalkyl are each unsubstituted or substituted. In some embodiments, one or both of R⁶ and R⁷ is aryl, optionally substituted with C₁₋₆alkyl or NO₂.

In some embodiments, the compound is selected from the group consisting of

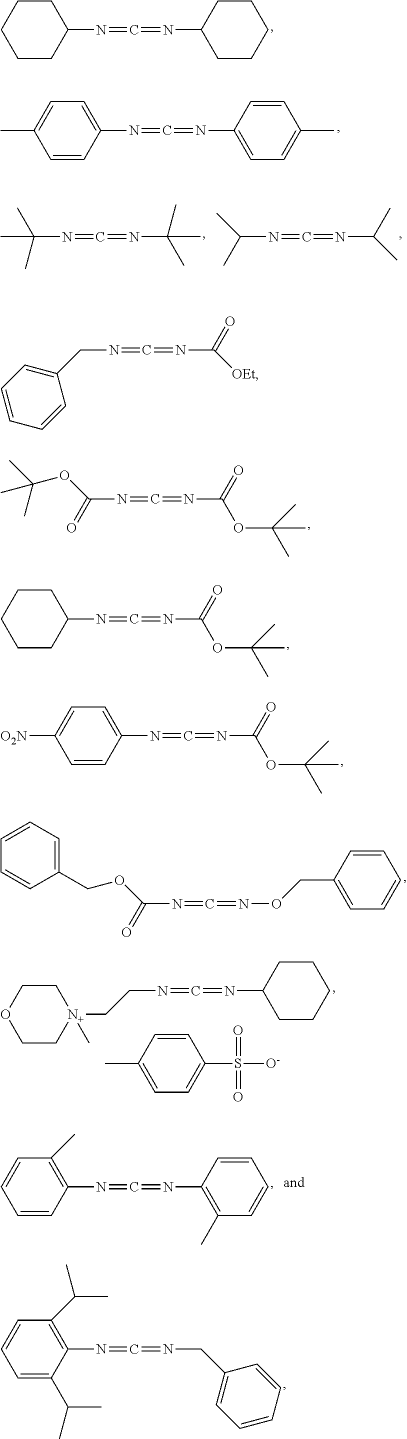

or a salt or conjugate thereof.

In some embodiments, the compound of Formula (IV) is prepared by desulfurization of the corresponding thiourea.

In some embodiments, the method comprises contacting with a reagent that additionally comprises Mukaiyama's reagent (2-chloro-1-methylpyridinium iodide). In some embodiments, the reagent additionally comprises a Lewis acid. In some embodiments, the Lewis acid selected from N-((aryl)imino-acenapthenone)ZnCb, Zn(OTf)₂, ZnCl₂, PdCl₂, CuCl, and CuCl₂.

In some embodiments, functionalization of the amino acid comprises contacting with a compound of Formula (IV) and the subsequent elimination are as depicted in the following exemplary scheme:

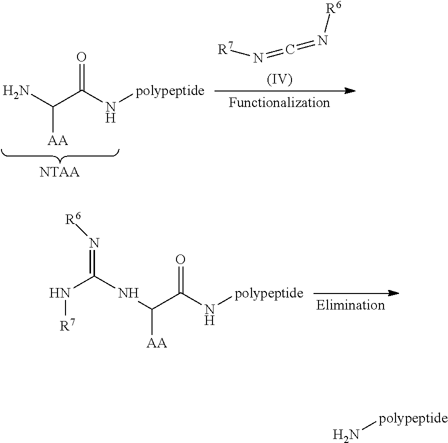

wherein R⁶ and R⁷ are as defined above and AA is the side chain of the NTAA.

In some embodiments, the elimination product of a terminal amino acid (e.g., NTAA) functionalized with a compound of Formula (IV) comprises

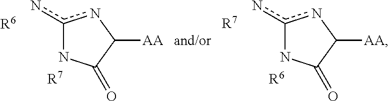

wherein R⁶ and R⁷ are as defined above and AA is the side chain of the NTAA. In some embodiments, the product of the functionalized NTAA that has been eliminated from the polypeptide is in linear form. In some embodiments, the product of the elimination step is comprised of two terminal amino acids.

In some embodiments, the NTAA of a polypeptide is functionalized via acylation. (See, e.g., Protein Science (1992), I, 582-589, incorporated by reference in their entireties).

In some embodiments, the functionalizing reagent comprises a compound selected from the group consisting of a compound of Formula (V):

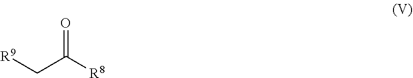

or a salt or conjugate thereof,
wherein

R⁸ is halo or —OR^{m};
R^{m} is H, C₁₋₆alkyl, or heterocyclyl; and

R⁹ is hydrogen, halo, or C₁₋₆haloalkyl.

In some embodiments of Formula (V), R⁸ is halo. In some embodiments, R⁸ is chloro. In some embodiments, R⁸

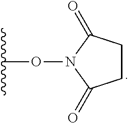 [SMILES: *ON1C(=O)CCC1=O]

In some embodiments, R⁹ is hydrogen. In some embodiments, R⁹ is halo, such as bromo. In some embodiments, the compound of Formula (V) is selected from acetyl chloride, acetyl anhydride, and acetyl-NHS. In some embodiments, the compound is not acetyl anhydride or acetyl-NHS.

In some embodiments, the method additionally comprises contacting with a peptide coupling reagent. In some embodiments, the peptide coupling reagent is a carbodiimide compound. In some embodiments, the carbodiimide compound is diisopropylcarbodiimide (DIC) or 1-ethyl-3-(3-dimethylaminopropyl)carbodiimide (EDC). In some embodiments, the method includes contacting with at least one compound of Formula (I) and a carbodiimide compounds, such as DIC or EDC.

In some embodiments, functionalization of the terminal amino acid (e.g., NTAA) using a compound of Formula (V) and the subsequent elimination are as depicted in the following exemplary scheme:

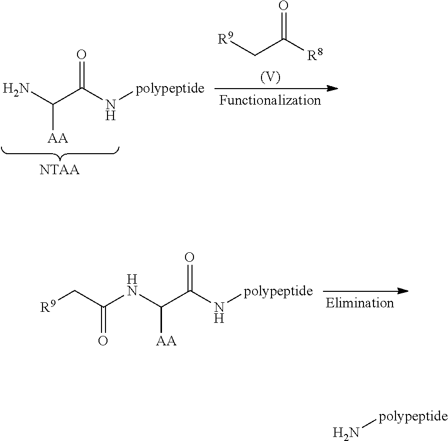

wherein R⁸ and R⁹ are as defined above and AA is the side chain of the NTAA.

In some embodiments, the elimination product of a NTAA functionalized with a compound of Formula (V) comprises

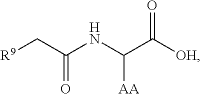

wherein R⁸ and R⁹ are as defined above and AA is the side chain of the NTAA.

In some embodiments, the reagent for eliminating the NTAA functionalized with a compound of Formula (V) comprises acylpeptide hydrolase (APH).

In some embodiments, a functionalizing reagent comprising a metal complex is used to functionalize the NTAA of a polypeptide. (See, e.g., Bentley et al., Biochem. J. 1973(135), 507-511; Bentley et al., Biochem. J. 1976(153), 137-138; Huo et al., J. Am. Chem. Soc. 2007, 139, 9819-9822; Wu et al., J. Am. Chem. Soc. 2016, 138(44), 14554-14557 incorporated by reference in their entireties). In some embodiments, the metal complex is a metal directing/chelating group. In some embodiments, the metal complex comprises one or more ligands chelated to a metal center. In some embodiments, the ligand is a monodentate ligand. In some embodiments, the ligand is a bidentate or polydentate ligand. In some embodiments, the metal complex comprises a metal selected from the group consisting of Co, Cu, Pd, Pt, Zn, and Ni.

In some embodiments, the functionalizing reagent comprises a compound selected from the group consisting of a compound of Formula (VI):

MLₙ (VI)

or a salt or conjugate thereof,

wherein

M is a metal selected from the group consisting of Co, Cu, Pd, Pt, Zn, and Ni;

L is a ligand selected from the group consisting of —OH, —OH₂, 2,2′-bipyridine (bpy), 1,5dithiacyclooctane (dtco), 1,2-bis(diphenylphosphino)ethane (dppe), ethylenediamine (en), and triethylenetetramine (trien); and

n is an integer from 1-8, inclusive;

wherein each L can be the same or different.

In some embodiments of Formula (VI), M is Co. In some embodiments, M is Cu. In some embodiments, M is Pd. In some embodiments, M is Pt. In some embodiments, M is Zn. In some embodiments, M is Ni. In some embodiments, the compound of Formula (VI) is anionic. In some embodiments, the compound of Formula (VI) is cationic. In some embodiments, the compound of Formula (VI) is neutral in charge.

In some embodiments of Formula (VI), n is 1. In some embodiments, n is 2. In some embodiments, n is 3. In some embodiments, n is 4. In some embodiments, n is 5. In some embodiments, n is 6. In some embodiments, n is 7. In some embodiments, n is 8. In some embodiments, M is Co and n is 3, 4, 5, 6, 7, or 8.

In some embodiments of Formula (VI), each L is selected from the group consisting of —OH, —OH₂, 2,2′-bipyridine (bpy), 1,5dithiacyclooctane (dtco), 1,2-bis(diphenylphosphino)ethane (dppe), ethylenediamine (en), and triethylenetetramine (trien).

In some embodiments, the compound is a cis-β-hydroxyaquo(triethylenetetramine)cobalt(III) complex. In some embodiments, the compound is β-[Co(trien)(OH)(OH₂)]²⁺.

In some embodiments, the compound of Formula (VI) activates the amide bond of the NTAA for intermolecular hydrolysis. In some embodiments, the intermolecular hydrolysis occurs in an aqueous solvent. In some embodiments, the intermolecular hydrolysis occurs in a nonaqueous solvent in the presence of water. In some embodiments, the elimination of the NTAA occurs by intramolecular delivery of hydroxide ligand from the metal species to the NTAA.

In some embodiments, functionalization of the NTAA using a compound of Formula (VI) and the subsequent elimination are as depicted in the following exemplary scheme:

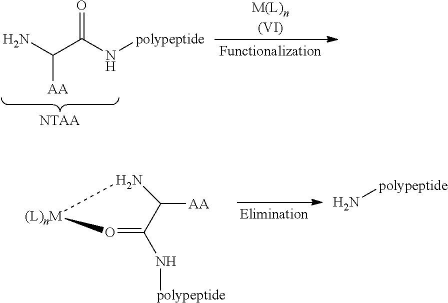

wherein M, L, and n are as defined above and AA is the side chain of the NTAA.

In some embodiments, the elimination product of a NTAA functionalized with a compound of Formula (VI) comprises

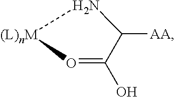

wherein M, L, and n are as defined above and AA is the side chain of the NTAA.

In some embodiments, a functionalizing reagent comprising a diketopiperazine (DKP) formation promoting group is used to functionalize the terminal amino acid (e.g., NTAA) of a polypeptide. In some embodiments, the DKP formation promoting group is an analog of proline. In some embodiments, the DKP formation promoting group is a cis peptide. In some embodiments, the cis peptide is conformationally restricted. In some embodiments, the DKP formation promoting group is a cis peptide mimetic (See, e.g., Tam et al., J. Am. Chem. Soc. 2007, 129, 12670-12671, incorporated by reference in its entirety). Diketopiperazine is a cyclic dipeptide that promotes the elimination reaction. In some embodiments, the NTAA is functionalized with a DKP formation promoting group. In some embodiments, functionalization of the NTAA with a DKP formation promoting group accelerates DKP formation. In some embodiments, after the NTAA is functionalized with a DKP formation promoting group, the NTAA is eliminated. In some embodiments, the NTAA is eliminated via DKP cyclo-elimination. In some embodiments, the elimination is assisted by a base or a lewis acid.

In some embodiments, the functionalizing reagent comprises a compound selected from the group consisting of a compound of Formula (VII):

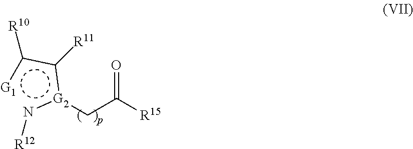

or a salt or conjugate thereof,
wherein

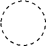

indicates that the ring is aromatic or nonaromatic;

G¹ is N, NR¹³, or CR¹³R¹⁴;

G² is N or CH;

p is 0 or 1;

R¹⁰, R¹¹, R¹², R¹³, and R¹⁴ are each independently selected from the group consisting of H, C₁₋₆ alkyl, C₁₋₆haloalkyl, C₁₋₆alkylamine, and C₁₋₆alkylhydroxylamine, wherein the C₁₋₆alkyl, C₁₋₆haloalkyl, C₁₋₆alkylamine, and C₁₋₆ alkylhydroxylamine are each unsubstituted or substituted, and R¹⁰ and R¹¹ can optionally come together to form a ring; and

R¹⁵ is H or OH.

In some embodiments of Formula (VII), G¹ is N or NR¹³. In some embodiments, Gi is CR¹³R¹⁴. In some embodiments, G¹ is CR¹³R¹⁴, and one of R¹³ and R¹⁴ is selected from the group consisting of H, C₁₋₆alkyl, C₁₋₆haloalkyl, C₁₋₆alkylamine, and C₁₋₆alkylhydroxylamine. In some embodiments, G¹ is CH₂. In some embodiments, G² is N. In some embodiments, G² is CH. In some embodiments, Gi is N or NR¹³. and G² is N. In some embodiments, G¹ is N or NR¹³. and G² is CH. In some embodiments, G¹ is CH₂ and G² is N. In some embodiments, Gi is CH₂ and G² is CH.

In some embodiments, R¹² is H. In some embodiments, R¹² is C₁₋₆alkyl, C₁₋₆haloalkyl, C₁₋₆alkylamine, or C₁₋₆alkylhydroxylamine. In some embodiments, R¹⁰ and R¹¹ are each H. In other embodiments, neither R¹⁰ nor R¹¹ are H. In some embodiments, R¹⁰ is H and R¹¹ is C₁₋₆alkyl, C₁₋₆haloalkyl, C₁₋₆alkylamine, or C₁₋₆alkylhydroxylamine. In some embodiments, R¹⁰ and R¹¹ come together to form a cycloalkyl, heterocyclyl, aryl, or heteroaryl ring. In some embodiments, R¹⁰ and R¹¹ come together to form a 5- or 6-membered ring. In some embodiments, R¹⁵ is H and p is 1. In some embodiments, R¹⁵ is H and p is 0. In some embodiments, R¹⁵ is OH and p is 1. In some embodiments, R¹⁵ is OH and p is 0.

In some embodiments, the compound is selected from the group consisting of

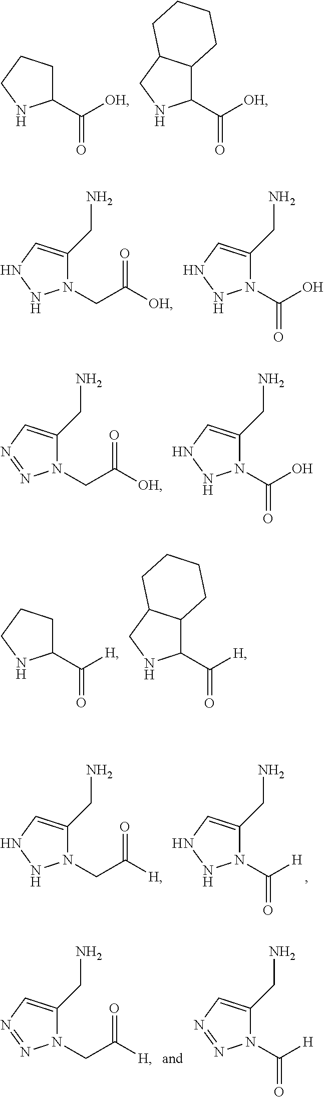

or a salt or conjugate thereof.

In some embodiments, functionalization of the NTAA using a reagent comprising a compound of Formula (VII) and the subsequent elimination are as depicted in the following exemplary scheme:

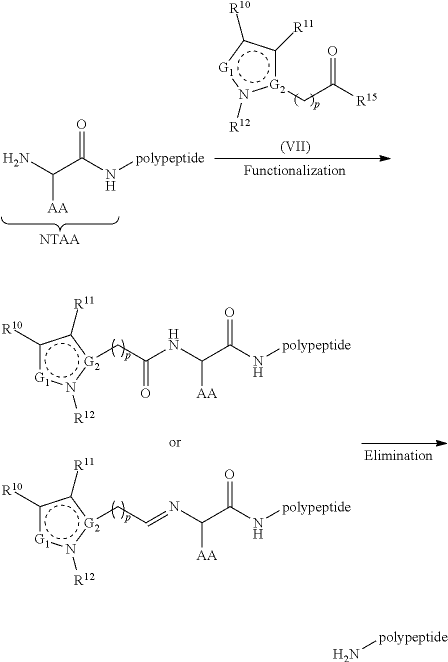

wherein R¹⁰, R¹¹, R¹², R¹⁵, G¹, G², and p are as defined above and AA is the side chain of the NTAA.

In some embodiments, the elimination product of a NTAA functionalized with a compound of Formula (VII) comprises

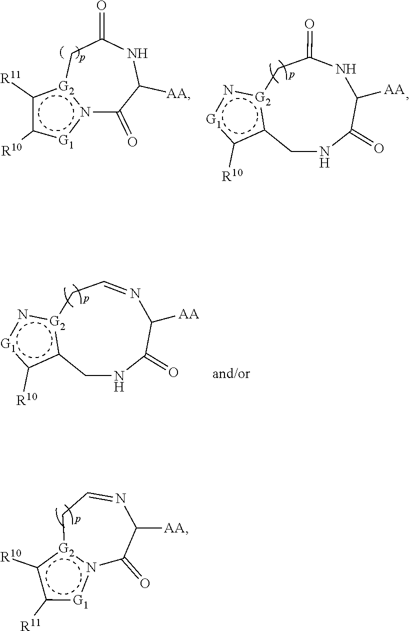

wherein R¹⁰, R¹¹, R¹², R¹⁵, G¹, G², and p are as defined above and AA is the side chain of the NTAA.

In some embodiments, the functionalizing reagent for modifying the terminal amino acid or a polypeptide comprises a conjugate of Formula (I), Formula (II), Formula (III), Formula (IV), Formula (V), Formula (VI), or Formula (VII). In some embodiments, the functionalizing reagent used to modify the terminal amino acid of a polypeptide comprises a compound of Formula (I), Formula (II), Formula (III), Formula (IV), Formula (V), Formula (VI), or Formula (VII) conjugated to a ligand.

In some embodiments, the functionalizing reagent for modifying the terminal amino acid of a polypeptide comprises a conjugate of Formula (I)-Q, Formula (II)-Q, Formula (III)-Q, Formula (IV)-Q, Formula (V)-Q, Formula (VI)-Q, or Formula (VII)-Q, wherein Formula (I)-(VII) are as defined above, and Q is a ligand.

In some embodiments, the ligand Q is a pendant group or binding site (e.g., the site to which the binding agent binds). In some embodiments, the polypeptide binds covalently to a binding agent. In some embodiments, the polypeptide comprises a functionalized NTAA which includes a ligand group that is capable of covalent binding to a binding agent. In certain embodiments, the polypeptide comprises a functionalized NTAA with a compound of Formula (I)-Q, Formula (II)-Q, Formula (III)-Q, Formula (IV)-Q, Formula (V)-Q, Formula (VI)-Q, or Formula (VII)-Q, wherein the Q binds covalently to a binding agent. In some embodiments, a coupling reaction is carried out to create a covalent linkage between the polypeptide and the binding agent (e.g., a covalent linkage between the ligand Q and a functional group on the binding agent).

In some embodiments, the functionalizing reagent for modifying the terminal amino acid of a polypeptide comprises a conjugate of Formula (I)-Q

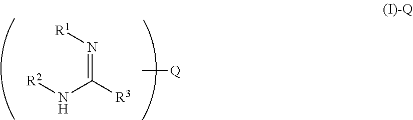

wherein R¹, R², and R³ are as defined above and Q is a ligand.

In some embodiments, the functionalizing reagent for modifying the terminal amino acid of a polypeptide comprises a conjugate of Formula (II)-Q

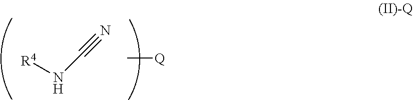

wherein R⁴ is as defined above, and Q is a ligand.

In some embodiments, the functionalizing reagent for modifying the terminal amino acid of a polypeptide comprises a conjugate of Formula (III)-Q

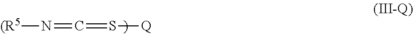

wherein R⁵ is as defined above and Q is a ligand.

In some embodiments, the functionalizing reagent for modifying the terminal amino acid of a polypeptide comprises a conjugate of Formula (IV)-Q

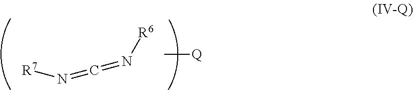

wherein R⁶ and R⁷ are as defined above and Q is a ligand.

In some embodiments, the functionalizing reagent for modifying the terminal amino acid of a polypeptide comprises a conjugate of Formula (V)-Q

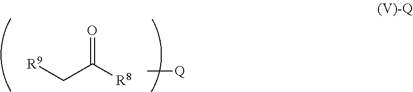

wherein R⁸ and R⁹ are as defined above and Q is a ligand.

In some embodiments, the functionalizing reagent for modifying the terminal amino acid of a polypeptide comprises a conjugate of Formula (VI)-Q

(MLₙ)-Q (VI)-Q

wherein M, L, and n are as defined above and Q is a ligand.

In some embodiments, the functionalizing reagent for modifying the terminal amino acid of a polypeptide comprises a conjugate of Formula (VII)-Q

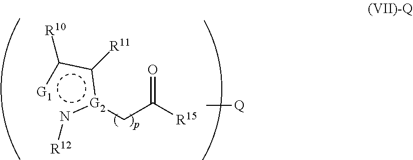

wherein R¹⁰, R¹¹, R¹², R¹⁵, G¹, G², and p are as defined above and Q is a ligand.

In some embodiments, Q is selected from the group consisting of —C₁₋₆alkyl, —C₂₋₆alkenyl, —C₂₋₆alkynyl, aryl, heteroaryl, heterocyclyl, —N═C═S, —CN, —C(O)Rⁿ, —C(O)OR^{o}, —SR^{p} or —S(O)₂R^{q}; wherein the —C₁₋₆alkyl, —C₂₋₆alkenyl, —C₂₋₆alkynyl, aryl, heteroaryl, and heterocyclyl are each unsubstituted or substituted, and Rⁿ, R^{o}, R^{p}, and R^{q} are each independently selected from the group consisting of —C₁₋₆ alkyl, —C₁₋₆haloalkyl, —C₂₋₆alkenyl, —C₂₋₆alkynyl, aryl, heteroaryl, and heterocyclyl. In some embodiments, Q is selected from the group consisting of

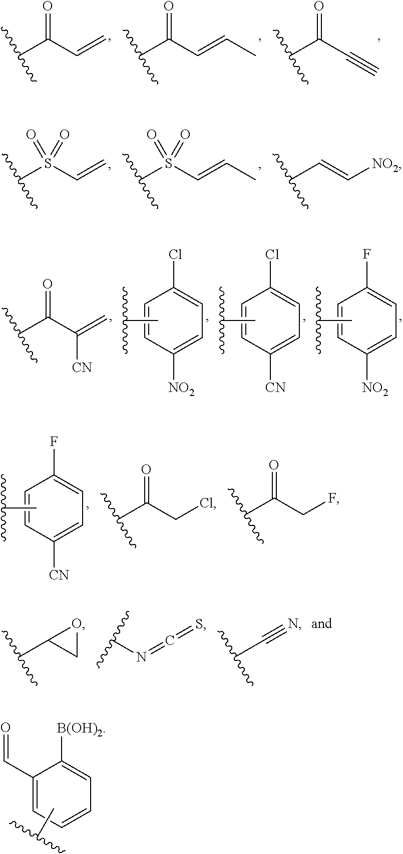

In some embodiments, Q is a fluorophore. In some embodiments, Q is selected from a lanthanide, europium, terbium, XL665, d2, quantum dots, green fluorescent protein, red fluorescent protein, yellow fluorescent protein, fluorescein, rhodamine, eosin, Texas red, cyanine, indocarbocyanine, ocacarbocyanine, thiacarbocyanine, merocyanine, pyridyloxadole, benzoxadiazole, cascade blue, nile red, oxazine 170, acridine orange, proflavin, auramine, malachite green crystal violet, porphine phtalocyanine, and bilirubin.

Provided in other aspects are reagents used in difunctionalizing the terminal amino acid. In some embodiments, the NTAA of the polypeptide is difunctionalized. In some embodiments, the CTAA of the polypeptide is difunctionalized.

In some embodiments, difunctionalizing the terminal amino acid (e.g., NTAA) includes using a first functionalizing reagent and a second functionalizing reagent. In some embodiments, the terminal amino acid is functionalized with the second functionalizing reagent prior to functionalizing with the first functionalizing reagent. In some embodiments, the terminal amino acid is functionalized with the first functionalizing reagent prior to functionalizing with the second functionalizing reagent. In some embodiments, the terminal amino acid is concurrently functionalized with the first functionalizing reagent and the second functionalizing reagent.

In some embodiments, the first functionalizing reagent comprises a compound selected from the group consisting of a compound of Formula (I), (II), (III), (IV), (V), (VI), and (VII), or a salt or conjugate thereof, as described herein.

In some embodiments, the second functionalizing reagent comprises a compound of Formula (VIIIa) or (VIIIb):

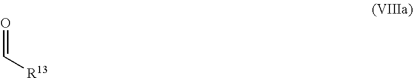

or a salt or conjugate thereof,
wherein
R¹³ is H, C₁₋₆ alkyl, aryl, heteroaryl, cycloalkyl, or heterocyclyl, wherein the C₁₋₆alkyl, aryl, heteroaryl, cycloalkyl, and heterocyclyl are each unsubstituted or substituted; or

R¹³—X (VIIIb)

wherein
R¹³ is C₁₋₆alkyl, aryl, heteroaryl, cycloalkyl, or heterocyclyl, each of which is unsubstituted or substituted; and
X is a halogen.

In some embodiments of Formula (VIIIa), R¹³ is H. In some embodiments, R¹³ is methyl. In some embodiments, R¹³ is ethyl, propyl, isopropyl, butyl, isobutyl, secbutyl, pentyl, or hexyl. In some embodiments, R¹³ is C₁₋₆alkyl, which is substituted. In some embodiments, R¹³ is C₁₋₆ alkyl, which is substituted with aryl, heteroaryl, cycloalkyl, or heterocyclyl. In some embodiments, R¹³ is C₁₋₆alkyl, which is substituted with aryl. In some embodiments, R¹³ is —CH₂CH₂Ph, —CH₂Ph, —CH(CH₃)Ph, or —CH(CH₃)Ph.

In some embodiments of Formula (VIIIb), R¹³ is methyl. In some embodiments, R¹³ is ethyl, propyl, isopropyl, butyl, isobutyl, secbutyl, pentyl, or hexyl. In some embodiments, R¹³ is C₁₋₆alkyl, which is substituted. In some embodiments, R¹³ is C₁₋₆alkyl, which is substituted with aryl, heteroaryl, cycloalkyl, or heterocyclyl. In some embodiments, R¹³ is C₁₋₆alkyl, which is substituted with aryl. In some embodiments, R¹³ is —CH₂CH₂Ph, —CH₂Ph, —CH(CH₃)Ph, or —CH(CH₃)Ph.

In some embodiments, the functionalizing reagent used to modify a terminal amino acid comprises formaldehyde. In some embodiments, the functionalizing reagent used to modify a terminal amino acid comprises methyl iodide.

In some embodiments, the method for modifying a polypeptide additionally comprises contacting the polypeptide with a reducing agent. In some embodiments, the reducing agent comprises a borohydride, such as NaBH₄, KBH₄, ZnBH₄, NaBH₃CN or LiBu₃BH. In some embodiments, the reducing agent comprises an aluminum or tin compound, such as LiAlH₄ or SnCl. In some embodiments, the reducing agent comprises a borane complex, such as B₂H₆ and dimethyamine borane. In some embodiments, the reagent additionally comprises NaBH₃CN.

In some embodiments, the terminal amino acid is modified with a functionalizing reagent comprising a compound of Formula (VIIIa) prior to functionalization with an additional reagent. In some embodiments, the terminal amino acid is functionalized with a functionalizing reagent comprising a compound of Formula (VIIIa) as depicted in the following exemplary scheme:

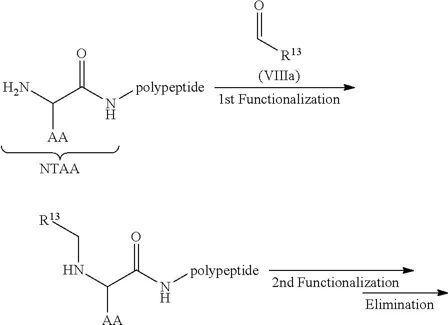

In some embodiments, the terminal amino acid is modified with a functionalizing reagent comprising a compound of Formula (VIIIb) as depicted in the following scheme:

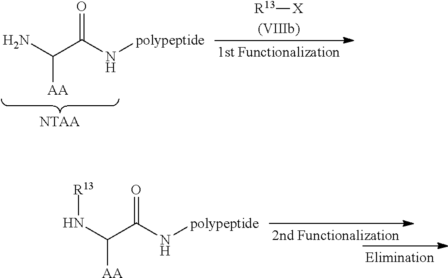

In some embodiments, the terminal amino acid is modified with a functionalizing reagent comprising a compound of Formula (VIIIa) or (VIIIb) and further modified with a functionalizing reagent comprising a compound of Formula (I). In some embodiments, the terminal amino acid is modified with a functionalizing reagent comprising a compound of Formula (VIIIa) or (VIIIb) and further modified with a functionalizing reagent comprising a compound of Formula (II). In some embodiments, the terminal amino acid is modified with a functionalizing reagent comprising a compound of Formula (VIIIa) or (VIIIb) and further modified with a functionalizing reagent comprising a compound of Formula (III). In some embodiments, the terminal amino acid is modified with a functionalizing reagent comprising a compound of Formula (VIIIa) or (VIIIb) and further f modified with a functionalizing reagent comprising a compound of Formula (IV). In some embodiments, the terminal amino acid is modified with a functionalizing reagent comprising a compound of Formula (VIIIa) or (VIIIb) and further modified with a functionalizing reagent comprising a compound of Formula (V). In some embodiments, the terminal amino acid is modified with a functionalizing reagent comprising a compound of Formula (VIIIa) or (VIIIb) and further modified with a functionalizing reagent comprising a compound of Formula (VI). In some embodiments, the terminal amino acid is modified with a functionalizing reagent comprising a compound of Formula (VIIIa) or (VIIIb) and further modified with a functionalizing reagent comprising a compound of Formula (VII).

In some embodiments, the terminal amino acid is first modified with a functionalizing reagent comprising a metal directing/chelating group prior to or concurrently with modification with a functionalizing reagent comprising a metal complex, such as a compound of Formula (VI). In some embodiments, the terminal amino acid is modified with a functionalizing reagent comprising a metal directing/chelating group to form an imine directing group formation. In some embodiments, the terminal amino acid is modified with a functionalizing reagent comprising a metal directing/chelating group to form an azo-methane ylide directing group. In some embodiments, the difunctionalization with a metal directing/chelating group and a compound of Formula (VI) activates the amide bond of the NTAA for intermolecular hydrolysis. In some embodiments, the intermolecular hydrolysis occurs in an aqueous solvent. In some embodiments, the intermolecular hydrolysis occurs in a nonaqueous solvent in the presence of water. In some embodiments, the elimination of the NTAA occurs by intramolecular delivery of hydroxide ligand from the metal species to the NTAA.

In some embodiments, the terminal amino acid is modified with a functionalizing reagent comprising a compound of Formula (VIIIa) or (VIIIb) and further modified with a functionalizing reagent comprising a compound of Formula (VI), such as depicted in the following exemplary scheme:

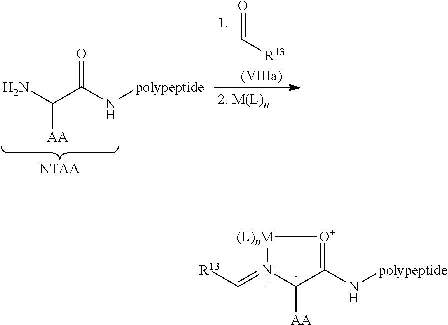

wherein R¹³, M, L, and n are as defined above and AA is the side chain of the NTAA.

In some embodiments, the reagents that may be used to functionalized the N-terminal amino acid (e.g., NTAA) include: 4-sulfophenyl isothiocyanate (sulfo-PITC), 4-nitrophenyl isothiocyanate (nitro-PITC), 3-pyridyl isothiocyanate (PYITC), 2-piperidinoethyl isothiocyanate (PEITC), 3-(4-morpholino) propyl isothiocyanate (MPITC), 3-(diethylamino)propyl isothiocyanate (DEPTIC) (Wang et al., 2009, Anal Chem 81: 1893-1900), (1-fluoro-2,4-dinitrobenzene (Sanger's reagent, DNFB), dansyl chloride (DNS-Cl, or 1-dimethylaminonaphthalene-5-sulfonyl chloride), 4-sulfonyl-2-nitrofluorobenzene (SNFB), acetylation reagents, amidination (guanidinylation) reagents (including PCA and PCA derivatives), 2-carboxy-4,6-dinitrochlorobenzene, 7-methoxycoumarin acetic acid, a thioacylation reagent, a thioacetylation reagent, and a thiobenzylation reagent. Many of these functionalization reagents are unreactive or minimally reactive with DNA including PITC, nitro-PITC, sulfo-PITC, PYITC, and guanidinylation reagents (e.g., PCA compounds). If the amino acid is blocked to labelling, there are a number of approaches to unblock the terminus, such as removing N-acetyl blocks with acyl peptide hydrolase (APH) (Farries, Harris et al., 1991, Eur. J. Biochem. 196:679-685). Methods of unblocking the N-terminus of a peptide are known in the art (see, e.g., Krishna et al., 1991, Anal. Biochem. 199:45-50; Leone et al., 2011, Curr. Protoc. Protein Sci., Chapter 11:Unit11.7; Fowler et al., 2001, Curr. Protoc. Protein Sci., Chapter 11: Unit 11.7, each of which is hereby incorporated by reference in its entirety).

Dansyl chloride reacts with the free amine group of a peptide to yield a dansyl derivative of the NTAA. DNFB and SNFB react the α-amine groups of a peptide to produce DNP-NTAA, and SNP-NTAA, respectively. Additionally, both DNFB and SNFB also react with the with ε-amine of lysine residues. DNFB also reacts with tyrosine and histidine amino acid residues. SNFB has better selectivity for amine groups than DNFB, and is preferred for amino acid functionalization (Carty et al., J Biol Chem (1968) 243(20): 5244-5253). In certain embodiments, lysine ε-amines are pre-blocked with an organic anhydride prior to polypeptide protease digestion into peptides.

Another useful NTAA modifier is an acetyl group since a known enzyme exists to eliminate acetylated NTAAs, namely acyl peptide hydrolases (APH) which eliminates the N-terminal acetylated amino acid, effectively shortening the peptide by a single amino acid (Chang et al., Sci Rep (2015) 5: 8673; Friedmann et al., F (2013) 280(22): 5570-5581). The NTAA can be chemically acetylated with acetic anhydride, NHS-acetate, or enzymatically acetylated with N-terminal acetyltransferases (NAT) (Chang et al., Sci Rep (2015) 5: 8673; Friedmann et al., F (2013) 280(22): 5570-5581). Yet another useful NTAA modifier is an amidinyl (guanidinyl) moiety since a proven cleavage chemistry of the amidinated NTAA is known, namely base incubation of the N-terminal amidinated peptide with 0.5-2% NaOH results in elimination of the N-terminal amino acid (Hamada et al., Bioorg Med Chem Lett (2016) 26(7): 1690-1695). This effectively provides a mild Edman-like chemical N-terminal degradation peptide sequencing process. Moreover, certain amidination (guanidinylation) reagents and the downstream NaOH cleavage are quite compatible with DNA encoding.

The presence of the DNP/SNP, acetyl, or amidinyl (guanidinyl) group on the NTAA may provide a better handle for interaction with an engineered binding agent. A number of commercial DNP antibodies exist with low nM affinities. Other methods of functionalizing the NTAA include functionalizing with trypligase (Liebscher et al., 2014, Angew Chem Int Ed Engl 53:3024-3028) and amino acyl transferase (Wagner, et al., 2011, J Am Chem Soc 133:15139-15147).

Isothiocyanates, in the presence of ionic liquids, have been shown to have enhanced reactivity to primary amines. Ionic liquids are excellent solvents (and serve as a catalyst) in organic chemical reactions and can enhance the reaction of isothiocyanates with amines to form thioureas. Moreover, ionic liquids may act as absorbers of microwave radiation to further enhance reactivity (Martinez-Palou, J. Mex. Chem. Soc (2007) 51(4): 252-264). An example is the use of the ionic liquid 1-butyl-3-methyl-imidazolium tetraflouoraborate [Bmim][BF4] for rapid and efficient functionalization of aromatic and aliphatic amines by phenyl isothiocyanate (PITC) (Le, Chen et al. 2005). Edman degradation involves the reaction of isothiocyanates, such as PITC, with the amino N-terminus of peptides. As such, in one embodiment ionic liquids are used to improve the efficiency of the Edman elimination process by providing milder functionalization and elimination conditions. For instance, the use of 5% (vol./vol.) PITC in ionic liquid [Bmim][BF4] at 25° C. for 10 min. is more efficient than functionalization under standard Edman PITC derivatization conditions which employ 5% (vol./vol.) PITC in a solution containing pyridine, ethanol, and ddH2O (1:1:1 vol./vol./vol.) at 55° C. for 60 min (Wang, Fang et al. 2009). In a preferred embodiment, internal lysine, tyrosine, histidine, and cysteine amino acids are blocked within the polypeptide prior to fragmentation into peptides. In this way, only the peptide α-amine group of the NTAA is accessible for modification during the peptide sequencing reaction. This may be particularly relevant when using DNFB (Sanger' reagent) and dansyl chloride.

In certain embodiments, some or certain amino acid have been blocked prior to the functionalization step (particularly the original N-terminus of the protein). In some cases, there are a number of approaches to unblock the N-terminus, such as removing N-acetyl blocks with acyl peptide hydrolase (APH) (Farcies, Harris et al. 1991). A number of other methods of unblocking the N-terminus of a peptide are known in the art (see, e.g., Krishna et al., 1991, Anal. Biochem. 199:45-50; Leone et al., 2011, Curr. Protoc. Protein Sci., Chapter 11:Unit11.7; Fowler et al., 2001, Curr. Protoc. Protein Sci., Chapter 11: Unit 11.7, each of which is hereby incorporated by reference in its entirety).

The CTAA can be functionalized with a number of different carboxyl-reactive reagents. In another example, the CTAA is functionalized with a mixed anhydride and an isothiocyanate to generate a thiohydantoin ((Liu et al., J Protein Chem (2001) 20(7): 535-541 and U.S. Pat. No. 5,049,507). The thiohydantoin modified peptide can be eliminated at elevated temperature in base to expose the penultimate CTAA, effectively generating a C-terminal based peptide degradation sequencing approach (Liu and Liang 2001). Other functionalizations that can be made to the CTAA include addition of a para-nitroanilide group and addition of 7-amino-4-methylcoumarinyl group.

In certain embodiments relating to analyzing peptides, following binding of a terminal amino acid (N-terminal or C-terminal) by a binding agent and transfer of coding tag information to a recording tag, transfer of recording tag information to a coding tag, transfer of recording tag information and coding tag information to a di-tag construct, the terminal amino acid is eliminated from the polypeptide to expose a new terminal amino acid. In some embodiments, the terminal amino acid is an NTAA. In other embodiments, the terminal amino acid is a CTAA.

B. Polypeptide Binding

Provided herein are methods accelerating a sequencing reaction with a polypeptide comprising contacting the polypeptide with one or more binding agents capable of binding at least a portion of the polypeptide and applying microwave energy. Also provided herein are methods of accelerating a reaction with a polypeptide comprising contacting the polypeptide with one or more binding agents and applying microwave energy, wherein each binding agent comprises a binding moiety capable of binding to a terminal amino acid residue, terminal di-amino-acid residues, or terminal triple-amino-acid residues of the polypeptide.

Also provided is a method of accelerating a sequencing reaction with a polypeptide including contacting the polypeptide with one or more binding agents capable of binding at least a portion of the polypeptide and applying microwave energy; and determining the sequence of at least a portion of the polypeptide. In some cases, the method for treating a polypeptide for sequence analysis includes (a) preparing a mixture comprising one or more polypeptides and one or more binding agents capable of binding at least a portion of the polypeptide; (b) subjecting the mixture to microwave energy; and (c) determining the sequence of at least a portion of the polypeptide.

In some embodiments, provided is a method of treating a polypeptide for sequence analysis, including the steps of (a) preparing a mixture comprising one or more polypeptides and one or more binding agents, wherein each binding agent comprises a binding moiety capable of binding to a terminal amino acid residue, terminal di-amino-acid residues, or terminal triple-amino-acid residues; and (b) subjecting the mixture to microwave energy. In some embodiments, step (a) is conducted before step (b). In some embodiments, step (b) is conducted before step (a). In some embodiments, wherein the step (a) and the step (b) are conducted in the same step or simultaneously.

In some of any of the provided embodiments, the binding agent binds a functionalized amino acid of the polypeptide. For example, the amino acid is functionalized according to the methods described in Section IA. In some examples, the binding agent binds a guanidinylated amino acid. In some of any of the provided embodiments, the binding agent binds a functionalized terminal amino acid of the polypeptide (e.g., a functionalized NTAA or CTAA). In some embodiments, the binding agent binds a guanidinylated terminal amino acid of the polypeptide (e.g., a functionalized NTAA or CTAA).

In some embodiments, the contacting of the binding agent with one or more polypeptides may be performed at any acceptable reaction times, such as about 60 minutes or below. In some embodiments, the amount of time for binding is below about 30 minutes, such as below about 10 minutes. In some embodiments, the amount of time for binding is below about 20 minutes, below about 15 minutes, below about 10 minutes, or below about 5 minutes. In some embodiments, the amount of time for binding is below about 10 minutes, below about 8 minutes, below about 5 minutes, or below about 3 minutes. In some embodiments, the contacting of the binding agent with one or more polypeptides may be performed at any acceptable reaction times, such as from about 1 minute to about 60 minutes, or a subrange thereof. In some aspects, the reaction time may be shortened by optimization of microwave conditions.

In some embodiments, the microwave energy is applied at about 5 watts, about 10 watts, about 15 watts, about 20 watts, about 25 watts, about 30 watts, about 35 watts, about 40 watts, about 45 watts, about 50 watts, about 60 watts, about 70 watts, about 80 watts, about 90 watts, about 100 watts, about 110 watts, about 120 watts, about 130 watts, about 140 watts, about 150 watts, or about 300 or higher watts, or a subrange thereof. In some examples, the microwave energy applied to the polypeptide and binding agent at or about 30 watts.

In some embodiments, the contacting with the one or more binding agent with the polypeptide are performed in the presence of microwave energy that maintains the reaction at a fixed temperature. In some examples, the contacting with the binding agent is performed in the presence of microwave energy that maintains the reaction at a temperature of about at least about 10° C., 20° C., 30° C., 40° C., 50° C., 60° C., 70° C., 80° C., 90° C., or 100° C., or a subrange thereof.

In some embodiments, the binding agent comprises a binding moiety capable of binding an internal polypeptide. In some embodiments, the binding agent comprises a binding moiety capable of binding one or more terminal amino acid residue(s). In some embodiments, the binding agent comprises a binding moiety capable of binding terminal di-amino-acid residues. In some embodiments, the binding agent comprises a binding moiety capable of binding terminal triple-amino-acid residues. In some embodiments, the binding agent comprises a binding moiety capable of binding an N-terminal amino acid (NTAA). In some embodiments, the binding agent comprises a binding moiety capable of binding a C-terminal amino acid (CTAA). In some embodiments, the binding agent comprises a binding moiety capable of binding a functionalized NTAA. In some embodiments, the binding agent comprises a binding moiety capable of binding a functionalized CTAA.

In some embodiments, the binding agents each comprise or are attached to a coding polymer comprising identifying information regarding the first binding moiety. In some of any of the provided embodiments, the binding agent and the coding tag are joined by a linker or a binding pair.

1. Binding Agents

The methods described herein use a binding agent capable of binding to the polypeptide. A binding agent can be any molecule (e.g., peptide, polypeptide, protein, nucleic acid, carbohydrate, small molecule, and the like) capable of binding to a component or feature of a polypeptide. A binding agent can be a naturally occurring, synthetically produced, or recombinantly expressed molecule. A binding agent may bind to a single monomer or subunit of a polypeptide (e.g., a single amino acid) or bind to multiple linked subunits of a polypeptide (e.g., dipeptide, tripeptide, or higher order peptide of a longer polypeptide molecule). In some embodiments, the binding agent binds to a terminal amino acid residue, terminal di-amino-acid residues, or terminal tri-amino-acid residues. In some embodiments, the binding agent binds to a post-translationally modified amino acid. In some embodiments, the polypeptide is contacted with a plurality of binding agents. For example, the plurality of binding agents comprises one or more binding agents that is or are configured to binding to the polypeptide.

In some embodiments, each binding agent comprises a binding moiety capable of binding an internal polypeptide, a terminal amino acid residue, di-amino-acid residues, terminal triple-amino-acid residues, an N-terminal amino acid (NTAA), a C-terminal amino acid (CTAA), a functionalized NTAA; or a functionalized CTAA.

In certain embodiments, a binding agent may be designed to bind covalently. Covalent binding can be designed to be conditional or favored upon binding to the correct moiety. For example, a NTAA and its cognate NTAA-specific binding agent may each be modified with a reactive group such that once the NTAA-specific binding agent is bound to the cognate NTAA, a coupling reaction is carried out to create a covalent linkage between the two. Non-specific binding of the binding agent to other locations that lack the cognate reactive group would not result in covalent attachment. In some embodiments, the polypeptide comprises a ligand that is capable of forming a covalent bond to a binding agent. In some embodiments, the polypeptide comprises a functionalized NTAA which includes a ligand group that is capable of covalent binding to a binding agent. Covalent binding between a binding agent and its target allows for more stringent washing to be used to remove binding agents that are non-specifically bound, thus increasing the specificity of the assay.

In certain embodiments, a binding agent may be a selective binding agent. As used herein, selective binding refers to the ability of the binding agent to preferentially bind to a specific ligand (e.g., amino acid or class of amino acids) relative to binding to a different ligand (e.g., amino acid or class of amino acids). Selectivity is commonly referred to as the equilibrium constant for the reaction of displacement of one ligand by another ligand in a complex with a binding agent. Typically, such selectivity is associated with the spatial geometry of the ligand and/or the manner and degree by which the ligand binds to a binding agent, such as by hydrogen bonding or Van der Waals forces (non-covalent interactions) or by reversible or non-reversible covalent attachment to the binding agent. It should also be understood that selectivity may be relative, and as opposed to absolute, and that different factors can affect the same, including ligand concentration. Thus, in one example, a binding agent selectively binds one of the twenty standard amino acids. In an example of non-selective binding, a binding agent may bind to two or more of the twenty standard amino acids.

In some embodiments, the binding agent is partially specific or selective. In some aspects, the binding agent preferentially binds one or more amino acids. For example, a binding agent may preferentially bind the amino acids A, C, and G over other amino acids. In some other examples, the binding agent may selectively or specifically bind more than one amino acid. In some aspects, the binding agent may also have a preference for one or more amino acids at the second, third, fourth, fifth, etc. positions from the terminal amino acid. In some cases, the binding agent preferentially binds to a specific terminal amino acid and one or more penultimate amino acid. In some cases, the binding agent preferentially binds to one or more specific terminal amino acid(s) and one penultimate amino acid. For example, a binding agent may preferentially bind AA, AC, and AG or a binding agent may preferentially bind AA, CA, and GA. In some specific examples, binding agents with different specificities can share the same coding tag.

In the practice of the methods disclosed herein, the ability of a binding agent to selectively bind a feature or component of a polypeptide need only be sufficient to allow transfer of its coding tag information to the recording tag associated with the polypeptide, transfer of the recording tag information to the coding tag, or transferring of the coding tag information and recording tag information to a di-tag molecule. Thus, selectively need only be relative to the other binding agents to which the polypeptide is exposed. It should also be understood that selectivity of a binding agent need not be absolute to a specific amino acid, but could be selective to a class of amino acids, such as amino acids with nonpolar or non-polar side chains, or with electrically (positively or negatively) charged side chains, or with aromatic side chains, or some specific class or size of side chains, and the like.

In a particular embodiment, the binding agent has a high affinity and high selectivity for the polypeptide of interest. In particular, a high binding affinity with a low off-rate is efficacious for information transfer between the coding tag and recording tag. In certain embodiments, a binding agent has a Kd of <500 nM, <100 nM, <50 nM, <10 nM, <5 nM, <1 nM, <0.5 nM, or <0.1 nM. In a particular embodiment, the binding agent is added to the polypeptide at a concentration >10×, >100×, or >1000× its Kd to drive binding to completion. A detailed discussion of binding kinetics of an antibody to a single protein molecule is described in Chang et al. (Chang, Rissin et al. 2012).

To increase the affinity of a binding agent to small N-terminal amino acids (NTAAs) of peptides, the NTAA may be modified with an “immunogenic” hapten, such as dinitrophenol (DNP). This can be implemented in a cyclic sequencing approach using Sanger's reagent, dinitrofluorobenzene (DNFB), which attaches a DNP group to the amine group of the NTAA. Commercial anti-DNP antibodies have affinities in the low nM range (˜8 nM, LO-DNP-2) (Bilgicer, Thomas et al. 2009); as such it stands to reason that it should be possible to engineer high-affinity NTAA binding agents to a number of NTAAs modified with DNP (via DNFB) and simultaneously achieve good binding selectivity for a particular NTAA. In another example, an NTAA may be modified with sulfonyl nitrophenol (SNP) using 4-sulfonyl-2-nitrofluorobenzene (SNFB). Similar affinity enhancements may also be achieved with alternative NTAA modifiers, such as an acetyl group or an amidinyl (guanidinyl) group.

In certain embodiments, a binding agent may bind to an NTAA, a CTAA, an intervening amino acid, dipeptide (sequence of two amino acids), tripeptide (sequence of three amino acids), or higher order peptide of a peptide molecule. In some embodiments, each binding agent in a library of binding agents selectively binds to a particular amino acid, for example one of the twenty standard naturally occurring amino acids. The standard, naturally-occurring amino acids include Alanine (A or Ala), Cysteine (C or Cys), Aspartic Acid (D or Asp), Glutamic Acid (E or Glu), Phenylalanine (F or Phe), Glycine (G or Gly), Histidine (H or His), Isoleucine (I or Ile), Lysine (K or Lys), Leucine (L or Leu), Methionine (M or Met), Asparagine (N or Asn), Proline (P or Pro), Glutamine (Q or Gln), Arginine (R or Arg), Serine (S or Ser), Threonine (T or Thr), Valine (V or Val), Tryptophan (W or Trp), and Tyrosine (Y or Tyr). In some embodiments, the binding agent binds to an unmodified or native amino acid. In some examples, the binding agent binds to an unmodified or native dipeptide (sequence of two amino acids), tripeptide (sequence of three amino acids), or higher order peptide of a peptide molecule. A binding agent may be engineered for high affinity for a native or unmodified NTAA, high specificity for a native or unmodified NTAA, or both. In some embodiments, binding agents can be developed through directed evolution of promising affinity scaffolds using phage display.

In certain embodiments, a binding agent may bind to a post-translational modification of an amino acid. In some embodiments, a peptide comprises one or more post-translational modifications, which may be the same of different. The NTAA, CTAA, an intervening amino acid, or a combination thereof of a peptide may be post-translationally modified. Post-translational modifications to amino acids include acylation, acetylation, alkylation (including methylation), biotinylation, butyrylation, carbamylation, carbonylation, deamidation, deiminiation, diphthamide formation, disulfide bridge formation, eliminylation, flavin attachment, formylation, gamma-carboxylation, glutamylation, glycylation, glycosylation, glypiation, heme C attachment, hydroxylation, hypusine formation, iodination, isoprenylation, lipidation, lipoylation, malonylation, methylation, myristolylation, oxidation, palmitoylation, pegylation, phosphopantetheinylation, phosphorylation, prenylation, propionylation, retinylidene Schiff base formation, S-glutathionylation, S-nitrosylation, S-sulfenylation, selenation, succinylation, sulfination, ubiquitination, and C-terminal amidation (see, also, Seo and Lee, 2004, J. Biochem. Mol. Biol. 37:35-44).

In certain embodiments, a lectin is used as a binding agent for detecting the glycosylation state of a protein, polypeptide, or peptide. Lectins are carbohydrate-binding proteins that can selectively recognize glycan epitopes of free carbohydrates or glycoproteins. A list of lectins recognizing various glycosylation states (e.g., core-fucose, sialic acids, N-acetyl-D-lactosamine, mannose, N-acetyl-glucosamine) include: A, AAA, AAL, ABA, ACA, ACG, ACL, AOL, ASA, BanLec, BC2L-A, BC2LCN, BPA, BPL, Calsepa, CGL2, CNL, Con, ConA, DBA, Discoidin, DSA, ECA, EEL, F17AG, Gall, Gal1-S, Gal2, Gal3, Gal3C-S, Gal7-S, Gal9, GNA, GRFT, GS-I, GS-II, GSL-I, GSL-II, HHL, HIHA, HPA, I, II, Jacalin, LBA, LCA, LEA, LEL, Lentil, Lotus, LSL-N, LTL, MAA, MAH, MAL_I, Malectin, MOA, MPA, MPL, NPA, Orysata, PA-IIL, PA-IL, PALa, PHA-E, PHA-L, PHA-P, PHAE, PHAL, PNA, PPL, PSA, PSL1a, PTL, PTL-I, PWM, RCA120, RS-Fuc, SAMB, SBA, SJA, SNA, SNA-I, SNA-II, SSA, STL, TJA-I, TJA-II, TxLCI, UDA, UEA-I, UEA-II, VFA, VVA, WFA, WGA (see, Zhang et al., 2016, MABS 8:524-535).

In certain embodiments, a binding agent may bind to a modified or labeled NTAA (e.g., an NTAA that has been functionalized by a reagent comprising a compound of any one of Formula (I)-(VII) as described herein). In some embodiments, the binding agent binds to an amino acid modified or functionalized using the methods and reagents provided in Section IA. In some examples, a modified or labeled NTAA can be one that is functionalized with PITC, 1-fluoro-2,4-dinitrobenzene (Sanger's reagent, DNFB), dansyl chloride (DNS-Cl, or 1-dimethylaminonaphthalene-5-sulfonyl chloride), 4-sulfonyl-2-nitrofluorobenzene (SNFB), an acetylating reagent, a guanidinylation reagent, a thioacylation reagent, a thioacetylation reagent, or a thiobenzylation reagent, or a reagent comprising a compound of any one of Formula (I)-(VII) as described herein.

In certain embodiments, a binding agent can be an aptamer (e.g., peptide aptamer, DNA aptamer, or RNA aptamer), an antibody, an anticalin, an ATP-dependent Clp protease adaptor protein (ClpS or ClpS2) or variant, mutant, or modified protein thereof, an antibody binding fragment, an antibody mimetic, a peptide, a peptidomimetic, a protein, or a polynucleotide (e.g., DNA, RNA, peptide nucleic acid (PNA), a γPNA, bridged nucleic acid (BNA), xeno nucleic acid (XNA), glycerol nucleic acid (GNA), or threose nucleic acid (TNA), or a variant thereof).

As used herein, the terms antibody and antibodies are used in a broad sense, to include not only intact antibody molecules, for example but not limited to immunoglobulin A, immunoglobulin G, immunoglobulin D, immunoglobulin E, and immunoglobulin M, but also any immunoreactivity component(s) of an antibody molecule that immuno-specifically bind to at least one epitope. An antibody may be naturally occurring, synthetically produced, or recombinantly expressed. An antibody may be a fusion protein. An antibody may be an antibody mimetic. Examples of antibodies include but are not limited to, Fab fragments, Fab′ fragments, F(ab′)2 fragments, single chain antibody fragments (scFv), miniantibodies, diabodies, crosslinked antibody fragments, Affibody™, nanobodies, single domain antibodies, DVD-Ig molecules, alphabodies, affimers, affitins, cyclotides, molecules, and the like. Immunoreactive products derived using antibody engineering or protein engineering techniques are also expressly within the meaning of the term antibodies. Detailed descriptions of antibody and/or protein engineering, including relevant protocols, can be found in, among other places, J. Maynard and G. Georgiou, 2000, Ann. Rev. Biomed. Eng. 2:339-76; Antibody Engineering, R. Kontermann and S. Dubel, eds., Springer Lab Manual, Springer Verlag (2001); U.S. Pat. No. 5,831,012; and S. Paul, Antibody Engineering Protocols, Humana Press (1995).

As with antibodies, nucleic acid and peptide aptamers that specifically recognize a peptide can be produced using known methods. Aptamers bind target molecules in a highly specific, conformation-dependent manner, typically with very high affinity, although aptamers with lower binding affinity can be selected if desired. Aptamers have been shown to distinguish between targets based on very small structural differences such as the presence or absence of a methyl or hydroxyl group and certain aptamers can distinguish between D- and L-enantiomers. Aptamers have been obtained that bind small molecular targets, including drugs, metal ions, and organic dyes, peptides, biotin, and proteins, including but not limited to streptavidin, VEGF, and viral proteins. Aptamers have been shown to retain functional activity after biotinylation, fluorescein labeling, and when attached to glass surfaces and microspheres. (see, Jayasena, 1999, Clin Chem 45:1628-50; Kusser, J. Biotechnol. (2000) 74: 27-39; Colas, 2000, Curr Opin Chem Biol 4:54-59). Aptamers which specifically bind arginine and AMP have been described as well (see, Patel et al., J. Biotech. (2000) 74:39-60). In some examples, there are. oligonucleotide aptamers that bind to a specific amino acid (Gold et al. (1995) Ann. Rev. Biochem. 64:763-97) and RNA aptamers that bind amino acids (Ames et al., (2011) RNA Biol. 8; 82-89; Mannironi et al., (2000) RNA 6:520-27; Famulok, (1994) J. Am. Chem. Soc. 116:1698-1706).

A binding agent can be made by modifying naturally-occurring or synthetically-produced proteins by genetic engineering to introduce one or more mutations in the amino acid sequence to produce engineered proteins that bind to a specific component or feature of a polypeptide (e.g., NTAA, CTAA, or post-translationally modified amino acid or a peptide). For example, exopeptidases (e.g., aminopeptidases, carboxypeptidases, dipeptidylpeptidase, dipeptidyl aminopeptidase), exoproteases, mutated exoproteases, mutated anticalins, mutated ClpSs, antibodies, or tRNA synthetases can be modified to create a binding agent that selectively or specifically binds to a particular NTAA. In another example, carboxypeptidases can be modified to create a binding agent that selectively binds to a particular CTAA. A binding agent can also be designed or modified, and utilized, to specifically bind a modified NTAA or modified CTAA, for example one that has a post-translational modification (e.g., phosphorylated NTAA or phosphorylated CTAA) or one that has been modified with a label (e.g., PTC or derivatized PTC, 1-fluoro-2,4-dinitrobenzene (using Sanger's reagent, DNFB), dansyl chloride (using DNS-Cl, or 1-dimethylaminonaphthalene-5-sulfonyl chloride), or using a thioacylation reagent, a thioacetylation reagent, an acetylation reagent, an amidination (guanidinylation) reagent, or a thiobenzylation reagent). Strategies for directed evolution of proteins are known in the art (e.g., reviewed by Yuan et al., (2005) Microbiol. Mol. Biol. Rev. 69:373-392), and include phage display, ribosomal display, mRNA display, CIS display, CAD display, emulsions, cell surface display method, yeast surface display, bacterial surface display, etc.

In some embodiments, a binding agent that selectively or specifically binds to a functionalized NTAA can be utilized. For example, the NTAA may be reacted with phenylisothiocyanate (PITC) to form a phenylthiocarbamoyl-NTAA derivative. Other isothiocyanates such as nitro-PITC, sulfo-PITC, and other isothiocyanate derivatives can also be used. In this manner, the binding agent may be fashioned to selectively bind both the phenyl group of the phenylthiocarbamoyl moiety as well as the alpha-carbon R group of the NTAA. Use of PITC or PITC derivatives in this manner allows for subsequent elimination of the NTAA by Edman degradation as discussed below. In another embodiment, the NTAA may be reacted with Sanger's reagent (DNFB), to generate a DNP-labeled NTAA. Optionally, DNFB is used with an ionic liquid such as 1-ethyl-3-methylimidazolium bis[(trifluoromethyl)sulfonyl]imide ([emim][Tf2N]), in which DNFB is highly soluble. In this manner, the binding agent may be engineered to selectively bind the combination of the DNP and the R group on the NTAA. The addition of the DNP moiety provides a larger “handle” for the interaction of the binding agent with the NTAA, and should lead to a higher affinity interaction. In yet another embodiment, a binding agent may be an aminopeptidase that has been engineered to recognize the DNP-labeled NTAA providing cyclic control of aminopeptidase degradation of the peptide. Once the DNP-labeled NTAA is eliminated, another cycle of DNFB derivatization is performed in order to bind and eliminate the newly exposed NTAA. In preferred particular embodiment, the aminopeptidase is a monomeric metallo-protease, such an aminopeptidase activated by zinc (Calcagno et al., Appl Microbiol Biotechnol. (2016) 100(16):7091-102). In another example, a binding agent may selectively bind to an NTAA that is modified with sulfonyl nitrophenol (SNP), e.g., by using 4-sulfonyl-2-nitrofluorobenzene (SNFB). In yet another embodiment, a binding agent may selectively bind to an NTAA that is acetylated or amidinated. In another embodiment, a binding agent may selectively bind to an NTAA that is guanidinylated.

Other reagents that may be used to functionalize the NTAA include trifluoroethyl isothiocyanate, allyl isothiocyanate, and dimethylaminoazobenzene isothiocyanate.

A binding agent may be engineered for high affinity for a modified NTAA, high specificity for a modified NTAA, or both. In some embodiments, binding agents can be developed through directed evolution of promising affinity scaffolds using phage display.

Engineered aminopeptidase mutants that bind to and cleave individual or small groups of labelled (biotinylated) NTAAs have been described (see, PCT Publication No. WO2010/065322, incorporated by reference in its entirety). Aminopeptidases are enzymes that cleave amino acids from the N-terminus of proteins or peptides. Natural aminopeptidases have very limited specificity, and generically eliminate N-terminal amino acids in a processive manner, cleaving one amino acid off after another (Kishor et al., Anal. Biochem. (2015) 488:6-8). However, residue specific aminopeptidases have been identified (Eriquez et al., J. Clin. Microbiol. (1980)12:667-71; Wilce et al., Proc. Natl. Acad. Sci. USA (1998) 95:3472-3477; Liao et al., Prot. Sci. (2004) 13:1802-10). Aminopeptidases may be engineered to specifically bind to 20 different NTAAs representing the standard amino acids that are labeled with a specific moiety (e.g., PTC or derivatized PTC, DNP, SNP, guanidinyl moiety etc.). Control of the stepwise degradation of the N-terminus of the peptide is achieved by using engineered aminopeptidases, dipeptidyl peptidases, amino peptidyl hydrolases that are only active (e.g., binding activity or catalytic activity) in the presence of the label. In another example, Havranak et al. (U.S. Patent Publication 2014/0273004) describes engineering aminoacyl tRNA synthetases (aaRSs) as specific NTAA binders. The amino acid binding pocket of the aaRSs has an intrinsic ability to bind cognate amino acids, but generally exhibits poor binding affinity and specificity. Moreover, these natural amino acid binders don't recognize N-terminal labels. Directed evolution of aaRS scaffolds can be used to generate higher affinity, higher specificity binding agents that recognized the N-terminal amino acids in the context of an N-terminal label.

In another example, there are highly-selective engineered ClpSs and directed evolution of an E. coli ClpS protein via phage display, resulting in four different variants with the ability to selectively bind NTAAs for aspartic acid, arginine, tryptophan, and leucine residues (U.S. Pat. No. 9,566,335, incorporated by reference in its entirety). In one embodiment, the binding moiety of the binding agent comprises a member of the evolutionarily conserved ClpS family of adaptor proteins involved in natural N-terminal protein recognition and binding or a variant thereof. The ClpS family of adaptor proteins in bacteria are described in Schuenemann et al., (2009) EMBO Rep. (2009) 10(5):508-14; and Roman-Hernandez et al., Proc Natl Acad Sci USA. (2009) 106(22):8888-93. See also Guo et al., (2002), JBC 277(48): 46753-62, and Wang et al., Mol Cell. (2008) 32(3):406-414. In some embodiments, the amino acid residues corresponding to the ClpS hydrophobic binding pocket identified in Schuenemann et al. are modified in order to generate a binding moiety with the desired selectivity.

In one embodiment, the binding moiety comprises a member of the UBR box recognition sequence family, or a variant of the UBR box recognition sequence family. UBR recognition boxes are described in Tasaki et al., (2009), JBC 284(3): 1884-95. For example, the binding moiety may comprise UBR1, UBR2, or a mutant, variant, or homologue thereof.

In certain embodiments, the binding agent further comprises one or more detectable labels such as fluorescent labels, in addition to the binding moiety. In some embodiments, the binding agent does not comprise a polynucleotide such as a coding tag. Optionally, the binding agent comprises a synthetic or natural antibody. In some embodiments, the binding agent comprises an aptamer. In one embodiment, the binding agent comprises a polypeptide, such as a modified member of the ClpS family of adaptor proteins, such as a variant of a E. Coli ClpS binding polypeptide, and a detectable label. In one embodiment, the detectable label is optically detectable. In some embodiments, the detectable label comprises a fluorescently moiety, a color-coded nanoparticle, a quantum dot or any combination thereof. In one embodiment the label comprises a polystyrene dye encompassing a core dye molecule such as a FluoSphere™, Nile Red, fluorescein, rhodamine, derivatized rhodamine dyes, such as TAMRA, phosphor, polymethadine dye, fluorescent phosphoramidite, TEXAS RED, green fluorescent protein, acridine, cyanine, cyanine 5 dye, cyanine 3 dye, 5-(2′-aminoethyl)-aminonaphthalene-1-sulfonic acid (EDANS), BODIPY, 120 ALEXA or a derivative or modification of any of the foregoing. In one embodiment, the detectable label is resistant to photobleaching while producing lots of signal (such as photons) at a unique and easily detectable wavelength, with high signal-to-noise ratio.

In a particular embodiment, anticalins are engineered for both high affinity and high specificity to labeled NTAAs (e.g., PTC or derivatized PTC, DNP, SNP, acetylated, guanidinylated, etc.). Certain varieties of anticalin scaffolds have suitable shape for binding single amino acids, by virtue of their beta barrel structure. An N-terminal amino acid (either with or without modification) can potentially fit and be recognized in this “beta barrel” bucket. High affinity anticalins with engineered novel binding activities have been described (reviewed by Skerra, 2008, FEBS J. 275: 2677-2683). For example, anticalins with high affinity binding (low nM) to fluorescein and digoxygenin have been engineered (Gebauer et al., Methods Enzymol (2012) 503: 157-188). Engineering of alternative scaffolds for new binding functions has also been reviewed by Banta et al. (2013, Annu. Rev. Biomed. Eng. 15:93-113).

The functional affinity (avidity) of a given monovalent binding agent may be increased by at least an order of magnitude by using a bivalent or higher order multimer of the monovalent binding agent (Vauquelin and Charlton 2013). Avidity refers to the accumulated strength of multiple, simultaneous, non-covalent binding interactions. An individual binding interaction may be easily dissociated. However, when multiple binding interactions are present at the same time, transient dissociation of a single binding interaction does not allow the binding protein to diffuse away and the binding interaction is likely to be restored. An alternative method for increasing avidity of a binding agent is to include complementary sequences in the coding tag attached to the binding agent and the recording tag associated with the polypeptide.

In some embodiments, a binding agent can be utilized that selectively or specifically binds a modified C-terminal amino acid (CTAA). Carboxypeptidases are proteases that cleave/eliminate terminal amino acids containing a free carboxyl group. A number of carboxypeptidases exhibit amino acid preferences, e.g., carboxypeptidase B preferentially cleaves at basic amino acids, such as arginine and lysine. A carboxypeptidase can be modified to create a binding agent that selectively binds to particular amino acid. In some embodiments, the carboxypeptidase may be engineered to selectively bind both the modification moiety as well as the alpha-carbon R group of the CTAA. Thus, engineered carboxypeptidases may specifically recognize 20 different CTAAs representing the standard amino acids in the context of a C-terminal label. Control of the stepwise degradation from the C-terminus of the peptide is achieved by using engineered carboxypeptidases that are only active (e.g., binding activity or catalytic activity) in the presence of the label. In one example, the CTAA may be modified by a para-Nitroanilide or 7-amino-4-methylcoumarinyl group.

Other potential scaffolds that can be engineered to generate binders for use in the methods described herein include: an anticalin, an amino acid tRNA synthetase (aaRS), ClpS, ClpS2, an Affilin®, an Adnectin™, a T cell receptor, a zinc finger protein, a thioredoxin, GST A1-1, DARPin, an affimer, an affitin, an alphabody, an avimer, a Kunitz domain peptide, a monobody, a single domain antibody, EETI-II, HPSTI, intrabody, lipocalin, PHD-finger, V(NAR) LDTI, evibody, Ig(NAR), knottin, maxibody, neocarzinostatin, pVIII, tendamistat, VLR, protein A scaffold, MTI-II, ecotin, GCN4, Im9, kunitz domain, microbody, PBP, trans-body, tetranectin, WW domain, CBM4-2, DX-88, GFP, iMab, Ldl receptor domain A, Min-23, PDZ-domain, avian pancreatic polypeptide, charybdotoxin/10Fn3, domain antibody (Dab), a2p8 ankyrin repeat, insect defensing A peptide, Designed AR protein, C-type lectin domain, staphylococcal nuclease, Src homology domain 3 (SH3), or Src homology domain 2 (SH2).

A binding agent may be engineered to withstand higher temperatures and mild-denaturing conditions (e.g., presence of urea, guanidinium thiocyanate, ionic solutions, etc.). The use of denaturants helps reduce secondary structures in the surface bound peptides, such as α-helical structures, β-hairpins, β-strands, and other such structures, which may interfere with binding of binding agents to linear peptide epitopes. In one embodiment, an ionic liquid such as 1-ethyl-3-methylimidazolium acetate ([EMIM]+[ACE] is used to reduce peptide secondary structure during binding cycles (Lesch, Heuer et al., Phys Chem Chem Phys (2015) 17(39): 26049-26053).

2. Coding Tags

In some embodiments, any of the binding agent described can also comprise a coding tag containing identifying information regarding the binding agent. A coding tag is a nucleic acid molecule of about 3 bases to about 100 bases that provides unique identifying information for its associated binding agent. A coding tag may comprise about 3 to about 90 bases, or a subrange thereof, e.g., about 3 to about 80 bases, about 3 to about 70 bases, about 3 to about 60 bases, about 3 bases to about 50 bases, about 3 bases to about 40 bases, about 3 bases to about 30 bases, about 3 bases to about 20 bases, about 3 bases to about 10 bases, or about 3 bases to about 8 bases. In some embodiments, a coding tag is about 3 bases, 4 bases, 5 bases, 6 bases, 7 bases, 8 bases, 9 bases, 10 bases, 11 bases, 12 bases, 13 bases, 14 bases, 15 bases, 16 bases, 17 bases, 18 bases, 19 bases, 20 bases, 25 bases, 30 bases, 35 bases, 40 bases, 55 bases, 60 bases, 65 bases, 70 bases, 75 bases, 80 bases, 85 bases, 90 bases, 95 bases, or 100 bases in length. A coding tag may be composed of DNA, RNA, polynucleotide analogs, or a combination thereof. Polynucleotide analogs include PNA, γPNA, BNA, GNA, TNA, LNA, morpholino polynucleotides, 2′-O-Methyl polynucleotides, alkyl ribosyl substituted polynucleotides, phosphorothioate polynucleotides, and 7-deaza purine analogs.

A coding tag comprises an encoder sequence that provides identifying information regarding the associated binding agent. In some embodiments, the “encoder sequence” or “encoder barcode” refers to a nucleic acid molecule of about 2 bases to about 30 bases or a subrange thereof, (e.g., 2, 3, 4, 5, 6, 7, 8, 9, 10, 11, 12, 13, 14, 15, 16, 17, 18, 19, 20, 21, 22, 23, 24, 25, 26, 27, 28, 29 or 30 bases) in length that provides identifying information for its associated binding agent. The encoder sequence may uniquely identify its associated binding agent. In certain embodiments, an encoder sequence provides identifying information for its associated binding agent and for the binding cycle in which the binding agent is used. In other embodiments, an encoder sequence is combined with a separate binding cycle-specific barcode within a coding tag. Alternatively, the encoder sequence may identify its associated binding agent as belonging to a member of a set of two or more different binding agents. In some embodiments, this level of identification is sufficient for the purposes of analysis. For example, in some embodiments involving a binding agent that binds to an amino acid, it may be sufficient to know that a peptide comprises one of two possible amino acids at a particular position, rather than definitively identify the amino acid residue at that position. In another example, a common encoder sequence is used for polyclonal antibodies, which comprises a mixture of antibodies that recognize more than one epitope of a protein target, and have varying specificities. In other embodiments, where an encoder sequence identifies a set of possible binding agents, a sequential decoding approach can be used to produce unique identification of each binding agent. This is accomplished by varying encoder sequences for a given binding agent in repeated cycles of binding (see, Gunderson et al., 2004, Genome Res. 14:870-7). The partially identifying coding tag information from each binding cycle, when combined with coding information from other cycles, produces a unique identifier for the binding agent, e.g., the particular combination of coding tags rather than an individual coding tag (or encoder sequence) provides the uniquely identifying information for the binding agent. Preferably, the encoder sequences within a library of binding agents possess the same or a similar number of bases.

An encoder sequence is about 3 bases to about 30 bases, or a subrange thereof, e.g., about 3 bases to about 20 bases, about 3 bases to about 10 bases, or about 3 bases to about 8 bases. In some embodiments, an encoder sequence is about 3 bases, 4 bases, 5 bases, 6 bases, 7 bases, 8 bases, 9 bases, 10 bases, 11 bases, 12 bases, 13 bases, 14 bases, 15 bases, 20 bases, 25 bases, or 30 bases in length. The length of the encoder sequence determines the number of unique encoder sequences that can be generated. Shorter encoding sequences generate a smaller number of unique encoding sequences, which may be useful when using a small number of binding agents. Longer encoder sequences may be desirable when analyzing a population of polypeptides. For example, an encoder sequence may be made up of 5 bases selected from any naturally occurring nucleotide, or analog. Using the four naturally occurring nucleotides A, T, C, and G, the total number of unique encoder sequences having a length of 5 bases is 1,024. In some embodiments, the total number of unique encoder sequences may be reduced by excluding, for example, encoder sequences in which all the bases are identical, at least three contiguous bases are identical, or both. In a specific embodiment, a set of ≥50 unique encoder sequences are used for a binding agent library.

In some embodiments, identifying components of a coding tag or recording tag, e.g., the encoder sequence, barcode, UMI, compartment tag, partition barcode, sample barcode, spatial region barcode, cycle specific sequence or any combination thereof, is subject to Hamming distance, Lee distance, asymmetric Lee distance, Reed-Solomon, Levenshtein-Tenengolts, or similar methods for error-correction. Hamming distance refers to the number of positions that are different between two strings of equal length. It measures the minimum number of substitutions required to change one string into the other. Hamming distance may be used to correct errors by selecting encoder sequences that are reasonable distance apart. Thus, in the example where the encoder sequence is 5 base, the number of useable encoder sequences is reduced to 256 unique encoder sequences (Hamming distance of 1→4⁴ encoder sequences=256 encoder sequences). In another embodiment, the encoder sequence, barcode, UMI, compartment tag, cycle specific sequence, or any combination thereof is designed to be easily read out by a cyclic decoding process (Gunderson et al., (2004) Genome Res. 14:870-7). In another embodiment, the encoder sequence, barcode, UMI, compartment tag, partition barcode, spatial barcode, sample barcode, cycle specific sequence, or any combination thereof is designed to be read out by low accuracy nanopore sequencing, since rather than requiring single base resolution, words of multiple bases (˜5-20 bases in length) need to be read.

In some embodiments, each unique binding agent within a library of binding agents has a unique encoder sequence. For example, 20 unique encoder sequences may be used for a library of 20 binding agents that bind to the 20 standard amino acids. Additional coding tag sequences may be used to identify modified amino acids (e.g., post-translationally modified amino acids). In another example, 30 unique encoder sequences may be used for a library of 30 binding agents that bind to the 20 standard amino acids and 10 post-translational modified amino acids (e.g., phosphorylated amino acids, acetylated amino acids, methylated amino acids). In other embodiments, two or more different binding agents may share the same encoder sequence. For example, two binding agents that each bind to a different standard amino acid may share the same encoder sequence.

In certain embodiments, a coding tag further comprises a spacer sequence at one end or both ends. A spacer sequence is about 1 base to about 20 bases, or a subrange thereof, e.g., about 1 base to about 10 bases, about 5 bases to about 9 bases, or about 4 bases to about 8 bases. In some embodiments, a spacer is about 1 base, 2 bases, 3 bases, 4 bases, 5 bases, 6 bases, 7 bases, 8 bases, 9 bases, 10 bases, 11 bases, 12 bases, 13 bases, 14 bases, 15 bases or 20 bases in length. In some embodiments, a spacer within a coding tag is shorter than the encoder sequence, e.g., at least 1 base, 2, bases, 3 bases, 4 bases, 5 bases, 6, bases, 7 bases, 8 bases, 9 bases, 10 bases, 11 bases, 12 bases, 13 bases, 14 bases, 15 bases, 20 bases, or 25 bases shorter than the encoder sequence. In other embodiments, a spacer within a coding tag is the same length as the encoder sequence. In certain embodiments, the spacer is binding agent specific so that a spacer from a previous binding cycle only interacts with a spacer from the appropriate binding agent in a current binding cycle. An example would be pairs of cognate antibodies containing spacer sequences that only allow information transfer if both antibodies sequentially bind to the polypeptide. A spacer sequence may be used as the primer annealing site for a primer extension reaction, or a splint or sticky end in a ligation reaction. A 5′ spacer on a coding tag may optionally contain pseudo complementary bases to a 3′ spacer on the recording tag to increase Tₘ (Lehoud et al., 2008, Nucleic Acids Res. 36:3409-3419).

In some embodiments, the coding tags within a collection of binding agents share a common spacer sequence used in an assay (e.g. the entire library of binding agents used in a multiple binding cycle method possess a common spacer in their coding tags). In another embodiment, the coding tags are comprised of a binding cycle tags, identifying a particular binding cycle. In other embodiments, the coding tags within a library of binding agents have a binding cycle specific spacer sequence. In some embodiments, a coding tag comprises one binding cycle specific spacer sequence.

In some embodiments, “binding cycle specific tag”, “binding cycle specific barcode”, or “binding cycle specific sequence” refers to a unique sequence used to identify a library of binding agents used within a particular binding cycle. A binding cycle specific tag may comprise about 2 bases to about 8 bases or a subrange thereof, e.g., (e.g., 2, 3, 4, 5, 6, 7, or 8 bases) in length. A binding cycle specific tag may be incorporated within a binding agent's coding tag as part of a spacer sequence, part of an encoder sequence, part of a UMI, or as a separate component within the coding tag.

For example, a coding tag for binding agents used in the first binding cycle comprise a “cycle 1” specific spacer sequence, a coding tag for binding agents used in the second binding cycle comprise a “cycle 2” specific spacer sequence, and so on up to “n” binding cycles. In further embodiments, coding tags for binding agents used in the first binding cycle comprise a “cycle 1” specific spacer sequence and a “cycle 2” specific spacer sequence, coding tags for binding agents used in the second binding cycle comprise a “cycle 2” specific spacer sequence and a “cycle 3” specific spacer sequence, and so on up to “n” binding cycles. This embodiment is useful for subsequent PCR assembly of non-concatenated extended recording tags after the binding cycles are completed. In some embodiments, a spacer sequence comprises a sufficient number of bases to anneal to a complementary spacer sequence in a recording tag or extended recording tag to initiate a primer extension reaction or sticky end ligation reaction.

In preferred embodiment, binding cycle-specific encoder sequences are used in coding tags. Cycle-specific encoder sequences can greatly improve sequencing accuracy and mappability by informatically correctly positioning amino acid barcodes given encoding failures in some cycles. Binding cycle-specific encoder sequences may be accomplished either via the use of completely unique analyte (e.g., NTAA)-binding cycle encoder barcodes or through a combinatoric use of an analyte (e.g., NTAA) encoder sequence joined to a cycle-specific barcode. The advantage of using a combinatoric approach is that fewer total barcodes need to be designed. For a set of 20 analyte binding agents used across 10 cycles, only 20 analyte encoder sequence barcodes and 10 binding cycle specific barcodes need to be designed. In contrast, if the binding cycle is embedded directly in the binding agent encoder sequence, then a total of 200 independent encoder barcodes may need to be designed. An advantage of embedding binding cycle information directly in the encoder sequence is that the total length of the coding tag can be minimized when employing error-correcting barcodes on a nanopore readout. The use of error-tolerant barcodes allows highly accurate barcode identification using sequencing platforms and approaches that are more error-prone, but have other advantages such as rapid speed of analysis, lower cost, and/or more portable instrumentation. One such example is a nanopore-based sequencing readout.

In some embodiments, a coding tag comprises a cleavable or nickable DNA strand within the second (3′) spacer sequence proximal to the binding agent. For example, the 3′ spacer may have one or more uracil bases that can be nicked by uracil-specific excision reagent (USER). USER generates a single nucleotide gap at the location of the uracil. In another example, the 3′ spacer may comprise a recognition sequence for a nicking endonuclease that hydrolyzes only one strand of a duplex. Preferably, the enzyme used for cleaving or nicking the 3′ spacer sequence acts only on one DNA strand (the 3′ spacer of the coding tag), such that the other strand within the duplex belonging to the (extended) recording tag is left intact. These embodiments are particularly useful in assays analysing proteins in their native conformation, as it allows the non-denaturing removal of the binding agent from the (extended) recording tag after primer extension has occurred and leaves a single stranded DNA spacer sequence on the extended recording tag available for subsequent binding cycles.

The coding tags may also be designed to contain palindromic sequences. Inclusion of a palindromic sequence into a coding tag allows a nascent, growing, extended recording tag to fold upon itself as coding tag information is transferred. The extended recording tag is folded into a more compact structure, effectively decreasing undesired inter-molecular binding and primer extension events.

In some embodiments, a coding tag comprises analyte-specific spacer that is capable of priming extension only on recording tags previously extended with binding agents recognizing the same analyte. An extended recording tag can be built up from a series of binding events using coding tags comprising analyte-specific spacers and encoder sequences. In one embodiment, a first binding event employs a binding agent with a coding tag comprised of a generic 3′ spacer primer sequence and an analyte-specific spacer sequence at the 5′ terminus for use in the next binding cycle; subsequent binding cycles then use binding agents with encoded analyte-specific 3′ spacer sequences. This design results in amplifiable library elements being created only from a correct series of cognate binding events. Off-target and cross-reactive binding interactions will lead to a non-amplifiable extended recording tag. In one example, a pair of cognate binding agents to a particular polypeptide analyte is used in two binding cycles to identify the analyte. The first cognate binding agent contains a coding tag comprised of a generic spacer 3′ sequence for priming extension on the generic spacer sequence of the recording tag, and an encoded analyte-specific spacer at the 5′ end, which will be used in the next binding cycle. For matched cognate binding agent pairs, the 3′ analyte-specific spacer of the second binding agent is matched to the 5′ analyte-specific spacer of the first binding agent. In this way, only correct binding of the cognate pair of binding agents will result in an amplifiable extended recording tag. Cross-reactive binding agents will not be able to prime extension on the recording tag, and no amplifiable extended recording tag product generated. This approach greatly enhances the specificity of the methods disclosed herein. The same principle can be applied to triplet binding agent sets, in which 3 cycles of binding are employed. In a first binding cycle, a generic 3′ Sp sequence on the recording tag interacts with a generic spacer on a binding agent coding tag. Primer extension transfers coding tag information, including an analyte specific 5′ spacer, to the recording tag. Subsequent binding cycles employ analyte specific spacers on the binding agents' coding tags.

In certain embodiments, a coding tag may further comprise a unique molecular identifier for the binding agent to which the coding tag is linked. A UMI for the binding agent may be useful in embodiments utilizing extended coding tags or di-tag molecules for sequencing readouts, which in combination with the encoder sequence provides information regarding the identity of the binding agent and number of unique binding events for a polypeptide.

In another embodiment, a coding tag includes a randomized sequence (a set of N's, where N=a random selection from A, C, G, T, or a random selection from a set of words). After a series of “n” binding cycles and transfer of coding tag information to the (extended) recording tag, the final extended recording tag product will be composed of a series of these randomized sequences, which collectively form a “composite” unique molecule identifier (UMI) for the final extended recording tag. If for instance each coding tag contains an (NN) sequence (4*4=16 possible sequences), after 10 sequencing cycles, a combinatoric set of 10 distributed 2-mers is formed creating a total diversity of 16¹⁰˜10¹² possible composite UMI sequences for the extended recording tag products. Given that a peptide sequencing experiment uses ˜10⁹ molecules, this diversity is more than sufficient to create an effective set of UMIs for a sequencing experiment. Increased diversity can be achieved by simply using a longer randomized region (a sequence of three, four, or more N's etc.) within the coding tag.

A coding tag may include a terminator nucleotide incorporated at the 3′ end of the 3′ spacer sequence. After a binding agent binds to a polypeptide and their corresponding coding tag and recording tags anneal via complementary spacer sequences, it is possible for primer extension to transfer information from the coding tag to the recording tag, or to transfer information from the recording tag to the coding tag. Addition of a terminator nucleotide on the 3′ end of the coding tag prevents transfer of recording tag information to the coding tag. It is understood that for embodiments described herein involving generation of extended coding tags, it may be preferable to include a terminator nucleotide at the 3′ end of the recording tag to prevent transfer of coding tag information to the recording tag.

A coding tag may be a single stranded molecule, a double stranded molecule, or a partially double stranded. A coding tag may comprise blunt ends, overhanging ends, or one of each. In some embodiments, a coding tag is partially double stranded, which prevents annealing of the coding tag to internal encoder and spacer sequences in a growing extended recording tag. In some embodiments, the coding tag may comprise a hairpin. In certain embodiments, the hairpin comprises mutually complementary nucleic acid regions are connected through a nucleic acid strand. In some embodiments, the nucleic acid hairpin can also further comprise 3′ and/or 5′ single-stranded region(s) extending from the double-stranded stem segment. In some examples, the hairpin comprises a single strand of nucleic acid.

3. Binding Agent and Coding Tag Conjugate

A coding tag is joined to a binding agent directly or indirectly, by any means known in the art, including covalent and non-covalent interactions. In some embodiments, a coding tag may be joined to binding agent enzymatically or chemically. In some embodiments, a coding tag may be joined to a binding agent via ligation. In other embodiments, a coding tag is joined to a binding agent via affinity binding pairs (e.g., biotin and streptavidin).

In some embodiments, a binding agent is joined to a coding tag via SpyCatcher-SpyTag interaction. The SpyTag peptide forms an irreversible covalent bond to the SpyCatcher protein via a spontaneous isopeptide linkage, thereby offering a genetically encoded way to create peptide interactions that resist force and harsh conditions (Zakeri et al., (2012) Proc. Natl. Acad. Sci. 109:E690-697; Li et al., (2014) J. Mol. Biol. 426:309-317). A binding agent may be expressed as a fusion protein comprising the SpyCatcher protein. In some embodiments, the SpyCatcher protein is appended on the N-terminus or C-terminus of the binding agent. The SpyTag peptide can be coupled to the coding tag using standard conjugation chemistries (Bioconjugate Techniques, G. T. Hermanson, Academic Press (2013)).

In other embodiments, a binding agent is joined to a coding tag via SnoopTag-SnoopCatcher peptide-protein interaction. The SnoopTag peptide forms an isopeptide bond with the SnoopCatcher protein (Veggiani et al., Proc. Natl. Acad. Sci. USA, (2016) 113:1202-1207). A binding agent may be expressed as a fusion protein comprising the SnoopCatcher protein. In some embodiments, the SnoopCatcher protein is appended on the N-terminus or C-terminus of the binding agent. The SnoopTag peptide can be coupled to the coding tag using standard conjugation chemistries.

In yet other embodiments, a binding agent is joined to a coding tag via the HaloTag® protein fusion tag and its chemical ligand. HaloTag is a modified haloalkane dehalogenase designed to covalently bind to synthetic ligands (HaloTag ligands) (Los et al., (2008) ACS Chem. Biol. 3:373-382). The synthetic ligands comprise a chloroalkane linker attached to a variety of useful molecules. A covalent bond forms between the HaloTag and the chloroalkane linker that is highly specific, occurs rapidly under physiological conditions, and is essentially irreversible.

In certain embodiments, a polypeptide is also contacted with a non-cognate binding agent. As used herein, a non-cognate binding agent is referring to a binding agent that is selective for a different polypeptide feature or component than the particular polypeptide being considered. For example, if the n NTAA is phenylalanine, and the peptide is contacted with three binding agents selective for phenylalanine, tyrosine, and asparagine, respectively, the binding agent selective for phenylalanine would be first binding agent capable of selectively binding to the n^{th} NTAA (i.e., phenylalanine), while the other two binding agents would be non-cognate binding agents for that peptide (since they are selective for NTAAs other than phenylalanine). The tyrosine and asparagine binding agents may, however, be cognate binding agents for other peptides in the sample. If the n NTAA (phenylalanine) was then cleaved from the peptide, thereby converting the n-1 amino acid of the peptide to the n-1 NTAA (e.g., tyrosine), and the peptide was then contacted with the same three binding agents, the binding agent selective for tyrosine would be second binding agent capable of selectively binding to the n-1 NTAA (i.e., tyrosine), while the other two binding agents would be non-cognate binding agents (since they are selective for NTAAs other than tyrosine).

Thus, it should be understood that whether an agent is a binding agent or a non-cognate binding agent will depend on the nature of the particular polypeptide feature or component currently available for binding. Also, if multiple polypeptides are analyzed in a multiplexed reaction, a binding agent for one polypeptide may be a non-cognate binding agent for another, and vice versa. According, it should be understood that the following description concerning binding agents is applicable to any type of binding agent described herein (i.e., both cognate and non-cognate binding agents).

C. Removal of Amino Acid(s) from Polypeptide

Provided herein are methods accelerating a sequencing reaction with a polypeptide comprising contacting the polypeptide with a reagent (“removing reagent”) to remove one or more amino acid(s) from the polypeptide and applying microwave energy. Also provided herein are methods of accelerating a reaction with a polypeptide comprising contacting the polypeptide with a reagent to remove one or more N-terminal amino acids (NTAA) from the polypeptide and applying microwave energy.

Also provided is a method of accelerating a sequencing reaction with a polypeptide including contacting the polypeptide with a reagent (“removing reagent”) to remove one or more amino acid(s) from the polypeptide and applying microwave energy; and determining the sequence of at least a portion of the polypeptide.

In some of any of the provided embodiments, a functionalized amino acid of the polypeptide is removed by the reagent from the polypeptide. For example, the amino acid is functionalized according to the methods described in Section IA. In some examples, a guanidinylated amino acid is removed by the reagent from the polypeptide. In some of any of the provided embodiments, a functionalized terminal amino acid of the polypeptide (e.g., a functionalized NTAA or CTAA) is removed from the polypeptide. In some embodiments, a guanidinylated terminal amino acid of the polypeptide (e.g., NTAA) is removed from the polypeptide.

In some embodiments, the method for treating a polypeptide for sequence analysis includes the steps of (a) preparing a mixture comprising one or more polypeptides and reagents for removing one or more amino acids from the polypeptide; (b) subjecting the mixture to microwave energy; and (c) determining the sequence of at least a portion of the polypeptide. the removed amino acid comprises: an N-terminal amino acid (NTAA); an N-terminal dipeptide sequence; an N-terminal tripeptide sequence; an internal amino acid; an internal dipeptide sequence; an internal tripeptide sequence; a C-terminal amino acid (CTAA); a C-terminal dipeptide sequence; or a C-terminal tripeptide sequence, or any combination thereof. In some cases, the one or more of the amino acid residues are modified or functionalized. In some embodiments, the reagent removes one amino acid. In some embodiments, the reagent removes two amino acids.

Also provided is a method of accelerating a reaction with a polypeptide including contacting the polypeptide with a reagent to remove one or more N-terminal amino acids (NTAA) from the polypeptide and applying microwave energy. In some embodiments, provided is a method of treating a polypeptide for sequence analysis including the steps of (a) preparing a mixture comprising one or more polypeptides and reagents for removing one or more N-terminal amino acids (NTAA) from the polypeptide; and (b) subjecting the mixture to microwave energy. In some embodiments, step (a) is conducted before step (b). In some embodiments, step (b) is conducted before step (a). In some embodiments, wherein the step (a) and the step (b) are conducted in the same step or simultaneously.

In some embodiments, removal of one or more amino acid(s) may be performed at any acceptable reaction times, such as about 60 minutes or below. In some embodiments, the reaction time for removing one or more amino acid(s) is below about 30 minutes, such as below about 10 minutes. In some embodiments, the reaction time for removing one or more amino acid(s) is below about 20 minutes, below about 15 minutes, below about 10 minutes, or below about 5 minutes. In some aspects, the reaction time may be shortened by optimization of microwave conditions. In some embodiments, the microwave energy is applied for a duration of time effective to achieve removal of an amino acid in 20%, 30%, 40%, 50%, 60%, 70%, 80%, 90% or greater polypeptides.

In some embodiments, the microwave energy is applied at about 5 watts, about 10 watts, about 15 watts, about 20 watts, about 25 watts, about 30 watts, about 35 watts, about 40 watts, about 45 watts, about 50 watts, about 60 watts, about 70 watts, about 80 watts, about 90 watts, about 100 watts, about 110 watts, about 120 watts, about 130 watts, about 140 watts, or about 150 or higher watts, or a subrange thereof. In some examples, the microwave energy applied to the reaction for removing one or more amino acid(s) is at or about 30 watts.

In some embodiments, the contacting with the reagent to remove one or more amino acid(s) is performed in the presence of microwave energy that maintains the reaction at a fixed temperature. In some examples, the contacting with the reagent to remove one or more amino acids is performed in the presence of microwave energy that maintains the reaction at a temperature of about at least about 10° C., 20° C., 30° C., 40° C., 50° C., 60° C., 70° C., 80° C., 90° C., or 100° C. or a subrange thereof. In some cases, the methods provided herein are performed in a vessel that provides a microwave energy to maintain the reaction at a temperature of about 30° C., 60° C., or 80° C. or a subrange thereof.

In some embodiments, microwave-assisted removal of one or more amino acids (e.g., elimination) of one or more amino acid(s) achieves greater uniformity in removal of the amino acids compared to in the absence of microwave energy. In some embodiments, application of microwave energy reduces bias of removal of different amino acids. For example, in some cases, some amino acid residues may exhibit bias or show decreased removal compared to other residues when reactions are performed in the absence of microwave energy (e.g., based on hydrophobicity, charge, or other characteristics). In some cases, application of microwave energy eliminates or reduces the bias of amino acid removal (e.g., removal of hydrophobic vs non-hydrophobic residues).

Removal (e.g., elimination) of a terminal amino acid can be accomplished by any number of known techniques, including chemical cleavage and enzymatic cleavage. An example of chemical cleavage is Edman degradation. During Edman degradation of the peptide the n NTAA is reacted with phenyl isothiocyanate (PITC) under mildly alkaline conditions to form the phenylthiocarbamoyl-NTAA derivative. Next, under acidic conditions, the phenylthiocarbamoyl-NTAA derivative is cleaved generating a free thiazolinone derivative, and thereby converting the n-1 amino acid of the peptide to an N-terminal amino acid (n-1 NTAA). The steps in this process are illustrated below:

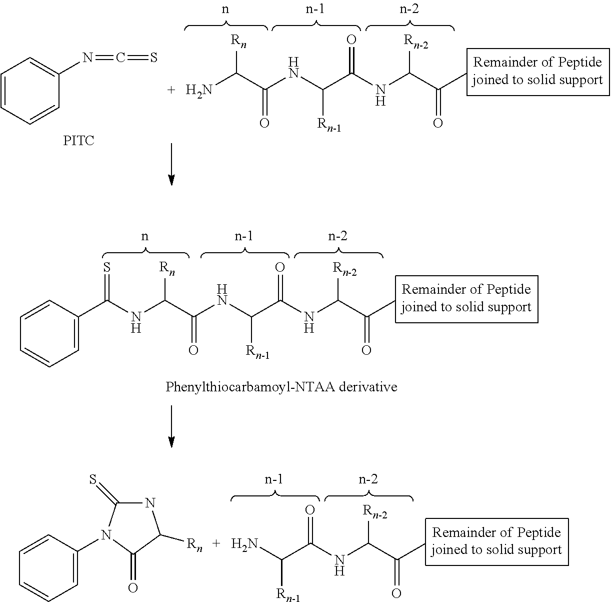

Typical Edman Degradation, as described above requires deployment of harsh high temperature chemical conditions (e.g., anhydrous TFA) for long incubation times. These conditions are generally not compatible with nucleic acid encoding of macromolecules.

To convert chemical Edman Degradation to a nucleic acid encoding-friendly approach, the harsh chemical steps are replaced with mild chemical degradation or efficient enzymatic steps. In one embodiment, chemical Edman degradation can be employed using milder conditions than original described. Several milder cleavage conditions for Edman degradation have been described in the literature, including replacing anhydrous TFA with triethylamine acetate in acetonitrile (see, e.g., Barrett, 1985, Tetrahedron Lett. 26:4375-4378, incorporated by reference in its entirety). Elimination of the NTAA may also be accomplished using thioacylation degradation, which uses milder elimination conditions as compared to Edman degradation (see, U.S. Pat. No. 4,863,870).

In another embodiment, amino acid removal by anhydrous TFA may be replaced with an “Edmanase”, an engineered enzyme that catalyzes the elimination of the PITC-derivatized N-terminal amino acid or modified PITC-derivatized NTAAs via nucleophilic attack of the thiourea sulfur atom on the carbonyl group of the scissile peptide bond under mild conditions (see, U.S. Patent Publication US2014/0273004, incorporated by reference in its entirety). Edmanase was made by modifying cruzain, a cysteine protease from Trypanosoma cruzi (Borgo et al., Protein Sci (2014) 23(3): 312-320). A C₂₅G mutation removes the catalytic cysteine residue while three mutations (G65S, A138C, L160Y) were selected to create steric fit with the phenyl moiety of the Edman reagent (PITC).

Enzymatic elimination or removal of a terminal amino acid may also be accomplished by an aminopeptidase. Aminopeptidases naturally occur as monomeric and multimeric enzymes, and may be metal or ATP-dependent. Natural aminopeptidases have very limited specificity, and generically eliminate N-terminal amino acids in a processive manner, removing one amino acid off after another. For the methods described here, aminopeptidases may be engineered to possess specific binding or catalytic activity to the NTAA only when functionalized with an N-terminal label. For example, an aminopeptidase may be engineered such than it only eliminates an N-terminal amino acid if it is functionalized by a group such as DNP/SNP, PTC, or derivatized PTC dansyl chloride, acetyl, amidinyl, guanidinyl, etc. In this way, the aminopeptidase removes only a single amino acid at a time from the N-terminus, and allows control of the degradation cycle. In some embodiments, the modified aminopeptidase is non-selective as to amino acid residue identity while being selective for the N-terminal label. In other embodiments, the modified aminopeptidase is selective for both amino acid residue identity and the N-terminal label. An example of a model of modifying the specificity of enzymatic NTAA degradation is illustrated by Borgo and Havranek, where through structure-function aided design, a methionine aminopeptidase was converted into a leucine aminopeptidase (Borgo and Havranek 2014). Engineered aminopeptidase mutants that bind to and eliminate individual or small groups of labelled (biotinylated) NTAAs have been described (see, PCT Publication No. WO2010/065322).

In certain embodiments, a compact monomeric metalloenzymatic aminopeptidase is engineered to recognize and eliminate DNP-labeled NTAAs. The use of a monomeric metallo-aminopeptidase has two key advantages: 1) compact monomeric proteins are much easier to display and screen using phage display; 2) a metallo-aminopeptidase has the unique advantage in that its activity can be turned on/off at will by adding or removing the appropriate metal cation. Exemplary aminopeptidases include the M28 family of aminopeptidases, such as Streptomyces sp. KK506 (SKAP) (Yoo et al., FEBS Lett. (2010) 584(19):4157-4162), Streptomyces griseus (SGAP), Vibrio proteolyticus (VPAP), (Spungin et al., Eur. J. Biochem. (1989) 183, 471-477; Ben-Meir, Spungin et al. Eur J Biochem. (1993) 212(1):107-12). These enzymes are stable, robust, and active at room temperature and pH 8.0, and thus compatible with mild conditions preferred for peptide analysis.

In another embodiment, cyclic elimination is attained by engineering the aminopeptidase to be active only in the presence of the N-terminal amino acid label. Moreover, the aminopeptidase may be engineered to be non-specific, such that it does not selectively recognize one particular amino acid over another, but rather just recognizes the functionalized N-terminus. In a preferred embodiment, a metallopeptidase monomeric aminopeptidase (e.g. Vibro leucine aminopeptidase) (Hernandez-Moreno et al., Int J Biol Macromol (2014) 64: 306-312), is engineered to eliminate only modified NTAAs (e.g., PTC or derivatized PTC, DNP, SNP, acetylated, acylated, guanidinylated, etc.)

In yet another embodiment, cyclic elimination is attained by using an engineered acylpeptide hydrolase (APH) to eliminate an acetylated NTAA. APH is a serine peptidase that is capable of catalyzing the removal of Na-acetylated amino acids from blocked peptides, and is a key regulator of N-terminally acetylated proteins in eukaryal, bacterial and archaeal cells. In certain embodiments, the APH is a dimeric and has only exopeptidase activity (Gogliettino, Balestrieri et al., PLoS One (2012) 7(5): e37921, Gogliettino, Riccio et al., FEBS J (2014) 281(1): 401-415). The engineered APH may have higher affinity and less selectivity than endogenous or wild type APHs.

In yet another embodiment, amidination (guanidinylation) of the NTAA is employed to enable mild elimination of the functionalized NTAA using NaOH (Hamada et al., Bioorg Med Chem Lett (2016) 26(7): 1690-1695) incorporated by reference in its entirety). A number of amidination (guanidinylation) reagents are known in the art including: S-methylisothiurea, 3,5-dimethylpyrazole-1-carboxamidine, S-ethylthiouronium bromide, S-ethylthiouronium chloride, O-methylisourea, O-methylisouronium sulfate, O-methylisourea hydrogen sulfate, 2-methyl-1-nitroisourea, aminoiminomethanesulfonic acid, cyanamide, cyanoguanide, dicyandiamide, 3,5-dimethyl-1-guanylpyrazole nitrate and 3,5-dimethyl pyrazole, N,N′-bis(ortho-chloro-Cbz)-S-methylisothiourea and N,N′-bis(ortho-bromo-Cbz)-S-methylisothiourea (Katritzky, 2005, incorporated by reference in its entirety).

Aminopeptidases with activity to a functionalized NTAAs may be selected using a screen combining tight-binding selection on the apo-enzyme (inactive in absence of metal cofactor) followed by a functional catalytic selection step, like the approach described by Ponsard et al. in engineering the metallo-beta-lactamase enzyme for benzylpenicillin (Ponsard et al., Chembiochem. (2001) 2(4):253-259, Femandez-Gacio et al., Trends Biotechnol. (2003) (9):408-414). This two-step selection is involves using a metallo-AP activated by addition of Zn2+ ions. After tight binding selection to an immobilized peptide substrate, Zn2+ is introduced, and catalytically active phage capable of hydrolyzing the NTAA functionalized with DNP or SNP leads to release of the bound phage into the supernatant. Repeated selection rounds are performed to enrich for active APs for DNP or SNP functionalized NTAA elimination.

In any of the embodiments provided herein, recruitment of a reagent to remove an amino acid, such as the NTAA, may be enhanced via a chimeric cleavage enzyme and chimeric NTAA modifier, wherein the chimeric cleavage enzyme and chimeric NTAA modifier each comprise a moiety capable of a tight binding reaction with each other (e.g., biotin-streptavidin). For example, an NTAA may be functionalized with biotin-PITC, and a chimeric cleavage enzyme (streptavidin-Edmanase) is recruited to the modified NTAA via the streptavidin-biotin interaction, improving the affinity and efficiency of the cleavage enzyme. The functionalized NTAA is eliminated and diffuses away from the peptide along with the associated cleavage enzyme. In the example of a chimeric Edmanase, this approach effectively increases the affinity K_{D} from μM to sub-picomolar.

For embodiments relating to CTAA binding agents, including methods of removing CTAA from peptides, See U.S. Pat. No. 6,046,053. In some embodiments, removing the CTAA includes reacting the peptide or protein with an alkyl acid anhydride to convert the carboxy-terminal into oxazolone, liberating the C-terminal amino acid by reaction with acid and alcohol or with ester. Enzymatic elimination of a CTAA may also be accomplished by a carboxypeptidase. Several carboxypeptidases exhibit amino acid preferences, e.g., carboxypeptidase B preferentially cleaves at basic amino acids, such as arginine and lysine. As described above, carboxypeptidases may also be modified in the same fashion as aminopeptidases to engineer carboxypeptidases that specifically bind to CTAAs having a C-terminal label. In this way, the carboxypeptidase eliminates only a single amino acid at a time from the C-terminus, and allows control of the degradation cycle. In some embodiments, the modified carboxypeptidase is non-selective as to amino acid residue identity while being selective for the C-terminal label. In other embodiments, the modified carboxypeptidase is selective for both amino acid residue identity and the C-terminal label.

In any of the embodiments provided herein, the NTAA is eliminated using a base. In some embodiments, the base is a hydroxide, an alkylated amine, a cyclic amine, a carbonate buffer, a trisodium phosphate buffer, or a metal salt. In some embodiments, the hydroxide is sodium hydroxide. In some embodiments, the alkylated amine is selected from methylamine, ethylamine, propylamine, dimethylamine, diethylamine, dipropylamine, trimethylamine, triethylamine, tripropylamine, cyclohexylamine, benzylamine, aniline, diphenylamine, N,N-diisopropylethylamine (DIPEA), and lithium diisopropylamide (LDA). In some embodiments, the NTAA can be eliminated using a cyclic amine. In some embodiments, the cyclic amine is selected from pyridine, pyrimidine, imidazole, pyrrole, indole, piperidine, prolidine, 1,8-diazabicyclo[5.4.0]undec-7-ene (DBU), and 1,5-diazabicyclo[4.3.0]non-5-ene (DBN). In some embodiments, the NTAA is eliminated using a carbonate buffer selected from the group consisting of sodium carbonate, potassium carbonate, calcium carbonate, sodium bicarbonate, potassium bicarbonate, or calcium bicarbonate. In some embodiments, the NTAA can be eliminated using a metal salt. In some embodiments, the metal salt comprises silver. In some embodiments, the NTAA is eliminated using AgClO₄.

In some embodiments, the NTAA is eliminated by a carboxypeptidase or aminopeptidase or variant, mutant, or modified protein thereof; a hydrolase or variant, mutant, or modified protein thereof; mild Edman degradation; Edmanase enzyme; TFA, a base; or any combination thereof.

In some embodiments, the NTAA is eliminated using mild Edman degradation. In some embodiments, mild Edman degradation comprises a dichloro or monochloro acid. In some embodiments, mild Edman degradation comprises TFA, TCA, or DCA. In some embodiments, mild Edman degradation comprises triethylamine, triethanolamine, or the mild Edman degradation uses triethylamine, triethanolamine, or triethylammonium acetate (Et₃NHOAc).

D. Exemplary Workflows

In some embodiments, one or more reactions described in Section I can be included in a workflow for treating one or more polypeptides. In some embodiments, a workflow comprising one or more of functionalization of amino acids, removal of amino acids, and binding of amino acids with a binding agent can be performed for polypeptide sequencing or analysis. In some embodiments, the modification by the functionalizing reagent is guanidinylation of an amino acid (e.g., guanidinylation of an terminal amino acid such as an NTAA). In some examples, the functionalized amino acid (e.g., guanidinylated amino acid) is bound by the binding agent. In some cases, the functionalized amino acid (e.g., guanidinylated amino acid) is removed by the reagent for removing one or more amino acids. In some embodiments, the guanidinylated amino acid is an NTAA of the polypeptide.

Provided herein is a method for preparing a plurality of polypeptides including (a) modifying the N-terminal amino acid (NTAA) of the polypeptide with a functionalizing reagent; and (b) contacting the polypeptide with a reagent to remove the NTAA. In some embodiments, step (a) and/or step (b) are performed in the presence of microwave energy. In some further embodiments, microwave energy is applied to the polypeptides before step (a) and/or step (b). In some embodiments, the method further includes step (a1) contacting the polypeptide with a binding agent that binds the functionalized NTAA, optionally in the presence of microwave energy. In some embodiments, the method further includes (c) determining the sequence of at least a portion of the polypeptide.

Provided herein is a method for analyzing a plurality of polypeptides, comprising (a) contacting a plurality of polypeptides with a functionalizing reagent to modify an amino acid of the polypeptide; (b) contacting the polypeptide with a reagent to remove the functionalized amino acid; and (c) determining the sequence of at least a portion of the polypeptide. In some embodiments, step (a) and/or step (b) are performed in the presence of microwave energy.

In some embodiments, the order in the steps in the process for a degradation-based peptide polypeptide sequencing assay can be reversed or moved around. For example, in some embodiments, the terminal amino acid functionalization can be conducted after the polypeptide is bound to the binding agent and/or associated coding tag. In some embodiments, the terminal amino acid functionalization can be conducted after the polypeptide is bound a support.

Provided here is a method for analyzing a polypeptide comprising the steps: (a) providing the polypeptide optionally associated directly or indirectly with a recording tag; (b) functionalizing the N-terminal amino acid (NTAA) of the polypeptide with a reagent to yield a functionalized NTAA, (c) contacting the polypeptide with a first binding agent comprising a first binding portion capable of binding to the functionalized NTAA and (c1) a first coding tag with identifying information regarding the first binding agent, or (c2) a first detectable label; (d) comprising (d1) transferring the information of the first coding tag to the recording tag to generate a first extended recording tag and analyzing the extended recording tag, or (d2) detecting the first detectable label, and (e) contacting the polypeptide with a reagent to remove the functionalized NTAA to expose a new NTAA. In some embodiments, any one or more of steps (b), (c), (d1), (d2) and/or (e) are performed in the presence of microwave energy. In some embodiments, between steps (d) and (e), steps (b) to (d) are repeated to determine the sequence of at least a portion of the polypeptide. In some embodiments, microwave energy is applied to the polypeptides prior to performing any of steps (a), (b), (c), (d) and/or (e).

In some embodiments, the method further comprises contacting the polypeptide with a proline aminopeptidase under conditions suitable to cleave an N-terminal proline before step (b). In some examples, a proline aminopeptidase (PAP) is an enzyme that is capable of specifically cleaving an N-terminal proline from a polypeptide. PAP enzymes that cleave N-terminal prolines are also referred to as proline iminopeptidases (PIPs). Known monomeric PAPs include family members from B. coagulans, L. delbrueckii, N.gonorrhoeae, F. meningosepticum, S. marcescens, T. acidophilum, L. plantarum (MEROPS S33.001) Nakajima et al., J Bacteriol. (2006) 188(4):1599-606; Kitazono et al., Bacteriol (1992) 174(24):7919-7925). Known multimeric PAPs including D. hansenii (Bolumar et al., (2003) 86(1-2):141-151) and similar homologues from other species (Basten et al., Mol Genet Genomics (2005) 272(6):673-679). Either native or engineered variants/mutants of PAPs may be employed.

In an exemplary workflow, functionalization of an amino acid, contacting the polypeptide with a binding agent, and removal of an amino acid is performed as follows: a large collection of recording tag labeled peptides (e.g., 50 million-1 billion or more) from a proteolytic digest are immobilized randomly on a single molecule sequencing substrate (e.g., beads) at an appropriate intramolecular spacing. In a cyclic manner, the N-terminal amino acid (NTAA) of each peptide are modified with a small chemical moiety (e.g., DNP, SNP, acetyl, guanidinyl) to provide cyclic control of the NTAA degradation process, and enhance binding affinity by a cognate binding agent, and microwave energy may be applied at this step. The functionalized N-terminal amino acid (e.g., DNP-NTAA, SNP-NTAA, acetyl-NTAA, guanidinylated-NTAA) of each immobilized peptide is bound by the cognate NTAA binding agent, and information from the coding tag associated with the bound NTAA binding agent is transferred to the recording tag associated with the immobilized peptide. Microwave energy may be applied to the interaction of the binding agent with the peptide. After NTAA recognition, binding, and transfer of coding tag information to the recording tag, the labelled NTAA is removed by exposure to an removing reagent that is capable of NTAA elimination only in the presence of the label (e.g., PTC or derivatized PTC, DNP, SNP, acetyl, guanidinyl), and microwave energy may be applied at this step. Other NTAA labels could also be employed with a suitably engineered aminopeptidase (AP) or dipeptidyl peptidase (DPP). In a particular embodiment, a single engineered AP, DPP, or APH universally eliminates all possible NTAAs (including post-translational modification variants) that possess the N-terminal amino acid label. In another particular embodiment, two, three, four, or more engineered AP, DPP, or APHs are used to eliminate the repertoire of labeled NTAAs.

As an alternative to NTAA elimination, a dipeptidyl amino peptidase (DAP) can be used to cleave the last two N-terminal amino acids from the peptide. In certain embodiments, a single functionalized NTAA can be eliminated. In some embodiments, an approach to N-terminal degradation includes the following: N-terminal ligation of a butelase I peptide substrate attaches a TEV endopeptidase substrate to the N-terminal of the peptide. After attachment, TEV endopeptidase cleaves the newly ligated peptide from the query peptide (peptide undergoing sequencing) leaving a single asparagine (N) attached to the NTAA. In some embodiments, incubation with DAP, which eliminates two amino acids from the N-terminus, results in a net removal of the original NTAA. This whole process can be cycled in the N-terminal degradation process.

### II. Polypeptides

In some aspects, the present disclosure relates to the treatment, modification, reactions with, and/or preparation of polypeptides. A polypeptide treated, modified, prepared, or analyzed according the methods disclosed herein may be obtained from a suitable source or sample, including but not limited to: biological samples, such as cells (both primary cells and cultured cell lines), cell lysates or extracts, cell organelles or vesicles, including exosomes, tissues and tissue extracts; biopsy; fecal matter; bodily fluids (such as blood, whole blood, serum, plasma, urine, lymph, bile, cerebrospinal fluid, interstitial fluid, aqueous or vitreous humor, colostrum, sputum, amniotic fluid, saliva, anal and vaginal secretions, perspiration and semen, a transudate, an exudate (e.g., fluid obtained from an abscess or any other site of infection or inflammation) or fluid obtained from a joint (normal joint or a joint affected by disease such as rheumatoid arthritis, osteoarthritis, gout or septic arthritis) of virtually any organism, with mammalian-derived samples, including microbiome-containing samples, being preferred and human-derived samples, including microbiome-containing samples, being particularly preferred; environmental samples (such as air, agricultural, water and soil samples); microbial samples including samples derived from microbial biofilms and/or communities, as well as microbial spores; research samples including extracellular fluids, extracellular supernatants from cell cultures, inclusion bodies in bacteria, cellular compartments including mitochondrial compartments, and cellular periplasm.

In certain embodiments, the polypeptide is a protein or a protein complex. Amino acid sequence information and post-translational modifications of the polypeptide are transduced into a nucleic acid encoded library that can be analyzed via next generation sequencing methods. A polypeptide may comprise L-amino acids, D-amino acids, or both. A polypeptide may comprise a standard, naturally occurring amino acid, a modified amino acid (e.g., post-translational modification), an amino acid analog, an amino acid mimetic, or any combination thereof. In some embodiments, the polypeptide is naturally occurring, synthetically produced, or recombinantly expressed. In any of the aforementioned embodiments, the polypeptide may further comprise a post-translational modification.

Standard, naturally occurring amino acids include Alanine (A or Ala), Cysteine (C or Cys), Aspartic Acid (D or Asp), Glutamic Acid (E or Glu), Phenylalanine (F or Phe), Glycine (G or Gly), Histidine (H or His), Isoleucine (I or Ile), Lysine (K or Lys), Leucine (L or Leu), Methionine (M or Met), Asparagine (N or Asn), Proline (P or Pro), Glutamine (Q or Gln), Arginine (R or Arg), Serine (S or Ser), Threonine (T or Thr), Valine (V or Val), Tryptophan (W or Trp), and Tyrosine (Y or Tyr). Non-standard amino acids include selenocysteine, pyrrolysine, and N-formylmethionine, n-amino acids, Homo-amino acids, Proline and Pyruvic acid derivatives, 3-substituted Alanine derivatives, Glycine derivatives, Ring-substituted Phenylalanine and Tyrosine Derivatives, Linear core amino acids, and N-methyl amino acids.

A post-translational modification (PTM) of a polypeptide may be a covalent modification or enzymatic modification. Examples of post-translation modifications include, but are not limited to, acylation, acetylation, alkylation (including methylation), biotinylation, butyrylation, carbamylation, carbonylation, deamidation, deiminiation, diphthamide formation, disulfide bridge formation, eliminylation, flavin attachment, formylation, gamma-carboxylation, glutamylation, glycylation, glycosylation (e.g., N-linked, O-linked, C-linked, phosphoglycosylation), glypiation, heme C attachment, hydroxylation, hypusine formation, iodination, isoprenylation, lipidation, lipoylation, malonylation, methylation, myristolylation, oxidation, palmitoylation, pegylation, phosphopantetheinylation, phosphorylation, prenylation, propionylation, retinylidene Schiff base formation, S-glutathionylation, S-nitrosylation, S-sulfenylation, selenation, succinylation, sulfination, ubiquitination, and C-terminal amidation. A post-translational modification includes modifications of the amino terminus and/or the carboxyl terminus of a peptide, polypeptide, or protein. Modifications of the terminal amino group include, but are not limited to, des-amino, N-lower alkyl, N-di-lower alkyl, and N-acyl modifications. Modifications of the terminal carboxy group include, but are not limited to, amide, lower alkyl amide, dialkyl amide, and lower alkyl ester modifications (e.g., wherein lower alkyl is C₁-C₄ alkyl). A post-translational modification also includes modifications, such as but not limited to those described above, of amino acids falling between the amino and carboxy termini of a peptide, polypeptide, or protein. Post-translational modification can regulate a protein's “biology” within a cell, e.g., its activity, structure, stability, or localization. Phosphorylation is the most common post-translational modification and plays an important role in regulation of protein, particularly in cell signaling (Prabakaran et al., (2012) Wiley Interdiscip Rev Syst Biol Med 4: 565-583). The addition of sugars to proteins, such as glycosylation, has been shown to promote protein folding, improve stability, and modify regulatory function. The attachment of lipids to proteins enables targeting to the cell membrane. A post-translational modification can also include modifications to include one or more detectable labels.

In certain embodiments, the polypeptide can be fragmented. For example, the fragmented polypeptide can be obtained by fragmenting a polypeptide, protein or protein complex from a sample, such as a biological sample. The polypeptide, protein or protein complex can be fragmented by any means known in the art, including fragmentation by a protease or endopeptidase. In some embodiments, fragmentation of a polypeptide, protein or protein complex is targeted by use of a specific protease or endopeptidase. A specific protease or endopeptidase binds and cleaves at a specific consensus sequence (e.g., TEV protease which is specific for ENLYFQ \S consensus sequence). In other embodiments, fragmentation of a peptide, polypeptide, or protein is non-targeted or random by use of a non-specific protease or endopeptidase. A non-specific protease may bind and cleave at a specific amino acid residue rather than a consensus sequence (e.g., proteinase K is a non-specific serine protease). Proteinases and endopeptidases are well known in the art, and examples of such that can be used to cleave a protein or polypeptide into smaller peptide fragments include proteinase K, trypsin, chymotrypsin, pepsin, thermolysin, thrombin, Factor Xa, furin, endopeptidase, papain, pepsin, subtilisin, elastase, enterokinase, Genenase™ I, Endoproteinase LysC, Endoproteinase AspN, Endoproteinase GluC, etc. (Granvogl et al., (2007) Anal Bioanal Chem 389: 991-1002). In certain embodiments, a peptide, polypeptide, or protein is fragmented by proteinase K, or optionally, a thermolabile version of proteinase K to enable rapid inactivation. Proteinase K is quite stable in denaturing reagents, such as urea and SDS, enabling digestion of completely denatured proteins. Protein and polypeptide fragmentation into peptides can be performed before or after attachment of a DNA tag or DNA recording tag.

In some embodiments, the polypeptide to be analyzed is first contacted with a proline aminopeptidase under conditions suitable to remove an N-terminal proline, if present.

Chemical reagents can also be used to digest proteins into peptide fragments. A chemical reagent may cleave at a specific amino acid residue (e.g., cyanogen bromide hydrolyzes peptide bonds at the C-terminus of methionine residues). Chemical reagents for fragmenting polypeptides or proteins into smaller peptides include cyanogen bromide (CNBr), hydroxylamine, hydrazine, formic acid, BNPS-skatole [2-(2-nitrophenylsulfenyl)-3-methylindole], iodosobenzoic acid, •NTCB+Ni (2-nitro-5-thiocyanobenzoic acid), etc.

In certain embodiments, following enzymatic or chemical elimination, the resulting polypeptide fragments are approximately the same desired length, e.g., from about 10 amino acids to about 70 amino acids, from about 10 amino acids to about 60 amino acids, from about 10 amino acids to about 50 amino acids, about 10 to about 40 amino acids, from about 10 to about 30 amino acids, from about 20 amino acids to about 70 amino acids, from about 20 amino acids to about 60 amino acids, from about 20 amino acids to about 50 amino acids, about 20 to about 40 amino acids, from about 20 to about 30 amino acids, from about 30 amino acids to about 70 amino acids, from about 30 amino acids to about 60 amino acids, from about 30 amino acids to about 50 amino acids, or from about 30 amino acids to about 40 amino acids. A elimination reaction may be monitored, preferably in real time, by spiking the protein or polypeptide sample with a short test FRET (fluorescence resonance energy transfer) polypeptide comprising a peptide sequence containing a proteinase or endopeptidase elimination site. In the intact FRET peptide, a fluorescent group and a quencher group are attached to either end of the peptide sequence containing the elimination site, and fluorescence resonance energy transfer between the quencher and the fluorophore leads to low fluorescence. Upon elimination of the test peptide by a protease or endopeptidase, the quencher and fluorophore are separated giving a large increase in fluorescence. An elimination reaction can be stopped when a certain fluorescence intensity is achieved, allowing a reproducible elimination end point to be achieved.

A sample of polypeptides can undergo protein fractionation methods prior to attachment to a solid support, where proteins or peptides are separated by one or more properties such as cellular location, molecular weight, hydrophobicity, or isoelectric point, or protein enrichment methods. Alternatively, or additionally, protein enrichment methods may be used to select for a specific protein or peptide (see, e.g., Whiteaker et al., (2007) Anal. Biochem. 362:44-54) or to select for a particular post translational modification (see, e.g., Huang et al., (2014) J. Chromatogr. A 1372:1-17). Alternatively, a particular class or classes of proteins such as immunoglobulins, or immunoglobulin (Ig) isotypes such as IgG, can be affinity enriched or selected for analysis. In the case of immunoglobulin molecules, analysis of the sequence and abundance or frequency of hypervariable sequences involved in affinity binding are of particular interest, particularly as they vary in response to disease progression or correlate with healthy, immune, and/or or disease phenotypes. Overly abundant proteins can also be subtracted from the sample using standard immunoaffinity methods. Depletion of abundant proteins can be useful for plasma samples where over 80% of the protein constituent is albumin and immunoglobulins. Several commercial products are available for depletion of plasma samples of overly abundant proteins, such as PROTIA and PROT20 (Sigma-Aldrich).

In certain embodiments, the polypeptide is comprised of a protein or a peptide. In one embodiment, the protein or polypeptide is labeled with DNA recording tags through standard amine coupling chemistries. The ε-amino group (e.g., of lysine residues) and the N-terminal amino group are particularly susceptible to labeling with amine-reactive coupling agents, depending on the pH of the reaction (Mendoza et al., Mass Spectrom Rev (2009) 28(5): 785-815). In a particular embodiment, the recording tag is comprised of a reactive moiety (e.g., for conjugation to a solid surface, a multifunctional linker, or a polypeptide), a linker, a universal priming sequence, a barcode (e.g., compartment tag, partition barcode, sample barcode, fraction barcode, or any combination thereof), an optional UMI, and a spacer (Sp) sequence for facilitating information transfer to/from a coding tag. In another embodiment, the protein can be first labeled with a universal DNA tag, and the barcode-Sp sequence (representing a sample, a compartment, a physical location on a slide, etc.) are attached to the protein later through and enzymatic or chemical coupling step. A universal DNA tag comprises a short sequence of nucleotides that are used to label a polypeptide and can be used as point of attachment for a barcode (e.g., compartment tag, recording tag, etc.). For example, a recording tag may comprise at its terminus a sequence complementary to the universal DNA tag. In certain embodiments, a universal DNA tag is a universal priming sequence. Upon hybridization of the universal DNA tags on the labeled protein to complementary sequence in recording tags (e.g., bound to beads), the annealed universal DNA tag may be extended via primer extension, transferring the recording tag information to the DNA tagged protein. In a particular embodiment, the protein is labeled with a universal DNA tag prior to proteinase digestion into peptides. The universal DNA tags on the labeled peptides from the digest can then be converted into an informative and effective recording tag.

In certain embodiments, a polypeptide can be immobilized to a solid support by an affinity capture reagent (and optionally covalently crosslinked), wherein the recording tag is associated with the affinity capture reagent directly, or alternatively, the protein can be directly immobilized to the solid support with a recording tag.

A. Attaching Recording Tags to Polypeptides

At least one recording tag is associated or co-localized directly or indirectly with the polypeptide and joined to the solid support. A recording tag may comprise DNA, RNA, or polynucleotide analogs including PNA, γPNA, GNA, BNA, XNA, TNA, any other polynucleotide analogs, or a combination thereof. A recording tag may be single stranded, or partially or completely double stranded. A recording tag may have a blunt end or overhanging end. In certain embodiments, upon binding of a binding agent to a polypeptide, identifying information of the binding agent's coding tag is transferred to the recording tag to generate an extended recording tag. Further extensions to the extended recording tag can be made in subsequent binding cycles.

A recording tag can be joined to the solid support, directly or indirectly (e.g., via a linker), by any means known in the art, including covalent and non-covalent interactions, or any combination thereof. For example, the recording tag may be joined to the solid support by a ligation reaction. Alternatively, the solid support can include an agent or coating to facilitate joining, either direct or indirectly, of the recording tag, to the solid support. Strategies for immobilizing nucleic acid molecules to solid supports (e.g., beads) have been described in U.S. Pat. No. 5,900,481; Steinberg et al. (2004) Biopolymers 73:597-605; Lund et al., (1988) Nucleic Acids Res. 16: 10861-10880; and Steinberg et al. (2004) Biopolymers 73:597-605).

In certain embodiments, the co-localization of a polypeptide and associated recording tag is achieved by conjugating polypeptide and recording tag to a bifunctional linker attached directly to the solid support surface (Steinberg et al. (2004) Biopolymers 73:597-605). In further embodiments, a trifunctional moiety is used to derivitize the solid support (e.g., beads), and the resulting bifunctional moiety is coupled to both the polypeptide and recording tag.

Methods and reagents (e.g., click chemistry reagents and photoaffinity labelling reagents) such as those described for attachment of polypeptides and solid supports, may also be used for attachment of recording tags.

In a particular embodiment, a single recording tag is attached to a polypeptide, preferably via the attachment to a de-blocked N- or C-terminal amino acid. In another embodiment, multiple recording tags are attached to the polypeptide, preferably to the lysine residues or peptide backbone. In some embodiments, a polypeptide labeled with multiple recording tags is fragmented or digested into smaller peptides, with each peptide labeled on average with one recording tag.

In certain embodiments, a recording tag comprises an optional, unique molecular identifier (UMI), which provides a unique identifier tag for each polypeptide to which the UMI is associated with. A UMI can be about 3 to about 40 bases, or a subrange thereof, e.g., about 3 to about 30 bases, about 3 to about 20 bases, or about 3 to about 10 bases, or about 3 to about 8 bases. In some embodiments, a UMI is about 3 bases, 4 bases, 5 bases, 6 bases, 7 bases, 8 bases, 9 bases, 10 bases, 11 bases, 12 bases, 13 bases, 14 bases, 15 bases, 16 bases, 17 bases, 18 bases, 19 bases, 20 bases, 25 bases, 30 bases, 35 bases, or 40 bases in length. A UMI can be used to de-convolute sequencing data from a plurality of extended recording tags to identify sequence reads from individual polypeptides. In some embodiments, within a library of polypeptides, each polypeptide is associated with a single recording tag, with each recording tag comprising a unique UMI. In other embodiments, multiple copies of a recording tag are associated with a single polypeptide, with each copy of the recording tag comprising the same UMI. In some embodiments, a UMI has a different base sequence than the spacer or encoder sequences within the binding agents' coding tags to facilitate distinguishing these components during sequence analysis.

In certain embodiments, a recording tag comprises a barcode, e.g., other than the UMI if present. A barcode is a nucleic acid molecule of about 3 to about 30 bases, or a subrange thereof, e.g., about 3 to about 25 bases, about 3 to about 20 bases, about 3 to about 10 bases, about 3 to about 10 bases, about 3 to about 8 bases in length. In some embodiments, a barcode is about 3 bases, 4 bases, 5 bases, 6 bases, 7 bases, 8 bases, 9 bases, 10 bases, 11 bases, 12 bases, 13 bases, 14 bases, 15 bases, 20 bases, 25 bases, or 30 bases in length. In one embodiment, a barcode allows for multiplex sequencing of a plurality of samples or libraries. A barcode may be used to identify a partition, a fraction, a compartment, a sample, a spatial location, or library from which the polypeptide derived. Barcodes can be used to de-convolute multiplexed sequence data and identify sequence reads from an individual sample or library. For example, a barcoded bead is useful for methods involving emulsions and partitioning of samples, e.g., for purposes of partitioning the proteome.

A barcode can represent a compartment tag in which a compartment, such as a droplet, microwell, physical region on a solid support, etc. is assigned a unique barcode. The association of a compartment with a specific barcode can be achieved in any number of ways such as by encapsulating a single barcoded bead in a compartment, e.g., by direct merging or adding a barcoded droplet to a compartment, by directly printing or injecting a barcode reagent to a compartment, etc. The barcode reagents within a compartment are used to add compartment-specific barcodes to the polypeptide or fragments thereof within the compartment. Applied to protein partitioning into compartments, the barcodes can be used to map analysed peptides back to their originating protein molecules in the compartment. This can greatly facilitate protein identification. Compartment barcodes can also be used to identify protein complexes.

In other embodiments, multiple compartments that represent a subset of a population of compartments may be assigned a unique barcode representing the subset.

Alternatively, a barcode may be a sample identifying barcode. A sample barcode is useful in the multiplexed analysis of a set of samples in a single reaction vessel or immobilized to a single solid substrate or collection of solid substrates (e.g., a planar slide, population of beads contained in a single tube or vessel, etc.). Polypeptides from many different samples can be labeled with recording tags with sample-specific barcodes, and then all the samples pooled together prior to immobilization to a solid support, cyclic binding, and recording tag analysis. Alternatively, the samples can be kept separate until after creation of a DNA-encoded library, and sample barcodes attached during PCR amplification of the DNA-encoded library, and then mixed together prior to sequencing. This approach could be useful when assaying analytes (e.g., proteins) of different abundance classes. For example, the sample can be split and barcoded, and one portion processed using binding agents to low abundance analytes, and the other portion processed using binding agents to higher abundance analytes. In a particular embodiment, this approach helps to adjust the dynamic range of a particular protein analyte assay to lie within the “sweet spot” of standard expression levels of the protein analyte.

In certain embodiments, polypeptides from multiple different samples are labeled with recording tags containing sample-specific barcodes. The multi-sample barcoded polypeptides can be mixed together prior to a cyclic binding reaction. In this way, a highly-multiplexed alternative to a digital reverse phase protein array (RPPA) is effectively created (Guo et al., Proteome Sci (2012) 10(1): 56; Assadi, Lamerz et al., Mol Cell Proteomics (2013) 12(9): 2615-2622; Akbani et al. 2014; Mol Cell Proteomics (2014) 13(7): 1625-1643; Creighton et al., Drug Des Devel Ther (2015) 9: 3519-3527). The creation of a digital RPPA-like assay has numerous applications in translational research, biomarker validation, drug discovery, clinical, and precision medicine.

In certain embodiments, a recording tag comprises a universal priming site, e.g., a forward or 5′ universal priming site. A universal priming site is a nucleic acid sequence that may be used for priming a library amplification reaction and/or for sequencing. A universal priming site may include, but is not limited to, a priming site for PCR amplification, flow cell adaptor sequences that anneal to complementary oligonucleotides on flow cell surfaces (e.g., Illumina next generation sequencing), a sequencing priming site, or a combination thereof. A universal priming site can be about 10 bases to about 60 bases. In some embodiments, a universal priming site comprises an Illumina P5 primer (5′-AATGATACGGCGACCACCGA-3′-SEQ ID NO:11) or an Illumina P7 primer (5′-CAAGCAGAAGACGGCATACGAGAT-3′-SEQ ID NO:12).

In certain embodiments, a recording tag comprises a spacer at its terminus, e.g., 3′ end. As used herein reference to a spacer sequence in the context of a recording tag includes a spacer sequence that is identical to the spacer sequence associated with its cognate binding agent, or a spacer sequence that is complementary to the spacer sequence associated with its cognate binding agent. The terminal, e.g., 3′, spacer on the recording tag permits transfer of identifying information of a cognate binding agent from its coding tag to the recording tag during the first binding cycle (e.g., via annealing of complementary spacer sequences for primer extension or sticky end ligation).

In one embodiment, the spacer sequence is about 1-20 bases in length or a subrange thereof, e.g., about 2-12 bases in length, or 5-10 bases in length. The length of the spacer may depend on factors such as the temperature and reaction conditions of the primer extension reaction for transferring coding tag information to the recording tag.

In a preferred embodiment, the spacer sequence in the recording is designed to have minimal complementarity to other regions in the recording tag; likewise, the spacer sequence in the coding tag should have minimal complementarity to other regions in the coding tag. In other words, the spacer sequence of the recording tags and coding tags should have minimal sequence complementarity to components such unique molecular identifiers, barcodes (e.g., compartment, partition, sample, spatial location), universal primer sequences, encoder sequences, cycle specific sequences, etc. present in the recording tags or coding tags.

As described for the binding agent spacers, in some embodiments, the recording tags associated with a library of polypeptides share a common spacer sequence. In other embodiments, the recording tags associated with a library of polypeptides have binding cycle specific spacer sequences that are complementary to the binding cycle specific spacer sequences of their cognate binding agents, which can be useful when using non-concatenated extended recording tags.

The collection of extended recording tags can be concatenated after the fact. After the binding cycles are complete, the bead solid supports, each bead comprising on average one or fewer than one polypeptide per bead, each polypeptide having a collection of extended recording tags that are co-localized at the site of the polypeptide, are placed in an emulsion. The emulsion is formed such that each droplet, on average, is occupied by at most 1 bead. An optional assembly PCR reaction is performed in-emulsion to amplify the extended recording tags co-localized with the polypeptide on the bead and assemble them in co-linear order by priming between the different cycle specific sequences on the separate extended recording tags (Xiong et al., FEMS Microbiol Rev (2008) 32(3): 522-540). Afterwards the emulsion is broken and the assembled extended recording tags are sequenced.

In another embodiment, the DNA recording tag is comprised of a universal priming sequence (U1), one or more barcode sequences (BCs), and a spacer sequence (Sp1) specific to the first binding cycle. In the first binding cycle, binding agents employ DNA coding tags comprised of an Sp 1 complementary spacer, an encoder barcode, and optional cycle barcode, and a second spacer element (Sp2). The utility of using at least two different spacer elements is that the first binding cycle selects one of potentially several DNA recording tags and a single DNA recording tag is extended resulting in a new Sp2 spacer element at the end of the extended DNA recording tag. In the second and subsequent binding cycles, binding agents contain just the Sp2′ spacer rather than Sp1′. In this way, only the single extended recording tag from the first cycle is extended in subsequent cycles. In another embodiment, the second and subsequent cycles can employ binding agent specific spacers.

In some embodiments, a recording tag comprises from 5′ to 3′ direction: a universal forward (or 5′) priming sequence, a UMI, and a spacer sequence. In some embodiments, a recording tag comprises from 5′ to 3′ direction: a universal forward (or 5′) priming sequence, an optional UMI, a barcode (e.g., sample barcode, partition barcode, compartment barcode, spatial barcode, or any combination thereof), and a spacer sequence. In some other embodiments, a recording tag comprises from 5′ to 3′ direction: a universal forward (or 5′) priming sequence, a barcode (e.g., sample barcode, partition barcode, compartment barcode, spatial barcode, or any combination thereof), an optional UMI, and a spacer sequence.

Combinatorial approaches may be used to generate UMIs from modified DNA and PNAs. In one example, a UMI may be constructed by “chemical ligating” together sets of short word sequences (4-15mers), which have been designed to be orthogonal to each other (Spiropulos and Heemstra 2012). A DNA template is used to direct the chemical ligation of the “word” polymers. The DNA template is constructed with hybridizing arms that enable assembly of a combinatorial template structure simply by mixing the sub-components together in solution. In certain embodiments, there are no “spacer” sequences in this design. The size of the word space can vary from 10's of words to 10,000's or more words or a subrange thereof. In certain embodiments, the words are chosen such that they differ from one another to not cross hybridize, yet possess relatively uniform hybridization conditions. In one embodiment, the length of the word will be on the order of 10 bases, with about 1000's words in the subset (this is only 0.1% of the total 10-mer word space ˜4¹⁰=1 million words). Sets of these words (1000 in subset) can be concatenated together to generate a final combinatorial UMI with complexity=1000n power. For 4 words concatenated together, this creates a UMI diversity of 10¹² different elements. These UMI sequences will be appended to the polypeptide at the single molecule level. In one embodiment, the diversity of UMIs exceeds the number of molecules of polypeptides to which the UMIs are attached. In this way, the UMI uniquely identifies the polypeptide of interest. The use of combinatorial word UMI's facilitates readout on high error rate sequencers, (e.g., nanopore sequencers, nanogap tunneling sequencing, etc.) since single base resolution is not required to read words of multiple bases in length. Combinatorial word approaches can also be used to generate other identity-informative components of recording tags or coding tags, such as compartment tags, partition barcodes, spatial barcodes, sample barcodes, encoder sequences, cycle specific sequences, and barcodes. Methods relating to nanopore sequencing and DNA encoding information with error-tolerant words (codes) are known in the art (see, e.g., Kiah et al., 2015, Codes for DNA sequence profiles. IEEE International Symposium on Information Theory (ISIT); Gabrys et al., 2015, Asymmetric Lee distance codes for DNA-based storage. IEEE Symposium on Information Theory (ISIT); Laure et al., 2016, Coding in 2D: Using Intentional Dispersity to Enhance the Information Capacity of Sequence-Coded Polymer Barcodes. Angew. Chem. Int. Ed. doi:10.1002/anie.201605279; Yazdi et al., 2015, IEEE Transactions on Molecular, Biological and Multi-Scale Communications 1:230-248; and Yazdi et al., 2015, Sci Rep 5:14138, each of which is incorporated by reference in its entirety). Thus, in certain embodiments, an extended recording tag, an extended coding tag, or a di-tag construct in any of the embodiments described herein is comprised of identifying components (e.g., UMI, encoder sequence, barcode, compartment tag, cycle specific sequence, etc.) that are error correcting codes. In some embodiments, the error correcting code is selected from: Hamming code, Lee distance code, asymmetric Lee distance code, Reed-Solomon code, and Levenshtein-Tenengolts code. For nanopore sequencing, the current or ionic flux profiles and asymmetric base calling errors are intrinsic to the type of nanopore and biochemistry employed, and this information can be used to design more robust DNA codes using the aforementioned error correcting approaches. An alternative to employing robust DNA nanopore sequencing barcodes, one can directly use the current or ionic flux signatures of barcode sequences (U.S. Pat. No. 7,060,507, incorporated by reference in its entirety), avoiding DNA base calling entirely, and immediately identify the barcode sequence by mapping back to the predicted current/flux signature as described by Laszlo et al. (2014, Nat. Biotechnol. 32:829-833, incorporated by reference in its entirety). For example, Laszlo et al. describe the current signatures generated by the biological nanopore, MspA, when passing different word strings through the nanopore, and the ability to map and identify DNA strands by mapping resultant current signatures back to an in silico prediction of possible current signatures from a universe of sequences (Laszlo et al., (2014) Nat. Biotechnol. 32:829-833). Similar concepts can be applied to DNA codes and the electrical signal generated by nanogap tunneling current-based DNA sequencing (Ohshiro et al., 2012, Sci Rep 2: 501).

Thus, in certain embodiments, the identifying components of a coding tag, recording tag, or both are capable of generating a unique current or ionic flux or optical signature, wherein the analysis step of any of the methods provided herein comprises detection of the unique current or ionic flux or optical signature in order to identify the identifying components. In some embodiments, the identifying components are selected from an encoder sequence, barcode, UMI, compartment tag, cycle specific sequence, or any combination thereof.

In certain embodiments, all or substantially amount of the polypeptides (e.g., at least 50%, 55%, 60%, 65%, 70%, 75%, 80%, 85%, 90%, 95%, 96%, 97%, 98%, 99%, or 100%) within a sample are labeled with a recording tag. Labeling of the polypeptides may occur before or after immobilization of the polypeptides to a solid support.

In other embodiments, a subset of polypeptides within a sample are labeled with recording tags. In a particular embodiment, a subset of polypeptides from a sample undergo targeted (analyte specific) labeling with recording tags. Targeted recording tag labeling of proteins may be achieved using target protein-specific binding agents (e.g., antibodies, aptamers, etc.) that are linked a short target-specific DNA capture probe, e.g., analyte-specific barcode, which anneal to complementary target-specific bait sequence, e.g., analyte-specific barcode, in recording tags. The recording tags comprise a reactive moiety for a cognate reactive moiety present on the target protein (e.g., click chemistry labeling, photoaffinity labeling). For example, recording tags may comprise an azide moiety for interacting with alkyne-derivatized proteins, or recording tags may comprise a benzophenone for interacting with native proteins, etc. Upon binding of the target protein by the target protein specific binding agent, the recording tag and target protein are coupled via their corresponding reactive. After the target protein is labeled with the recording tag, the target-protein specific binding agent may be removed by digestion of the DNA capture probe linked to the target-protein specific binding agent. For example, the DNA capture probe may be designed to contain uracil bases, which are then targeted for digestion with a uracil-specific excision reagent (e.g., USERTM), and the target-protein specific binding agent may be dissociated from the target protein.

In one example, antibodies specific for a set of target proteins can be labeled with a DNA capture probe that hybridizes with recording tags designed with complementary bait sequence. Sample-specific labeling of proteins can be achieved by employing DNA-capture probe labeled antibodies hybridizing with complementary bait sequence on recording tags comprising of sample-specific barcodes.

In another example, target protein-specific aptamers are used for targeted recording tag labeling of a subset of proteins within a sample. A target specific-aptamer is linked to a DNA capture probe that anneals with complementary bait sequence in a recording tag. The recording tag comprises a reactive chemical or photo-reactive chemical probes (e.g. benzophenone (BP)) for coupling to the target protein having a corresponding reactive moiety. The aptamer binds to its target protein molecule, bringing the recording tag into close proximity to the target protein, resulting in the coupling of the recording tag to the target protein.

Photoaffinity (PA) protein labeling using photo-reactive chemical probes attached to small molecule protein affinity ligands has been previously described (Park, Koh et al. 2016). Typical photo-reactive chemical probes include probes based on benzophenone (reactive diradical, 365 nm), phenyldiazirine (reactive carbon, 365 nm), and phenylazide (reactive nitrene free radical, 260 nm), activated under irradiation wavelengths as previously described (Smith et al., Future Med Chem. (2015) 7(2): 159-183). In a preferred embodiment, target proteins within a protein sample are labeled with recording tags comprising sample barcodes using the method disclosed by Li et al., in which a bait sequence in a benzophenone labeled recording tag is hybridized to a DNA capture probe attached to a cognate binding agent (e.g., nucleic acid aptamer (Li et al., Angew Chem Int Ed Engl (2013) 52(36): 9544-9549). For photoaffinity labeled protein targets, the use of DNA/RNA aptamers as target protein-specific binding agents are preferred over antibodies since the photoaffinity moiety can self-label the antibody rather than the target protein. In contrast, photoaffinity labeling is less efficient for nucleic acids than proteins, making aptamers a better vehicle for DNA-directed chemical or photo-labeling. Similar to photo-affinity labeling, one can also employ DNA-directed chemical labeling of reactive lysine's (or other moieties) in the proximity of the aptamer binding site in a manner similar to that described by Rosen et al. (Rosen et al, Nature Chemistry volume (2014) 6:804-809; Kodal et al., ChemBioChem (2016) 17:1338-1342).

In the aforementioned embodiments, other types of linkages besides hybridization can be used to link the target specific binding agent and the recording tag. For example, the two moieties can be covalently linked, using a linker that is designed to be cleaved and release the binding agent once the captured target protein (or other polypeptide) is covalently linked to the recording tag. A suitable linker can be attached to various positions of the recording tag, such as the 3′ end, or within the linker attached to the 5′ end of the recording tag.

B. Providing the Polypeptide Joined to a Support or in Solution

In some embodiments, polypeptides of the present disclosure are joined to a surface of a solid support (also referred to as “substrate surface”). The solid support can be any porous or non-porous support surface including, but not limited to, a bead, a microbead, an array, a glass surface, a silicon surface, a plastic surface, a filter, a membrane, nylon, a silicon wafer chip, a flow cell, a flow through chip, a biochip including signal transducing electronics, a microtiter well, an ELISA plate, a spinning interferometry disc, a nitrocellulose membrane, a nitrocellulose-based polymer surface, a nanoparticle, or a microsphere. Materials for a solid support include but are not limited to acrylamide, agarose, cellulose, nitrocellulose, glass, gold, quartz, polystyrene, polyethylene vinyl acetate, polypropylene, polymethacrylate, polyethylene, polyethylene oxide, polysilicates, polycarbonates, Teflon, fluorocarbons, nylon, silicon rubber, polyanhydrides, polyglycolic acid, polyactic acid, polyorthoesters, functionalized silane, polypropylfumerate, collagen, glycosaminoglycans, polyamino acids, or any combination thereof. Solid supports further include thin film, membrane, bottles, dishes, fibers, woven fibers, shaped polymers such as tubes, particles, beads, microparticles, or any combination thereof. For example, when solid surface is a bead, the bead can include, but is not limited to, a polystyrene bead, a polymer bead, an agarose bead, an acrylamide bead, a solid core bead, a porous bead, a paramagnetic bead, glass bead, or a controlled pore bead.

In certain embodiments, a solid support is a flow cell. Flow cell configurations may vary among different next generation sequencing platforms. For example, the Illumina flow cell is a planar optically transparent surface similar to a microscope slide, which contains a lawn of oligonucleotide anchors bound to its surface. Template DNA, comprise adapters ligated to the ends that are complimentary to oligonucleotides on the flow cell surface. Adapted single-stranded DNAs are bound to the flow cell and amplified by solid-phase “bridge” PCR prior to sequencing. The 454 flow cell (454 Life Sciences) supports a “picotiter” plate, a fiber optic slide with ˜1.6 million 75-picoliter wells. Each individual molecule of sheared template DNA is captured on a separate bead, and each bead is compartmentalized in a private droplet of aqueous PCR reaction mixture within an oil emulsion. Template is clonally amplified on the bead surface by PCR, and the template-loaded beads are then distributed into the wells of the picotiter plate for the sequencing reaction, ideally with one or fewer beads per well. SOLiD (Supported Oligonucleotide Ligation and Detection) instrument from Applied Biosystems, like the 454 system, amplifies template molecules by emulsion PCR. After a step to cull beads that do not contain amplified template, bead-bound template is deposited on the flow cell. A flow cell may also be a simple filter frit, such as a TWIST™ DNA synthesis column (Glen Research).

In certain embodiments, a solid support is a bead, which may refer to an individual bead or a plurality of beads. In some embodiments, the bead is compatible with a selected next generation sequencing platform that will be used for downstream analysis (e.g., SOLiD or 454). In some embodiments, a solid support is an agarose bead, a paramagnetic bead, a polystyrene bead, a polymer bead, an acrylamide bead, a solid core bead, a porous bead, a glass bead, or a controlled pore bead. In further embodiments, a bead may be coated with a binding functionality (e.g., amine group, affinity ligand such as streptavidin for binding to biotin labeled polypeptide, antibody) to facilitate binding to a polypeptide.

Proteins, polypeptides, or peptides can be joined to the solid support, directly or indirectly, by any means known in the art, including covalent and non-covalent interactions, or any combination thereof (see, e.g., Chan et al., 2007, PLoS One 2:e1164; Cazalis et al., Bioconj. Chem. 15:1005-1009; Soellner et al., 2003, J. Am. Chem. Soc. 125:11790-11791; Sun et al., 2006, Bioconjug. Chem. 17-52-57; Decreau et al., 2007, J. Org. Chem. 72:2794-2802; Camarero et al., 2004, J. Am. Chem. Soc. 126:14730-14731; Girish et al., 2005, Bioorg. Med. Chem. Lett. 15:2447-2451; Kalia et al., 2007, Bioconjug. Chem. 18:1064-1069; Watzke et al., 2006, Angew Chem. Int. Ed. Engl. 45:1408-1412; Parthasarathy et al., 2007, Bioconjugate Chem. 18:469-476; and Bioconjugate Techniques, G. T. Hermanson, Academic Press (2013), and are each hereby incorporated by reference in their entirety). For example, the peptide may be joined to the solid support by a ligation reaction. Alternatively, the solid support can include an agent or coating to facilitate joining, either direct or indirectly, the peptide to the solid support. Any suitable molecule or materials may be employed for this purpose, including proteins, nucleic acids, carbohydrates and small molecules. For example, in one embodiment the agent is an affinity molecule. In another example, the agent is an azide group, which group can react with an alkynyl group in another molecule to facilitate association or binding between the solid support and the other molecule.

Proteins, polypeptides, or peptides can be joined to the solid support using methods referred to as “click chemistry.” For this purpose, any reaction which is rapid and substantially irreversible can be used to attach proteins, polypeptides, or peptides to the solid support. Exemplary reactions include the copper catalyzed reaction of an azide and alkyne to form a triazole (Huisgen 1, 3-dipolar cycloaddition), strain-promoted azide alkyne cycloaddition (SPAAC), reaction of a diene and dienophile (Diels-Alder), strain-promoted alkyne-nitrone cycloaddition, reaction of a strained alkene with an azide, tetrazine or tetrazole, alkene and azide [3+2] cycloaddition, alkene and tetrazine inverse electron demand Diels-Alder (IEDDA) reaction (e.g., m-tetrazine (mTet) or phenyl tetrazine (pTet) and trans-cyclooctene (TCO); or pTet and an alkene), alkene and tetrazole photoreaction, Staudinger ligation of azides and phosphines, and various displacement reactions, such as displacement of a leaving group by nucleophilic attack on an electrophilic atom (Horisawa, Front Physiol (2014). 5: 457; Knall, Hollauf et al., Tetrahedron Lett (2014) 55(34): 4763-4766). Exemplary displacement reactions include reaction of an amine with: an activated ester; an N-hydroxysuccinimide ester; an isocyanate; an isothioscyanate, an aldehyde, an epoxide, or the like.

In some embodiments, the polypeptide and solid support are joined by a functional group capable of formation by reaction of two complementary reactive groups, for example a functional group which is the product of one of the foregoing “click” reactions. In various embodiments, functional group can be formed by reaction of an aldehyde, oxime, hydrazone, hydrazide, alkyne, amine, azide, acylazide, acylhalide, nitrile, nitrone, sulfhydryl, disulfide, sulfonyl halide, isothiocyanate, imidoester, activated ester (e.g., N-hydroxysuccinimide ester, pentynoic acid STP ester), ketone, α,β-unsaturated carbonyl, alkene, maleimide, α-haloimide, epoxide, aziridine, tetrazine, tetrazole, phosphine, biotin or thiirane functional group with a complementary reactive group. An exemplary reaction is a reaction of an amine (e.g., primary amine) with an N-hydroxysuccinimide ester or isothiocyanate.

In some embodiments, the functional group comprises an alkene, ester, amide, thioester, disulfide, carbocyclic, heterocyclic or heteroaryl group. In further embodiments, the functional group comprises an alkene, ester, amide, thioester, thiourea, disulfide, carbocyclic, heterocyclic or heteroaryl group. In other embodiments, the functional group comprises an amide or thiourea. In some more specific embodiments, functional group is a triazolyl functional group, an amide, or thiourea functional group.

In some embodiments, iEDDA click chemistry is used for immobilizing polypeptides to a solid support since it is rapid and delivers high yields at low input concentrations. In another embodiment, m-tetrazine rather than tetrazine is used in an iEDDA click chemistry reaction, as m-tetrazine has improved bond stability. In another embodiment, phenyl tetrazine (pTet) is used in an iEDDA click chemistry reaction.

In some embodiments, the substrate surface is functionalized with TCO, and the recording tag-labeled protein, polypeptide, peptide is immobilized to the TCO coated substrate surface via an attached m-tetrazine moiety.

In some embodiments, polypeptides are immobilized to a surface of a solid support by its C-terminus, N-terminus, or an internal amino acid, for example, via an amine, carboxyl, or sulfydryl group. Standard activated supports used in coupling to amine groups include CNBr-activated, NHS-activated, aldehyde-activated, azlactone-activated, and CDI-activated supports. Standard activated supports used in carboxyl coupling include carbodiimide-activated carboxyl moieties coupling to amine supports. Cysteine coupling can employ maleimide, idoacetyl, and pyridyl disulfide activated supports. An alternative mode of peptide carboxy terminal immobilization uses anhydrotrypsin, a catalytically inert derivative of trypsin that binds peptides containing lysine or arginine residues at their C-termini without cleaving them.

In certain embodiments, a polypeptide is immobilized to a solid support via covalent attachment of a solid surface bound linker to a lysine group of the protein, polypeptide, or peptide.

Recording tags can be attached to the protein, polypeptide, or peptides pre- or post-immobilization to the solid support. For example, proteins, polypeptides, or peptides can be first labeled with recording tags and then immobilized to a solid surface via a recording tag comprising at two functional moieties for coupling. One functional moiety of the recording tag couples to the protein, and the other functional moiety immobilizes the recording tag-labeled protein to a solid support.

In other embodiments, polypeptides are immobilized to a solid support prior to labeling of the proteins, polypeptides or peptides with recording tags. For example, proteins can first be derivatized with reactive groups such as click chemistry moieties. The activated protein molecules can then be attached to a suitable solid support and then labeled with recording tags using the complementary click chemistry moiety. As an example, proteins derivatized with alkyne and mTet moieties may be immobilized to beads derivatized with azide and TCO and attached to recording tags labeled with azide and TCO.

It is understood that the methods provided herein for attaching polypeptides to the solid support may also be used to attach recording tags to the solid support or attach recording tags to polypeptides.

In certain embodiments, the surface of a solid support is passivated (blocked) to minimize non-specific absorption to binding agents. A “passivated” surface refers to a surface that has been treated with outer layer of material to minimize non-specific binding of a binding agent. Methods of passivating surfaces include standard methods from the fluorescent single molecule analysis literature, including passivating surfaces with polymer like polyethylene glycol (PEG) (Pan et al., 2015, Phys. Biol. 12:045006), polysiloxane (e.g., Pluronic F-127), star polymers (e.g., star PEG) (Groll et al., 2010, Methods Enzymol. 472:1-18), hydrophobic dichlorodimethylsilane (DDS)+self-assembled Tween-20 (Hua et al., 2014, Nat. Methods 11:1233-1236), and diamond-like carbon (DLC), DLC+PEG (Stavis et al., 2011, Proc. Natl. Acad. Sci. USA 108:983-988), and zwitterionic moiety (e.g., U.S. Patent Application Publication US 2006/0183863). In addition to covalent surface modifications, a number of passivating agents can be employed as well including surfactants like Tween-20, polysiloxane in solution (Pluronic series), poly vinyl alcohol, (PVA), and proteins like BSA and casein. Alternatively, density of proteins, polypeptide, or peptides can be titrated on the surface or within the volume of a solid substrate by spiking a competitor or “dummy” reactive molecule when immobilizing the proteins, polypeptides or peptides to the solid substrate.

In certain embodiments where multiple polypeptides are immobilized on the same solid support, the polypeptides can be spaced appropriately to reduce the occurrence of or prevent a cross-binding or inter-molecular event, e.g., where a binding agent binds to a first polypeptides and its coding tag information is transferred to a recording tag associated with a neighboring polypeptides rather than the recording tag associated with the first polypeptide. To control polypeptide spacing on the solid support, the density of functional coupling groups (e.g., TCO) may be titrated on the substrate surface. In some embodiments, multiple polypeptides are spaced apart on the surface or within the volume (e.g., porous supports) of a solid support at a distance of about 50 nm to about 500 nm, or a subrange thereof, e.g., or about 50 nm to about 400 nm, or about 50 nm to about 300 nm, or about 50 nm to about 200 nm, or about 50 nm to about 100 nm. In some embodiments, multiple polypeptides are spaced apart on the surface of a solid support with an average distance of at least 50 nm, at least 60 nm, at least 70 nm, at least 80 nm, at least 90 nm, at least 100 nm, at least 150 nm, at least 200 nm, at least 250 nm, at least 300 nm, at least 350 nm, at least 400 nm, at least 450 nm, or at least 500 nm. In some embodiments, multiple polypeptides are spaced apart on the surface of a solid support with an average distance of at least 50 nm. In some embodiments, polypeptides are spaced apart on the surface or within the volume of a solid support such that, empirically, the relative frequency of inter- to intra-molecular events is <1:10; <1:100; <1:1,000; or <1:10,000. A suitable spacing frequency can be determined empirically using a functional assay (see, Example 31 of International Patent Publication No. WO 2017/192633), and can be accomplished by dilution and/or by spiking a “dummy” spacer molecule that competes for attachments sites on the substrate surface.

For example, PEG-5000 (MW˜5000) is used to block the interstitial space between peptides on the substrate surface (e.g., bead surface). In addition, the peptide is coupled to a functional moiety that is also attached to a PEG-5000 molecule. In some embodiments, this is accomplished by coupling a mixture of NHS-PEG-5000-TCO+NHS-PEG-5000-Methyl to amine-derivatized beads. The stoichiometric ratio between the two PEGs (TCO vs. methyl) is titrated to generate an appropriate density of functional coupling moieties (TCO groups) on the substrate surface; the methyl-PEG is inert to coupling. The effective spacing between TCO groups can be calculated by measuring the density of TCO groups on the surface. In certain embodiments, the mean spacing between coupling moieties (e.g., TCO) on the solid surface is at least 50 nm, at least 100 nm, at least 250 nm, or at least 500 nm. After PEG5000-TCO/methyl derivatization of the beads, the excess NH2 groups on the surface are quenched with a reactive anhydride (e.g. acetic or succinic anhydride).

In some embodiments, the spacing is accomplished by titrating the ratio of available attachment molecules on the substrate surface. In some examples, the substrate surface (e.g., bead surface) is functionalized with a carboxyl group (COOH) which is treated with an activating agent (e.g., activating agent is EDC and Sulfo-NHS). In some examples, the substrate surface (e.g., bead surface) comprises NHS moieties. In some embodiments, a mixture of mPEGₙ-NH₂ and NH₂-PEGₙ-mTet is added to the activated beads (wherein n is any number, such as 1-100). The ratio between the mPEG₃-NH₂ (not available for coupling) and NH₂-PEG₂₄-mTet (available for coupling) is titrated to generate an appropriate density of functional moieties available to attach the analyte on the substrate surface. In certain embodiments, the mean spacing between or among coupling moieties (e.g., NH₂-PEG₄-mTet) on the solid surface is at least 50 nm, at least 100 nm, at least 250 nm, or at least 500 nm. In some specific embodiments, the ratio of NH₂-PEGₙ-mTet to mPEG₃-NH₂ is about or greater than 1:1000, about or greater than 1:10,000, about or greater than 1:100,000, or about or greater than 1:1,000,000. In some further embodiments, the capture nucleic acid attaches to the NH₂-PEGₙ-mTet.

In particular embodiments, the polypeptide(s) and/or the recording tag(s) are immobilized on a substrate or support at a density such that the interaction between (i) a coding agent bound to a first polypeptide (particularly, the coding tag in that bound coding agent), and (ii) a second polypeptide and/or its recording tag, is reduced, minimized, or completely eliminated. Therefore, false positive assay signals resulting from “intermolecular” engagement can be reduced, minimized, or eliminated.

In certain embodiments, the density of the polypeptides and/or the recording tags on a substrate is determined for each type of polypeptide. For example, the longer a denatured polypeptide chain is, the lower the density should be in order to reduce, minimize, or prevent “intermolecular” interactions. In certain aspects, increasing the spacing between the polypeptide molecules and/or the recording tags (i.e., lowering the density) increases the signal to background ratio of the presently disclosed assays.

In some embodiments, the polypeptide molecules and/or the recording tags are deposited or immobilized on a substrate at any suitable average density, e.g., at an average density of about 0.0001 molecule/μm², 0.001 molecule/μm², 0.01 molecule/μm², 0.1 molecule/μm², 1 molecule/μm², about 2 molecules/μm², about 3 molecules/μm², about 4 molecules/μm², about 5 molecules/μm², about 6 molecules/μm², about 7 molecules/μm², about 8 molecules/μm², about 9 molecules/μm², or about 10 molecules/μm². In other embodiments, the polypeptide(s) and/or the recording tag(s) are deposited or immobilized at an average density of about 15, about 20, about 25, about 30, about 35, about 40, about 45, about 50, about 55, about 60, about 65, about 70, about 75, about 80, about 85, about 90, about 95, about 100, about 105, about 110, about 115, about 120, about 125, about 130, about 135, about 140, about 145, about 150, about 155, about 160, about 165, about 170, about 175, about 180, about 185, about 190, about 195, about 200, or about 200 molecules/μm² on a substrate. In other embodiments, the polypeptide(s) and/or the recording tag(s) are deposited or immobilized at an average density of about 1 molecule/mm², about 10 molecules/mm², about 50 molecules/mm², about 100 molecules/mm², about 150 molecules/mm², about 200 molecules/mm², about 250 molecules/mm², about 300 molecules/mm², about 350 molecules/mm², 400 molecules/mm², about 450 molecules/mm², about 500 molecules/mm², about 550 molecules/mm², about 600 molecules/mm², about 650 molecules/mm², about 700 molecules/mm², about 750 molecules/mm², about 800 molecules/mm², about 850 molecules/mm², about 900 molecules/mm², about 950 molecules/mm², or about 1000 molecules/mm². In still other embodiments, the polypeptide(s) and/or the recording tag(s) are deposited or immobilized on a substrate at an average density between about 1×10³ and about 0.5×10⁴ molecules/mm², between about 0.5×10⁴ and about 1×10⁴ molecules/mm², between about 1×10⁴ and about 0.5×10⁵ molecules/mm², between about 0.5×10⁵ and about 1×10⁵ molecules/mm², between about 1×10⁵ and about 0.5×10⁶ molecules/mm², or between about 0.5×10⁶ and about 1×10⁶ molecules/mm². In other embodiments, the average density of the polypeptide(s) and/or the recording tag(s) deposited or immobilized on a substrate can be, for example, between about 1 molecule/cm² and about 5 molecules/cm², between about 5 and about 10 molecules/cm², between about 10 and about 50 molecules/cm², between about 50 and about 100 molecules/cm², between about 100 and about 0.5×10³ molecules/cm², between about 0.5×10³ and about 1×10³ molecules/cm², 1×10³ and about 0.5×10⁴ molecules/cm², between about 0.5×10⁴ and about 1×10⁴ molecules/cm², between about 1×10⁴ and about 0.5×10⁵ molecules/cm², between about 0.5×10⁵ and about 1×10⁵ molecules/cm², between about 1×10⁵ and about 0.5×10⁶ molecules/cm², or between about 0.5×10⁶ and about 1×10⁶ molecules/cm².

In certain embodiments, the concentration of the binding agents in a solution is controlled to reduce background and/or false positive results of the assay.

In some embodiments, the concentration of a binding agent can be at any suitable concentration, e.g., at about 0.0001 nM, about 0.001 nM, about 0.01 nM, about 0.1 nM, about 1 nM, about 2 nM, about 5 nM, about 10 nM, about 20 nM, about 50 nM, about 100 nM, about 200 nM, about 500 nM, or about 1000 nM. In other embodiments, the concentration of a soluble conjugate used in the assay is between about 0.0001 nM and about 0.001 nM, between about 0.001 nM and about 0.01 nM, between about 0.01 nM and about 0.1 nM, between about 0.1 nM and about 1 nM, between about 1 nM and about 2 nM, between about 2 nM and about 5 nM, between about 5 nM and about 10 nM, between about 10 nM and about 20 nM, between about 20 nM and about 50 nM, between about 50 nM and about 100 nM, between about 100 nM and about 200 nM, between about 200 nM and about 500 nM, between about 500 nM and about 1000 nM, or more than about 1000 nM.

In some embodiments, the ratio between the soluble binding agent molecules and the immobilized polypeptides and/or the recording tags can be at any suitable range, e.g., at about 0.00001:1, about 0.0001:1, about 0.001:1, about 0.01:1, about 0.1:1, about 1:1, about 2:1, about 5:1, about 10:1, about 15:1, about 20:1, about 25:1, about 30:1, about 35:1, about 40:1, about 45:1, about 50:1, about 55:1, about 60:1, about 65:1, about 70:1, about 75:1, about 80:1, about 85:1, about 90:1, about 95:1, about 100:1, about 10⁴:1, about 10⁵:1, about 10⁶:1, or higher, or any ratio in between the above listed ratios. Higher ratios between the soluble binding agent molecules and the immobilized polypeptide(s) and/or the recording tag(s) can be used to drive the binding and/or the coding tag/recoding tag information transfer to completion. This may be particularly useful for detecting and/or analyzing low abundance polypeptides in a sample.

C. Protein Normalization Via Fractionation, Compartmentalization, and Limited Binding Capacity Resins

In some embodiments, the methods provided herein may be performed on polypeptides that have been normalized. In some embodiments, subtraction of certain protein species (e.g., highly abundant proteins) from the sample is performed prior to analysis. This can be accomplished, for example, using commercially available protein depletion reagents such as Sigma's PROT20 immuno-depletion kit, which deplete the top 20 plasma proteins. Additionally, it would be useful to have an approach that greatly reduced the dynamic range even further to a manageable 3-4 orders. In certain embodiments, a protein sample dynamic range can be modulated by fractionating the protein sample using standard fractionation methods, including electrophoresis and liquid chromatography (Zhou et al., Anal Chem (2012) 84(2): 720-734), or partitioning the fractions into compartments (e.g., droplets) loaded with limited capacity protein binding beads/resin (e.g. hydroxylated silica particles) (McCormick, Anal Biochem (1989) 181(1): 66-74) and eluting bound protein. Excess protein in each compartmentalized fraction is washed away.

Examples of electrophoretic methods include capillary electrophoresis (CE), capillary isoelectric focusing (CIEF), capillary isotachophoresis (CITP), free flow electrophoresis, gel-eluted liquid fraction entrapment electrophoresis (GELFrEE). Examples of liquid chromatography protein separation methods include reverse phase (RP), ion exchange (IE), size exclusion (SE), hydrophilic interaction, etc. Examples of compartment partitions include emulsions, droplets, microwells, physically separated regions on a flat substrate, etc. Exemplary protein binding beads/resins include silica nanoparticles derivitized with phenol groups or hydroxyl groups (e.g., StrataClean Resin from Agilent Technologies, RapidClean from LabTech, etc.). By limiting the binding capacity of the beads/resin, highly-abundant proteins eluting in a given fraction will only be partially bound to the beads, and excess proteins removed.

D. Partitioning of Proteome of a Single Cell or Molecular Subsampling

In some aspects, provided are methods modifying polypeptides in the presence of microwave energy, wherein the methods are for analysis of proteins in a sample including barcoding and partitioning techniques. In some embodiments, the proteins are labeled with DNA tags comprising barcodes used for spatial segmentation of a tissue on the surface an array of spatially distributed DNA barcode sequences. In another embodiment, spatial barcoding can be used within a cell to identify the protein constituents/PTMs within the cellular organelles and cellular compartments (Christoforou et al., 2016, Nat. Commun. 7:8992, incorporated by reference in its entirety).

Current approaches to protein analysis involve fragmentation of protein polypeptides into shorter peptide molecules suitable for peptide sequencing. Information obtained using such approaches is therefore limited by the fragmentation step and excludes, e.g., long range continuity information of a protein, including post-translational modifications, protein-protein interactions occurring in each sample, the composition of a protein population present in a sample, or the origin of the protein polypeptide, such as from a particular cell or population of cells. In some embodiments, long range information of post-translation modifications within a protein molecule (e.g., proteoform characterization) provides a more complete picture of biology, and long range information on what peptides belong to what protein molecule provides a more robust mapping of peptide sequence to underlying protein sequence.

In some embodiments, by using the partitioning methods disclosed herein, combined with information from a number of peptides originating from the same protein molecule, the identity of the protein molecule (e.g. proteoform) can be more accurately assessed. In some aspects, association of compartment tags with proteins and peptides derived from same compartment(s) facilitates reconstruction of molecular and cellular information. In some embodiments, cells are lysed and proteins digested into short peptides, disrupting global information on which proteins derive from which cell or cell type, and which peptides derive from which protein or protein complex. This global information is important to understanding the biology and biochemistry within cells and tissues.

Partitioning refers to the assignment, e.g., a random assignment of a unique barcode to a subpopulation of polypeptides from a population of polypeptides within a sample. In some embodiments, partitioning may be achieved by distributing polypeptides into compartments. A partition may be comprised of the polypeptides within a single compartment or the polypeptides within multiple compartments from a population of compartments.

A subset of polypeptides or a subset of a protein sample that has been separated into or on the same physical compartment or group of compartments from a plurality (e.g., millions to billions) of compartments are identified by a unique compartment tag. Thus, a compartment tag can be used to distinguish constituents derived from one or more compartments having the same compartment tag from those in another compartment (or group of compartments) having a different compartment tag, even after the constituents are pooled together.

In some embodiments, the present disclosure provides methods of enhancing protein analysis by partitioning a complex proteome sample (e.g., a plurality of protein complexes, proteins, or polypeptides) or complex cellular sample into a plurality of compartments, wherein each compartment comprises a plurality of compartment tags that are the same within an individual compartment and are different from the compartment tags of other compartments. The compartments optionally comprise a solid support (e.g., bead) to which the plurality of compartment tags are joined thereto. In some aspects, the plurality of protein complexes, proteins, or polypeptides are fragmented into a plurality of peptides, which are then contacted to the plurality of compartment tags under conditions sufficient to permit annealing or joining of the plurality of peptides with the plurality of compartment tags within the plurality of compartments, thereby generating a plurality of compartment tagged peptides. Alternatively, in some cases, the plurality of protein complexes, proteins, or polypeptides are joined to a plurality of compartment tags under conditions sufficient to permit annealing or joining of the plurality of protein complexes, proteins or polypeptides with the plurality of compartment tags within a plurality of compartments, thereby generating a plurality of compartment tagged protein complexes, proteins, polypeptides. In some embodiments, compartment tagged protein complexes, proteins, or polypeptides are then collected from the plurality of compartments and optionally fragmented into a plurality of compartment tagged peptides. In some embodiments, one or more compartment tagged peptides are analyzed according to any of the methods described herein.

In some embodiments, the compartment tags are free in solution within the compartments. In other embodiments, the compartment tags are joined directly to the surface of the compartment (e.g., well bottom of microtiter or picotiter plate) or a bead or bead within a compartment.

A compartment can be an aqueous compartment (e.g., microfluidic droplet) or a solid compartment. A solid compartment includes, for example, a nanoparticle, a microsphere, a microtiter or picotiter well or a separated region on an array, a glass surface, a silicon surface, a plastic surface, a filter, a membrane, nylon, a silicon wafer chip, a flow cell, a flow through chip, a biochip including signal transducing electronics, an ELISA plate, a spinning interferometry disc, a nitrocellulose membrane, or a nitrocellulose-based polymer surface. In certain embodiments, each compartment contains, on average, a single cell.

A solid support can be any support surface including, but not limited to, a bead, a microbead, an array, a glass surface, a silicon surface, a plastic surface, a filter, a membrane, a PTFE membrane, a PTFE membrane, a nitrocellulose membrane, a nitrocellulose-based polymer surface, nylon, a silicon wafer chip, a flow cell, a flow through chip, a biochip including signal transducing electronics, a microtiter well, an ELISA plate, a spinning interferometry disc, a nitrocellulose membrane, a nitrocellulose-based polymer surface, a nanoparticle, or a microsphere. Materials for a solid support include but are not limited to acrylamide, agarose, cellulose, dextran, nitrocellulose, glass, gold, quartz, polystyrene, polyethylene vinyl acetate, polypropylene, polyester, polymethacrylate, polyacrylate, polyethylene, polyethylene oxide, polysilicates, polycarbonates, poly vinyl alcohol (PVA), Teflon, fluorocarbons, nylon, silicon rubber, polyanhydrides, polyglycolic acid, polyvinylchloride, polylactic acid, polyorthoesters, functionalized silane, polypropylfumerate, collagen, glycosaminoglycans, polyamino acids, or any combination thereof. In certain embodiments, a solid support is a bead, for example, a polyacrylate bead, a polystyrene bead, a polymer bead, an agarose bead, a cellulose bead, a dextran bead, an acrylamide bead, a solid core bead, a porous bead, a paramagnetic bead, a glass bead, a silica-based bead, a controlled pore bead, or any combinations thereof.

Various methods of partitioning samples into compartments with compartment tagged beads are available (Shembekar et al., Lab Chip (2016) 16(8): 1314-1331). In one example, the proteome is partitioned into droplets via an emulsion to enable global information on protein molecules and protein complexes to be recorded using the methods disclosed herein. In certain embodiments, the proteome is partitioned in compartments (e.g., droplets) along with compartment tagged beads, an activate-able protease (directly or indirectly via heat, light, etc.), and a peptide ligase engineered to be protease-resistant (e.g., modified lysines, pegylation, etc.). In certain embodiments, the proteome can be treated with a denaturant to assess the peptide constituents of a protein or polypeptide. If information regarding the native state of a protein is desired, an interacting protein complex can be partitioned into compartments for subsequent analysis of the peptides derived therefrom.

In certain embodiments, the plurality of proteins or polypeptides within the plurality of compartments is fragmented into a plurality of peptides with a protease. A protease can be a metalloprotease. In certain embodiments, the activity of the metalloprotease is modulated by photo-activated release of metallic cations. Examples of endopeptidases that can be used include: trypsin, chymotrypsin, elastase, thermolysin, pepsin, clostripan, glutamyl endopeptidase (GluC), endopeptidase ArgC, peptidyl-asp metallo-endopeptidase (AspN), endopeptidase LysC and endopeptidase LysN. Their mode of activation varies depending on buffer and divalent cation requirements. Optionally, following sufficient digestion of the proteins or polypeptides into peptide fragments, the protease is inactivated (e.g., heat, fluoro-oil or silicone oil soluble inhibitor, such as a divalent cation chelation agent).

In certain embodiments of peptide barcoding with compartment tags, a protein molecule (optionally, denatured polypeptide) is labeled with DNA tags by conjugation of the DNA tags to ε-amine moieties of the protein's lysine groups or indirectly via click chemistry attachment to a protein/polypeptide pre-labeled with a reactive click moiety such as alkyne. The DNA tag-labeled polypeptides are then partitioned into compartments comprising compartment tags (e.g., DNA barcodes bound to beads contained within droplets) wherein a compartment tag contains a barcode that identifies each compartment. In one embodiment, a single protein/polypeptide molecule is co-encapsulated with a single species of DNA barcodes associated with a bead. In another embodiment, the compartment can constitute the surface of a bead with attached compartment (bead) tags similar to that described in PCT Publication WO2016/061517 (incorporated by reference in its entirety), except as applied to proteins rather than DNA. The compartment tag can comprise a barcode (BC) sequence, a universal priming site (U1′), a UMI sequence, and a spacer sequence (Sp). In one embodiment, concomitant with or after partitioning, the compartment tags are cleaved from the bead and hybridize to the DNA tags attached to the polypeptide, for example via the complementary U1 and U1′ sequences on the DNA tag and compartment tag, respectively. For partitioning on beads, the DNA tag-labeled protein can be directly hybridized to the compartment tags on the bead surface. After this hybridization step, the polypeptides with hybridized DNA tags are extracted from the compartments (e.g., emulsion “cracked”, or compartment tags cleaved from bead), and a polymerase-based primer extension step is used to write the barcode and UMI information to the DNA tags on the polypeptide to yield a compartment barcoded recording tag. A LysC protease digestion may be used to cleave the polypeptide into constituent peptides labeled at their C-terminal lysine with a recording tag containing universal priming sequences, a compartment tag, and a UMI. In one embodiment, the LysC protease is engineered to tolerate DNA-tagged lysine residues. The resultant recording tag labeled peptides are immobilized to a solid substrate (e.g., bead) at an appropriate density to minimize intermolecular interactions between recording tagged peptides.

Attachment of the peptide to the compartment tag (or vice versa) can be directly to an immobilized compartment tag, or to its complementary sequence (if double stranded). Alternatively, the compartment tag can be detached from the solid support or surface of the compartment, and the peptide and solution phase compartment tag joined within the compartment. In one embodiment, the functional moiety on the compartment tag (e.g., on the terminus of oligonucleotide) is an aldehyde which is coupled directly to the amine N-terminus of the peptide through a Schiff base.

Approaches for compartmental-based partitioning include droplet formation through microfluidic devices using T-junctions and flow focusing, emulsion generation using agitation or extrusion through a membrane with small holes (e.g., track etch membrane), etc. A challenge with compartmentalization is addressing the interior of the compartment. In certain embodiments, it may be difficult to conduct a series of different biochemical steps within a compartment since exchanging fluid components is challenging. As previously described, one can modify a limited feature of the droplet interior, such as pH, chelating agent, reducing agents, etc. by addition of the reagent to the fluoro-oil of the emulsion.

After labeling of the proteins/peptides with recording tags comprised of compartment tags (barcodes), the protein/peptides are immobilized on a solid-support at a suitable density to favor intramolecular transfer of information from the coding tag of a bound cognate binding agent to the corresponding recording tag/tags attached to the bound peptide or protein molecule. Intermolecular information transfer is minimized by controlling the intermolecular spacing of molecules on the surface of the solid-support.

In certain embodiments, the compartment tags need not be unique for each compartment in a population of compartments. A subset of compartments (two, three, four, or more) in a population of compartments may share the same compartment tag. For instance, each compartment may be comprised of a population of bead surfaces which act to capture a subpopulation of polypeptides from a sample (many molecules are captured per bead). Moreover, the beads comprise compartment barcodes which can be attached to the captured polypeptides. Each bead has only a single compartment barcode sequence, but this compartment barcode may be replicated on other beads with in the compartment (many beads mapping to the same barcode). There can be (although not required) a many-to-one mapping between physical compartments and compartment barcodes, moreover, there can be (although not required) a many-to-one mapping between polypeptides within a compartment. A partition barcode is defined as an assignment of a unique barcode to a subsampling of polypeptides from a population of polypeptides within a sample. This partition barcode may be comprised of identical compartment barcodes arising from the partitioning of polypeptides within compartments labeled with the same barcode. The use of physical compartments effectively subsamples the original sample to provide assignment of partition barcodes. For instance, a set of beads labeled with 10,000 different compartment barcodes is provided. Furthermore, suppose in a given assay, that a population of 1 million beads are used in the assay. On average, there are 100 beads per compartment barcode (Poisson distribution). Further suppose that the beads capture an aggregate of 10 million polypeptides. On average, there are 10 polypeptides per bead, with 100 compartments per compartment barcode, there are effectively 1000 polypeptides per partition barcode (comprised of 100 compartment barcodes for 100 distinct physical compartments).

### III. Polypeptide Sequence Analysis

In some embodiments, the methods for treating a polypeptide in the presence of microwave energy are used for determining the sequence of at least a portion of the polypeptide. In some embodiments, the provided methods are for accelerating sequencing reactions that include treatment of polypeptides. In some embodiments, determining the sequence of at least a portion of the polypeptide includes performing any of the methods as described in International Patent Publication No. WO 2017/192633.

In some embodiments, the provided methods can be used in the context of a degradation-based polypeptide sequencing assay. In some cases, the sequence of the polypeptide is analyzed by construction of an extended recording tag (e.g., DNA sequence) representing the polypeptide sequence, such as an extended recording tag. In some embodiments, the assay includes an Edman degradation-like approach using a cyclic process such as amino acid functionalization (e.g., terminal amino acid, N-terminal amino acid (NTAA) functionalization, C-terminal amino acid (CTAA)). In some embodiments, the assay includes transfer of coding tag information (e.g., joined to a binding agent) to a recording tag attached to the polypeptide. In some embodiments, the assay includes removal of a terminal amino acid (e.g., NTAA or CTAA). In some embodiments, one or more steps of the assay of polypeptide analysis is repeated in a cyclic manner, for example, the steps are all on a solid support. FIG. 1 depicts an exemplary schematic of the steps in the polypeptide analysis assay.

In some embodiments, the construction of an extended recording tag from N-terminal degradation of a peptide includes functionalizing the N-terminal amino acid of a polypeptide (e.g., with a phenylthiocarbamoyl (PTC or derivatized PTC), dinitrophenyl (DNP), sulfonyl nitrophenyl (SNP), acetyl, guanidinyl moiety, or any modification described in Section IA). In some embodiment, the assay includes contacting a binding agent that is associated with a coding tag bound to the functionalized NTAA (e.g., a guanidinylated NTAA). In some cases, the polypeptide is bound to a solid support (e.g., bead) and associated with a recording tag (e.g., via a trifunctional linker). In some embodiments, upon binding of the binding agent to the NTAA of the polypeptide, information of the coding tag is transferred to the recording tag (e.g., via primer extension) to generate an extended recording tag. In some aspects, the functionalized NTAA is eliminated via chemical or biological (e.g., enzymatic) means to expose a new NTAA. In some embodiments, cycling of the steps described is repeated “n” times to generate a final extended recording tag, as shown in FIG. 1. In some examples, the final extended recording tag is optionally flanked by universal priming sites to facilitate downstream amplification and/or DNA sequencing. The forward universal priming site (e.g., Illumina's P5-S1 sequence) can be part of the original recording tag design and the reverse universal priming site (e.g., Illumina's P7-S2′ sequence) can be added as a final step in the extension of the recording tag. In some embodiments, the addition of forward and reverse priming sites can be done independently of a binding agent.

In some embodiments, the order in the steps in the process for a degradation-based peptide polypeptide sequencing assay can be reversed or moved around. For example, in some embodiments, the terminal amino acid functionalization can be conducted before and/or after the polypeptide is bound to the binding agent and/or associated coding tag. In some embodiments, the terminal amino acid functionalization can be conducted before or after the polypeptide is bound a support. In some embodiments, the terminal amino acid removal can be conducted before and/or after the polypeptide is bound to the binding agent and/or associated coding tag.

A. Cyclic Transfer of Coding Tag Information to Recording Tags

In the methods described herein, upon binding of a binding agent to a polypeptide, identifying information of its linked coding tag is transferred to a recording tag associated with the polypeptide, thereby generating an “extended recording tag.” An extended recording tag may comprise information from a binding agent's coding tag representing each binding cycle performed. However, an extended recording tag may also experience a “missed” binding cycle, e.g., because a binding agent fails to bind to the polypeptide, because the coding tag was missing, damaged, or defective, because the primer extension reaction failed. Even if a binding event occurs, transfer of information from the coding tag to the recording tag may be incomplete or less than 100% accurate, e.g., because a coding tag was damaged or defective, because errors were introduced in the primer extension reaction). Thus, an extended recording tag may represent 100%, or up to 95%, 90%, 85%, 80%, 75%, 70%, 65%, 60%, 65%, 55%, 50%, 45%, 40%, 35%, 30%, or any subrange thereof, of binding events that have occurred on its associated polypeptide. Moreover, the coding tag information present in the extended recording tag may have at least 30%, 35%, 40%, 45%, 50%, 55%, 60%, 65%, 70%, 75%, 80%, 85%, 90%, 95%, or 100% identity the corresponding coding tags.

In certain embodiments, an extended recording tag may comprise information from multiple coding tags representing multiple, successive binding events. In these embodiments, a single, concatenated extended recording tag can be representative of a single polypeptide. As referred to herein, transfer of coding tag information to a recording tag also includes transfer to an extended recording tag as would occur in methods involving multiple, successive binding events.

In certain embodiments, the binding event information is transferred from a coding tag to a recording tag in a cyclic fashion (see FIG. 1). Cross-reactive binding events can be informatically filtered out after sequencing by requiring that at least two different coding tags, identifying two or more independent binding events, map to the same class of binding agents (cognate to a particular protein). An optional sample or compartment barcode can be included in the recording tag, as well an optional UMI sequence. The coding tag can also contain an optional UMI sequence along with the encoder and spacer sequences. Universal priming sequences may also be included in extended recording tags for amplification and NGS sequencing.

Coding tag information associated with a specific binding agent may be transferred to a recording tag using a variety of methods. In certain embodiments, information of a coding tag is transferred to a recording tag via primer extension (Chan, McGregor et al. 2015). A spacer sequence on the 3′-terminus of a recording tag or an extended recording tag anneals with complementary spacer sequence on the 3′ terminus of a coding tag and a polymerase (e.g., strand-displacing polymerase) extends the recording tag sequence, using the annealed coding tag as a template. In some embodiments, oligonucleotides complementary to coding tag encoder sequence and 5′ spacer can be pre-annealed to the coding tags to prevent hybridization of the coding tag to internal encoder and spacer sequences present in an extended recording tag. The 3′ terminal spacer, on the coding tag, remaining single stranded, preferably binds to the terminal 3′ spacer on the recording tag. In other embodiments, a nascent recording tag can be coated with a single stranded binding protein to prevent annealing of the coding tag to internal sites. Alternatively, the nascent recording tag can also be coated with RecA (or related homologues such as uvsX) to facilitate invasion of the 3′ terminus into a completely double stranded coding tag (Bell et al., 2012, Nature 491:274-278). This configuration prevents the double stranded coding tag from interacting with internal recording tag elements, yet is susceptible to strand invasion by the RecA coated 3′ tail of the extended recording tag (Bell, et al., 2015, Elife 4: e08646). The presence of a single-stranded binding protein can facilitate the strand displacement reaction.

In some embodiments, a DNA polymerase that is used for primer extension possesses strand-displacement activity and has limited or is devoid of 3′-5 exonuclease activity. Several of many examples of such polymerases include Klenow exo- (Klenow fragment of DNA Pol 1), T4 DNA polymerase exo-, T7 DNA polymerase exo (Sequenase 2.0), Pfu exo-, Vent exo-, Deep Vent exo-, Bst DNA polymerase large fragment exo-, Bca Pol, 9° N Pol, and Phi29 Pol exo-. In a preferred embodiment, the DNA polymerase is active at room temperature and up to 45° C. In another embodiment, a “warm start” version of a thermophilic polymerase is employed such that the polymerase is activated and is used at about 40° C.-50° C. An exemplary warm start polymerase is Bst 2.0 Warm Start DNA Polymerase (New England Biolabs).

Additives useful in strand-displacement replication include any of a number of single-stranded DNA binding proteins (SSB proteins) of bacterial, viral, or eukaryotic origin, such as SSB protein of E. coli, phage T4 gene 32 product, phage 17 gene 2.5 protein, phage Pf3 SSB, replication protein A RPA32 and RPA14 subunits (Wold, 1997); other DNA binding proteins, such as adenovirus DNA-binding protein, herpes simplex protein ICP8, BMRF1 polymerase accessory subunit, herpes virus UL29 SSB-like protein; any of a number of replication complex proteins known to participate in DNA replication, such as phage 17 helicase/primase, phage T4 gene 41 helicase, E. coli Rep helicase, E. coli recBCD helicase, recA, E. coli and eukaryotic topoisomerases (Annu Rev Biochem. (2001) 70:369-413).

Mis-priming or self-priming events, such as when the terminal spacer sequence of the recoding tag primes extension self-extension may be minimized by inclusion of single stranded binding proteins (T4 gene 32, E. coli SSB, etc.), DMSO (1-10%), formamide (1-10%), BSA(10-100 ug/ml), TMACl (1-5 mM), ammonium sulfate (10-50 mM), betaine (1-3 M), glycerol (5-40%), or ethylene glycol (5-40%), in the primer extension reaction.

Most type A polymerases are devoid of 3′ exonuclease activity (endogenous or engineered removal), such as Klenow exo-, 17 DNA polymerase exo-(Sequenase 2.0), and Taq polymerase catalyzes non-templated addition of a nucleotide, preferably an adenosine base (to lesser degree a G base, dependent on sequence context) to the 3′ blunt end of a duplex amplification product. For Taq polymerase, a 3′ pyrimidine (C>T) minimizes non-templated adenosine addition, whereas a 3′ purine nucleotide (G>A) favours non-templated adenosine addition. In some embodiments, using Taq polymerase for primer extension, placement of a thymidine base in the coding tag between the spacer sequence distal from the binding agent and the adjacent barcode sequence (e.g., encoder sequence or cycle specific sequence) accommodates the sporadic inclusion of a non-templated adenosine nucleotide on the 3′ terminus of the spacer sequence of the recording tag. In this manner, the extended recording tag (with or without a non-templated adenosine base) can anneal to the coding tag and undergo primer extension.

Alternatively, addition of non-templated base can be reduced by employing a mutant polymerase (mesophilic or thermophilic) in which non-templated terminal transferase activity has been greatly reduced by one or more point mutations, especially in the 0-helix region (see U.S. Pat. No. 7,501,237) (Yang et al., Nucleic Acids Res. (2002) 30(19): 4314-4320). Pfu exo-, which is 3′ exonuclease deficient and has strand-displacing ability, also does not have non-templated terminal transferase activity.

In another embodiment, polymerase extension buffers are comprised of 40-120 mM buffering agent such as Tris-Acetate, Tris-HCl, HEPES, etc. at a pH of 6-9.

Self-priming/mis-priming events initiated by self-annealing of the terminal spacer sequence of the extended recording tag with internal regions of the extended recording tag may be minimized by including pseudo-complementary bases in the recording/extended recording tag (Lahoud et al., Nucleic Acids Res. (2008) 36:3409-3419), (Hoshika et al., Angew Chem Int Ed Engl (2010) 49(32): 5554-5557). Pseudo-complementary bases show significantly reduced hybridization affinities for the formation of duplexes with each other due the presence of chemical modification. However, many pseudo-complementary modified bases can form strong base pairs with natural DNA or RNA sequences. In certain embodiments, the coding tag spacer sequence is comprised of multiple A and T bases, and commercially available pseudo-complementary bases 2-aminoadenine and 2-thiothymine are incorporated in the recording tag using phosphoramidite oligonucleotide synthesis. Additional pseudocomplementary bases can be incorporated into the extended recording tag during primer extension by adding pseudo-complementary nucleotides to the reaction (Gamper et al., Biochemistry. (2006) 45(22):6978-86).

In some embodiments, to minimize non-specific interaction of the coding tag labeled binding agents in solution with the recording tags of immobilized proteins, competitor (also referred to as blocking) oligonucleotides complementary to recording tag spacer sequences can be added to binding reactions to minimize non-specific interactions. In some embodiments, blocking oligonucleotides are relatively short. In some embodiments the blocking oligonucleotide is integrated into the coding tag via a hairpin structure. Excess competitor oligonucleotides are washed from the binding reaction prior to primer extension, which effectively dissociates the annealed competitor oligonucleotides from the recording tags, especially when exposed to slightly elevated temperatures (e.g., 30-50° C.). Blocking oligonucleotides may comprise a terminator nucleotide at its 3′ end to prevent primer extension.

In certain embodiments, the annealing of the spacer sequence on the recording tag to the complementary spacer sequence on the coding tag is metastable under the primer extension reaction conditions (i.e., the annealing Tm is similar to the reaction temperature). This allows the spacer sequence of the coding tag to displace any blocking oligonucleotide annealed to the spacer sequence of the recording tag.

Coding tag information associated with a specific binding agent may also be transferred to a recording tag via ligation. Ligation may be a blunt end ligation or sticky end ligation. Ligation may be an enzymatic ligation reaction. Examples of ligases include, but are not limited to CV DNA ligase (e.g. U.S. Patent Application Publication US 20140378315A1), T4 DNA ligase, 17 DNA ligase, T3 DNA ligase, Taq DNA ligase, E. coli DNA ligase, 9° N DNA ligase, Electroligase®. Alternatively, a ligation may be a chemical ligation reaction. In some embodiments, a spacer-less ligation is accomplished by using hybridization of a “recording helper” sequence with an arm on the coding tag. The annealed complement sequences are chemically ligated using standard chemical ligation or “click chemistry” (Gunderson et al., Genome Res (1998) 8(11): 1142-1153; Peng et al., European J Org Chem (2010) (22): 4194-4197; El-Sagheer et al., Proc Natl Acad Sci USA (2011) 108(28): 11338-11343; El-Sagheer et al., Org Biomol Chem (2011) 9(1): 232-235; Sharma et al., Anal Chem (2012) 84(14): 6104-6109; Roloff et al., Bioorg Med Chem (2013) 21(12): 3458-3464; Litovchick et al., Artif DNA PNA XNA (2014) 5(1): e27896; Roloff et al., Methods Mol Biol (2014) 1050:131-141).

In another embodiment, transfer of PNAs can be accomplished with chemical ligation using published techniques. The structure of PNA is such that it has a 5′ N-terminal amine group and an unreactive 3′ C-terminal amide. Chemical ligation of PNA requires that the termini be modified to be chemically active. This is typically done by derivitizing the 5′ N-terminus with a cysteinyl moiety and the 3′ C-terminus with a thioester moiety. Such modified PNAs easily couple using standard native chemical ligation conditions (Roloff et al., (2013) Bioorgan. Med. Chem. 21:3458-3464).

In some embodiments, coding tag information can be transferred using topoisomerase. Topoisomerase can be used be used to ligate a topo-charged 3′ phosphate on the recording tag to the 5′ end of the coding tag, or complement thereof (Shuman et al., 1994, J. Biol. Chem. 269:32678-32684).

As described herein, a binding agent may bind to a post-translationally modified amino acid. Thus, in certain embodiments, an extended recording tag comprises coding tag information relating to amino acid sequence and post-translational modifications of the polypeptide. In some embodiments, detection of internal post-translationally modified amino acids (e.g., phosphorylation, glycosylation, succinylation, ubiquitination, S-Nitrosylation, methylation, N-acetylation, lipidation, etc.) is be accomplished prior to detection and elimination of terminal amino acids (e.g., NTAA or CTAA). In one example, a peptide is contacted with binding agents for PTM modifications, and associated coding tag information are transferred to the recording tag. Once the detection and transfer of coding tag information relating to amino acid modifications is complete, the PTM modifying groups can be removed before detection and transfer of coding tag information for the primary amino acid sequence using N-terminal or C-terminal degradation methods. Thus, resulting extended recording tags indicate the presence of post-translational modifications in a peptide sequence, though not the sequential order, along with primary amino acid sequence information.

In some embodiments, detection of internal post-translationally modified amino acids may occur concurrently with detection of primary amino acid sequence. In one example, an NTAA (or CTAA) is contacted with a binding agent specific for a post-translationally modified amino acid, either alone or as part of a library of binding agents (e.g., library composed of binding agents for the 20 standard amino acids and selected post-translational modified amino acids). Successive cycles of terminal amino acid elimination and contact with a binding agent (or library of binding agents) follow. Thus, resulting extended recording tags indicate the presence and order of post-translational modifications in the context of a primary amino acid sequence.

In certain embodiments, an ensemble of recording tags may be employed per polypeptide to improve the overall robustness and efficiency of coding tag information transfer. The use of an ensemble of recording tags associated with a given polypeptide rather than a single recording tag improves the efficiency of library construction due to potentially higher coupling yields of coding tags to recording tags, and higher overall yield of libraries. The yield of a single concatenated extended recording tag is directly dependent on the stepwise yield of concatenation, whereas the use of multiple recording tags capable of accepting coding tag information does not suffer the exponential loss of concatenation.

For embodiments involving analysis of denatured proteins, polypeptides, and peptides, the bound binding agent and annealed coding tag can be removed following primer extension by using highly denaturing conditions (e.g., 0.1-0.2 N NaOH, 6M Urea, 2.4 M guanidinium isothiocyanate, 95% formamide, etc.).

B. Characterization of Polypeptides Via Cyclic Rounds of Amino Acid Recognition, Recording Tag Extension, and Amino Acid Elimination

In certain embodiments, the methods for analyzing a polypeptide provided in the present disclosure comprise multiple binding cycles, where the polypeptide is contacted with a plurality of binding agents, and successive binding of binding agents transfers historical binding information in the form of a nucleic acid based coding tag to at least one recording tag associated with the polypeptide. In this way, a historical record containing information about multiple binding events is generated in a nucleic acid format.

In embodiments relating to methods of analyzing peptide polypeptides using an N-terminal degradation based approach (see, FIG. 1), following contacting and binding of a first binding agent to an n NTAA of a peptide of n amino acids and transfer of the first binding agent's coding tag information to a recording tag associated with the peptide, thereby generating a first order extended recording tag, the n NTAA is eliminated as described herein. Elimination of the n NTAA converts the n-1 amino acid of the peptide to an N-terminal amino acid, which is referred to herein as an n-1 NTAA. As described herein, the n NTAA may optionally be functionalized with a moiety (e.g., PTC or derivatized PTC, DNP, SNP, acetyl, amidinyl, guanidinyl, etc.), which is particularly useful in conjunction with cleavage enzymes that are engineered to bind to a functionalized form of NTAA. Some or all of the steps including functionalization, binding, and elimination may be performed in the presence of microwave energy. In some embodiments, the functionalized NTAA includes a ligand group that is capable of covalent binding to a binding agent. If the n NTAA was functionalized, the n-1 NTAA is then functionalized with the same moiety. A second binding agent is contacted with the peptide and binds to the n-1 NTAA, and the second binding agent's coding tag information is transferred to the first order extended recording tag thereby generating a second order extended recording tag (e.g., for generating a concatenated n^{th} order extended recording tag representing the peptide), or to a different recording tag (e.g., for generating multiple extended recording tags, which collectively represent the peptide). Elimination of the n-1 NTAA converts the n-2 amino acid of the peptide to an N-terminal amino acid, which is referred to herein as n-2 NTAA. Additional binding, transfer, elimination, and optionally NTAA functionalization, can occur as described above up to n amino acids to generate an n^{th} order extended recording tag or n separate extended recording tags, which collectively represent the peptide. As used herein, an n “order” when used in reference to a binding agent, coding tag, or extended recording tag, refers to the n binding cycle, wherein the binding agent and its associated coding tag is used or the n binding cycle where the extended recording tag is created. In some embodiments, steps including the NTAA in the described exemplary approach can be performed instead with a CTAA.

In some embodiments, contacting of the first binding agent and second binding agent to the polypeptide, and optionally any further binding agents (e.g., third binding agent, fourth binding agent, fifth binding agent, and so on), are performed at the same time. For example, the first binding agent and second binding agent, and optionally any further order binding agents, can be pooled together, for example to form a library of binding agents. In another example, the first binding agent and second binding agent, and optionally any further order binding agents, rather than being pooled together, are added simultaneously to the polypeptide. In one embodiment, a library of binding agents comprises at least 20 binding agents that selectively bind to the 20 standard, naturally occurring amino acids.

In other embodiments, the first binding agent and second binding agent, and optionally any further order binding agents, are each contacted with the polypeptide in separate binding cycles, added in sequential order. In certain embodiments, multiple binding agents are used at the same time, in parallel. This parallel approach saves time and reduces non-specific binding by non-cognate binding agents to a site that is bound by a cognate binding agent (because the binding agents are in competition).

The length of the final extended recording tags generated by the methods described herein is dependent upon multiple factors, including the length of the coding tag (e.g., encoder sequence and spacer), the length of the recording tag (e.g., unique molecular identifier, spacer, universal priming site, bar code), the number of binding cycles performed, and whether coding tags from each binding cycle are transferred to the same extended recording tag or to multiple extended recording tags. In an example for a concatenated extended recording tag representing a peptide and produced by an Edman degradation like elimination method, if the coding tag has an encoder sequence of 5 bases that is flanked on each side by a spacer of 5 bases, the coding tag information on the final extended recording tag, which represents the peptide's binding agent history, is 10 bases x number of Edman Degradation cycles. For a 20-cycle run, the extended recording is at least 200 bases (not including the initial recording tag sequence). This length is compatible with standard next generation sequencing instruments.

After the final binding cycle and transfer of the final binding agent's coding tag information to the extended recording tag, the recorder tag can be capped by addition of a universal reverse priming site via ligation, primer extension or other methods known in the art. In some embodiments, the universal forward priming site in the recording tag is compatible with the universal reverse priming site that is appended to the final extended recording tag. In some embodiments, a universal reverse priming site is an Illumina P7 primer (5′-CAAGCAGAAGACGGCATACGAGAT-3′-SEQ ID NO:12) or an Illumina P5 primer (5′-AATGATACGGCGACCACCGA-3′-SEQ ID NO:11). The sense or antisense P7 may be appended, depending on strand sense of the recording tag. An extended recording tag library can be cleaved or amplified directly from the solid support (e.g., beads) and used in traditional next generation sequencing assays and protocols.

In some embodiments, a primer extension reaction is performed on a library of single stranded extended recording tags to copy complementary strands thereof. In some embodiments, the NGPS peptide sequencing assay (e.g., ProteoCode assay), comprises several chemical and enzymatic steps in a cyclical progression. In some cases, one advantage of a single molecule assay is the robustness to inefficiencies in the various cyclical chemical/enzymatic steps. In some embodiments, the use of cycle-specific barcodes present in the coding tag sequence allows an advantage to the assay.

Using cycle-specific coding tags, information is tracked from each cycle. Since this is a single molecule sequencing approach, even 70% efficiency at each binding/transfer cycle in the sequencing process is more than sufficient to generate mappable sequence information. As an example, a ten-base peptide sequence “CPVQLWVDST” (SEQ ID NO:13) might be read as “CPXQXWXDXT” (SEQ ID NO:10) (where X=any amino acid; the presence an amino acid is inferred by cycle number tracking). In some embodiments, this partial amino acid sequence read is sufficient to uniquely map it back to the human p53 protein using BLASTP. In some embodiments, when cycle-specific barcodes are combined with partitioning methods, absolute identification of the protein can be accomplished with only a few amino acids identified out of 10 positions since partitioning provides information regarding what set of peptides map to the original protein molecule (via compartment barcodes).

C. Processing and Analysis of Tags

Extended recording tag, extended coding tag, and di-tag libraries representing the polypeptide(s) of interest can be processed and analysed using a variety of nucleic acid sequencing methods. Examples of sequencing methods include, but are not limited to, chain termination sequencing (Sanger sequencing); next generation sequencing methods, such as sequencing by synthesis, sequencing by ligation, sequencing by hybridization, polony sequencing, ion semiconductor sequencing, and pyrosequencing; and third generation sequencing methods, such as single molecule real time sequencing, nanopore-based sequencing, duplex interrupted sequencing, and direct imaging of DNA using advanced microscopy.

Suitable sequencing methods for use in the invention include, but are not limited to, sequencing by hybridization, sequencing by synthesis technology (e.g., HiSeg™ and Solexa™, Illumina), SMRT™ (Single Molecule Real Time) technology (Pacific Biosciences), true single molecule sequencing (e.g., HeliScope™, Helicos Biosciences), massively parallel next generation sequencing (e.g., SOLiD™, Applied Biosciences; Solexa and HiSeg™, Illumina), massively parallel semiconductor sequencing (e.g., Ion Torrent), and pyrosequencing technology (e.g., GS FLX and GS Junior Systems, Roche/454) and nanopore sequence (e.g., Oxford Nanopore Technologies).

A library of extended recording tags, extended coding tags, or di-tags may be amplified in a variety of ways. A library of extended recording tags, extended coding tags, or di-tags may undergo exponential amplification, e.g., via PCR or emulsion PCR. Emulsion PCR is known to produce more uniform amplification (Hori, Fukano et al., Biochem Biophys Res Commun (2007) 352(2): 323-328). Alternatively, a library of extended recording tags, extended coding tags, or di-tags may undergo linear amplification, e.g., via in vitro transcription of template DNA using 17 RNA polymerase. The library of extended recording tags, extended coding tags, or di-tags can be amplified using primers compatible with the universal forward priming site and universal reverse priming site contained therein. A library of extended recording tags, extended coding tags, or di-tags can also be amplified using tailed primers to add sequence to either the 5′-end, 3′-end or both ends of the extended recording tags, extended coding tags, or di-tags. Sequences that can be added to the termini of the extended recording tags, extended coding tags, or di-tags include library specific index sequences to allow multiplexing of multiple libraries in a single sequencing run, adaptor sequences, read primer sequences, or any other sequences for making the library of extended recording tags, extended coding tags, or di-tags compatible for a sequencing platform. An example of a library amplification in preparation for next generation sequencing is as follows: a 20 μl PCR reaction volume is set up using an extended recording tag library eluted from ˜1 mg of beads (˜10 ng), 200 μM dNTP, 1 μM of each forward and reverse amplification primers, 0.5 μl (1U) of Phusion Hot Start enzyme (New England Biolabs) and subjected to the following cycling conditions: 98° C. for 30 sec followed by 20 cycles of 98° C. for 10 sec, 60° C. for 30 sec, 72° C. for 30 sec, followed by 72° C. for 7 min, then hold at 4° C.

In certain embodiments, either before, during or following amplification, the library of extended recording tags, extended coding tags, or di-tags can undergo target enrichment. In some embodiments, target enrichment can be used to selectively capture or amplify extended recording tags representing polypeptides of interest from a library of extended recording tags, extended coding tags, or di-tags before sequencing. In some aspects, target enrichment for protein sequencing is challenging because of the high cost and difficulty in producing highly-specific binding agents for target proteins. In some cases, antibodies are notoriously non-specific and difficult to scale production across thousands of proteins. In some embodiments, the methods of the present disclosure circumvent this problem by converting the protein code into a nucleic acid code which can then make use of a wide range of targeted DNA enrichment strategies available for DNA libraries. In some cases, peptides of interest can be enriched in a sample by enriching their corresponding extended recording tags. Methods of targeted enrichment are known in the art, and include hybrid capture assays, PCR-based assays such as TruSeq custom Amplicon (Illumina), padlock probes (also referred to as molecular inversion probes), and the like (see, Mamanova et al., (2010) Nature Methods 7: 111-118; Bodi et al., J. Biomol. Tech. (2013) 24:73-86; Ballester et al., (2016) Expert Review of Molecular Diagnostics 357-372; Mertes et al., (2011) Brief Funct. Genomics 10:374-386; Nilsson et al., (1994) Science 265:2085-8; each of which are incorporated herein by reference in their entirety).

In one embodiment, a library of extended recording tags, extended coding tags, or di-tags is enriched via a hybrid capture-based assay. In a hybrid-capture based assay, the library of extended recording tags, extended coding tags, or di-tags is hybridized to target-specific oligonucleotides or “bait oligonucleotide” that are labelled with an affinity tag (e.g., biotin). Extended recording tags, extended coding tags, or di-tags hybridized to the target-specific oligonucleotides are “pulled down” via their affinity tags using an affinity ligand (e.g., streptavidin coated beads), and background (non-specific) extended recording tags are washed away. The enriched extended recording tags, extended coding tags, or di-tags are then obtained for positive enrichment (e.g., eluted from the beads).

For bait oligonucleotides synthesized by array-based “in situ” oligonucleotide synthesis and subsequent amplification of oligonucleotide pools, competing baits can be engineered into the pool by employing several sets of universal primers within a given oligonucleotide array. For each type of universal primer, the ratio of biotinylated primer to non-biotinylated primer controls the enrichment ratio. The use of several primer types enables several enrichment ratios to be designed into the final oligonucleotide bait pool.

A bait oligonucleotide can be designed to be complementary to an extended recording tag, extended coding tag, or di-tag representing a polypeptide of interest. The degree of complementarity of a bait oligonucleotide to the spacer sequence in the extended recording tag, extended coding tag, or di-tag can be from 0% to 100%, and any integer in between. This parameter can be easily optimized by a few enrichment experiments. In some embodiments, the length of the spacer relative to the encoder sequence is minimized in the coding tag design or the spacers are designed such that they unavailable for hybridization to the bait sequences. One approach is to use spacers that form a secondary structure in the presence of a cofactor. An example of such a secondary structure is a G-quadruplex, which is a structure formed by two or more guanine quartets stacked on top of each other (Bochman et al., Nat Rev Genet (2012) 13(11):770-780). A guanine quartet is a square planar structure formed by four guanine bases that associate through Hoogsteen hydrogen bonding. The G-quadruplex structure is stabilized in the presence of a cation, e.g., K+ ions vs. Li+ ions.

To minimize the number of bait oligonucleotides employed, a set of relatively unique peptides from each protein can be bioinformatically identified, and only those bait oligonucleotides complementary to the corresponding extended recording tag library representations of the peptides of interest are used in the hybrid capture assay. In some embodiments, sequential rounds or enrichment can also be carried out, with the same or different bait sets.

To enrich the entire length of a polypeptide in a library of extended recording tags, extended coding tags, or di-tags representing fragments thereof (e.g., peptides), “tiled” bait oligonucleotides can be designed across the entire nucleic acid representation of the protein.

In another embodiment, primer extension and ligation-based mediated amplification enrichment (AmpliSeq, PCR, TruSeq TSCA, etc.) can be used to select and module fraction enriched of library elements representing a subset of polypeptides. Competing oligonucleotides can also be employed to tune the degree of primer extension, ligation, or amplification. In the simplest implementation, this can be accomplished by having a mix of target specific primers comprising a universal primer tail and competing primers lacking a 5′ universal primer tail. After an initial primer extension, only primers with the 5′ universal primer sequence can be amplified. The ratio of primer with and without the universal primer sequence controls the fraction of target amplified. In other embodiments, the inclusion of hybridizing but non-extending primers can be used to modulate the fraction of library elements undergoing primer extension, ligation, or amplification.

Targeted enrichment methods can also be used in a negative selection mode to selectively remove extended recording tags, extended coding tags, or di-tags from a library before sequencing. Thus, in the example described above using biotinylated bait oligonucleotides and streptavidin coated beads, the supernatant is retained for sequencing while the bait-oligonucleotide:extended recording tag, extended coding tag, or di-tag hybrids bound to the beads are not analysed. Examples of undesirable extended recording tags, extended coding tags, or di-tags that can be removed are those representing over abundant polypeptide species, e.g., for proteins, albumin, immunoglobulins, etc.

A competitor oligonucleotide bait, hybridizing to the target but lacking a biotin moiety, can also be used in the hybrid capture step to modulate the fraction of any particular locus enriched. The competitor oligonucleotide bait competes for hybridization to the target with the standard biotinylated bait effectively modulating the fraction of target pulled down during enrichment. The ten orders dynamic range of protein expression can be compressed by several orders using this competitive suppression approach, especially for the overly abundant species such as albumin. Thus, the fraction of library elements captured for a given locus relative to standard hybrid capture can be modulated from 100% down to 0% enrichment.

Additionally, library normalization techniques can be used to remove overly abundant species from the extended recording tag, extended coding tag, or di-tag library. This approach works best for defined length libraries originating from peptides generated by site-specific protease digestion such as trypsin, LysC, GluC, etc. In one example, normalization can be accomplished by denaturing a double-stranded library and allowing the library elements to re-anneal. The abundant library elements re-anneal more quickly than less abundant elements due to the second-order rate constant of bimolecular hybridization kinetics (Bochman, Paeschke et al. 2012). The ssDNA library elements can be separated from the abundant dsDNA library elements using methods known in the art, such as chromatography on hydroxyapatite columns (VanderNoot, et al., 2012, Biotechniques 53:373-380) or treatment of the library with a duplex-specific nuclease (DSN) from Kamchatka crab (Shagin et al., (2002) Genome Res. 12:1935-42) which destroys the dsDNA library elements.

Any combination of fractionation, enrichment, and subtraction methods, of the polypeptides before attachment to the solid support and/or of the resulting extended recording tag library can economize sequencing reads and improve measurement of low abundance species.

In some embodiments, a library of extended recording tags, extended coding tags, or di-tags is concatenated by ligation or end-complementary PCR to create a long DNA molecule comprising multiple different extended recorder tags, extended coding tags, or di-tags, respectively (Du et al., (2003) BioTechniques 35:66-72; Muecke et al., (2008) Structure 16:837-841; U.S. Pat. No. 5,834,252, each of which is incorporated by reference in its entirety). This embodiment is preferable for nanopore sequencing in which long strands of DNA are analyzed by the nanopore sequencing device.

In some embodiments, direct single molecule analysis is performed on an extended recording tag, extended coding tag, or di-tag (see, e.g., Harris et al., (2008) Science 320:106-109). The extended recording tags, extended coding tags, or di-tags can be analysed directly on the solid support, such as a flow cell or beads that are compatible for loading onto a flow cell surface (optionally microcell patterned), wherein the flow cell or beads can integrate with a single molecule sequencer or a single molecule decoding instrument. For single molecule decoding, hybridization of several rounds of pooled fluorescently-labelled of decoding oligonucleotides (Gunderson et al., (2004) Genome Res. 14:970-7) can be used to ascertain both the identity and order of the coding tags within the extended recording tag. To deconvolute the binding order of the coding tags, the binding agents may be labelled with cycle-specific coding tags as described above (see also, Gunderson et al., (2004) Genome Res. 14:970-7). Cycle-specific coding tags will work for both a single, concatenated extended recording tag representing a single polypeptide, or for a collection of extended recording tags representing a single polypeptide.

Following sequencing of the extended reporter tag, extended coding tag, or di-tag libraries, the resulting sequences can be collapsed by their UMIs and then associated to their corresponding polypeptides and aligned to the totality of the proteome. Resulting sequences can also be collapsed by their compartment tags and associated to their corresponding compartmental proteome, which in a particular embodiment contains only a single or a very limited number of protein molecules. Both protein identification and quantification can easily be derived from this digital peptide information.

In some embodiments, the coding tag sequence can be optimized for the particular sequencing analysis platform. In a particular embodiment, the sequencing platform is nanopore sequencing. In some embodiments, the sequencing platform has a per base error rate of >1%, >5%, >10%, >15%, >20%, >25%, or >30%. For example, if the extended recording tag is to be analyzed using a nanopore sequencing instrument, the barcode sequences (e.g., encoder sequences) can be designed to be optimally electrically distinguishable in transit through a nanopore. Peptide sequencing according to the methods described herein may be well-suited for nanopore sequencing, given that the single base accuracy for nanopore sequencing is still rather low (75%-85%), but determination of the “encoder sequence” should be much more accurate (>99%). Moreover, a technique called duplex interrupted nanopore sequencing (DI) can be employed with nanopore strand sequencing without the need for a molecular motor, greatly simplifying the system design (Derrington et al., Proc Natl Acad Sci USA (2010) 107(37): 16060-16065). Readout of the extended recording tag via DI nanopore sequencing requires that the spacer elements in the concatenated extended recording tag library be annealed with complementary oligonucleotides. The oligonucleotides used herein may comprise LNAs, or other modified nucleic acids or analogs to increase the effective Tm of the resultant duplexes. As the single-stranded extended recording tag decorated with these duplex spacer regions is passed through the pore, the double strand region will become transiently stalled at the constriction zone enabling a current readout of about three bases adjacent to the duplex region. In a particular embodiment for DI nanopore sequencing, the encoder sequence is designed in such a way that the three bases adjacent to the spacer element create maximally electrically distinguishable nanopore signals (Derrington et al., Proc Natl Acad Sci USA (2010) 107(37): 16060-16065). As an alternative to motor-free DI sequencing, the spacer element can be designed to adopt a secondary structure such as a G-quartet, which will transiently stall the extended recording tag, extended coding tag, or di-tag as it passes through the nanopore enabling readout of the adjacent encoder sequence (Shim et al., Nucleic Acids Res (2009) 37(3): 972-982; Zhang et al., mAbs (2016) 8, 524-535). After proceeding past the stall, the next spacer will again create a transient stall, enabling readout of the next encoder sequence, and so forth.

The methods disclosed herein can be used for analysis, including detection, quantitation and/or sequencing, of a plurality of polypeptides simultaneously (multiplexing). Multiplexing as used herein refers to analysis of a plurality of polypeptides in the same assay. The plurality of polypeptides can be derived from the same sample or different samples. The plurality of polypeptides can be derived from the same subject or different subjects. The plurality of polypeptides that are analyzed can be different polypeptides, or the same polypeptide derived from different samples. A plurality of polypeptides includes 2 or more polypeptides, 5 or more polypeptides, 10 or more polypeptides, 50 or more polypeptides, 100 or more polypeptides, 500 or more polypeptides, 1000 or more polypeptides, 5,000 or more polypeptides, 10,000 or more polypeptides, 50,000 or more polypeptides, 100,000 or more polypeptides, 500,000 or more polypeptides, or 1,000,000 or more polypeptides.

Sample multiplexing can be achieved by upfront barcoding of recording tag labeled polypeptide samples. Each barcode represents a different sample, and samples can be pooled prior to cyclic binding assays or sequence analysis. In this way, many barcode-labeled samples can be simultaneously processed in a single tube. This approach is a significant improvement on immunoassays conducted on reverse phase protein arrays (RPPA) (Akbani et al., Mol Cell Proteomics (2014) 13(7): 1625-1643; Creighton et al., Drug Des Devel Ther (2015) 9: 3519-3527; Nishizuka et al., Drug Metab Pharmacokinet (2016) 31(1): 35-45). In this way, the present disclosure essentially provides a highly digital sample and analyte multiplexed alternative to the RPPA assay with a simple workflow.

### IV. Exemplary Application of Microwave Energy and Instruments for Use

Provided herein are exemplary methods of treating a polypeptide performed with the application of radiation, e.g., electromagnetic radiation or microwave energy. In some embodiments, the provided methods are performed in an exemplary system comprising a microwave source for performing chemical and physical processes within a microwave radiation field. In some cases, an exemplary apparatus permitting a plurality of different chemical and physical processes is used.

In some embodiments, the contacting of the polypeptide with a functionalizing reagent, with a binding agent, or with a reagent to remove one or more amino acid(s) is performed in a cavity in communication with or connected to a microwave radiation source. In some examples, the contacting of the contacting of the polypeptide with any of the reagents or binding agents provided herein is performed in a microwave chamber (See U.S. Patent Application Publication Number US 2013/0001221; International Patent Publication No. WO 2012/075570). In some embodiments, the provided methods are performed in a single-mode cavity. In some cases, the provided methods are performed in a multimode microwave cavity.

Equipment and reagents of standard type may be used in the present method. In one embodiment, the method is performed in a vessel wherein the temperature and/or pressure may be monitored and optionally moderated. In some aspects, the method is performed on a sample in a vessel. In some embodiments, the temperature of the sample within the vessel is monitored. In some embodiments, the pressure of the sample-containing vessel vented via a pressure vent in the vessel. In some examples, a control system controls and adjusts the microwave source based on feedback such as temperature, pressure, of the sample. In some embodiments, the temperature is monitored and/or controlled at any or all step(s) of the methods provided herein. For example, the temperature may be adjusted to a suitable value or maintained at a suitable level determined by the skilled person. In some embodiments, the method is performed in a vessel that may have cooling applied. For example, active cooling (e.g., air cooling) may be applied to the vessel. In some embodiments, temperature is controlled within the range of about 10° C. to 200° C., about 10° C. to 150° C., about 10° C. to 100° C., about 20° C. to 200° C., about 20° C. to 150° C., about 20° C. to 125° C., about 20° C. to 100° C., or about 25° C. to 125° C. In some cases, the temperature is moderated (e.g. cooled) such that the sample in the vessel is rapidly cooled. In some examples, the moderation of the temperature is performed using air, chilled air, a chilled surface in contact with the sample vessel, or liquid cooling. In some cases, thermoelectric cooling or heating is used to moderate or modulate temperature of the sample. For example, a Peltier cooler or heater can be used to moderate or modulate temperature of the sample.

In some embodiments of the provided methods, the reactions may also be quenched, such as by reducing the overall reaction temperature. In some further embodiments, agitation, such as by electromagnetic stirring at various speeds, may be applied to the reaction mixture. There are a number of parameters that can be controlled and specified with the microwave source or vessel containing the microwave source. For example, parameters may include time, temperature, pressure, cooling, power, stirring rate, pre-stirring, initial power, dielectric of solution, vial type or material, and/or absorption. In some embodiments, microwave instruments may provide controllable, reproducible and fast application of energy under conditions where rapid cooling down of the reaction can take place.

Various microwave reactors suitable for performing the method of the present invention and the operation of these apparatus will be apparent to those skilled in the art. Such microwave reactors may for example include monomodal microwave reactors e.g., Emrys Liberator (Biotage), Discover SP system (CEM), Ethos TouchControl (Milestone Inc.) and MicroCure2100 BatchSystem (Lambda Technologies).

In some embodiments, the microwave energy is generated by a solid-state microwave power amplifier. In some examples, the power amplifier can vary both the microwave power (e.g., 0-10 W or 0-100 W or 0-1000 W) and frequency (e.g., 2.3-2.7 GHz). In some examples, the microwave energy is applied to a sample in a single mode resonant cavity. For example, the dimensions of the cavity are designed to enable excitation of a single-mode of the cavity to create a single standing wave with the time-averaged electric field (E field) maximal at the sample positioned in the center of the cavity (See e.g., Koyama et al., Journal of Flow Chemistry (2018) 8(3): 147-156; Barham et al., Chem Rec (2019) 19(1): 188-203; Odajima et al. Chem rec (2019 19(1):204-211). In a preferred embodiment, a single-mode microwave irradiation system in which microwave excitation is radiated as a single standing wave, and the time-averaged electric field is maximal at a sample-containing vessel positioned in the center of the cavity, is used to uniformly heat the volume of the sample.

In some embodiments, the microwave energy generator is in communication with a control unit. In some embodiments, the electric field and/or cavity exposed to the microwave energy is in communication with the microwave energy generator and/or the control unit. In some cases, the control unit and/or microwave generator is in communication with an electric field sensing element and a thermal sensing element. In some embodiments, the power and frequency of the microwave radiation are controlled automatically by feedback from an electric field sensing element and a thermal sensing element (Koyama et al., Journal of Flow Chemistry (2018) 8(3): 147-156; Barham et al., Chem Rec (2019) 19(1): 188-203; Odajima et al. Chem rec (2019 19(1):204-211). An autotuning of frequency feature from these feedback elements, can be used to adjust the microwave frequency to stay in tune with the changing resonant modes of cavity/vessel system (e.g. the resonant frequency of cavity/vessel system shifts with changes in solution type, i.e. dielectric/permitivity differences between solutions, in the vessel containing the sample and with temperature of the vessel).

In some embodiments, the microwave energy has a wavelength from about one meter to about one millimeter, e.g., a wavelength from about 0.3 m to about 3 mm. In some cases, the microwave energy has a frequency from about 300 MHz (1 m) to about 300 GHz (1 mm). In some embodiments, the microwave energy has a frequency from about 1 GHz to about 100 GHz. In some embodiments, the microwave energy has a frequency from about 0.5 GHz to 500 GHz, from about 0.5 Ghz to 100 GHz, from about 0.5 GHz to 50 GHz, from about 0.5 GHz to 25 GHz, from about 0.5 GHz to 10 GHz, from about 0.5 GHz to 5 GHz, or from about 0.5 GHz to 2.5 GHz, 2 GHz to 500 GHz, from about 2 GHz to 100 GHz, from about 2 GHz to 50 GHz, from about 2 GHz to 25 GHz, from about 2 GHz to 10 GHz, from about 2 GHz to 5 GHz, or from about 2 GHz to 2.5 GIL. In one example, the microwave generator operates at about 902-928 MHz. In a preferred embodiment, the microwave energy has a frequency from about 2.44 GHz to 2.46 GHz. In one example, the microwave generator operates at 2.45 GHz+-0.2 GHz. In some specific cases, a solid-state microwave generator is used to apply microwave energy to a single mode resonant cavity. In a preferred mode, the microwave Generator operates at 2.45 GHz+-0.-0.05 GHz.

In some embodiments, the microwave energy has a frequency with an IEEE radar band designation of S, C, X, Kᵤ, K or Kₐ band. In some embodiments, the microwave energy has a photon energy (eV) from about 1.24 μeV to about 1.24 meV, e.g., at about 1.24 μeV to about 12.4 μeV, about 12.4 μeV to about 124 μeV, about 124 μeV to about 1.24 meV. In some examples, the microwave energy is applied at about 5 watts, about 10 watts, about 15 watts, about 20 watts, about 25 watts, about 30 watts, about 35 watts, about 40 watts, about 45 watts, about 50 watts, about 60 watts, about 70 watts, about 80 watts, about 90 watts, about 100 watts, about 110 watts, about 120 watts, about 130 watts, about 140 watts, about 150 watts, about 300 watts or higher watts, or a subrange thereof. In some embodiments, the microwave is generated by an amplifier capable of delivering between about 0 W to 10 W, 0 W to 50 W, between about 0 W to 100 W, between about 0 W to 200 W, between about 0 W to 300 W, between about 0 W to 400 W, between about 0 W to 500 W, or between about 25 W to 200 W. The microwave energy may be adjusted to a suitable value or level determined by the skilled person based on the characteristics of the sample, for example, volume of the sample.

In some embodiments, the microwave energy is applied for a time period of about 1 minute, 2 minutes, 3 minutes, 4 minutes, 5 minutes, 10 minutes, 15 minutes, 20 minutes, 25 minutes, 30 minutes, 35 minutes, 40 minutes, 45 minutes, 50 minutes, 1 hour, or a loner time period, or a subrange thereof, for any or each of the step(s) of any of the methods provided herein. In some embodiments, the microwave energy is applied to the polyp eptides prior to or after any or each of the steps(s) of any of the methods provided herein. In some embodiments, the microwave energy is applied for a duration of time effective to achieve modification of, binding to and/or removal of an amino acid in at least 20%, 30%, 40%, 50%, 60%, 70%, 80%, 90% or greater percentage of the polypeptides.

In some embodiments, the microwave energy is applied by a non-uniform microwave field. In some embodiments, the microwave energy is applied by a uniform microwave field, e.g., applied by microwave volumetric heating (MVH).

In some embodiments, the microwave energy is applied or delivered uniformly to a sample in a vessel. In some cases, the sample in the vessel exposed to microwave energy comprises aqueous and/or organic material.

In some embodiments, the microwave energy is applied in the presence of ionic liquids. For example, the microwave energy is applied to the mixture of the polypeptides in ionic liquids.

In some embodiments, the methods provided herein are performed in a vessel that provides a microwave energy to maintain the reaction at a fixed temperature. In some examples, the methods provided herein are performed in a vessel that provides a microwave energy to maintain the reaction at a temperature of about at least 10° C., 20° C., 30° C., 40° C., 50° C., 60° C., 70° C., 80° C., 90° C., or 100° C., or a subrange thereof. In some cases, the methods provided herein are performed in a vessel that provides a microwave energy to maintain the reaction at a temperature of about 30° C., 60° C., or 80° C., or a subrange thereof.

### V. Kits and Articles of Manufacture

Also provided herein are exemplary articles of manufacture for use with the methods provided herein. Also provided are kits containing components such as reagents, buffers, and containers for performing the methods described herein in suitable packaging. In some embodiments, provided is a kit or system for treating or preparing a polypeptide comprising a functionalizing reagent to modify an amino acid of a polypeptide, a binding agent capable of binding to said polypeptide, and/or a removing reagent to remove an amino acid from said polypeptide. In some embodiments, the kit or system also includes a microwave energy source, e.g., a microwave energy source configured for applying a microwave energy to said polypeptide. In some examples, the functionalizing reagent modifies an N-terminal amino acid (NTAA), the binding agent binds to an N-terminal amino acid (NTAA), and/or the removing reagent removes an N-terminal amino acid (NTAA). In some further embodiments, the kit or system includes a reagent or a device for determining the sequence of at least a portion of said polypeptide. In some embodiments, the kit or system is for sequencing one or more polypeptides or preparing polypeptides for sequencing.

Provided herein is a kit or system for analyzing a polypeptide. In some embodiments, the kit or system comprises (a) a recording tag configured to be associated directly or indirectly with a polypeptide; (b) a functionalizing reagent for modifying the N-terminal amino acid (NTAA) of said polypeptide to yield a functionalized NTAA, (c) a first binding agent comprising a first binding portion capable of binding to said functionalized NTAA and (c1) a first coding tag with identifying information regarding said first binding agent, or (c2) a first detectable label; and (d) a microwave energy source, e.g., a microwave energy source configured for applying a microwave energy to said polypeptide. In some embodiments, the kit or system further comprises a reagent or a device for (d1) transferring the information of the first coding tag to the recording tag to generate a first extended recording tag and/or analyzing said extended recording tag, or (d2) detecting the first detectable label.

In some embodiments, the kit comprises reagents for preparing samples, such as for preparing polypeptides from a sample and joining to a support. In some embodiments, the kits optionally include instructions for performing the reactions and applying microwave energy. In some embodiments, the kits comprise one or more of the following components: binding agent(s), solid support(s), recoding tag(s), functionalizing reagent(s), removing reagent(s), reagent(s) for transferring information, sequencing reagent(s), and/or any needed buffer(s), etc.

The reagents, buffers, and other components may be provided in vials (such as sealed vials), vessels, ampules, bottles, jars, flexible packaging (e.g., sealed Mylar or plastic bags), and the like. These articles of manufacture may further be sterilized and/or sealed.

In some embodiments, the kits or articles of manufacture may further comprise instruction(s) on the methods and uses described herein. In some embodiments, the instructions are directed to microwave-assisted methods of preparing and treating polypeptides. In some examples, the examples are directed to microwave assisted methods of treating polypeptides with functionalizing reagents, binding agents, and removing reagents. The kits described herein may also include other materials desirable from a commercial and user standpoint, including other buffers, diluents, filters, syringes, and package inserts with instructions for performing any methods described herein.

### VI. Exemplary Embodiments

Among the provided embodiments are:

1. A method for sequencing a polypeptide, which method comprises:

a) contacting a polypeptide with a functionalizing reagent to modify an amino acid of said polypeptide, a binding agent capable of binding to said polypeptide, and/or a removing reagent to remove an amino acid from said polypeptide;

b) applying a microwave energy to said polypeptide; and

c) determining the sequence of at least a portion of said polypeptide.

2. A method for treating a polypeptide, which method comprises:

a) contacting a polypeptide with a functionalizing reagent to modify an amino acid of said polypeptide, a binding agent capable of binding to said polypeptide, and/or a removing reagent to remove an amino acid from said polypeptide; and

b) applying a microwave energy to said polypeptide;

wherein the functionalizing reagent modifies an N-terminal amino acid (NTAA), the binding agent binds to an N-terminal amino acid (NTAA), and/or the removing reagent removes an N-terminal amino acid (NTAA).

3. The method of embodiment 1 or embodiment 2, wherein:
the step a) is conducted before the step b); or
the step a) is conducted after the step b).

4. The method of embodiment 1 or embodiment 2, wherein the step a) and the step b) are conducted in the same step or simultaneously.

5. The method of embodiment 4, wherein the polypeptide is contacted with the functionalizing reagent, the binding agent, and/or the removing reagent in the presence of the microwave energy.

6. The method of any one of embodiments 1-5, wherein the polypeptide is contacted with the functionalizing reagent.

7. The method of embodiment 6, wherein the polypeptide is contacted with the functionalizing reagent to modify a single amino acid of the polypeptide.

8. The method of embodiment 6, wherein the polypeptide is contacted with the functionalizing reagent to modify multiple amino acids of the polypeptide.

9. The method of any one of embodiments 1-8, which comprises:

(1) preparing a mixture comprising one or more of polypeptides and a functionalizing reagent to modify one or more amino acids of the one or more of polypeptides;

(2) subjecting the mixture to a microwave energy; and

(3) determining the sequence of at least a portion of the one or more of polypeptides.

10. The method of any one of embodiments 1-9, wherein the modified amino acid is an amino acid at a terminus of the polypeptide, e.g., an N-terminal amino acid (NTAA), or a C-terminal amino acid (CTAA).

11. The method of any one of embodiments 1-10, which comprises contacting the polypeptide with a functionalizing reagent to modify an N-terminal amino acid (NTAA) of the polypeptide and applying a microwave energy.

12. The method of any one of embodiments 1-11, which comprises:

(1) preparing a mixture comprising one or more polypeptides and a functionalizing reagent to modify an N-terminal amino acid (NTAA); and

(2) subjecting the mixture to a microwave energy.

13. The method of any one of embodiments 1-12, wherein the functionalizing reagent comprises a chemical agent, an enzyme, and/or a biological agent.

14. The method of any one of embodiments 1-13, wherein the functionalizing reagent adds a chemical moiety to an amino acid of the polypeptide.

15. The method of any one of embodiments 1-14, wherein the functionalizing reagent selectively or specifically modifies the N-terminal amino acid (NTAA) of the polypeptide.

16. The method of embodiment 14 or embodiment 15, wherein the chemical moiety is added via a chemical reaction or an enzymatic reaction.

17. The method of any one of embodiments 14-16, wherein the chemical moiety is a phenylthiocarbamoyl (PTC or derivatized PTC) moiety, a dinitrophenol (DNP) moiety, a sulfonyloxynitrophenyl (SNP) moiety, a dansyl moiety, a 7-methoxy coumarin moiety, a thioacyl moiety, a thioacetyl moiety, an acetyl moiety, a guanidinyl moiety, or a thiobenzyl moiety.

18. The method of any one of embodiments 1-17, wherein the functionalizing reagent comprises an isothiocyanate derivative, 2,4-dinitrobenzenesulfonic (DNBS), 4-sulfonyl-2-nitrofluorobenzene (SNFB) 1-fluoro-2,4-dinitrobenzene, dansyl chloride, 7-methoxycoumarin acetic acid, a thioacylation reagent, a thioacetylation reagent, and/or a thiobenzylation reagent.

19. The method of any one of embodiments 1-18, wherein the functionalizing reagent comprises a compound selected from the group consisting of:

(i) a compound of Formula (I):

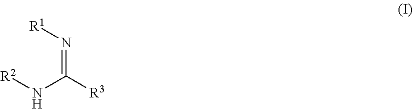

or a salt or conjugate thereof,

wherein
R¹ and R² are each independently H, C₁₋₆ alkyl, cycloalkyl, —C(O)R^{a}, —C(O)OR^{b}, or —S(O)₂R^{c};
   R^{a}, R^{b}, and R^{c} are each independently H, C₁₋₆alkyl, C₁₋₆haloalkyl, arylalkyl, aryl, or heteroaryl, wherein the C₁₋₆alkyl, C₁₋₆haloalkyl, arylalkyl, aryl, and heteroaryl are each unsubstituted or substituted;
R³ is heteroaryl, —NR^{d}C(O)OR^{e}, or —SR^{f}, wherein the heteroaryl is unsubstituted or substituted;
   R^{d}, R^{e}, and R^{f} are each independently H or C₁₋₆alkyl; and

optionally wherein when R³ is

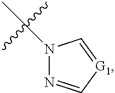

wherein Gi is N, CH, or CX where X is halo, C₁₋₃ alkyl, C₁₋₃ haloalkyl, or nitro, R¹ and R² are not both H;

(ii) a compound of Formula (II):

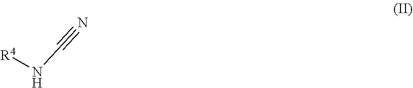

or a salt or conjugate thereof,

wherein

R⁴ is H, C₁₋₆ alkyl, cycloalkyl, —C(O)R^{g}, or —C(O)OR^{g}; and
R^{g} is H, C₁₋₆alkyl, C₂₋₆alkenyl, C₁₋₆haloalkyl, or arylalkyl, wherein the C₁₋₆alkyl, C₂₋₆alkenyl, C₁₋₆haloalkyl, and arylalkyl are each unsubstituted or substituted;

(iii) a compound of Formula (III):

R⁵—N═C═S (III)

or a salt or conjugate thereof,

wherein

R⁵ is C₁₋₆alkyl, C₂₋₆alkenyl, cycloalkyl, heterocycloalkyl, aryl or heteroaryl;
wherein the C₁₋₆alkyl, C₂₋₆alkenyl, cycloalkyl, heterocycloalkyl, aryl or heteroaryl are each unsubstituted or substituted with one or more groups selected from the group consisting of halo, —NR^{h}Rⁱ, —S(O)₂R^{j}, or heterocyclyl;
R^{h}, Rⁱ, and R^{j} are each independently H, C₁₋₆alkyl, C₁₋₆haloalkyl, arylalkyl, aryl, or heteroaryl, wherein the C₁₋₆alkyl, C₁₋₆haloalkyl, arylalkyl, aryl, and heteroaryl are each unsubstituted or substituted;

(iv) a compound of Formula (IV):

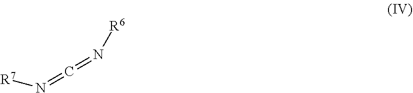

or a salt or conjugate thereof,

wherein

R⁶ and R⁷ are each independently H, C₁₋₆ alkyl, —CO₂C₁₋₄ alkyl, —OR^{k}, aryl, or cycloalkyl, wherein the C₁₋₆alkyl, —CO₂C₁₋₄ alkyl, —OR^{k}, aryl, and cycloalkyl are each unsubstituted or substituted; and

R^{k} is H, C₁₋₆alkyl, or heterocyclyl, wherein the C₁₋₆alkyl and heterocyclyl are each unsubstituted or substituted;

(v) a compound of Formula (V):

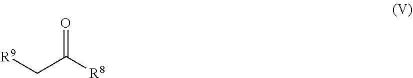

or a salt or conjugate thereof,

wherein

R⁸ is halo or —OR^{m};
R^{m} is H, C₁₋₆alkyl, or heterocyclyl; and

R⁹ is hydrogen, halo, or C₁₋₆haloalkyl;

(vi) a metal complex of Formula (VI):

MLₙ (VI)

or a salt or conjugate thereof,

wherein

M is a metal selected from the group consisting of Co, Cu, Pd, Pt, Zn, and Ni;

L is a ligand selected from the group consisting of —OH, —OH₂, 2,2′-bipyridine (bpy), 1,5dithiacyclooctane (dtco), 1,2-bis(diphenylphosphino)ethane (dppe), ethylenediamine (en), and triethylenetetramine (trien); and

n is an integer from 1-8, inclusive;

wherein each L can be the same or different; and

(vii) a compound of Formula (VII):

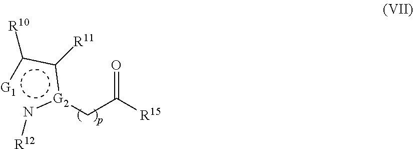

or a salt or conjugate thereof,

wherein

G¹ is N, NR¹³, or CR¹³R¹⁴;

G² is N or CH;

p is 0 or 1;

R¹⁰, R¹¹, R¹², R¹³, and R¹⁴ are each independently selected from the group consisting of H, C₁₋₆alkyl, C₁₋₆haloalkyl, C₁₋₆alkylamine, and C₁₋₆alkylhydroxylamine, wherein the C₁₋₆alkyl, C₁₋₆haloalkyl, C₁₋₆alkylamine, and C₁₋₆ alkylhydroxylamine are each unsubstituted or substituted, and R¹⁰ and R¹¹ can optionally come together to form a ring; and

R¹⁵ is H or OH.

20. The method of any one of embodiments 1-19, which comprises contacting the polypeptide with a reagent for removing the functionalized amino acid from the polypeptide to expose the immediately adjacent amino acid residue in the polypeptide.

21. The method of any one of embodiments 1-20, wherein modification of the amino acid of the polypeptide is accelerated due to the application of the microwave energy to the polypeptide.

22. The method of embodiment 21, wherein the modification of the amino acid of the polypeptide due to the application of the microwave energy to the polypeptide is accelerated by at least 5% as compared to modification of the amino acid of the polypeptide without application of the microwave energy to the polypeptide.

23. The method of any one of embodiments 1-22, wherein the polypeptide is contacted with a binding agent capable of binding to the polypeptide.

24. The method of embodiment 23, wherein the polypeptide is contacted with a single binding agent capable of binding to the polypeptide.

25. The method of embodiment 23, wherein the polypeptide is contacted with multiple binding agents capable of binding to the polypeptide.

26. The method of any one of embodiments 23-25, which comprises:

(1) preparing a mixture comprising one or more polypeptides and one or more binding agents capable of binding to at least a portion of the one or more polypeptides;

(2) subjecting the mixture to a microwave energy; and

(3) determining the sequence of at least a portion of the one or more polypeptides.

27. The method of any one of embodiments 23-26, wherein each binding agent comprises a binding moiety capable of binding to:

an internal polypeptide;

a terminal amino acid residue;

terminal di-amino-acid residues;

terminal triple-amino-acid residues;

an N-terminal amino acid (NTAA);

a C-terminal amino acid (CTAA),

a functionalized NTAA; or

a functionalized CTAA.

28. The method of any one of embodiments 23-27, which comprises contacting the polypeptide with one or more binding agents and applying a microwave energy, wherein each of the binding agents comprises a binding moiety capable of binding to a terminal amino acid residue, terminal di-amino-acid residues, or terminal triple-amino-acid residues of the polypeptide.

29. The method of any one of embodiments 23-28, which comprises:

(1) preparing a mixture comprising one or more polypeptides and one or more binding agents, wherein each of the binding agents comprises a binding moiety capable of binding to a terminal amino acid residue, terminal di-amino-acid residues, or terminal triple-amino-acid residues; and

(2) subjecting the mixture to a microwave energy.

30. The method of any one of embodiments 23-29, wherein each of the binding agents further comprises a coding tag comprising identifying information regarding the binding moiety.

31. The method of embodiment 30, wherein the binding agent and the coding tag are joined by a linker or a binding pair.

32. The method of any one of embodiments 28-31, wherein the binding agent binds to an N-terminal amino acid (NTAA), a C-terminal amino acid (CTAA) or a functionalized NTAA or CTAA of the polypeptide.

33. The method of any one of embodiments 23-32, wherein the binding agent binds to a post-translationally modified amino acid.

34. The method of any one of embodiments 23-33, wherein the binding agent is a polypeptide or a protein.

35. The method of any one of embodiments 23-34, wherein the binding agent comprises an aminopeptidase or a variant, mutant, or modified protein thereof; an aminoacyl tRNA synthetase or a variant, mutant, or modified protein thereof; an anticalin or a variant, mutant, or modified protein thereof; a ClpS (such as ClpS2) or a variant, mutant, or modified protein thereof; a UBR box protein or a variant, mutant, or modified protein thereof; or a small molecule that binds to an amino acid, i.e. vancomycin or a variant, mutant, or modified molecule thereof; or an antibody or a binding fragment thereof; or any combination thereof.

36. The method of any one of embodiments 23-35, wherein the binding agent binds to a single amino acid residue (e.g., an N-terminal amino acid residue, a C-terminal amino acid residue, or an internal amino acid residue), a dipeptide (e.g., an N-terminal dipeptide, a C-terminal dipeptide, or an internal dipeptide), a tripeptide (e.g., an N-terminal tripeptide, a C-terminal tripeptide, or an internal tripeptide), or a post-translational modification of the analyte or polypeptide.

37. The method of any one of embodiments 23-36, wherein binding between or among the binding agent and the polypeptide is accelerated due to the application of the microwave energy to the polypeptide.

38. The method of embodiment 37, wherein binding between or among the binding agent and the polypeptide due to the application of the microwave energy to the polypeptide is accelerated by at least 5% as compared to binding between or among the binding agent and the polypeptide without application of the microwave energy to the polypeptide.

39. The method of any one of embodiments 1-38, wherein the polypeptide is contacted with a removing reagent to remove an amino acid from the polypeptide.

40. The method of embodiment 39, wherein the polypeptide is contacted with the removing reagent to remove a single amino acid from the polypeptide.

41. The method of embodiment 39, wherein the polypeptide is contacted with the removing reagent to remove multiple amino acids from the polypeptide.

42. The method of any one of embodiments 39-41, which comprises:

(1) contacting the polypeptide with a reagent to remove one or more amino acids from the polypeptide and applying a microwave energy; and

(2) determining the sequence of at least a portion of the polypeptide.

43. The method of any one of embodiments 39-41, which comprises:

(1) preparing a mixture comprising one or more polypeptides and reagents for removing one or more amino acids from the one or more polypeptides;

(2) subjecting the mixture to a microwave energy; and

(3) determining the sequence of at least a portion of the one or more polypeptides.

44. The method of any one of embodiments 39-43, wherein the removed amino acid comprises:

(i) an N-terminal amino acid (NTAA);

(ii) an N-terminal dipeptide sequence;

(iii) an N-terminal tripeptide sequence;

(iv) an internal amino acid;

(v) an internal dipeptide sequence;

(vi) an internal tripeptide sequence;

(vii) a C-terminal amino acid (CTAA);

(viii) a C-terminal dipeptide sequence; or

(ix) a C-terminal tripeptide sequence,

or any combination thereof,

optionally wherein any one or more of the amino acid residues in (i)-(ix) are modified or functionalized.

45. The method of any one of embodiments 39-43, which comprises contacting the polypeptide with a reagent to remove one or more N-terminal amino acids (NTAA) from the polypeptide and applying a microwave energy.

46. The method of any one of embodiments 39-43, which comprises:

(1) preparing a mixture comprising one or more polypeptides and one or more reagents for removing one or more N-terminal amino acids (NTAA) from the one or more polypeptides; and

(2) subjecting the mixture to a microwave energy.

47. The method of any one of embodiments 39-46, wherein the removing reagent selectively or specifically removes the N-terminal amino acid (NTAA) of the polypeptide.

48. The method of any one of embodiments 39-47, wherein the removing reagent removes one amino acid.

49. The method of any one of embodiments 39-47, wherein the removing reagent removes two amino acids.

50. The method of any one of embodiments 39-49, wherein removing the one or more amino acids exposes a new N-terminal amino acid of the polypeptide.

51. The method of any one of embodiments 39-50, wherein the amino acid is removed from the polypeptide by a chemical cleavage or an enzymatic cleavage.

52. The method of any one of embodiments 39-51, wherein the removing reagent removes a functionalized amino acid residue from the polypeptide.

53. The method of embodiment 44 or embodiment 52, wherein the removing reagent comprises trifluoroacetic acid or hydrochloric acid.

54. The method of embodiment 44 or embodiment 52, wherein the removing reagent comprises an acylpeptide hydrolase (APH), a dipeptidylpeptidase (DPP) and/or a dipeptidyl aminopeptidase enzyme.

55. The method of any of one embodiments 39-52, wherein the removing reagent comprises a carboxypeptidase or an aminopeptidase or a variant, mutant, or modified protein thereof; a hydrolase or a variant, mutant, or modified protein thereof; a mild Edman degradation reagent; an Edmanase enzyme; anhydrous TFA, a base; or any combination thereof.

56. The method of embodiment 55, wherein:

the mild Edman degradation uses a dichloro or monochloro acid;

the mild Edman degradation uses TFA, TCA, or DCA; or

the mild Edman degradation uses triethylammonium acetate (Et₃NHOAc).

57. The method of any one of embodiments 39-55, wherein the reagent for removing the amino acid comprises a base.

58. The method of embodiment 57, wherein the base is a hydroxide, an alkylated amine, a cyclic amine, a carbonate buffer, a trisodium phosphate buffer, or a metal salt.

59. The method of embodiment 58, wherein:

the hydroxide is sodium hydroxide;

the alkylated amine is selected from methylamine, ethylamine, propylamine, dimethylamine, diethylamine, dipropylamine, trimethylamine, triethylamine, tripropylamine, cyclohexylamine, benzylamine, aniline, diphenylamine, N,N-Diisopropylethylamine (DIPEA), and lithium diisopropylamide (LDA);

the cyclic amine is selected from pyridine, pyrimidine, imidazole, pyrrole, indole, piperidine, prolidine, 1,8-diazabicyclo[5.4.0]undec-7-ene (DBU), and 1,5-diazabicyclo[4.3.0]non-5-ene (DBN);

the carbonate buffer comprises sodium carbonate, potassium carbonate, calcium carbonate, sodium bicarbonate, potassium bicarbonate, or calcium bicarbonate;

the metal salt comprises silver; or

the metal salt is AgClO₄.

60. The method of any one of embodiments 39-59, further comprising contacting the polypeptide with a peptide coupling reagent.

61. The method of embodiment 60, wherein the peptide coupling reagent is a carbodiimide compound.

62. The method of embodiment 61, wherein the carbodiimide compound is diisopropylcarbodiimide (DIC) or 1-ethyl-3-(3-dimethylaminopropyl)carbodiimide (EDC).

63. The method of any one of embodiments 39-62, wherein the removed amino acid is an amino acid modified using the method of any one of embodiments 1-22.

64. The method of any one of embodiments 39-63, wherein removal of an amino acid from the polypeptide is accelerated due to the application of the microwave energy to the polypeptide.

65. The method of embodiment 64, wherein removal of an amino acid from the polypeptide due to the application of the microwave energy to the polypeptide is accelerated by at least 5% as compared to removal of an amino acid from the polypeptide without application of the microwave energy to the polypeptide.

66. The method of any one of embodiments 1-65, wherein the sequence of at least a portion of the polypeptide is determined by Edman degradation.

67. The method of any one of embodiments 1-66, which comprises:

(a) modifying the N-terminal amino acid (NTAA) of a polypeptide with a functionalizing reagent; and

(b) contacting the polypeptide with a reagent to remove the modified NTAA;

wherein step (a) and/or step (b) are performed in the presence of a microwave energy.

68. The method of embodiment 67, further comprising:

(a1) contacting the polypeptide with a binding agent that binds to the modified NTAA, optionally in the presence of the microwave energy.

69. The method of embodiment 67 or embodiment 68, which further comprises:

(c) determining the sequence of at least a portion of the polypeptide.

70. The method of any one of embodiments 1-69, which comprises:

(a) contacting a plurality of polypeptides with a functionalizing reagent to modify an amino acid of each of the polypeptides;

(b) contacting the polypeptides with a removing reagent to remove the modified amino acids; and

(c) determining the sequence of at least a portion of each of the polypeptides; wherein step (a) and/or step (b) are performed in the presence of a microwave energy.

71. The method of embodiment 70, which further comprises:

(a1) contacting the polypeptides with a binding agent, optionally in the presence of a microwave energy.

72. The method of embodiment 70 or embodiment 71, wherein at least one of the modified and removed amino acids is an N-terminal amino acid (NTAA) or a C-terminal amino acid (CTAA) of the polypeptide.

73. The method of any one of embodiments 67-72, wherein:

step (a) and step (b) are performed sequentially;

step (a), (a1), and step (b) are performed sequentially;

step (a), (a1), step (b) and step (c) are performed sequentially;

step (a) is performed before step (a1);

step (a) is performed before step (b);

step (a1) is performed before step (b);

step (a) is performed before step (c);

step (a1) is performed before step (c);

step (a) and step (b) are repeated;

step (a), (a1), and step (b) are repeated; or

step (b) is performed before step (c).

74. A method for analyzing a polypeptide, which comprises the steps:

(a) providing a polypeptide optionally associated directly or indirectly with a recording tag;

(b) functionalizing the N-terminal amino acid (NTAA) of said polypeptide with a functionalizing reagent to yield a functionalized NTAA,

(c) contacting said polypeptide with a first binding agent comprising a first binding portion capable of binding to said functionalized NTAA and
(c1) a first coding tag with identifying information regarding said first binding agent, or
(c2) a first detectable label;

(d) (d1) transferring the information of said first coding tag to said recording tag to generate a first extended recording tag and analyzing said extended recording tag, or
(d2) detecting said first detectable label, and

wherein:

said polypeptide is contacted with a microwave energy before any of said steps (b), (c), (d1) and (d2), or

any one or more of steps (b), (c), (d1), and/or (d2) are performed in the presence of a microwave energy.

75. The method of embodiment 74, which further comprises contacting the polypeptide with a proline aminopeptidase under conditions suitable to cleave an N-terminal proline before step (b).

76. The method of embodiment 74 or 75, which further comprises:

(e) contacting the polypeptide with a removing reagent to remove the functionalized NTAA to expose a new NTAA.

77. The method of embodiment 76, which further comprises, between steps (d) and (e), repeating steps (b) to (d) to determine the sequence of at least a portion of the polypeptide.

78. The method of any one of embodiments 74-77, wherein the binding agent binds to the N-terminal amino acid residue of the polypeptide and the N-terminal amino acid residue is removed after each binding cycle.

79. The method of embodiment 78, wherein the N-terminal amino acid residue is removed via Edman degradation.

80. The method of any one of embodiments 74-79, wherein the functionalizing reagent comprises a chemical agent, an enzyme, and/or a biological agent.

81. The method of any one of embodiments 74-80, wherein the functionalizing reagent adds a chemical moiety to the amino acid.

82. The method of any one of embodiments 74-81, wherein the functionalizing reagent selectively or specifically modifies the N-terminal amino acid (NTAA) of the polypeptide.

83. The method of embodiment 81 or embodiment 82, wherein the chemical moiety is added via a chemical reaction or an enzymatic reaction.

84. The method of any one of embodiments 81-83, wherein the chemical moiety is a phenylthiocarbamoyl (PTC or derivatized PTC), a dinitrophenol (DNP) moiety; a sulfonyloxynitrophenyl (SNP) moiety, a dansyl moiety; a 7-methoxy coumarin moiety; a thioacyl moiety; a thioacetyl moiety; an acetyl moiety; a guanidinyl moiety; or a thiobenzyl moiety.

85. The method of any one of embodiments 74-84, wherein the functionalizing reagent comprises an isothiocyanate derivative, 2,4-dinitrobenzenesulfonic (DNBS), 4-sulfonyl-2-nitrofluorobenzene (SNFB) 1-fluoro-2,4-dinitrobenzene, dansyl chloride, 7-methoxycoumarin acetic acid, a thioacylation reagent, a thioacetylation reagent, and/or a thiobenzylation reagent.

86. The method of any one of embodiments 74-85, wherein the functionalizing reagent comprises a compound selected from the group consisting of:

(i) a compound of Formula (I):

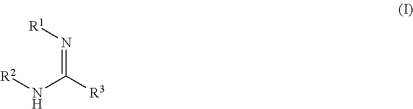

or a salt or conjugate thereof,

wherein
R¹ and R² are each independently H, C₁₋₆ alkyl, cycloalkyl, —C(O)R^{a}, —C(O)OR^{b}, or —S(O)₂R^{c};
   R^{a}, R^{b}, and R^{c} are each independently H, C₁₋₆alkyl, C₁₋₆haloalkyl, arylalkyl, aryl, or heteroaryl, wherein the C₁₋₆alkyl, C₁₋₆haloalkyl, arylalkyl, aryl, and heteroaryl are each unsubstituted or substituted;

R³ is heteroaryl, —NR^{d}C(O)OR^{e}, or —SR^{f}, wherein the heteroaryl is unsubstituted or substituted;
R^{d}, R^{e}, and R^{f} are each independently H or C₁₋₆alkyl; and optionally wherein when R³ is

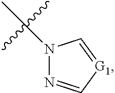

wherein G₁ is N, CH, or CX where X is halo, C₁₋₃ alkyl, C₁₋₃ haloalkyl, or nitro, R¹ and R² are not both H;

(ii) a compound of Formula (II):

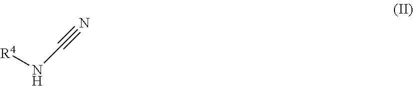

or a salt or conjugate thereof,

wherein

R⁴ is H, C₁₋₆ alkyl, cycloalkyl, —C(O)R^{g}, or —C(O)OR^{g}; and
R^{g} is H, C₁₋₆alkyl, C₂₋₆alkenyl, C₁₋₆haloalkyl, or arylalkyl, wherein the C₁₋₆alkyl, C₂₋₆alkenyl, C₁₋₆haloalkyl, and arylalkyl are each unsubstituted or substituted;

(iii) a compound of Formula (III):

R⁵—N═C═S (III)

or a salt or conjugate thereof,

wherein

R⁵ is C₁₋₆alkyl, C₂₋₆alkenyl, cycloalkyl, heterocycloalkyl, aryl or heteroaryl;
wherein the C₁₋₆alkyl, C₂₋₆alkenyl, cycloalkyl, heterocycloalkyl, aryl or heteroaryl are each unsubstituted or substituted with one or more groups selected from the group consisting of halo, —NR^{h}Rⁱ, —S(O)₂R^{j}, or heterocyclyl;
R^{h}, Rⁱ, and R^{j} are each independently H, C₁₋₆alkyl, C₁₋₆haloalkyl, arylalkyl, aryl, or heteroaryl, wherein the C₁₋₆alkyl, C₁₋₆haloalkyl, arylalkyl, aryl, and heteroaryl are each unsubstituted or substituted;

(iv) a compound of Formula (IV):

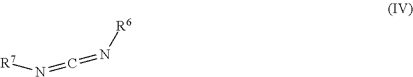

or a salt or conjugate thereof,

wherein

R⁶ and R⁷ are each independently H, C₁₋₆alkyl, —CO₂C₁₋₄alkyl, —OR^{k}, aryl, or cycloalkyl, wherein the C₁₋₆alkyl, —CO₂C₁₋₄ alkyl, —OR^{k}, aryl, and cycloalkyl are each unsubstituted or substituted; and

R^{k} is H, C₁₋₆alkyl, or heterocyclyl, wherein the C₁₋₆alkyl and heterocyclyl are each unsubstituted or substituted;

(v) a compound of Formula (V):

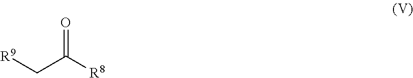

or a salt or conjugate thereof,

wherein

R⁸ is halo or —OR^{m};
R^{m} is H, C₁₋₆alkyl, or heterocyclyl; and

R⁹ is hydrogen, halo, or C₁₋₆haloalkyl;

(vi) a metal complex of Formula (VI):

MLₙ(VI)

or a salt or conjugate thereof,

wherein

M is a metal selected from the group consisting of Co, Cu, Pd, Pt, Zn, and Ni;

L is a ligand selected from the group consisting of —OH, —OH₂, 2,2′-bipyridine (bpy), 1,5dithiacyclooctane (dtco), 1,2-bis(diphenylphosphino)ethane (dppe), ethylenediamine (en), and triethylenetetramine (trien); and

n is an integer from 1-8, inclusive;

wherein each L can be the same or different; and

(vii) a compound of Formula (VII):

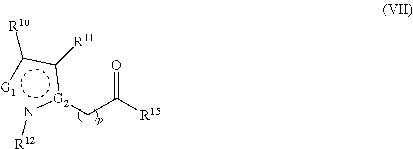

or a salt or conjugate thereof,

wherein

G¹ is N, NR¹³, or CR¹³R¹⁴;

G² is N or CH;

p is 0 or 1;

R¹⁰, R¹¹, R¹², R¹³, and R¹⁴ are each independently selected from the group consisting of H, C₁₋₆alkyl, C₁₋₆ haloalkyl, C₁₋₆alkylamine, and C₁₋₆alkylhydroxylamine, wherein the C₁₋₆alkyl, C₁₋₆haloalkyl, C₁₋₆alkylamine, and C₁₋₆ alkylhydroxylamine are each unsubstituted or substituted, and R¹⁰ and R¹¹ can optionally come together to form a ring; and

R¹⁵ is H or OH.

87. The method of any one of embodiments 68-86, wherein the binding agents each further comprises a coding polymer comprising identifying information regarding the first binding moiety.

88. The method of embodiment 87, wherein the binding agent and the coding tag are joined by a linker or a binding pair.

89. The method of any one of embodiments 68-88, wherein the binding agent binds to an N-terminal amino acid (NTAA), a C-terminal amino acid (CTAA) or a functionalized NTAA or CTAA of the polypeptide.

90. The method of any one of embodiments 68-88, wherein the binding agent binds to a post-translationally modified amino acid.

91. The method of any one of embodiments 68-90, wherein the binding agent is a polypeptide or a protein.

92. The method of any one of embodiments 68-90, wherein the binding agent comprises an aminopeptidase or a variant, mutant, or modified protein thereof; an aminoacyl tRNA synthetase or a variant, mutant, or modified protein thereof; an anticalin or a variant, mutant, or modified protein thereof; a ClpS (such as ClpS2) or a variant, mutant, or modified protein thereof; a UBR box protein or a variant, mutant, or modified protein thereof; or a small molecule that binds to an amino acid, i.e. vancomycin or a variant, mutant, or modified molecule thereof; or an antibody or a derivative or binding fragment thereof; or any combination thereof.

93. The method of any one of embodiments 68-92, wherein the binding agent binds to a single amino acid residue (e.g., an N-terminal amino acid residue, a C-terminal amino acid residue, or an internal amino acid residue), a dipeptide (e.g., an N-terminal dipeptide, a C-terminal dipeptide, or an internal dipeptide), a tripeptide (e.g., an N-terminal tripeptide, a C-terminal tripeptide, or an internal tripeptide), or a post-translational modification of the analyte or polypeptide.

94. The method of any one of embodiments 67-93, further comprising determining the sequence of at least a portion of the polypeptide.

95. The method of any one of embodiments 66-94, wherein the removing reagent selectively removes the N-terminal amino acid (NTAA) of the polypeptide.

96. The method of any one of embodiments 66-95, wherein the removing reagent removes one amino acid.

97. The method of any one of embodiments 66-95, wherein the removing reagent removes two amino acids.

98. The method of any one of embodiments 66-97, wherein removing the one or more amino acid(s) exposes a new N-terminal amino acid of the polypeptide.

99. The method of any one of embodiments 66-98, wherein the amino acid is removed from the polypeptide by a chemical cleavage or an enzymatic cleavage.

100. The method of any one of embodiments 66-99, wherein the removing reagent is for removing a functionalized amino acid residue from the polypeptide.

101. The method of embodiment 100, wherein the removing reagent for removing the functionalized amino acid residue comprises trifluoroacetic acid or hydrochloric acid.

102. The method of embodiment 100, wherein the removing reagent for removing the functionalized NTAA comprises an acylpeptide hydrolase (APH), a dipeptidylpeptidase (DPP), and/or a dipeptidyl aminopeptidase enzyme.

103. The method of any one of embodiments 66-102, wherein the removing reagent to remove an amino acid comprises a carboxypeptidase or an aminopeptidase or a variant, mutant, or modified protein thereof; a hydrolase or a variant, mutant, or modified protein thereof; a mild Edman degradation reagent; an Edmanase enzyme; anhydrous TFA, a base; or any combination thereof.

104. The method of embodiment 103, wherein:

the mild Edman degradation uses a dichloro or monochloro acid;

the mild Edman degradation uses TFA, TCA, or DCA; or

the mild Edman degradation uses triethylammonium acetate (Et₃NHOAc).

105. The method of any one of embodiments 66-104, wherein the removing reagent for removing the amino acid(s) comprises a base.

106. The method of embodiment 105, wherein the base is a hydroxide, an alkylated amine, a cyclic amine, a carbonate buffer, a trisodium phosphate buffer, or a metal salt.

107. The method of embodiment 106, wherein:

the hydroxide is sodium hydroxide;

the alkylated amine is selected from methylamine, ethylamine, propylamine, dimethylamine, diethylamine, dipropylamine, trimethylamine, triethylamine, tripropylamine, cyclohexylamine, benzylamine, aniline, diphenylamine, N,N-Diisopropylethylamine (DIPEA), and lithium diisopropylamide (LDA);

the cyclic amine is selected from pyridine, pyrimidine, imidazole, pyrrole, indole, piperidine, prolidine, 1,8-diazabicyclo[5.4.0]undec-7-ene (DBU), and 1,5-diazabicyclo[4.3.0]non-5-ene (DBN);

the carbonate buffer comprises sodium carbonate, potassium carbonate, calcium carbonate, sodium bicarbonate, potassium bicarbonate, or calcium bicarbonate; or

the metal salt comprises silver; or

the metal salt is AgClO₄.

108. The method of any one of embodiments 66-107, further comprising contacting the polypeptide with a peptide coupling reagent.

109. The method of embodiment 108, wherein the peptide coupling reagent is a carbodiimide compound.

110. The method of embodiment 109, wherein the carbodiimide compound is diisopropylcarbodiimide (DIC) or 1-ethyl-3-(3-dimethylaminopropyl)carbodiimide (EDC).

111. The method of any one of embodiments 1-110, wherein the microwave energy has a wavelength from about one meter to about one millimeter, e.g., a wavelength from about 0.3 m to about 3 mm.

112. The method of any one of embodiments 1-111, wherein the microwave energy has a frequency from about 300 MHz (1 m) to about 300 GHz (1 mm).

113. The method of embodiment 112, wherein the microwave energy has a frequency from about 1 GHz to about 100 GHz.

114. The method of embodiment 112, wherein the microwave energy has a frequency with an IEEE radar band designation of S, C, X, Kᵤ, K or Kₐ band.

115. The method of any one of embodiments 1-114, wherein the microwave energy has a photon energy (eV) from about 1.24 μeV to about 1.24 meV.

116. The method of any one of embodiments 1-115, wherein the microwave energy is applied at about 5 watts, about 10 watts, about 15 watts, about 20 watts, about 25 watts, about 30 watts, about 35 watts, about 40 watts, about 45 watts, about 50 watts, about 60 watts, about 70 watts, about 80 watts, about 90 watts, about 100 watts, about 110 watts, about 120 watts, about 130 watts, about 140 watts, about 150 watts or higher watts.

117. The method of any one of embodiments 1-116, wherein the microwave energy is applied for a time period of about 1 minute, 2 minutes, 3 minutes, 4 minutes, 5 minutes, 10 minutes, 15 minutes, 20 minutes, 25 minutes, 30 minutes, 35 minutes, 40 minutes, 45 minutes, 50 minutes, 1 hour, or a loner time period for any or each of the step(s).

118. The method of any one of embodiments 1-117, wherein the microwave energy is applied for a duration of time effective to achieve modification of, binding to and/or removal of an amino acid in at least 20%, 30%, 40%, 50%, 60%, 70%, 80%, 90% or greater percentage of the polypeptides.

119. The method of any one of embodiments 1-118, wherein the microwave energy is applied by a non-uniform microwave field.

120. The method of any one of embodiments 1-118, wherein the microwave energy is applied by a uniform microwave field, e.g., applied by microwave volumetric heating (MVII).

121. The method of any one of embodiments 1-120, wherein the microwave energy is applied in the presence of ionic liquids.

122. The method of any one of embodiments 1-121, further comprising monitoring and/or controlling the temperature at which any or all step(s) of the method is or are conducted.

123. The method of any one of embodiments 1-122, which further comprises applying cooling.

124. The method of any one of embodiments 1-122, which further comprises applying active cooling.

125. The method of any one of embodiments 1-124, which is performed in a vessel.

126. The method of any one of embodiments 1-125, which is performed in a cavity in communication with a microwave radiation source.

127. The method of any one of embodiments 1-126, which is performed in a microwave chamber.

128. The method of any one of embodiments 1-127, wherein the polypeptide is directly or indirectly joined to a support.

129. The method of embodiment 128, wherein the polypeptide is joined to the support via a linker.

130. The method of embodiment 128 or embodiment 129, wherein the polypeptide is joined to the support at the N-terminal end of the polypeptide.

131. The method of embodiment 128 or embodiment 129, wherein the polypeptide is joined to the support at the C-terminal end of the polypeptide.

132. The method of embodiment 128 or embodiment 129, wherein the polypeptide is joined to the support via a side chain of the polypeptide.

133. The method of any one of embodiments 1-132, wherein the polypeptide is joined to a recording tag.

134. The method of embodiment 133, wherein the recording tag is a sequenceable polymer.

135. The method of embodiment 133 or embodiment 134, wherein the recording tag comprises a polynucleotide or a non-nucleic acid sequenceable polymer.

136. The method of any one of embodiments 133-135, wherein the polypeptide and associated recording tag are covalently immobilized to the support (e.g., via a linker), or non-covalently immobilized to the support (e.g., via a binding pair).

137. The method of any one of embodiments 133-136, wherein the polypeptide and associated recording tag are directly or indirectly attached to an immobilizing linker.

138. The method of embodiment 137, wherein the immobilizing linker is immobilized directly or indirectly to the support, thereby immobilizing the at least one polypeptide and/or its associated recording tag to the support.

139. The method of any one of embodiments 128-138, wherein the support comprises a bead, a porous bead, a porous matrix, an array, a glass surface, a silicon surface, a plastic surface, a filter, a membrane, a nylon, a silicon wafer chip, a flow through chip, a biochip including signal transducing electronic, a microtitre well, an ELISA plate, a spinning interferometry disc, a nitrocellulose membrane, a nitrocellulose-based polymer surface, a nanoparticle, or a microsphere.

140. The method of any one of embodiments 128-139, wherein the support comprises a polystyrene bead, a polymer bead, an agarose bead, an acrylamide bead, a solid core bead, a porous bead, a paramagnetic bead, glass bead, or a controlled pore bead.

141. The method of any one of embodiments 133-140, further comprising analyzing the recording tag, e.g., using nucleic acid sequence analysis.

142. The method of embodiment 141, wherein the nucleic acid sequence analysis comprises sequencing by synthesis, sequencing by ligation, sequencing by hybridization, polony sequencing, ion semiconductor sequencing, pyrosequencing, single molecule real-time sequencing, nanopore-based sequencing, or direct imaging of DNA using advanced microscopy, or any combination thereof.

143. The method of any one of embodiments 1-142, which comprises contacting a polypeptide with a functionalizing reagent to modify an amino acid of the polypeptide, a binding agent capable of binding to the polypeptide, and a removing reagent to remove an amino acid from the polypeptide.

144. The method of embodiment 143, wherein modification of the amino acid of the polypeptide, binding between or among the binding agent and the polypeptide and/or removal of an amino acid from the polypeptide is accelerated due to the application of the microwave energy to the polypeptide.

145. The method of any one of embodiments 1-144, wherein a time required for conducting any or all steps of the method is shortened due to the application of the microwave energy to the polypeptide.

146. The method of embodiment 145, wherein a time required for conducting any or all steps of the method due to the application of the microwave energy to the polypeptide is shortened by at least 5% as compared to a time required for conducting any or all steps of the method without application of the microwave energy to the polypeptide.

147. The method of any one of embodiments 1-146, wherein the level or percentage of modification of the amino acid of the polypeptide, binding between or among the binding agent and the polypeptide and/or removal of an amino acid from the polypeptide is enhanced or increased due to the application of the microwave energy to the polypeptide.

148. The method of embodiment 147, wherein the level or percentage of modification of the amino acid of the polypeptide, binding between or among the binding agent and the polypeptide and/or removal of an amino acid from the polypeptide due to the application of the microwave energy to the polypeptide is enhanced or increased by at least 5% as compared to the level or percentage of modification of the amino acid of the polypeptide, binding between or among the binding agent and the polypeptide and/or removal of an amino acid from the polypeptide without application of the microwave energy to the polypeptide.

149. The method of any one of embodiments 1-148, wherein bias of functionalization and/or removal of different amino acids is reduced or eliminated due to the application of the microwave energy to the polypeptide.

150. The method of embodiment 149, wherein the bias of functionalization and/or removal between hydrophobic amino acids and non-hydrophobic amino acids is reduced or eliminated due to the application of the microwave energy to the polypeptide.

151. The method of embodiment 149 or 150, wherein the bias of functionalization and/or removal of different amino acids due to the application of the microwave energy to the polypeptide is reduced by at least 5% as compared to the bias of functionalization and/or removal of different amino acids without application of the microwave energy to the polypeptide.

152. A kit or system for sequencing a polypeptide, which comprises:

a) a functionalizing reagent to modify an amino acid of a polypeptide, a binding agent capable of binding to said polypeptide, and/or a removing reagent to remove an amino acid from said polypeptide;

b) a microwave energy source, e.g., a microwave energy source configured for applying a microwave energy to said polypeptide; and

c) a reagent or a device for determining the sequence of at least a portion of said polypeptide.

153. A kit or system for treating a polypeptide, which comprises:

a) a functionalizing reagent to modify an amino acid of a polypeptide, a binding agent capable of binding to said polypeptide, and/or a removing reagent to remove an amino acid from said polypeptide; and

b) a microwave energy source, e.g., a microwave energy source configured for applying a microwave energy to said polypeptide;

wherein the functionalizing reagent modifies an N-terminal amino acid (NTAA), the binding agent binds to an N-terminal amino acid (NTAA), and/or the removing reagent removes an N-terminal amino acid (NTAA).

154. A kit or system for analyzing a polypeptide, which comprises:

(a) a recording tag configured to be associated directly or indirectly with a polypeptide;

(b) a functionalizing reagent for modifying the N-terminal amino acid (NTAA) of said polypeptide to yield a functionalized NTAA,

(c) a first binding agent comprising a first binding portion capable of binding to said functionalized NTAA and
(c1) a first coding tag with identifying information regarding said first binding agent, or
(c2) a first detectable label; and

(d) a microwave energy source, e.g., a microwave energy source configured for applying a microwave energy to said polypeptide.

155. The kit or system of embodiment 154, which further comprises a reagent or a device for (d 1) transferring the information of the first coding tag to the recording tag to generate a first extended recording tag and/or analyzing said extended recording tag, or

(d2) detecting the first detectable label.

### VII. Examples

The following examples are offered to illustrate but not to limit the methods, compositions, and uses provided herein.

### Example 1: Assessment of Microwave-Assisted Reactions with Polypeptides

This example describes the assessment of reactions performed with polypeptides in the presence and absence of microwave energy, including functionalization of the N-terminal amino acid (NTAA) of peptides and removal (e.g. elimination) of said functionalized NTAA.

Functionalization and elimination of the NTAA was performed on polypeptides with sequences AALAY, YFAGVAMG, FWAALAWK, and FFAALAWK (SEQ ID NO: 14-17. The polypeptides were prepared and treated in solution as follows. To a microwave vessel equipped with a magnetic stir bar, 0.2M N-ethylmorpholinium acetate (NEMA; pH 8.0) was added. A peptide solution was then added to the vessel, bringing the concentration of peptide within the vessel to 0.75 mM, followed by an aliquot of a functionalization reagent (e.g., guanidinylating reagents) in dimethylsulfoxide (DMSO), bringing the final concentration of reagent to 7.5 mM (10:1 reagent-to-peptide). Various pyrazole carboxamidine (PCA) derivatives as guanidinylating reagents were tested (PCA 1-5). Reactions with 1,2,4-triazole carboxamidine (TCA) was also tested for microwave-assisted functionalization at various wattages. The vessel was sealed, placed in a microwave synthesizer (Discover SP, CEM Corporation, USA), and set to react at 30 W for up to 5 minutes. In some cases, the reactions were allowed to react at a fixed temperature (e.g., 60° C.). The reaction was quenched by the addition of an aliquot of a 1.0 M glycine or ethanolamine solution paired with additional microwave irradiation 30 W (60° C.) for up to 5 minutes. For comparison, functionalization was performed essentially as described except in the absence of microwave energy with application of conventional thermal heating at 60° C. for various times.

For elimination of the functionalized NTAA, a volume of 2.0M sodium hydroxide solution (NaOH; pH 13.7) or 0.4M sodium carbonate/sodium bicarbonate buffer (CBc; pH 10.5) was added to the solution to bring the final concentration to 0.5M NaOH (pH 13.7) or 0.1M CBc (pH 10.5). The vessel was then placed back into the microwave cavity and reacted 30 W (95° C.) for up to 10 minutes. Once cooled, the solution was acidified to pH 5.0 using 5.0M acetic acid (AcOH). For comparison, elimination was performed essentially as described except in the absence of microwave energy with application of conventional thermal heating at 60° C. or 80° C. Sample preparation for analysis was achieved by removal of salts by use of reversed-phase C18, solid-phase extraction (SPE). The desalted peptide was then eluted using 80% acetonitrile (ACN).

For analysis, a portion of the eluted material was injected into an LCMS (grad. 5-95% B/12 min; A: water and 0.1% formic acid, B: acetonitrile and 0.1% formic acid; column Agilent InfiniteLab Poroshell 120 EC-C18 3.0×150 mm, 2.7 μm) and monitored by UV (wavelength 216 nm).

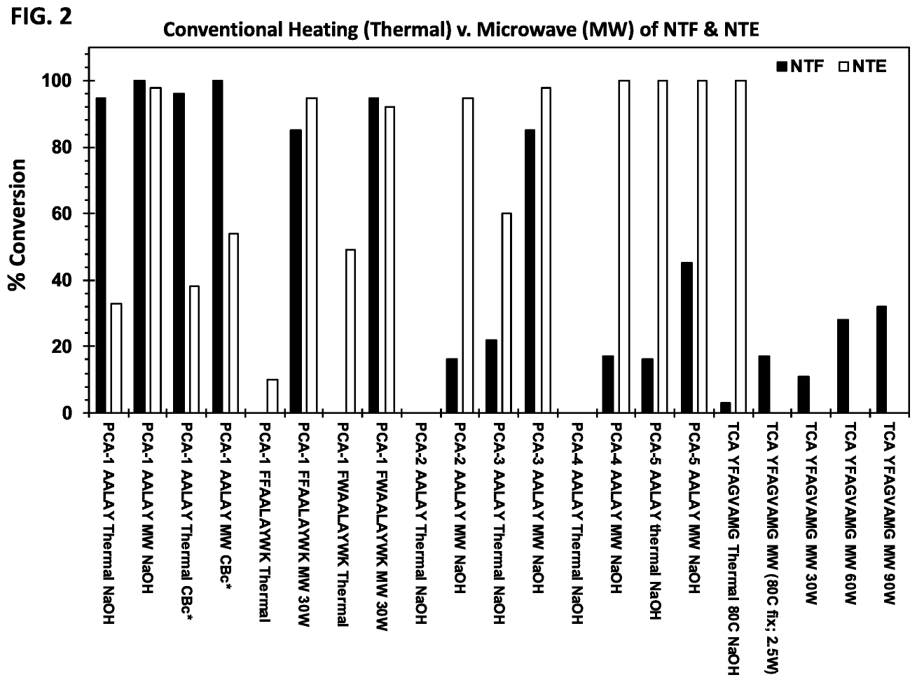

In FIG. 2, the dark bars show the results of NTAA functionalization in the presence of microwave energy (MW) compared to conventional heating applied (thermal) and the light bars show the results of NTAA elimination in the presence of microwave energy (MW) compared to conventional heating applied (thermal). In summary, application of microwave energy resulted in similar or increased functionalization and elimination of the NTAA of the tested exemplary polypeptides. In some aspects, the data supports the conclusion that application of microwave energy reduced bias of functionalization and removal of different amino acids. For example, in some cases, hydrophobic residues may exhibit elimination bias or show decreased removal compared to other residues when reactions are performed in the absence of microwave energy. In some cases, application of microwave energy eliminated this bias and removed hydrophobic and non-hydrophobic residues similarly.

### Example 2: Peptide Sequencing Assays Involving Microwave-Assisted Reactions

This Example describes the application of microwave irradiation for reactions for peptide sequencing using a ProteoCode NGPS assay, including N-terminal amino acid functionalization (NTF) and N-Terminal amino acid removal (e.g., elimination) (NTE). For the sequencing assay, microwave-assisted reactions were carried out as described in Example 1 except that peptides were bound to a substrate.

Peptides labelled with a DNA recording tag were immobilized on a substrate. Exemplary peptides tested in the assay included peptides with an amino AF-terminal peptide (AF-peptide, AFAGVAMPGAEDDVVGSGSK set forth in SEQ ID NO: 1); peptides with an amino AA-terminal peptide (AA-peptide, AAGVAMPGAEDDVVGSGSK set forth in SEQ ID NO: 2), and peptides with an amino FA-terminal peptide (FA-peptide, FAG VAMPGAEDDVVGSGSK set forth in SEQ ID NO: 3). Recording tags without a peptide attached were also tested as a control. Each peptide was individually attached to a recording tag oligonucleotide as set forth in SEQ ID NOs: 4-7. In some cases, the recording tag oligonucleotides included 5′ or other modifications as shown in Table 1.

TABLE 1
Peptide Based and DNA Based Assay Sequences

| Description | Sequence (5′-3′) and modifications | SEQ ID NO: |
|---|---|---|
| Barcode | /5AmMC6/TTT/i5OctdU/TTTTTTUCGTAGTCCGCGACACTAGNN | 4 |
| | NNNNNNNNTTAAGTCGACTGAGTG | |
| Barcode | /5AmMC6/TTT/i5OctdU/TTTTTTUCGTAGTCCGCGACACTAGNN | 5 |
| | NNNNNNNNGTTAATGGACTGAGTG | |
| Barcode | /5AmMC6/TTT/i5OctdU/TTTTTTUCGTAGTCCGCGACACTAGNN | 6 |
| | NNNNNNNNCAGTACCGACTGAGTG | |
| Barcode | /5AmMC6/TTT/i5OctdU/TTTTTTUCGTAGTCCGCGACACTAGNN | 7 |
| | NNNNNNNNGTTGGTTAACTGAGTG | |
| Coding Tag | /5AmMC6//iSP18/CACTCAGTTTTTCCTGTCACTCAGT/3SpC3/ | 8 |
| Coding Tag | /5AmMC6//iSP18/CACTCAGTCAGACTATTCACTCAGT/3SpC3/ | 9 |

/5AmMC6/ = 5′ amino modification
/i5OctdU/ = 5′-Octadiynyl dU
/3SpC3/ = 3′ C3 (three carbon) spacer
/iSP18/ = 18-atom hexa-ethyleneglycol spacer

An exemplary binding agent that binds phenylalanine when it is the N-terminal amino acid residue (F-binder) was conjugated with coding tags set forth in SEQ ID NO: 8 or 9. In some cases, the coding tag oligonucleotides included 5′, 3′, or other modifications as shown in Table 1.

For the assay, two cycles of F binding and encoding were performed, pre-NTF/NTE chemistry and post-NTF/NTE chemistry. After 1st cycle F-binder binding/encoding assay, the assay beads were subjected to treatment with NTF/NTE reagents to remove the NTAA. A pyrazole carboxamidine derivative from Example 1 (PCA-1) was used as the guanidinylating reagent for functionalization of the NTAA. For NTF treatment, the assay beads were incubated with 500 μL of 15 mM PCA-1 in 0.18 M NEMA, 10% DMSO, pH 8, 0.005% Tween 80 at 60° C. for 1 hour. For microwave-assisted NTF treatment, the assay beads were incubated with 500 μL of 15 mM PCA-1 in 0.18 M NEMA, 10% DMSO, pH 8, 0.005% Tween 80 at 60° C. for 5 minutes. The beads were washed 3× with 1 ml of 0.18 M NEMA, 10% DMSO, pH 8, 0.005% Tween 80. The NTE treatment was performed by incubating the assay beads with 500 μL of 0.1 M carbonate/sodium bicarbonate buffer (CBc; pH 10.5) containing 0.005% Tween 80 at 80° C. for 1 hour. For microwave-assisted NTE treatment, assay beads were incubated with 500 μL of 0.1 M CBc, pH 10.5 containing 0.005% Tween 80 at 30 W for 5 minutes. The beads were washed with 1 ml of PBST containing 10% formamide and used for 2nd cycle F-binder binding assay with F-binder-coding tag. The F-binder was conjugated with different cycle-specific barcode coding tags for the pre-chemistry vs. post-chemistry binding/encoding cycle. The two-cycle binding/encoding assay was performed twice.

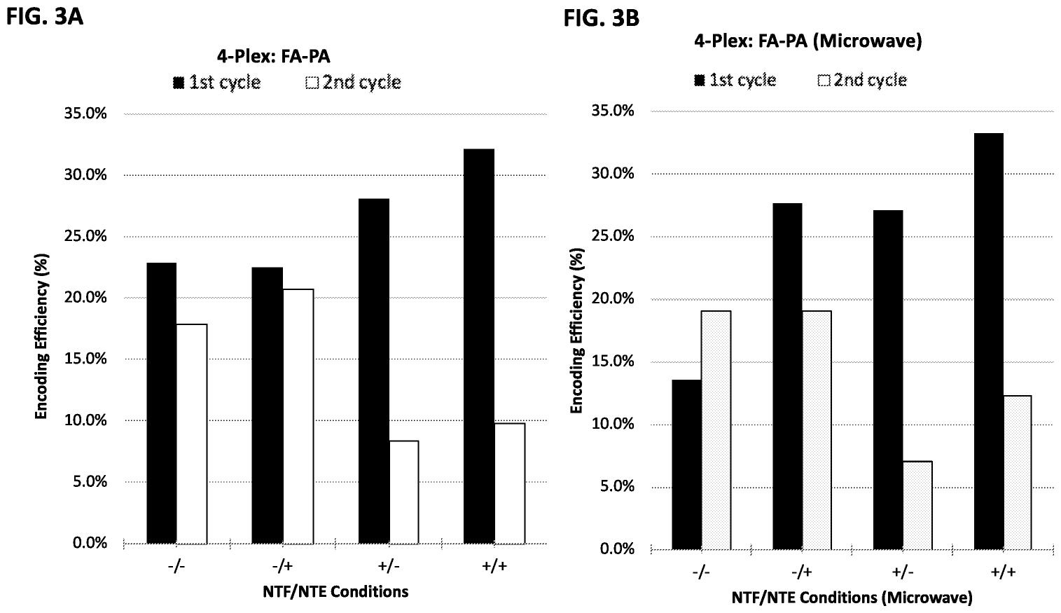

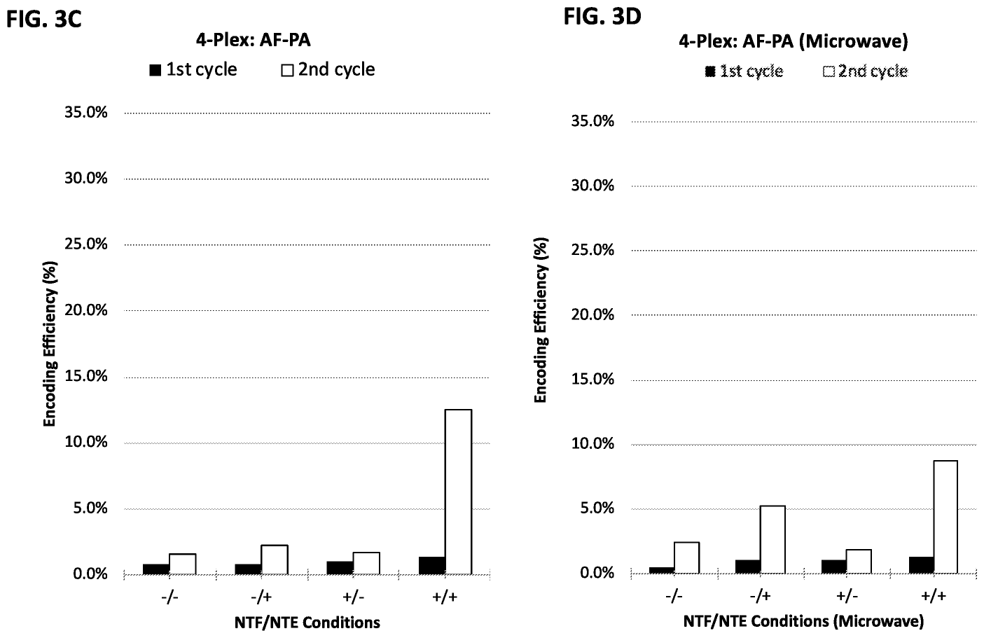

The extended recording tag of the assay was subjected to PCR amplification and analyzed by next-generation sequencing (NGS). In FIG. 3A-3D, the dark bars indicate results from the 1^{st} cycle of binding and encoding and the white bars indicate results from the 2^{nd} cycle of binding and encoding. On the x-axis of FIG. 3A-3D, the presence or absence of functionalization (NTF) and elimination (NTE) steps is indicated. The NGS results indicate that the F-binder detected the FA peptide in the 1st cycle but minimally the AF peptide (FIG. 3A-3D). It was also observed that F-binder detected the AF peptide in the 2nd cycle after NTF/NTE treatment for removal of the A residue which exposes F residue (FIGS. 3C and 3D).

In summary, an increase in F-binder encoding after functionalization (NTF) and elimination (NTE) detected on AF peptide-recording tag demonstrates single cycle peptide sequencing using DNA encoding (FIGS. 3C and 3D). The results of a decrease in F-binder encoding on FA peptide after functionalization (NTF) and elimination (NTE) demonstrates loss of signal as expected when the F peptide is effectively removed (FIGS. 3A and 3B). As shown in FIGS. 3B and 3D, microwave-assisted NTF and NTE resulted in similar DNA encoding of FA and AF peptides in the two-cycle assay, albeit with much faster cycle times using microwave heating.

### Example 3: Assessment of Oligonucleotide Stability from Microwave-Assisted Reactions

As described in Example 2, peptide sequencing using a ProteoCode NGPS assay involves applying microwave energy to N-terminal amino acid functionalization and N-terminal amino acid removal (e.g., elimination) reactions on peptides labeled with a DNA recording tag. To test the effect of microwave on oligonucleotide stability, the conditions for microwave-assisted treatments were tested on oligonucleotides in solution as follows.

To a microwave vessel equipped with a magnetic stir bar, a solution of oligonucleotide (a 49-nucleotide single stranded oligonucleotide) in water (2 μL, 1 mM) was added. To this solution was added 200 μL 0.5 M sodium hydroxide solution (NaOH; pH 13.7) or 0.1 M lithium hydroxide solution (LiOH; pH 12.5) or 0.1M trisodium phosphate solution (Na₃PO₄; pH 12.1) or 0.1M potassium carbonate solution (K₂CO₃; pH 11.3) or 0.1M sodium carbonate/sodium bicarbonate buffer (CBc; pH 10.5). The vessel was sealed, placed in a microwave synthesizer (Discover SP, CEM Corporation, USA), and set to react at 60 W for 15 minutes. For comparison, functionalization was performed essentially as described except in the absence of microwave energy with application of conventional thermal heating at 80° C. for 60 minutes.

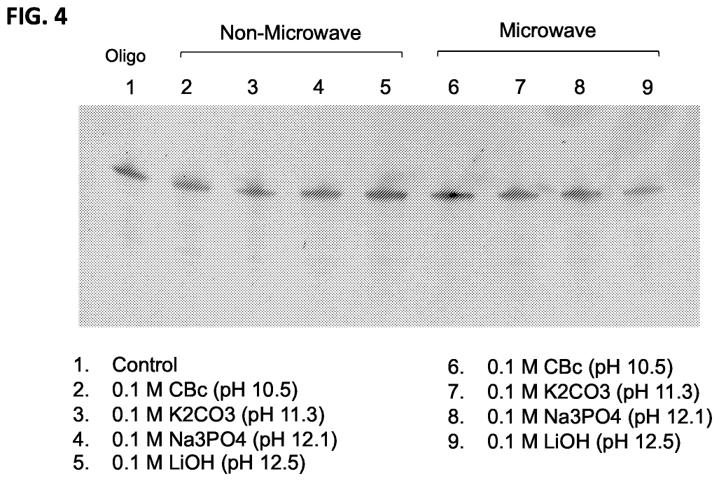

For analysis, the reaction solution (1 μL) was added to a gel loading solution (4 μL water, 5 μL loading dye) and analyzed by gel electrophoresis (200v, 80 minutes). As shown in FIG. 4, microwave treatment and heat treatment with various reagents did not show observable differences.

### Example 4: Assessment of Functionalization and Elimination of Various NTAA in Microwave Treated Peptides Compared to Conventional Heat-Treated Peptides

This example describes the assessment of applying microwave treatment in aiding the selective removal of the N-terminal amino acid (NTAA) in comparison to conventional heating techniques (i.e., thermomixer/heating block), several pools of peptides with varying P1- and P2-position amino acids composed of the same backbone (P1-P2-AALAWK, SEQ ID NO: 18) were evaluated. The P1 residues covered all classes (i.e., hydrophobic, hydrophilic, charged) of the amino acids; specifically residues: E, F, G, H, L, M, N, P, R, S, W, and Y. The P2 residues covered were: E, F, G, H, L, M, N, P, R, S, and W. For ease of analysis and chromatographic separation, the peptides containing the same P1 amino acid were pooled and treated with the reagents for NTAA functionalization and removal in both the microwave and conventional heating samples.

Microwave Treatment: To a microwave vessel equipped with a magnetic stir bar, no less than 0.2 mL of 0.2M N-ethylmorpholinium acetate (NEMA; pH 8.0) was added. To this, 0.01 mL of a solution of peptides with varying P1 and P2 amino acids (dissolved in either dimethylsulfoxide, N,N′-dimethylformamide, N,N′-dimethylacetamide, N-methyl-2-pyrollidone, or acetonitrile; in a 1 mM concentration) was added to the vessel. Subsequently, a guanidinylating reagent (pyrazole carboxamidine (PCA) derivative) for functionalization of the NTAA was dissolved in DMSO (to a concentration of 150 mM) and 0.02 mL was added to the reaction vessel. The vessel was sealed, placed in a microwave synthesizer and set to react at 30 W, 40 W, 50 W, or 60 W (60° C.) for up to 15 minutes. The reaction was quenched by the addition of an aliquot of a 1.0M glycine or ethanolamine solution paired with microwave irradiation 30 W (60° C.) for up to 5 minutes. To then remove the N-terminal amino acid, a volume of 0.4M sodium carbonate/sodium bicarbonate buffer (CBc; pH 10.5) was added to the solution to bring the final concentration to 0.1M CBc (pH 10.5). The vessel was then placed back into the microwave cavity and reacted 60 W (90° C.) for up to 15 minutes. Once cooled, the solution was acidified to pH 5.0 using 5.0M acetic acid (AcOH). Sample preparation for analysis was achieved by removal of salts by use of reversed-phase C18, solid-phase extraction (SPE). The desalted peptide reaction was then eluted using 80% acetonitrile (ACN).

Conventional Heating: To a 1.5 mL Eppendorf tube, no more than 0.5 mL of 0.2M N-ethylmorpholinium acetate (NEMA; pH 8.0) was added. To this, 0.01 mL of a solution of peptides with varying P1 and P2 amino acids (dissolved in either dimethylsulfoxide, N,N′-dimethylformamide, N,N′-dimethylacetamide, N-methyl-2-pyrollidone, or acetonitrile; in a 1 mM concentration) was added to the tube. Subsequently, a guanidinylating reagent (PCA derivative) for functionalization of the NTAA was dissolved in DMSO (to a concentration of 150 mM) and 0.02 mL was added to the reaction tube. The tube was capped, placed in a ThermoMixer and set to react at 40° C. for up to 60 minutes. The reaction was quenched by the addition of an aliquot of a 1.0M glycine or ethanolamine solution and heated in the thermomixer at 40° C. for up to 60 minutes. To then remove the N-terminal amino acid, a volume of 0.4M sodium carbonate/sodium bicarbonate buffer (CBc; pH 10.5) was added to the solution to bring the final concentration to 0.1M CBc (pH 10.5). The tube was then placed back into the thermomixer and reacted at 70° C. for 60 minutes. Once cooled, the solution was acidified to pH 5.0 using 5.0M acetic acid (AcOH). Sample preparation for analysis was achieved by removal of salts by use of reversed-phase C18, solid-phase extraction (SPE). The desalted peptide reaction was then eluted using 80% acetonitrile (ACN).

For analysis, a portion of the eluted material was injected into an LCMS (grad. 2-60% B/30 min; A: water and 0.1% formic acid, B: acetonitrile and 0.1% formic acid; column Agilent AdvanceBio Peptide Plus column; 2.1×150 mm, 2.7 μm) and monitored by UV (wavelength 216 nm). Complete functionalization (100%) of all tested peptides in both microwave and conventional heat treatment was observed and data showing elimination of NTAAs from peptides with varying P1- and P2-position amino acids is shown in TABLE 2A and TABLE 2B. In both tables, the amino acid in the P1 position is indicated in the first column and the amino acid in the P2 position is set forth across the first row. In summary, application of microwave energy resulted in similar or improved elimination of the NTAA (with the exception of proline in the P1 position) and reduced bias in removal of different amino acids.

TABLE 2A
Conventional NTE

| | P2 | | | | | | | | | | |
|---|---|---|---|---|---|---|---|---|---|---|---|
| P1 | H | R | G | S | E | P | M | L | F | W | N* |
| H | 100 | 100 | 78 | 100 | 100 | 100 | 100 | 66 | 69 | 77 | 100 |
| R | 100 | 100 | 100 | 100 | 100 | 100 | 100 | 98 | 98 | 95 | 100 |
| G | 29 | 43 | 65 | 91 | 24 | 26 | 16 | 23 | 15 | 12 | 100 |
| S | 100 | 100 | 100 | 100 | 80 | 100 | 90 | 80 | 80 | 85 | 100 |
| E | 39 | 56 | 75 | 72 | 39 | 84 | 27 | 49 | 26 | 21 | 100 |
| P | 0 | 0 | 0 | 0 | 0 | 0 | 0 | 0 | 0 | 0 | 100 |
| M | 44 | 79 | 100 | 91 | 44 | 94 | 46 | 46 | 35 | 48 | 100 |
| L | 47 | 75 | 100 | 82 | 31 | 72 | 39 | 57 | 29 | 36 | 100 |
| Y | 46 | 58 | 60 | 66 | 30 | 70 | 29 | 34 | 30 | 30 | 100 |
| F | 91 | 92 | 100 | 87 | 29 | 88 | 59 | 49 | 49 | 51 | 100 |
| W | 22 | 49 | 66 | 50 | 23 | 73 | 30 | 31 | 34 | 27 | 100 |

N*: Performed only with varied P1 and constant P2 as N (P1-NAALAWK)

TABLE 2B
Microwave NTE

| | P2 | | | | | | | | | | |
|---|---|---|---|---|---|---|---|---|---|---|---|
| P1 | H | R | G | S | E | P | M | L | F | W | N* |
| H | 100 | 100 | 95 | 95 | 95 | 95 | 95 | 95 | 95 | 95 | 100 |
| R | 100 | 100 | 95 | 95 | 95 | 95 | 95 | 95 | 95 | 95 | 100 |
| G | 100 | 100 | 100 | 100 | 100 | 100 | 85 | 80 | 80 | 82 | 100 |
| S | 100 | 100 | 90 | 90 | 90 | 90 | 90 | 90 | 90 | 90 | 100 |
| E | 82 | 89 | 83 | 100 | 71 | 95 | 80 | 72 | 70 | 76 | 100 |
| P | 0 | 0 | 0 | 0 | 0 | 0 | 0 | 0 | 0 | 0 | 100 |
| M | 100 | 100 | 95 | 95 | 95 | 95 | 95 | 95 | 95 | 95 | 100 |
| L | 100 | 100 | 100 | 100 | 95 | 100 | 74 | 70 | 81 | 84 | 100 |
| Y | 100 | 100 | 95 | 95 | 95 | 95 | 90 | 85 | 82 | 84 | 100 |
| F | 100 | 100 | 100 | 100 | 90 | 100 | 100 | 100 | 100 | 100 | 100 |
| W | 100 | 100 | 95 | 95 | 95 | 95 | 60 | 76 | 75 | 80 | 100 |

N*: Performed only with varied P1 and constant P2 as N (P1-NAALAWK)

The present disclosure is not intended to be limited in scope to the particular disclosed embodiments, which are provided, for example, to illustrate various aspects of the invention. Various modifications to the compositions and methods described will become apparent from the description and teachings herein. Such variations may be practiced without departing from the true scope and spirit of the disclosure and are intended to fall within the scope of the present disclosure. These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

SEQUENCE TABLE

| SEQ | | |
|---|---|---|
| ID NO | Sequence (5′-3′) | Description |
| 1 | AFAGVAMPGAEDDVVGSGSK | AF-PA |
| | | Peptide |
| 2 | AAGVAMPGAEDDVVGSGSK | AA-PA |
| | | Peptide |
| 3 | FAGVAMPGAEDDVVGSGSK | FA-PA |
| | | Peptide |
| 4 | /5AmMC6/TTT/i5OctdU/TTTTTTUCGTAGTCCGCGACAC | Barcode |
| | TAGNNNNNNNNNNTTAAGTCGACTGAGTG | |
| 5 | /5AmMC6/TTT/i5OctdU/TTTTTTUCGTAGTCCGCGACAC | Barcode |
| | TAGNNNNNNNNNNGTTAATGGACTGAGTG | |
| 6 | /5AmMC6/TTT/i5OctdU/TTTTTTUCGTAGTCCGCGACAC | Barcode |
| | TAGNNNNNNNNNNCAGTACCGACTGAGTG | |
| 7 | /5AmMC6/TTT/i5OctdU/TTTTTTUCGTAGTCCGCGACAC | Barcode |
| | TAGNNNNNNNNNNGTTGGTTAACTGAGTG | |
| 8 | /5AmMC6//iSP18/CACTCAGTTTTTCCTGTCACTCAGT/ | Coding Tag |
| | 3SpC3/ | |
| 9 | /5AmMC6//iSP18/CACTCAGTCAGACTATTCACTCAGT/ | Coding Tag |
| | 3SpC3/ | |
| 10 | CPXQXWXDXT | Coding Tag |
| | X = any amino acid | |
| 11 | AATGATACGGCGACCACCGA | P5 primer |
| 12 | CAAGCAGAAGACGGCATACGAGAT | P7 primer |
| 13 | CPVQLWVDST | Coding Tag |
| 14 | AALAY | Test Peptide |
| 15 | YFAGVAMG | Test Peptide |
| 16 | FWAALAWK | Test Peptide |
| 17 | FFAALAWK | Test Peptide |
| 18 | AALAWK | Test Peptide |
| | | Backbone |

1. (canceled)

2. A method for treating a polypeptide, the method comprises the steps of:
a) contacting a polypeptide with a functionalizing reagent to modify an N-terminal amino acid (NTAA) of the polypeptide, thereby forming a modified NTAA;
b) optionally contacting the polypeptide with a removing reagent to remove the modified NTAA from the polypeptide; and
c) applying microwave energy to the polypeptide;
wherein either step a) or step b) is performed in the presence of the microwave energy.

3. The method of claim 2, further comprising: d) contacting the modified NTAA with a binding agent that binds to the modified NTAA.

4. The method of claim 3, wherein the step d) is conducted after step a) and before step b).

5. (canceled)

6. The method of claim 2, wherein the polypeptide is contacted with the functionalizing reagent and then with the removing reagent.

7. The method of claim 6, wherein both step a) and step b) are performed in the presence of the microwave energy.

8. The method of claim 2, wherein the polypeptide is directly or indirectly joined to a support before contacting the polypeptide with the functionalizing reagent.

9-13. (canceled)

14. The method of claim 2, wherein the functionalizing reagent adds a chemical moiety to the NTAA of the polypeptide.

15. The method of claim 2, wherein the functionalizing reagent selectively or specifically modifies the N-terminal amino acid (NTAA) of the polypeptide.

16-18. (canceled)

19. The method of claim 2, wherein the functionalizing reagent comprises a compound selected from the group consisting of:
(i) a compound of Formula (I): 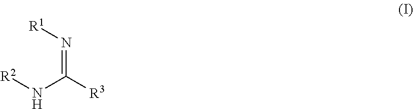
or a salt or conjugate thereof,
wherein
R¹ and R² are each independently H, C₁₋₆alkyl, cycloalkyl, —C(O)R^{a}, —C(O)OR^{b}, or —S(O)₂R^{c};
R^{a}, R^{b}, and R^{c} are each independently H, C₁₋₆alkyl, C₁₋₆haloalkyl, arylalkyl, aryl, or heteroaryl, wherein the C₁₋₆alkyl, C₁₋₆haloalkyl, arylalkyl, aryl, and heteroaryl are each unsubstituted or substituted;
R³ is heteroaryl, —NR^{d}C(O)OR^{e}, or —SR^{f}, wherein the heteroaryl is unsubstituted or substituted;
R^{d}, R^{e}, and R^{f} are each independently H or C₁₋₆alkyl; and
optionally wherein when R³ is 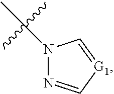
wherein G₁ is N, CH, or CX where X is halo, C₁₋₃ alkyl, C₁₋₃ haloalkyl, or nitro, R¹ and R² are not both H;
(ii) a compound of Formula (II): 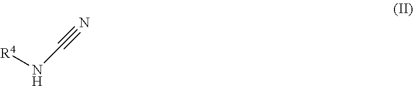
or a salt or conjugate thereof,
wherein
R⁴ is H, C₁₋₆alkyl, cycloalkyl, —C(O)R^{g}, or —C(O)OR^{g}; and
R^{g} is H, C₁₋₆alkyl, C₂₋₆alkenyl, C₁₋₆haloalkyl, or arylalkyl, wherein the C₁₋₆alkyl, C₂₋₆alkenyl, C₁₋₆ haloalkyl, and arylalkyl are each unsubstituted or substituted;
(iii) a compound of Formula (III):
R⁵—N═C═S (III)
or a salt or conjugate thereof,
wherein
R⁵ is C₁₋₆alkyl, C₂₋₆ alkenyl, cycloalkyl, heterocycloalkyl, aryl or heteroaryl;
wherein the C₁₋₆alkyl, C₂₋₆ alkenyl, cycloalkyl, heterocycloalkyl, aryl or heteroaryl are each unsubstituted or substituted with one or more groups selected from the group consisting of halo, —NR^{h}Rⁱ, —S(O)₂R^{j}, or heterocyclyl;
R^{h}, Rⁱ, and R^{j} are each independently H, C₁₋₆alkyl, C₁₋₆haloalkyl, arylalkyl, aryl, or heteroaryl, wherein the C₁₋₆alkyl, C₁₋₆haloalkyl, arylalkyl, aryl, and heteroaryl are each unsubstituted or substituted;
(iv) a compound of Formula (IV): 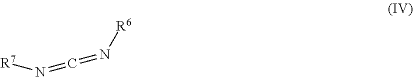
or a salt or conjugate thereof,
wherein
R⁶ and R⁷ are each independently H, C₁₋₆alkyl, —CO₂C₁₋₄ alkyl, —OR^{k}, aryl, or cycloalkyl, wherein the C₁₋₆alkyl, —CO₂C₁₋₄ alkyl, —OR^{k}, aryl, and cycloalkyl are each unsubstituted or substituted; and
R^{k} is H, C₁₋₆alkyl, or heterocyclyl, wherein the C₁₋₆alkyl and heterocyclyl are each unsubstituted or substituted;
(v) a compound of Formula (V): 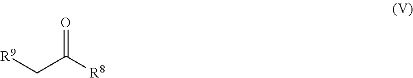
or a salt or conjugate thereof,
wherein
R⁸ is halo or —OR^{m};
R^{m} is H, C₁₋₆alkyl, or heterocyclyl; and
R⁹ is hydrogen, halo, or C₁₋₆haloalkyl;
(vi) a metal complex of Formula (VI):
MLₙ (VI)
or a salt or conjugate thereof,
wherein
M is a metal selected from the group consisting of Co, Cu, Pd, Pt, Zn, and Ni;
L is a ligand selected from the group consisting of —OH, —OH₂, 2,2′-bipyridine (bpy), 1,5 dithiacyclooctane (dtco), 1,2-bis(diphenylphosphino)ethane (dppe), ethylenediamine (en), and triethylenetetramine (trien); and
n is an integer from 1-8, inclusive;
wherein each L can be the same or different; and
(vii) a compound of Formula (VII): 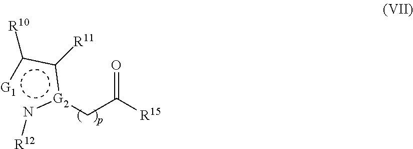
or a salt or conjugate thereof,
wherein
G¹ is N, NR¹³, or CR¹³R¹⁴;
G² is N or CH;
p is 0 or 1;
R¹⁰, R¹¹, R¹², R¹³, and R¹⁴ are each independently selected from the group consisting of H, C₁₋₆ alkyl, C₁₋₆ haloalkyl, C₁₋₆ alkylamine, and C₁₋₆alkylhydroxylamine, wherein the C₁₋₆ alkyl, C₁₋₆haloalkyl, C₁₋₆ alkylamine, and C₁₋₆ alkylhydroxylamine are each unsubstituted or substituted, and R¹⁰ and R¹¹ can optionally come together to form a ring; and
R¹⁵ is H or OH.

20. The method of claim 2, which wherein removing the modified NTAA comprises contacting the polypeptide with a removing reagent, wherein the removing reagent comprises an acylpeptide hydrolase (APH), a dipeptidyl peptidase (DPP) and/or a dipeptidyl aminopeptidase enzyme.

21. The method of claim 2, wherein modification of the NTAA of the polypeptide is accelerated due to the application of the microwave energy to the polypeptide by at least 5% as compared to modification of the amino acid of the polypeptide without application of the microwave energy to the polypeptide.

22-59. (canceled)

60. The method of claim 2, further comprising contacting the polypeptide with a peptide coupling reagent at step a).

61-73. (canceled)

74. A method for analyzing a polypeptide, which comprises the steps:
(a) providing a polypeptide optionally associated directly or indirectly with a recording tag;
(b) modifying an N-terminal amino acid (NTAA) of the polypeptide with a functionalizing reagent to yield a modified NTAA,
(c) contacting the polypeptide with a first binding agent comprising a first binding portion capable of binding to the modified NTAA and
(c1) a first coding tag with identifying information regarding the first binding agent, or
(c2) a first detectable label;
(d) (d1) transferring the information of the first coding tag to the recording tag to generate a first extended recording tag and analyzing the extended recording tag, or
(d2) detecting the first detectable label; and
(e) optionally contacting the polypeptide with a removing reagent to remove the modified NTAA to expose a new NTAA;
wherein
any one or more of steps (b), (c), (d1), (d2) and/or (e) are performed in the presence of microwave energy.

75. (canceled)

76. The method of claim 74, wherein the polypeptide is contacted with the removing reagent at step (e).

77. The method of claim 76, further comprising repeating steps (b) to (e) at least one more time to determine a sequence of at least a portion of the polypeptide.

78. (canceled)

79. The method of claim 76, wherein the steps (b) and (e) are performed in the presence of microwave energy.

80-127. (canceled)

128. The method of claim 74, wherein the polypeptide is directly or indirectly joined to a support at step (a).

129-152. (canceled)

153. A kit or system for treating a polypeptide, which comprises:
a) a functionalizing reagent to modify an amino acid of a polypeptide, a binding agent capable of binding to said polypeptide, and/or a removing reagent to remove an amino acid from said polypeptide; and
b) a microwave energy source, e.g., a microwave energy source configured for applying a microwave energy to said polypeptide;
wherein the functionalizing reagent modifies an N-terminal amino acid (NTAA), the binding agent binds to an N-terminal amino acid (NTAA), and/or the removing reagent removes an N-terminal amino acid (NTAA).

154-155. (canceled)

156. A method for treating a polypeptide, the method comprises the steps of:
a) contacting a polypeptide with a functionalizing reagent to modify an amino acid of the polypeptide, thereby forming a modified amino acid;
b) contacting the modified amino acid with a binding agent that binds to the modified amino acid; and
c) applying microwave energy to the polypeptide, wherein step a) is performed in the presence of the microwave energy.

157. The method of claim 156, further comprising: d) removing the modified amino acid.

158. The method of claim 157, wherein both step a) and step d) are performed in the presence of the microwave energy.


SEQUENCE LISTING (18 sequences)

SEQ ID NO: 1 - protein, 20 aa, Artificial Sequence
  AF-PA Peptide
AFAGVAMPGAEDDVVGSGSK

SEQ ID NO: 2 - protein, 19 aa, Artificial Sequence
  AA-PA Peptide
AAGVAMPGAEDDVVGSGSK

SEQ ID NO: 3 - protein, 19 aa, Artificial Sequence
  FA-PA Peptide
FAGVAMPGAEDDVVGSGSK

SEQ ID NO: 4 - DNA, 54 bp, Artificial Sequence
  Barcode
  misc_feature (1)..(1): 5′ amino modification
  variation (3)..(4): 5′-octadiynyl dU
  misc_feature (29)..(38): n is a, c, g, t or u
tttttttttucgtagtccgcgacactagnnnnnnnnnnttaagtcgactgagtg

SEQ ID NO: 5 - DNA, 54 bp, Artificial Sequence
  Barcode
  misc_feature (1)..(1): 5′ amino modification
  variation (3)..(4): 5′ -octadiynyl dU
  misc_feature (29)..(38): n is a, c, g, t or u
tttttttttucgtagtccgcgacactagnnnnnnnnnngttaatggactgagtg

SEQ ID NO: 6 - DNA, 54 bp, Artificial Sequence
  Barcode
  misc_feature (1)..(1): 5′ amino modification
  variation (3)..(4): 5′-Octadiynyl dU
  misc_feature (29)..(38): n is a, c,, g, t, or u
tttttttttucgtagtccgcgacactagnnnnnnnnnncagtaccgactgagtg

SEQ ID NO: 7 - DNA, 54 bp, Artificial Sequence
  Barcode
  misc_feature (1)..(1): 5′ amino modification
  variation (3)..(4): 5′-Octadiynyl dU
  misc_feature (29)..(38): n is a, c, g, t or u
tttttttttucgtagtccgcgacactagnnnnnnnnnngttggttaactgagtg

SEQ ID NO: 8 - DNA, 25 bp, Artificial Sequence
  Coding Tag
  misc_feature (1)..(1): 5′ amino modification and an 18-atom hexa-ethyleneglycol spacer
  misc_feature (25)..(25): 3′′-C3 (three carbon) spacer
cactcagtttttcctgtcactcagt

SEQ ID NO: 9 - DNA, 25 bp, Artificial Sequence
  Coding Tag
  misc_feature (1)..(1): 5′ amino modification and an 18-atom hexa-ethyleneglycol spacer
  misc_feature (25)..(25): 3′ C3  (three carbon) spacer
cactcagtcagactattcactcagt

SEQ ID NO: 10 - protein, 10 aa, Artificial Sequence
  Coding Tag
  MISC_FEATURE (3)..(3): Xaa = any amino acid
  MISC_FEATURE (5)..(5): Xaa = any amino acid
  MISC_FEATURE (7)..(7): Xaa = any amino acid
  MISC_FEATURE (9)..(9): Xaa = any amino acid
CPXQXWXDXT

SEQ ID NO: 11 - DNA, 20 bp, Artificial Sequence
  P5 primer
aatgatacggcgaccaccga

SEQ ID NO: 12 - DNA, 24 bp, Artificial Sequence
  P7 primer
caagcagaagacggcatacgagat

SEQ ID NO: 13 - protein, 10 aa, Artificial Sequence
  Coding Tag
CPVQLWVDST

SEQ ID NO: 14 - protein, 5 aa, Artificial Sequence
  Test Peptide
AALAY

SEQ ID NO: 15 - protein, 8 aa, Artificial Sequence
  Test Peptide
YFAGVAMG

SEQ ID NO: 16 - protein, 8 aa, Artificial Sequence
  Test Peptide
FWAALAWK

SEQ ID NO: 17 - protein, 8 aa, Artificial Sequence
  Test Peptide
FFAALAWK

SEQ ID NO: 18 - protein, 6 aa, Artificial Sequence
  Test Peptide Backbone
AALAWK